(19) United States
(12) Patent Application Publication
(10) Pub. No.: US 2024/0057478 A1
(43) Pub. Date: Feb. 15, 2024

(54) ORGANIC ELECTROLUMINESCENCE ELEMENT AND ELECTRONIC DEVICE

(71) Applicant: IDEMITSU KOSAN CO.,LTD., Chiyoda-ku (JP)

(72) Inventors: Kazuki TERADA, Chiyoda-ku (JP); Hisato MATSUMOTO, Chiyoda-ku (JP); Keiichi YASUKAWA, Chiyoda-ku (JP); Yuichiro KAWAMURA, Chiyoda-ku (JP); Toshinari OGIWARA, Chiyoda-ku (JP); Yuichi NISHIMAE, Basel (CH)

(73) Assignee: IDEMITSU KOSAN CO.,LTD., Chiyoda-ku (JP)

(21) Appl. No.: 18/258,060

(22) PCT Filed: Dec. 16, 2021
(86) PCT No.: PCT/JP2021/046598

§ 371 Date: Jun. 16, 2023

(30) Foreign Application Priority Data
Dec. 17, 2020 (JP) 2020-209668
Jan. 18, 2021 (JP) 2021-005799

(51) Int. Cl. H10K 85/60; C07D 495/04; C09K 11/06; C07F 5/02

(52) U.S. Cl. CPC H10K 85/657; C07D 495/04; C09K 11/06; C07F 5/027; H10K 85/658; H10K 50/11

(57) ABSTRACT

An organic electroluminescence device includes an anode, a cathode, and an emitting layer, in which the emitting layer contains a first compound represented by a formula (1) and a delayed fluorescent second compound represented by a formula (2), and singlet energy S1(M1) of the first compound and singlet energy S1(M2) of the second compound satisfy a relationship of Numerical Formula 1, S1(M2)>S1(M1) (Numerical Formula 1). In the formula, rings A, B, D, E, and F are each independently a cyclic structure selected from the group consisting of a substituted or unsubstituted aryl ring having 6 to 30 ring carbon atoms and a 10 substituted or unsubstituted heterocyclic ring having 5 to 30 ring atoms; one or both of the rings B and D are present; and one or both of the rings E and F are present

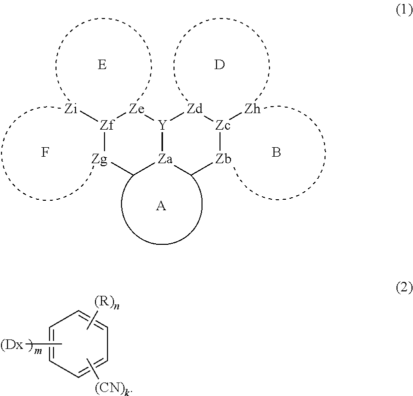

### TECHNICAL FIELD

The present invention relates to an organic electroluminescence device and an electronic device.

### BACKGROUND ART

When a voltage is applied to an organic electroluminescence device (hereinafter, occasionally referred to as “organic EL device”), holes are injected from an anode and electrons are injected from a cathode into an emitting layer. The injected electrons and holes are recombined in the emitting layer to form excitons. Specifically, according to the electron spin statistics theory, singlet excitons and triplet excitons are generated at a ratio of 25%:75%.

A fluorescent organic EL device using light emission from singlet excitons has been applied to a full-color display such as a mobile phone and a television set, but an internal quantum efficiency is said to be at a limit of 25%. Studies have thus been made to improve performance of the organic EL device.

For instance, the organic EL device is expected to emit light more efficiently using triplet excitons in addition to singlet excitons. In view of the above, a highly-efficient fluorescent organic EL device using thermally activated delayed fluorescence (hereinafter simply referred to as “delayed fluorescence” in some cases) has been proposed and studied.

A thermally activated delayed fluorescence (TADF) mechanism uses such a phenomenon in which inverse intersystem crossing from triplet excitons to singlet excitons thermally occurs when a material having a small energy difference (ΔST) between singlet energy level and triplet energy level is used. Thermally activated delayed fluorescence is explained in “Yuki Hando-tai no Debaisu Bussei (Device Physics of Organic Semiconductors)” (edited by ADACHI Chihaya, published by Kodansha, issued on Apr. 1, 2012, on pages 261-268).

As a compound exhibiting TADF properties (hereinafter also referred to as a TADF compound), for instance, a compound in which a donor moiety and an acceptor moiety are bonded in a molecule is known.

For instance, Patent Literature 1 describes an organic electroluminescence device using a TADF compound.

Moreover, for instance, Patent Literatures 2 and 3 describe an organic electroluminescence device using a fused ring compound containing a nitrogen atom and a boron atom.

### CITATION LIST

### Patent Literature(s)

Patent Literature 1 International Publication No. WO 2018/181188
Patent Literature 2 International Publication No. WO 2020/106032
Patent Literature 3 International Publication No. WO 2020/135953

### SUMMARY OF THE INVENTION

### Problem(s) to be Solved by the Invention

An object of the invention is to provide an organic electroluminescence device having an improved performance, particularly, having a prolonged lifetime, and an electronic device including the organic electroluminescence device.

### Means for Solving the Problem(s)

An aspect of the invention provides an organic electroluminescence device including an anode, a cathode, and an emitting layer provided between the anode and the cathode, in which the emitting layer contains a first compound represented by a formula (1) below and a second compound that exhibits delayed fluorescence and is represented by a formula (2) below, and a singlet energy S₁(M1) of the first compound and a singlet energy S₁(M2) of the second compound satisfy a relationship of a numerical formula (Numerical Formula 1) below.

S₁(M2)>S₁(M1) (Numerical Formula 1)

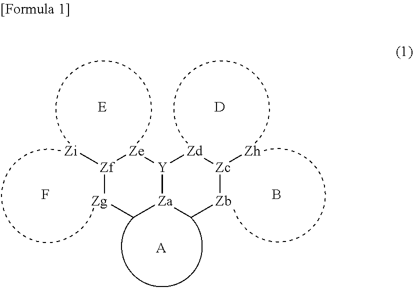

In the formula (1):
a ring A, a ring B, a ring D, a ring E, and a ring F are each independently a cyclic structure selected from the group consisting of a substituted or unsubstituted aryl ring having 6 to 30 ring carbon atoms, and a substituted or unsubstituted heterocyclic ring having 5 to 30 ring atoms;
one of the ring B and the ring D is present or both of the ring B and the ring D are present;
when both of the ring B and the ring D are present, the ring B and the ring D share a bond connecting Zc to Zh;
one of the ring E and the ring F is present or both of the ring E and the ring F are present;
when both of the ring E and the ring F are present, the ring E and the ring F share a bond connecting Zf to Zi;
Za is a nitrogen atom or a carbon atom;
Zb is a nitrogen atom or a carbon atom when the ring B is present, and Zb is an oxygen atom, a sulfur atom, NRb, C(Rb₁)(Rb₂), or S₁(Rb₃)(Rb₄) when the ring B is not present;
Zc is a nitrogen atom or a carbon atom;
Zd is a nitrogen atom or a carbon atom when the ring D is present, and Zd is an oxygen atom, a sulfur atom, or NRd when the ring D is not present;
Ze is a nitrogen atom or a carbon atom when the ring E is present, and Ze is an oxygen atom, a sulfur atom, or NRe when the ring E is not present;
Zf is a nitrogen atom or a carbon atom;
Zg is a nitrogen atom or a carbon atom when the ring F is present, and Zg is an oxygen atom, a sulfur atom, NRg, C(Rg₁)(Rg₂), or Si(Rg₃)(Rg₄) when the ring F is not present;
Zh is a nitrogen atom or a carbon atom;
Zi is a nitrogen atom or a carbon atom;
Y is a boron atom, a phosphorus atom, SiRh, P═O, or P═S;
Rb, Rb₁, Rb₂, Rb₃, Rb₄, Rd, Re, Rg, Rg₁, Rg₂, Rg₃, Rg₄, and Rh are each independently a hydrogen atom or a substituent;
Rb, Rb₁, Rb₂, Rb₃, Rb₄, Rd, Re, Rg, Rg₁, Rg₂, Rg₃, Rg₄, and Rh as a substituent are each independently a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted cycloalkyl group having 3 to 30 ring carbon atoms, a group represented by —Si(R₉₁₁)(R₉₁₂)(R₉₁₃), a group represented by —O—(R₉₁₄), a group represented by —S—(R₉₁₅), or a group represented by —N(R₉₁₆)(R₉₁₇); and
a bond between Y and Za, a bond between Y and Zd, and a bond between Y and Ze are each a single bond.

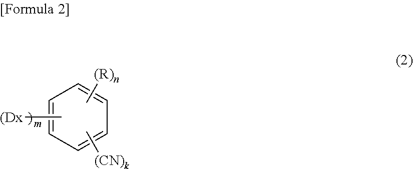

In the formula (2):
k is 1, 2, 3, 4, or 5;
Dx is a group represented by a formula (21), (22), or (23) below, at least one Dx being a group represented by the formula (22) or (23);
m is 1, 2, 3, or 4;
when m is 2, 3, or 4, a plurality of Dx are mutually the same or different;
n is 0, 1, 2, or 3;
when n is 2 or 3, a plurality of R are mutually the same or different;
a sum of m and n is 2 or more;
each R is independently a halogen atom or a substituent; and
each R as a substituent is independently a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted cycloalkyl group having 3 to 30 ring carbon atoms, a group represented by —Si(R₉₁₁)(R₉₁₂)(R₉₁₃), a group represented by —O—(R₉₁₄), a group represented by —S—(R₉₁₅), or a group represented by —N(R₉₁₆)(R₉₁₇).

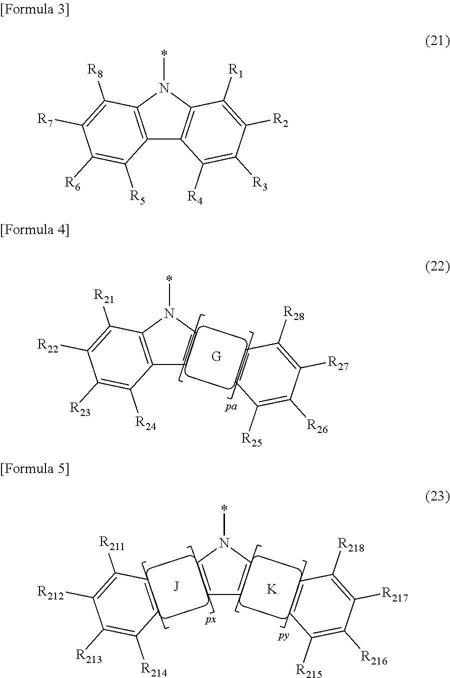

R₁ to R₈ in the formula (21) are each independently a hydrogen atom, a halogen atom, or a substituent.

R₂₁ to R₂₈ in the formula (22) are each independently a hydrogen atom, a halogen atom, or a substituent, or at least one combination of a combination of R₂₁ and R₂₂, a combination of R₂₂ and R₂₃, a combination of R₂₃ and R₂₄, a combination of R₂s and R₂₆, a combination of R₂₆ and R₂₇, or a combination of R₂₇ and R₂₅ are mutually bonded to form a ring.

R₂₁₁ to R₂₁₈ in the formula (23) are each independently a hydrogen atom, a halogen atom, or a substituent, or at least one combination of a combination of R₂₁₁ and R₂₁₂, a combination of R₂₁₂ and R₂₁₃, a combination of R₂₁₃ and R₂₁₄, a combination of R₂₁₅ and R₂₁₆, a combination of R₂₁₆ and R₂₁₇, or a combination of R₂₁₇ and R₂₁₈ are mutually bonded to form a ring.

R₁ to R₈ as a substituent, R₂₁ to R₂₈ as a substituent, and R₂₁₁ to R₂₁₈ as a substituent are each independently a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted cycloalkyl group having 3 to 30 ring carbon atoms, a group represented by —Si(R₉₁₁)(R₉₁₂)(R₉₁₃), a group represented by —O—(R₉₁₄), a group represented by —S—(R₉₁₅), or a group represented by —N(R₉₁₆)(R₉₁₇).

In the formulae (22) and (23):
a ring G, a ring J, and a ring K are each independently any cyclic structure selected from the group consisting of cyclic structures represented by formulae (24), (25), and (26) below;
the ring G, the ring J, and the ring K are fused with adjacent rings at any positions;
pa, px and py are each independently 1, 2, 3, or 4;
when pa is 2, 3, or 4, a plurality of rings G are mutually the same or different;
when px is 2, 3, or 4, a plurality of rings J are mutually the same or different;
when py is 2, 3, or 4, a plurality of rings K are mutually the same or different;
at least one Dx is a group represented by the formula (22) in which pa is 2, 3, or 4 and each ring G has any cyclic structure selected from the group consisting of cyclic structures represented by formulae (25) and (26) below, or at least one Dx is a group represented by the formula (23) in which at least one of px or py is 2, 3, or 4 and at least one ring J, at least one ring K, or both of at least one ring J and at least one ring K have any cyclic structure selected from the group consisting of cyclic structures represented by the formulae (25) and (26); and
each * in the formulae (21) to (23) represents a bonding position to a benzene ring in the formula (2).

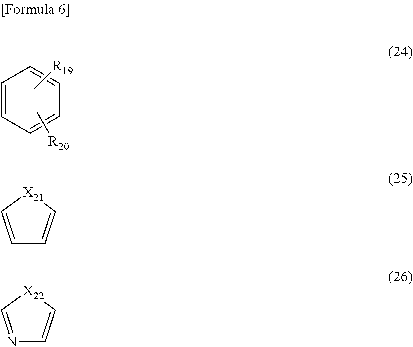

In the formula (24): R₁₉ and R₂₀ are each independently a hydrogen atom, a halogen atom, or a substituent, or a combination of R₁₉ and R₂₀ are mutually bonded to form a ring.

In the formulae (25) and (26):
X₂₁ and X₂₂ are each independently a sulfur atom, an oxygen atom, NR₂₂₀, CR₂₂₁R₂₂₂, or SiR₂₂₃R₂₂₄;
R₂₂₀ is a hydrogen atom, a halogen atom, or a substituent;
R₂₂₁ and R₂₂₂ are each independently a hydrogen atom, a halogen atom, or a substituent, or a combination of R₂₂₁ and R₂₂₂ are mutually bonded to form a ring;
R₂₂₃ and R₂₂₄ are each independently a hydrogen atom, a halogen atom, or a substituent, or a combination of R₂₂₃ and R₂₂₄ are mutually bonded to form a ring; and
R₁₉, R₂₀, R₂₂₀, R₂₂₁, R₂₂₂, R₂₂₃, and R₂₂₄ as a substituent each independently represent the same as R₁ to R₈ as a substituent.

In the first compound and the second compound, R₉₁₁ to R₉₁₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₁₁ are present, the plurality of R₁₁ are mutually the same or different;
when a plurality of R₉₁₂ are present, the plurality of R₉₁₂ are mutually the same or different;
when a plurality of R₉₁₃ are present, the plurality of R₉₁₃ are mutually the same or different;
when a plurality of R₉₁₄ are present, the plurality of R₉₁₄ are mutually the same or different;
when a plurality of R₉₁₅ are present, the plurality of R₉₁₅ are mutually the same or different;
when a plurality of R₉₁₆ are present, the plurality of R₉₁₆ are mutually the same or different; and
when a plurality of R₉₁₇ are present, the plurality of R₉₁₇ are mutually the same or different.

According to another aspect of the invention, there is provided an electronic device including the organic electroluminescence device according to the above aspect of the invention.

According to the above aspects of the invention, there can be provided an organic electroluminescence device having an improved performance, particularly having a prolonged lifetime, and an electronic device including the organic electroluminescence device.

### BRIEF EXPLANATION OF DRAWINGS

FIG. 1 schematically illustrates an exemplary arrangement of an organic electroluminescence device according to a first exemplary embodiment of the invention.

FIG. 2 schematically depicts an apparatus for measuring transient PL.

FIG. 3 illustrates an example of decay curves of the transient PL.

FIG. 4 schematically illustrates a relationship in energy level between a first compound and a second compound in an emitting layer of an exemplary organic electroluminescence device according to the first exemplary embodiment of the invention.

FIG. 5 schematically illustrates a relationship in energy level and energy transfer between the first compound, the second compound and a third compound in an emitting layer of an exemplary organic electroluminescence device according to a second exemplary embodiment of the invention.

### DESCRIPTION OF EMBODIMENT(S)

### Definitions

Herein, a hydrogen atom includes isotope having different specifically, protium, deuterium and tritium.

In chemical formulae herein, it is assumed that a hydrogen atom (i.e. protium, deuterium and tritium) is bonded to each of bondable positions that are not annexed with signs “R” or the like or “D” representing a deuterium.

Herein, the ring carbon atoms refer to the number of carbon atoms among atoms forming a ring of a compound (e.g., a monocyclic compound, fused-ring compound, crosslinking compound, carbon ring compound, and heterocyclic compound) in which the atoms are bonded to each other to form the ring. When the ring is substituted by a substituent(s), carbon atom(s) included in the substituent(s) is not counted in the ring carbon atoms. Unless specifically described, the same applies to the “ring carbon atoms” described later. For instance, a benzene ring has 6 ring carbon atoms, a naphthalene ring has 10 ring carbon atoms, a pyridine ring has 5 ring carbon atoms, and a furan ring 4 ring carbon atoms. For instance, a 9,9-diphenylfluorenyl group has 13 ring carbon atoms and a 9,9′-spirobifluorenyl group has 25 ring carbon atoms.

When a benzene ring is substituted by a substituent (e.g., an alkyl group), the number of carbon atoms of the alkyl group is not counted in the number of the ring carbon atoms. Accordingly, the benzene ring substituted by an alkyl group has 6 ring carbon atoms. When a naphthalene ring is substituted by a substituent in a form of, for instance, an alkyl group, the number of carbon atoms of the alkyl group is not counted in the number of the ring carbon atoms of the naphthalene ring. Accordingly, the naphthalene ring substituted by an alkyl group has 10 ring carbon atoms.

Herein, the ring atoms refer to the number of atoms forming a ring of a compound (e.g., a monocyclic compound, fused-ring compound, cross-linking compound, carbon ring compound, and heterocyclic compound) in which the atoms are bonded to each other to form the ring (e.g., monocyclic ring, fused ring, and ring assembly). Atom(s) not forming the ring (e.g., hydrogen atom(s) for saturating the valence of the atom which forms the ring) and atom(s) in a substituent by which the ring is substituted are not counted as the ring atoms. Unless otherwise specified, the same applies to the “ring atoms” described later. For instance, a pyridine ring has 6 ring atoms, a quinazoline ring has 10 ring atoms, and a furan ring has 5 ring atoms. For instance, the number of hydrogen atom(s) bonded to a pyridine ring or the number of atoms forming a substituent is not counted as the pyridine ring atoms. Accordingly, a pyridine ring bonded to a hydrogen atom(s) or a substituent(s) has 6 ring atoms. For instance, the hydrogen atom(s) bonded to carbon atom(s) of a quinazoline ring or the atoms forming a substituent are not counted as the quinazoline ring atoms. Accordingly, a quinazoline ring bonded to hydrogen atom(s) or a substituent(s) has 10 ring atoms.

Herein, “XX to YY carbon atoms” in the description of “substituted or unsubstituted ZZ group having XX to YY carbon atoms” represent carbon atoms of an unsubstituted ZZ group and do not include carbon atoms of a substituent(s) of the substituted ZZ group. Herein, “YY” is larger than “XX,” “XX” representing an integer of 1 or more and “YY” representing an integer of 2 or more.

Herein, “XX to YY atoms” in the description of “substituted or unsubstituted ZZ group having XX to YY atoms” represent atoms of an unsubstituted ZZ group and does not include atoms of a substituent(s) of the substituted ZZ group. Herein, “YY” is larger than “XX,” “XX” representing an integer of 1 or more and “YY” representing an integer of 2 or more.

Herein, an unsubstituted ZZ group refers to an “unsubstituted ZZ group” in a “substituted or unsubstituted ZZ group,” and a substituted ZZ group refers to a “substituted ZZ group” in a “substituted or unsubstituted ZZ group.”

Herein, the term “unsubstituted” used in a “substituted or unsubstituted ZZ group” means that a hydrogen atom(s) in the ZZ group is not substituted with a substituent(s). The hydrogen atom(s) in the “unsubstituted ZZ group” is protium, deuterium, or tritium.

Herein, the term “substituted” used in a “substituted or unsubstituted ZZ group” means that at least one hydrogen atom in the ZZ group is substituted with a substituent. Similarly, the term “substituted” used in a “BB group substituted by AA group” means that at least one hydrogen atom in the BB group is substituted with the AA group.

### Substituent Mentioned Herein

Substituent mentioned herein will be described below.

An “unsubstituted aryl group” mentioned herein has, unless otherwise specified herein, 6 to 50, preferably 6 to 30, more preferably 6 to 18 ring carbon atoms.

An “unsubstituted heterocyclic group” mentioned herein has, unless otherwise specified herein, 5 to 50, preferably 5 to 30, more preferably 5 to 18 ring atoms.

An “unsubstituted alkyl group” mentioned herein has, unless otherwise specified herein, 1 to 50, preferably 1 to 20, more preferably 1 to 6 carbon atoms.

An “unsubstituted alkenyl group” mentioned herein has, unless otherwise specified herein, 2 to 50, preferably 2 to 20, more preferably 2 to 6 carbon atoms.

An “unsubstituted alkynyl group” mentioned herein has, unless otherwise specified herein, 2 to 50, preferably 2 to 20, more preferably 2 to 6 carbon atoms.

An “unsubstituted cycloalkyl group” mentioned herein has, unless otherwise specified herein, 3 to 50, preferably 3 to 20, more preferably 3 to 6 ring carbon atoms.

An “unsubstituted arylene group” mentioned herein has, unless otherwise specified herein, 6 to 50, preferably 6 to 30, more preferably 6 to 18 ring carbon atoms.

An “unsubstituted divalent heterocyclic group” mentioned herein has, unless otherwise specified herein, 5 to 50, preferably 5 to 30, more preferably 5 to 18 ring atoms.

An “unsubstituted alkylene group” mentioned herein has, unless otherwise specified herein, 1 to 50, preferably 1 to 20, more preferably 1 to 6 carbon atoms.

### Substituted or Unsubstituted Aryl Group

Specific examples (specific example group G1) of the “substituted or unsubstituted aryl group” mentioned herein include unsubstituted aryl groups (specific example group G1A) below and substituted aryl groups (specific example group G1B). (Herein, an unsubstituted aryl group refers to an “unsubstituted aryl group” in a “substituted or unsubstituted aryl group”, and a substituted aryl group refers to a “substituted aryl group” in a “substituted or unsubstituted aryl group.”) A simply termed “aryl group” herein includes both of an “unsubstituted aryl group” and a “substituted aryl group”.

The “substituted aryl group” refers to a group derived by substituting at least one hydrogen atom in an “unsubstituted aryl group” with a substituent. Examples of the “substituted aryl group” include a group derived by substituting at least one hydrogen atom in the “unsubstituted aryl group” in the specific example group G1A below with a substituent, and examples of the substituted aryl group in the specific example group G1B below. It should be noted that the examples of the “unsubstituted aryl group” and the “substituted aryl group” mentioned herein are merely exemplary, and the “substituted aryl group” mentioned herein includes a group derived by further substituting a hydrogen atom bonded to a carbon atom of a skeleton of a “substituted aryl group” in the specific example group G1B below, and a group derived by further substituting a hydrogen atom of a substituent of the “substituted aryl group” in the specific example group G1B below.

Unsubstituted Aryl Group (Specific Example Group G1A): a phenyl group, p-biphenyl group, m-biphenyl group, o-biphenyl group, p-terphenyl-4-yl group, p-terphenyl-3-yl group, p-terphenyl-2-yl group, m-terphenyl-4-yl group, m-terphenyl-3-yl group, m-terphenyl-2-yl group, o-terphenyl-4-yl group, o-terphenyl-3-yl group, o-terphenyl-2-yl group, 1-naphthyl group, 2-naphthyl group, anthryl group, benzanthryl group, phenanthryl group, benzophenanthryl group, phenalenyl group, pyrenyl group, chrysenyl group, benzochrysenyl group, triphenylenyl group, benzotriphenylenyl group, tetracenyl group, pentacenyl group, fluorenyl group, 9,9′-spirobifluorenyl group, benzofluorenyl group, dibenzofluorenyl group, fluoranthenyl group, benzofluoranthenyl group, perylenyl group, and monovalent aryl group derived by removing one hydrogen atom from cyclic structures represented by formulae (TEMP-1) to (TEMP-15) below.

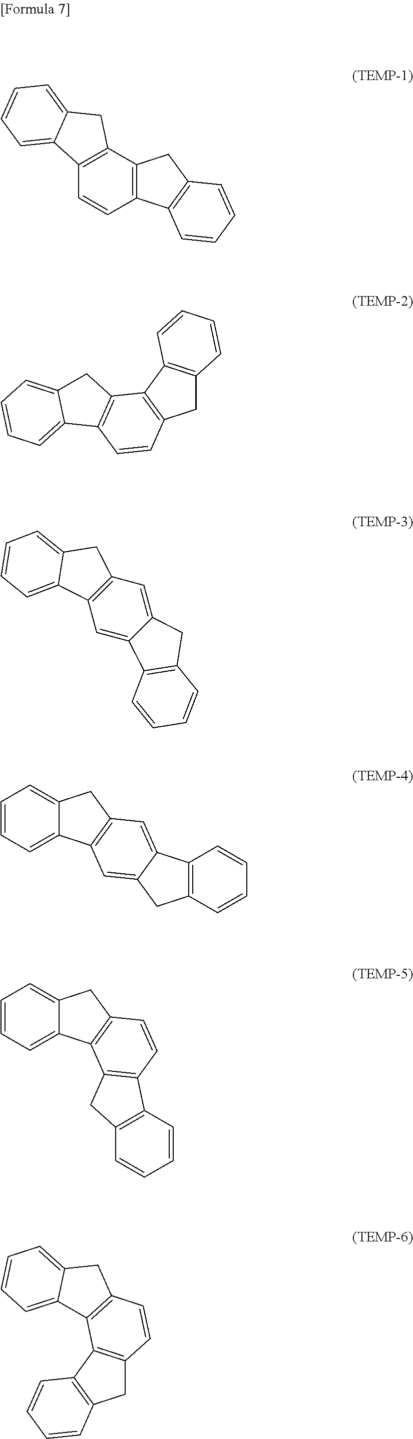

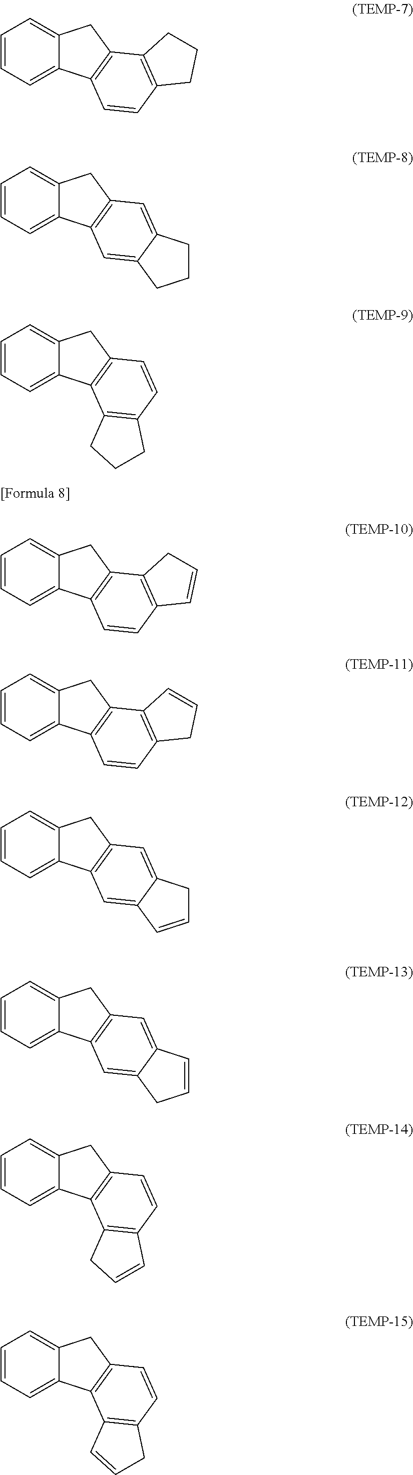

### Substituted Aryl Group (Specific Example Group G1B):

an o-tolyl group, m-tolyl group, p-tolyl group, para-xylyl group, meta-xylyl group, ortho-xylyl group, para-isopropylphenyl group, meta-isopropylphenyl group, ortho-isopropylphenyl group, para-t-butylphenyl group, meta-t-butylphenyl group, ortho-t-butylphenyl group, 3,4,5-trimethylphenyl group, 9,9-dimethylfluorenyl group, 9,9-diphenylfluorenyl group, 9,9-bis(4-methylphenyl)fluorenyl group, 9,9-bis(4-isopropylphenyl)fluorenyl group, 9,9-bis(4-t-butylphenyl)fluorenyl group, cyanophenyl group, triphenylsilylphenyl group, trimethylsilylphenyl group, phenylnaphthyl group, naphthylphenyl group, and group derived by substituting at least one hydrogen atom of a monovalent group derived from one of the cyclic structures represented by the formulae (TEMP-1) to (TEMP-15) with a substituent.

### Substituted or Unsubstituted Heterocyclic Group

The “heterocyclic group” mentioned herein refers to a cyclic group having at least one hetero atom in the ring atoms. Specific examples of the hetero atom include a nitrogen atom, oxygen atom, sulfur atom, silicon atom, phosphorus atom, and boron atom.

The “heterocyclic group” mentioned herein is a monocyclic group or a fused-ring group.

The “heterocyclic group” mentioned herein is an aromatic heterocyclic group or a non-aromatic heterocyclic group.

Specific examples (specific example group G2) of the “substituted or unsubstituted heterocyclic group” mentioned herein include unsubstituted heterocyclic groups (specific example group G2A) and substituted heterocyclic groups (specific example group G2B). (Herein, an unsubstituted heterocyclic group refers to an “unsubstituted heterocyclic group” in a “substituted or unsubstituted heterocyclic group,” and a substituted heterocyclic group refers to a “substituted heterocyclic group” in a “substituted or unsubstituted heterocyclic group.”) A simply termed “heterocyclic group” herein includes both of an “unsubstituted heterocyclic group” and a “substituted heterocyclic group.”

The “substituted heterocyclic group” refers to a group derived by substituting at least one hydrogen atom in an “unsubstituted heterocyclic group” with a substituent. Specific examples of the “substituted heterocyclic group” include a group derived by substituting at least one hydrogen atom in the “unsubstituted heterocyclic group” in the specific example group G2A below with a substituent, and examples of the substituted heterocyclic group in the specific example group G2B below. It should be noted that the examples of the “unsubstituted heterocyclic group” and the “substituted heterocyclic group” mentioned herein are merely exemplary, and the “substituted heterocyclic group” mentioned herein includes a group derived by further substituting a hydrogen atom bonded to a ring atom of a skeleton of a “substituted heterocyclic group” in the specific example group G2B below, and a group derived by further substituting a hydrogen atom of a substituent of the “substituted heterocyclic group” in the specific example group G2B below.

The specific example group G2A includes, for instance, unsubstituted heterocyclic groups including a nitrogen atom (specific example group G2A1) below, unsubstituted heterocyclic groups including an oxygen atom (specific example group G2A2) below, unsubstituted heterocyclic groups including a sulfur atom (specific example group G2A3) below, and monovalent heterocyclic groups (specific example group G2A4) derived by removing a hydrogen atom from cyclic structures represented by formulae (TEMP-16) to (TEMP-33) below.

The specific example group G2B includes, for instance, substituted heterocyclic groups including a nitrogen atom (specific example group G2B1) below, substituted heterocyclic groups including an oxygen atom (specific example group G2B2) below, substituted heterocyclic groups including a sulfur atom (specific example group G2B3) below, and groups derived by substituting at least one hydrogen atom of the monovalent heterocyclic groups (specific example group G2B4) derived from the cyclic structures represented by formulae (TEMP-16) to (TEMP-33) below.

### Unsubstituted Heterocyclic Groups Including Nitrogen Atom (Specific Example Group G2A1):

a pyrrolyl group, imidazolyl group, pyrazolyl group, triazolyl group, tetrazolyl group, oxazolyl group, isoxazolyl group, oxadiazolyl group, thiazolyl group, isothiazolyl group, thiadiazolyl group, pyridyl group, pyridazynyl group, pyrimidinyl group, pyrazinyl group, triazinyl group, indolyl group, isoindolyl group, indolizinyl group, quinolizinyl group, quinolyl group, isoquinolyl group, cinnolyl group, phthalazinyl group, quinazolinyl group, quinoxalinyl group, benzimidazolyl group, indazolyl group, phenanthrolinyl group, phenanthridinyl group, acridinyl group, phenazinyl group, carbazolyl group, benzocarbazolyl group, morpholino group, phenoxazinyl group, phenothiazinyl group, azacarbazolyl group, and diazacarbazolyl group.

### Unsubstituted Heterocyclic Groups Including Oxygen Atom (Specific Example Group G2A2):

a furyl group, oxazolyl group, isoxazolyl group, oxadiazolyl group, xanthenyl group, benzofuranyl group, isobenzofuranyl group, dibenzofuranyl group, naphthobenzofuranyl group, benzoxazolyl group, benzisoxazolyl group, phenoxazinyl group, morpholino group, dinaphthofuranyl group, azadibenzofuranyl group, diazadibenzofuranyl group, azanaphthobenzofuranyl group, and diazanaphthobenzofuranyl group.

### Unsubstituted Heterocyclic Groups Including Sulfur Atom (Specific Example Group G2A3):

a thienyl group, thiazolyl group, isothiazolyl group, thiadiazolyl group, benzothiophenyl group (benzothienyl group), isobenzothiophenyl group (isobenzothienyl group), dibenzothiophenyl group (dibenzothienyl group), naphthobenzothiophenyl group (nahthobenzothienyl group), benzothiazolyl group, benzisothiazolyl group, phenothiazinyl group, dinaphthothiophenyl group (dinaphthothienyl group), azadibenzothiophenyl group (azadibenzothienyl group), diazadibenzothiophenyl group (diazadibenzothienyl group), azanaphthobenzothiophenyl group (azanaphthobenzothienyl group), and diazanaphthobenzothiophenyl group (diazanaphthobenzothienyl group).

Monovalent Heterocyclic Groups Derived by Removing One Hydrogen Atom from Cyclic Structures Represented by Formulae (TEMP-16) to (TEMP-33) (Specific Example Group G2A4):

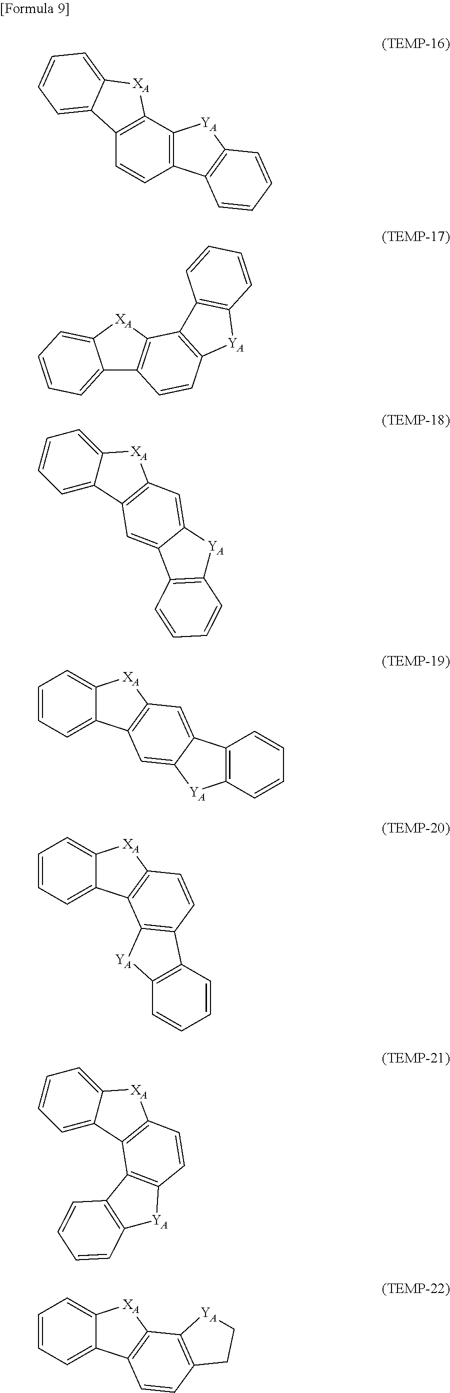

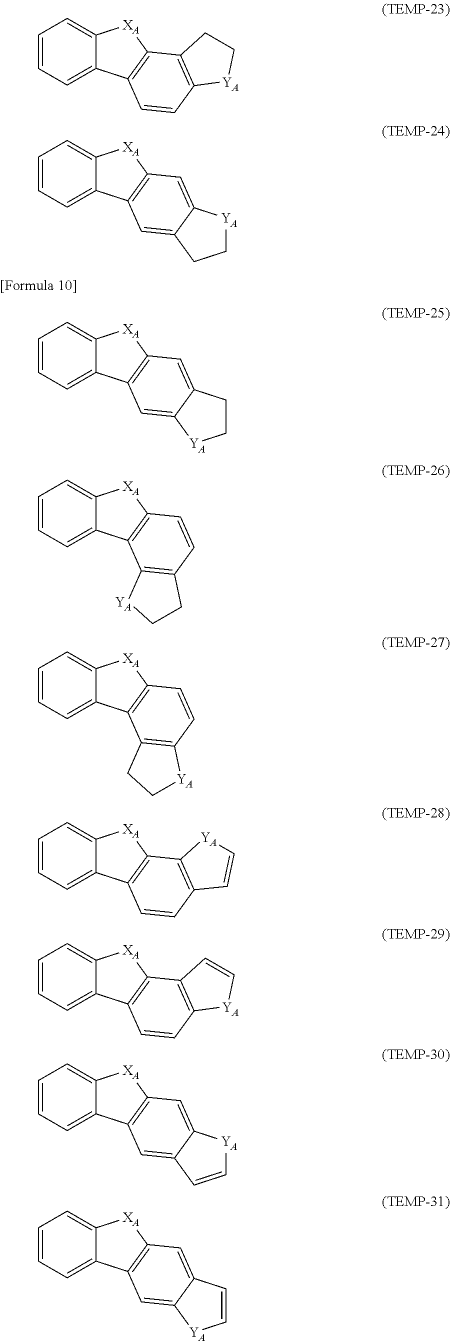

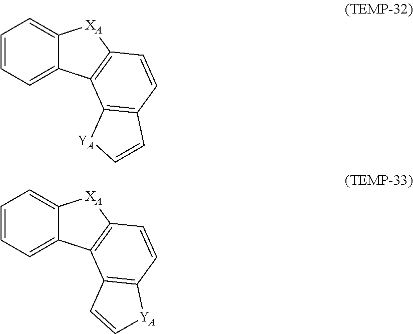

In the formulae (TEMP-16) to (TEMP-33), X_{A} and Y_{A} are each independently an oxygen atom, a sulfur atom, NH or CH₂, with a proviso that at least one of X_{A} or Y_{A} is an oxygen atom, a sulfur atom, or NH.

When at least one of X_{A} or Y_{A} in the formulae (TEMP-16) to (TEMP-33) is NH or CH₂, the monovalent heterocyclic groups derived from the cyclic structures represented by the formulae (TEMP-16) to (TEMP-33) include a monovalent group derived by removing one hydrogen atom from NH or CH₂.

### Substituted Heterocyclic Groups Including Nitrogen Atom (Specific Example Group G2B1):

a (9-phenyl)carbazolyl group, (9-biphenylyl)carbazolyl group, (9-phenyl)phenylcarbazolyl group, (9-naphthyl)carbazolyl group, diphenylcarbazole-9-yl group, phenylcarbazole-9-yl group, methylbenzimidazolyl group, ethylbenzimidazolyl group, phenyltriazinyl group, biphenylyltriazinyl group, diphenyltriazinyl group, phenylquinazolinyl group, and biphenylquinazolinyl group.

### Substituted Heterocyclic Groups Including Oxygen Atom (Specific Example Group G2B2):

a phenyldibenzofuranyl group, methyldibenzofuranyl group, t-butyldibenzofuranyl group, and monovalent residue of spiro[9H-xanthene-9,9′-[9H]fluorene].

### Substituted Heterocyclic Groups Including Sulfur Atom (Specific Example Group G2B3):

a phenyldibenzothiophenyl group, methyldibenzothiophenyl group, t-butyldibenzothiophenyl group, and monovalent residue of spiro[9H-thioxanthene-9,9′-[9H]fluorene].

Groups Obtained by Substituting at Least One Hydrogen Atom of Monovalent Heterocyclic Group Derived from Cyclic Structures Represented by Formulae (TEMP-16) to (TEMP-33) with Substituent (Specific Example Group G2B4):

The “at least one hydrogen atom of a monovalent heterocyclic group” means at least one hydrogen atom selected from a hydrogen atom bonded to a ring carbon atom of the monovalent heterocyclic group, a hydrogen atom bonded to a nitrogen atom of at least one of X_{A} or Y_{A} in a form of NH, and a hydrogen atom of one of X_{A} and Y_{A} in a form of a methylene group (CH₂).

### Substituted or Unsubstituted Alkyl Group

Specific examples (specific example group G3) of the “substituted or unsubstituted alkyl group” mentioned herein include unsubstituted alkyl groups (specific example group G3A) and substituted alkyl groups (specific example group G3B) below. (Herein, an unsubstituted alkyl group refers to an “unsubstituted alkyl group” in a “substituted or unsubstituted alkyl group,” and a substituted alkyl group refers to a “substituted alkyl group” in a “substituted or unsubstituted alkyl group.”) A simply termed “alkyl group” herein includes both of an “unsubstituted alkyl group” and a “substituted alkyl group”.

The “substituted alkyl group” refers to a group derived by substituting at least one hydrogen atom in an “unsubstituted alkyl group” with a substituent. Specific examples of the “substituted alkyl group” include a group derived by substituting at least one hydrogen atom of an “unsubstituted alkyl group” (specific example group G3A) below with a substituent, and examples of the substituted alkyl group (specific example group G3B) below. Herein, the alkyl group for the “unsubstituted alkyl group” refers to a chain alkyl group. Accordingly, the “unsubstituted alkyl group” include linear “unsubstituted alkyl group” and branched “unsubstituted alkyl group.” It should be noted that the examples of the “unsubstituted alkyl group” and the “substituted alkyl group” mentioned herein are merely exemplary, and the “substituted alkyl group” mentioned herein includes a group derived by further substituting a hydrogen atom of a skeleton of the “substituted alkyl group” in the specific example group G3B, and a group derived by further substituting a hydrogen atom of a substituent of the “substituted alkyl group” in the specific example group G3B.

### Unsubstituted Alkyl Group (Specific Example Group G3A):

a methyl group, ethyl group, n-propyl group, isopropyl group, n-butyl group, isobutyl group, s-butyl group, and t-butyl group.

### Substituted Alkyl Group (Specific Example Group G3B):

a heptafluoropropyl group (including isomer thereof), pentafluoroethyl group, 2,2,2-trifluoroethyl group, and trifluoromethyl group.

### Substituted or Unsubstituted Alkenyl Group

Specific examples (specific example group G4) of the “substituted or unsubstituted alkenyl group” mentioned herein include unsubstituted alkenyl groups (specific example group G4A) and substituted alkenyl groups (specific example group G4B). (Herein, an unsubstituted alkenyl group refers to an “unsubstituted alkenyl group” in a “substituted or unsubstituted alkenyl group,” and a substituted alkenyl group refers to a “substituted alkenyl group” in a “substituted or unsubstituted alkenyl group.”) A simply termed “alkenyl group” herein includes both of an “unsubstituted alkenyl group” and a “substituted alkenyl group”.

The “substituted alkenyl group” refers to a group derived by substituting at least one hydrogen atom in an “unsubstituted alkenyl group” with a substituent. Specific examples of the “substituted alkenyl group” include an “unsubstituted alkenyl group” (specific example group G4A) substituted by a substituent, and examples of the substituted alkenyl group (specific example group G4B) below. It should be noted that the examples of the “unsubstituted alkenyl group” and the “substituted alkenyl group” mentioned herein are merely exemplary, and the “substituted alkenyl group” mentioned herein includes a group derived by further substituting a hydrogen atom of a skeleton of the “substituted alkenyl group” in the specific example group G4B with a substituent, and a group derived by further substituting a hydrogen atom of a substituent of the “substituted alkenyl group” in the specific example group G4B with a substituent.

### Unsubstituted Alkenyl Group (Specific Example Group G4A):

a vinyl group, allyl group, 1-butenyl group, 2-butenyl group, and 3-butenyl group.

### Substituted Alkenyl Group (Specific Example Group G4B):

a 1,3-butanedienyl group, 1-methylvinyl group, 1-methylallyl group, 1,1-dimethylallyl group, 2-methylallyl group, and 1,2-dimethylallyl group.

### Substituted or Unsubstituted Alkynyl Group

Specific examples (specific example group G5) of the “substituted or unsubstituted alkynyl group” mentioned herein include unsubstituted alkynyl groups (specific example group G5A) below. (Herein, an unsubstituted alkynyl group refers to an “unsubstituted alkynyl group” in a “substituted or unsubstituted alkynyl group.”) A simply termed “alkynyl group” herein includes both of “unsubstituted alkynyl group” and “substituted alkynyl group”.

The “substituted alkynyl group” refers to a group derived by substituting at least one hydrogen atom in an “unsubstituted alkynyl group” with a substituent. Specific examples of the “substituted alkynyl group” include a group derived by substituting at least one hydrogen atom of the “unsubstituted alkynyl group” (specific example group G5A) below with a substituent.

Unsubstituted Alkynyl Group (Specific Example Group G5A): an ethynyl group.

### Substituted or Unsubstituted Cycloalkyl Group

Specific examples (specific example group G6) of the “substituted or unsubstituted cycloalkyl group” mentioned herein include unsubstituted cycloalkyl groups (specific example group G6A) and substituted cycloalkyl groups (specific example group G6B). (Herein, an unsubstituted cycloalkyl group refers to an “unsubstituted cycloalkyl group” in a “substituted or unsubstituted cycloalkyl group,” and a substituted cycloalkyl group refers to a “substituted cycloalkyl group” in a “substituted or unsubstituted cycloalkyl group.”) A simply termed “cycloalkyl group” herein includes both of “unsubstituted cycloalkyl group” and “substituted cycloalkyl group”.

The “substituted cycloalkyl group” refers to a group derived by substituting at least one hydrogen atom of an “unsubstituted cycloalkyl group” with a substituent. Specific examples of the “substituted cycloalkyl group” include a group derived by substituting at least one hydrogen atom of the “unsubstituted cycloalkyl group” (specific example group G6A) below with a substituent, and examples of the substituted cycloalkyl group (specific example group G6B) below. It should be noted that the examples of the “unsubstituted cycloalkyl group” and the “substituted cycloalkyl group” mentioned herein are merely exemplary, and the “substituted cycloalkyl group” mentioned herein includes a group derived by substituting at least one hydrogen atom bonded to a carbon atom of a skeleton of the “substituted cycloalkyl group” in the specific example group G6B with a substituent, and a group derived by further substituting a hydrogen atom of a substituent of the “substituted cycloalkyl group” in the specific example group G6B with a substituent.

### Unsubstituted Cycloalkyl Group (Specific Example Group G6A):

a cyclopropyl group, cyclobutyl group, cyclopentyl group, cyclohexyl group, 1-adamantyl group, 2-adamantyl group, 1-norbornyl group, and 2-norbornyl group.

Substituted Cycloalkyl Group (Specific Example Group G6B): a 4-methylcyclohexyl group.
Group Represented by —Si(R₉₀₁)(R₉₀₂)(R₉₀₃)

Specific examples (specific example group G7) of the group represented herein by —Si(R₉₀₁)(R₉₀₂)(R₉₀₃) include: —Si(G1)(G1)(G1); —Si(G1)(G2)(G2); —Si(G1)(G1)(G2); —Si(G2)(G2)(G2); —Si(G3)(G3)(G3); and —Si(G6)(G6)(G6),

where: G1 represents a “substituted or unsubstituted aryl group” in the specific example group G1;
G2 represents a “substituted or unsubstituted heterocyclic group” in the specific example group G2;
G3 represents a “substituted or unsubstituted alkyl group” in the specific example group G3;
G6 represents a “substituted or unsubstituted cycloalkyl group” in the specific example group G6;
a plurality of G1 in —Si(G1)(G1)(G1) are mutually the same or different;
a plurality of G2 in —Si(G1)(G2)(G2) are mutually the same or different;
a plurality of G2 in —Si(G1)(G2)(G2) are mutually the same or different;
a plurality of G2 in —Si(G2)(G2)(G2) are mutually the same or different;
a plurality of G3 in —Si(G3)(G3)(G3) are mutually the same or different; and
a plurality of G6 in —Si(G6)(G6)(G6) are mutually the same or different.

### Group Represented by —O—(R₉₀₄)

Specific examples (specific example group G8) of a group represented by —O—(R₉₀₄) herein include: —O(G1); —O(G2); —O(G3); and —O(G6),
where:
G1 represents a “substituted or unsubstituted aryl group” in the specific example group G1;
G2 represents a “substituted or unsubstituted heterocyclic group” in the specific example group G2;
G3 represents a “substituted or unsubstituted alkyl group” in the specific example group G3; and
G6 represents a “substituted or unsubstituted cycloalkyl group” in the specific example group G6.

### Group Represented by —S—(R₉₀₅)

Specific examples (specific example group G9) of a group represented herein by —S—(R₉₀₅) include: —S(G1); —S(G2); —S(G3); and —S(G6),
where:
G1 represents a “substituted or unsubstituted aryl group” in the specific example group G1;
G2 represents a “substituted or unsubstituted heterocyclic group” in the specific example group G2;
G3 represents a “substituted or unsubstituted alkyl group” in the specific example group G3; and
G6 represents a “substituted or unsubstituted cycloalkyl group” in the specific example group G6.
   Group Represented by —N(R₉₀₆)(R₉₀₇)

Specific examples (specific example group G10) of a group represented herein by —N(R₉₀₆)(R₉₀₇) include: —N(G1)(G1); —N(G2)(G2); —N(G1)(G2); —N(G3)(G3); and —N(G6)(G6),
where:
G1 represents a “substituted or unsubstituted aryl group” in the specific example group G1;
G2 represents a “substituted or unsubstituted heterocyclic group” in the specific example group G2;
G3 represents a “substituted or unsubstituted alkyl group” in the specific example group G3;
G6 represents a “substituted or unsubstituted cycloalkyl group” in the specific example group G6;
a plurality of G1 in —N(G1)(G1) are mutually the same or different;
a plurality of G2 in —N(G2)(G2) are mutually the same or different;
a plurality of G3 in —N(G3)(G3) are mutually the same or different; and
a plurality of G6 in —N(G6)(G6) are mutually the same or different.

### Halogen Atom

Specific examples (specific example group G11) of “halogen atom” mentioned herein include a fluorine atom, chlorine atom, bromine atom, and iodine atom.

### Substituted or Unsubstituted Fluoroalkyl Group

The “substituted or unsubstituted fluoroalkyl group” mentioned herein refers to a group derived by substituting at least one hydrogen atom bonded to at least one of carbon atoms forming an alkyl group in the “substituted or unsubstituted alkyl group” with a fluorine atom, and also includes a group (perfluoro group) derived by substituting all of hydrogen atoms bonded to carbon atoms forming the alkyl group in the “substituted or unsubstituted alkyl group” with fluorine atoms. An “unsubstituted fluoroalkyl group” has, unless otherwise specified herein, 1 to 50, preferably 1 to 30, more preferably 1 to 18 carbon atoms. The “substituted fluoroalkyl group” refers to a group derived by substituting at least one hydrogen atom in a “fluoroalkyl group” with a substituent. It should be noted that the examples of the “substituted fluoroalkyl group” mentioned herein include a group derived by further substituting at least one hydrogen atom bonded to a carbon atom of an alkyl chain of a “substituted fluoroalkyl group” with a substituent, and a group derived by further substituting at least one hydrogen atom of a substituent of the “substituted fluoroalkyl group” with a substituent. Specific examples of the “unsubstituted fluoroalkyl group” include a group derived by substituting at least one hydrogen atom of the “alkyl group” (specific example group G3) with a fluorine atom.

### Substituted or Unsubstituted Haloalkyl Group

The “substituted or unsubstituted haloalkyl group” mentioned herein refers to a group derived by substituting at least one hydrogen atom bonded to carbon atoms forming the alkyl group in the “substituted or unsubstituted alkyl group” with a halogen atom, and also includes a group derived by substituting all hydrogen atoms bonded to carbon atoms forming the alkyl group in the “substituted or unsubstituted alkyl group” with halogen atoms. An “unsubstituted haloalkyl group” has, unless otherwise specified herein, 1 to 50, preferably 1 to 30, and more preferably 1 to 18 carbon atoms. The “substituted haloalkyl group” refers to a group derived by substituting at least one hydrogen atom in a “haloalkyl group” with a substituent. It should be noted that the examples of the “substituted haloalkyl group” mentioned herein include a group derived by further substituting at least one hydrogen atom bonded to a carbon atom of an alkyl chain of a “substituted haloalkyl group” with a substituent, and a group derived by further substituting at least one hydrogen atom of a substituent of the “substituted haloalkyl group” with a substituent. Specific examples of the “unsubstituted haloalkyl group” include a group derived by substituting at least one hydrogen atom of the “alkyl group” (specific example group G3) with a halogen atom. The haloalkyl group is sometimes referred to as a halogenated alkyl group.

### Substituted or Unsubstituted Alkoxy Group

Specific examples of a “substituted or unsubstituted alkoxy group” mentioned herein include a group represented by —O(G3), G3 being the “substituted or unsubstituted alkyl group” in the specific example group G3. An “unsubstituted alkoxy group” has, unless otherwise specified herein, 1 to 50, preferably 1 to 30, more preferably 1 to 18 carbon atoms.

### Substituted or Unsubstituted Alkylthio Group

Specific examples of a “substituted or unsubstituted alkylthio group” mentioned herein include a group represented by —S(G3), G3 being the “substituted or unsubstituted alkyl group” in the specific example group G3. An “unsubstituted alkylthio group” has, unless otherwise specified herein, 1 to 50, preferably 1 to 30, more preferably 1 to 18 carbon atoms.

### Substituted or Unsubstituted Aryloxy Group

Specific examples of a “substituted or unsubstituted aryloxy group” mentioned herein include a group represented by —O(G1), G1 being the “substituted or unsubstituted aryl group” in the specific example group G1. An “unsubstituted aryloxy group” has, unless otherwise specified herein, 6 to 50, preferably 6 to 30, more preferably 6 to 18 ring carbon atoms.

### Substituted or Unsubstituted Arylthio Group

Specific examples of a “substituted or unsubstituted arylthio group” mentioned herein include a group represented by —S(G1), G1 being the “substituted or unsubstituted aryl group” in the specific example group G1. An “unsubstituted arylthio group” has, unless otherwise specified herein, 6 to 50, preferably 6 to 30, more preferably 6 to 18 ring carbon atoms.

### Substituted or Unsubstituted Trialkylsilyl Group

Specific examples of a “trialkylsilyl group” mentioned herein include a group represented by —Si(G3)(G3)(G3), G3 being the “substituted or unsubstituted alkyl group” in the specific example group G3. A plurality of G3 in —Si(G3)(G3)(G3) are mutually the same or different. Each of the alkyl groups in the “trialkylsilyl group” has, unless otherwise specified herein, 1 to 50, preferably 1 to 20, more preferably 1 to 6 carbon atoms.

### Substituted or Unsubstituted Aralkyl Group

Specific examples of a “substituted or unsubstituted aralkyl group” mentioned herein include a group represented by -(G3)-(G1), G3 being the “substituted or unsubstituted alkyl group” in the specific example group G3, G1 being the “substituted or unsubstituted aryl group” in the specific example group G1. Accordingly, the “aralkyl group” is a group derived by substituting a hydrogen atom of the “alkyl group” with a substituent in a form of the “aryl group,” which is an example of the “substituted alkyl group.” An “unsubstituted aralkyl group,” which is an “unsubstituted alkyl group” substituted by an “unsubstituted aryl group,” has, unless otherwise specified herein, 7 to 50 carbon atoms, preferably 7 to 30 carbon atoms, more preferably 7 to 18 carbon atoms.

Specific examples of the “substituted or unsubstituted aralkyl group” include a benzyl group, 1-phenylethyl group, 2-phenylethyl group, 1-phenylisopropyl group, 2-phenylisopropyl group, phenyl-t-butyl group, α-naphthylmethyl group, 1-α-naphthylethyl group, 2-α-naphthylethyl group, 1-α-naphthylisopropyl group, 2-α-naphthylisopropyl group, β-naphthylmethyl group, 1-β-naphthylethyl group, 2-β-naphthylethyl group, 1-β-naphthylisopropyl group, and 2-β-naphthylisopropyl group.

Preferable examples of the substituted or unsubstituted aryl group mentioned herein include, unless otherwise specified herein, a phenyl group, p-biphenyl group, m-biphenyl group, o-biphenyl group, p-terphenyl-4-yl group, p-terphenyl-3-yl group, p-terphenyl-2-yl group, m-terphenyl-4-yl group, m-terphenyl-3-yl group, m-terphenyl-2-yl group, o-terphenyl-4-yl group, o-terphenyl-3-yl group, o-terphenyl-2-yl group, 1-naphthyl group, 2-naphthyl group, anthryl group, phenanthryl group, pyrenyl group, chrysenyl group, triphenylenyl group, fluorenyl group, 9,9′-spirobifluorenyl group, 9,9-dimethylfluorenyl group, and 9,9-diphenylfluorenyl group.

Preferable examples of the substituted or unsubstituted heterocyclic group mentioned herein include, unless otherwise specified herein, a pyridyl group, pyrimidinyl group, triazinyl group, quinolyl group, isoquinolyl group, quinazolinyl group, benzimidazolyl group, phenanthrolinyl group, carbazolyl group (1-carbazolyl group, 2-carbazolyl group, 3-carbazolyl group, 4-carbazolyl group, or 9-carbazolyl group), benzocarbazolyl group, azacarbazolyl group, diazacarbazolyl group, dibenzofuranyl group, naphthobenzofuranyl group, azadibenzofuranyl group, diazadibenzofuranyl group, dibenzothiophenyl group, naphthobenzothiophenyl group, azadibenzothiophenyl group, diazadibenzothiophenyl group, (9-phenyl)carbazolyl group ((9-phenyl)carbazole-1-yl group, (9-phenyl)carbazole-2-yl group, (9-phenyl)carbazole-3-yl group, or (9-phenyl)carbazole-4-yl group), (9-biphenylyl)carbazolyl group, (9-phenyl)phenylcarbazolyl group, diphenylcarbazole-9-yl group, phenylcarbazole-9-yl group, phenyltriazinyl group, biphenylyltriazinyl group, diphenyltriazinyl group, phenyldibenzofuranyl group, and phenyldibenzothiophenyl group.

The carbazolyl group mentioned herein is, unless otherwise specified herein, specifically a group represented by one of formulae below.

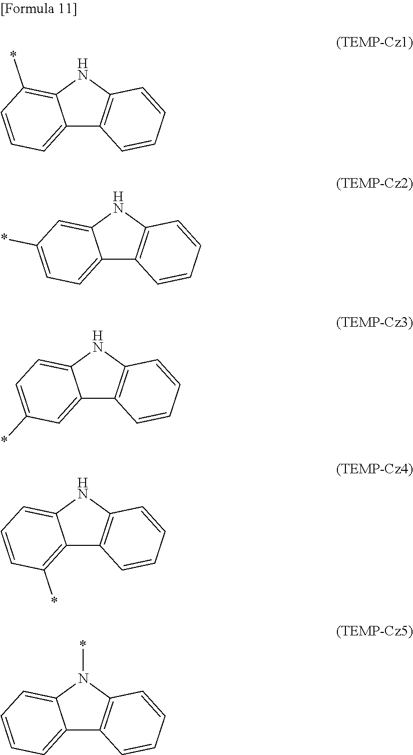 [SMILES: *c1cccc2c1[nH]c1ccccc12 ; *c1ccc2c(c1)[nH]c1ccccc12 ; *c1ccc2[nH]c3ccccc3c2c1 ; *c1cccc2[nH]c3ccccc3c12 ; *n1c2ccccc2c2ccccc21]

The (9-phenyl)carbazolyl group mentioned herein is, unless otherwise specified herein, specifically a group represented by one of formulae below.

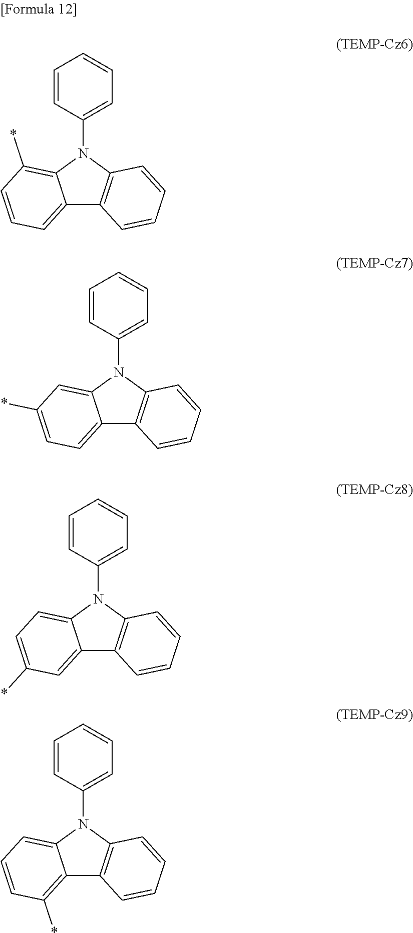

In the formulae (TEMP-Cz1) to (TEMP-Cz9), each * represents a bonding position.

The dibenzofuranyl group and dibenzothiophenyl group mentioned herein are, unless otherwise specified herein, each specifically represented by one of formulae below.

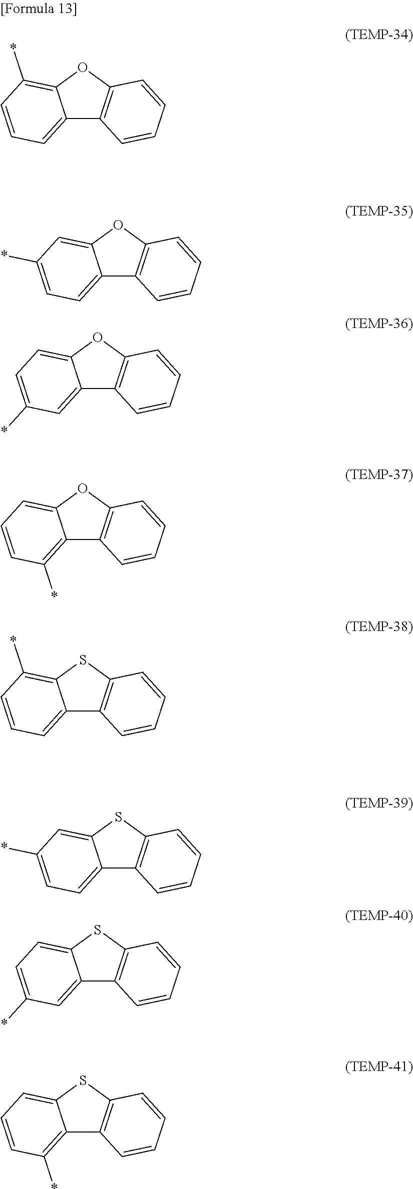

In the formulae (TEMP-34) to (TEMP-41), each * represents a bonding position.

Preferable examples of the substituted or unsubstituted alkyl group mentioned herein include, unless otherwise specified herein, a methyl group, ethyl group, propyl group, isopropyl group, n-butyl group, isobutyl group, and t-butyl group.

### Substituted or Unsubstituted Arylene Group

The “substituted or unsubstituted arylene group” mentioned herein is, unless otherwise specified herein, a divalent group derived by removing one hydrogen atom on an aryl ring of the “substituted or unsubstituted aryl group.” Specific examples of the “substituted or unsubstituted arylene group” (specific example group G12) include a divalent group derived by removing one hydrogen atom on an aryl ring of the “substituted or unsubstituted aryl group” in the specific example group G1.

### Substituted or Unsubstituted Divalent Heterocyclic Group

The “substituted or unsubstituted divalent heterocyclic group” mentioned herein is, unless otherwise specified herein, a divalent group derived by removing one hydrogen atom on a heterocyclic ring of the “substituted or unsubstituted heterocyclic group.” Specific examples of the “substituted or unsubstituted divalent heterocyclic group” (specific example group G13) include a divalent group derived by removing one hydrogen atom on a heterocyclic ring of the “substituted or unsubstituted heterocyclic group” in the specific example group G2.

### Substituted or Unsubstituted Alkylene Group

The “substituted or unsubstituted alkylene group” mentioned herein is, unless otherwise specified herein, a divalent group derived by removing one hydrogen atom on an alkyl chain of the “substituted or unsubstituted alkyl group.” Specific examples of the “substituted or unsubstituted alkylene group” (specific example group G14) include a divalent group derived by removing one hydrogen atom on an alkyl chain of the “substituted or unsubstituted alkyl group” in the specific example group G3.

The substituted or unsubstituted arylene group mentioned herein is, unless otherwise specified herein, preferably any one of groups represented by formulae (TEMP-42) to (TEMP-68) below.

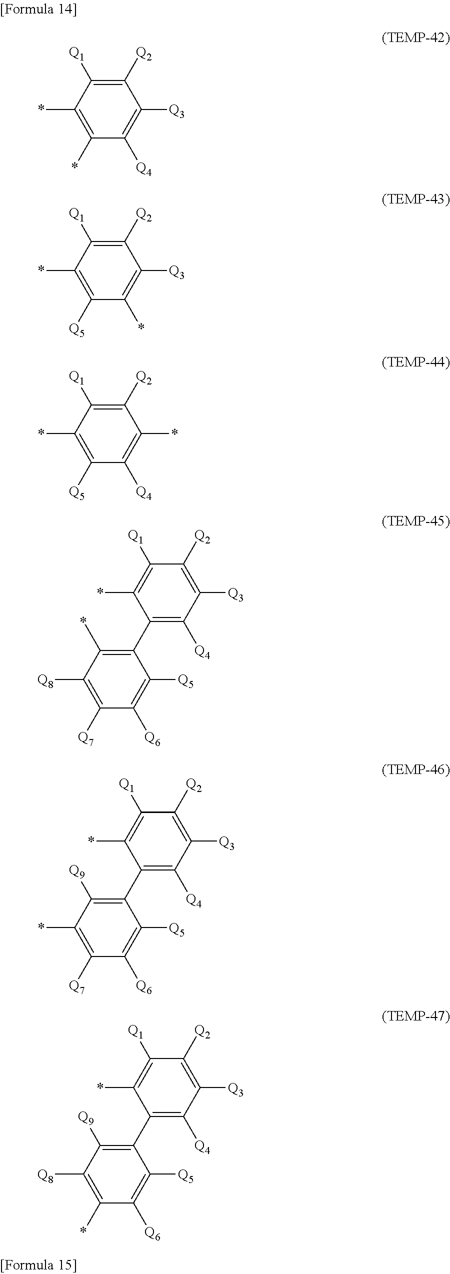

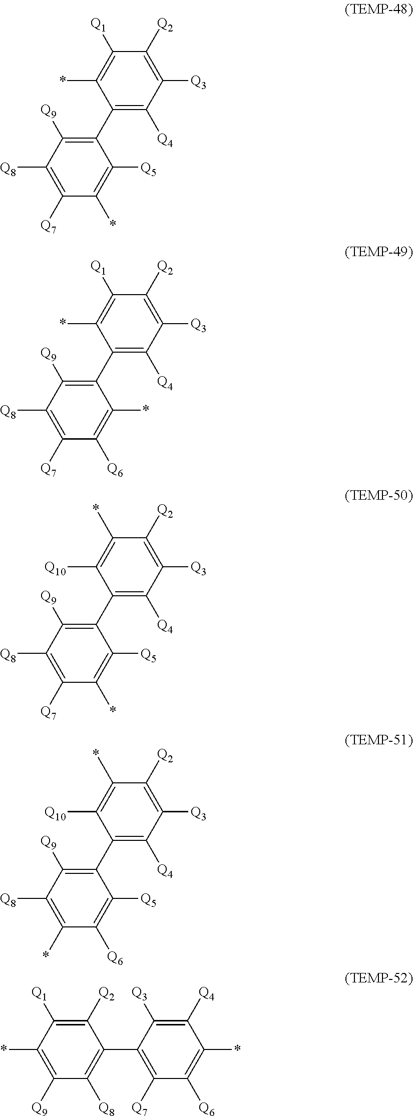

In the formulae (TEMP-42) to (TEMP-52), Q₁ to Q₁₀ are each independently a hydrogen atom or a substituent.

In the formulae (TEMP-42) to (TEMP-52), each * represents a bonding position.

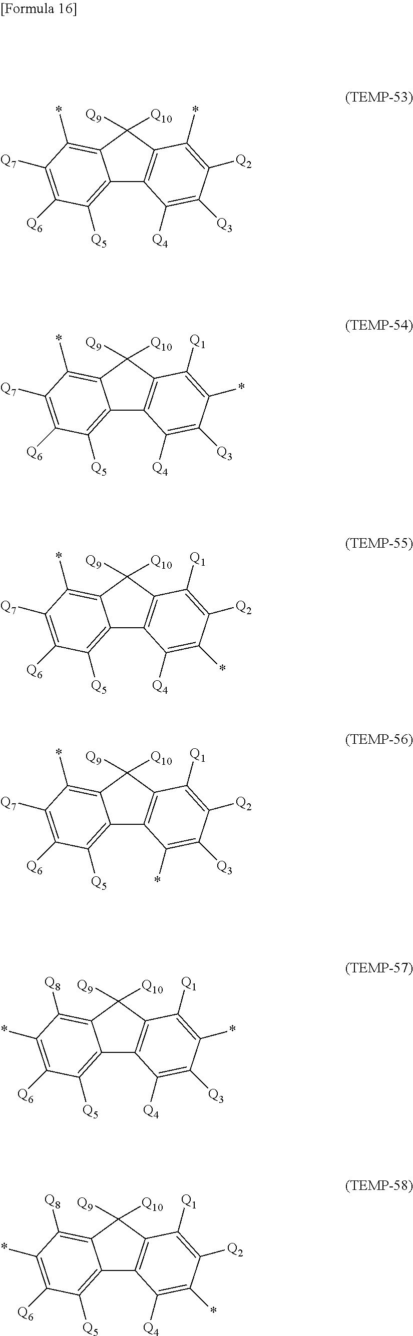

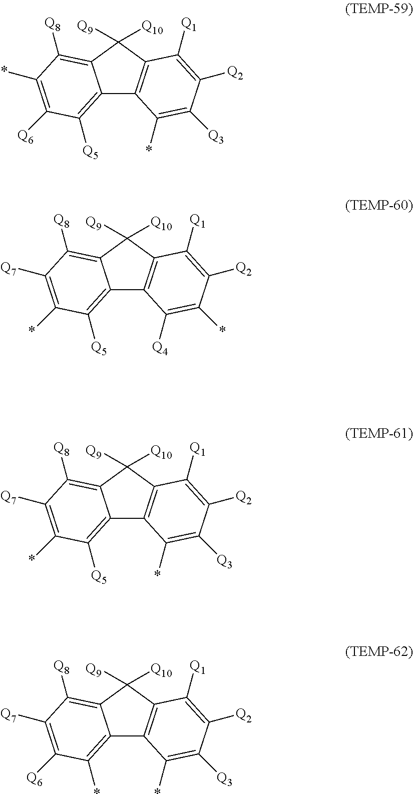

In the formulae (TEMP-53) to (TEMP-62), Q₁ to Q₁₀ are each independently a hydrogen atom or a substituent.

In the formulae, Q₉ and Q₁₀ may be mutually bonded through a single bond to form a ring.

In the formulae (TEMP-53) to (TEMP-62), each represents a bonding position.

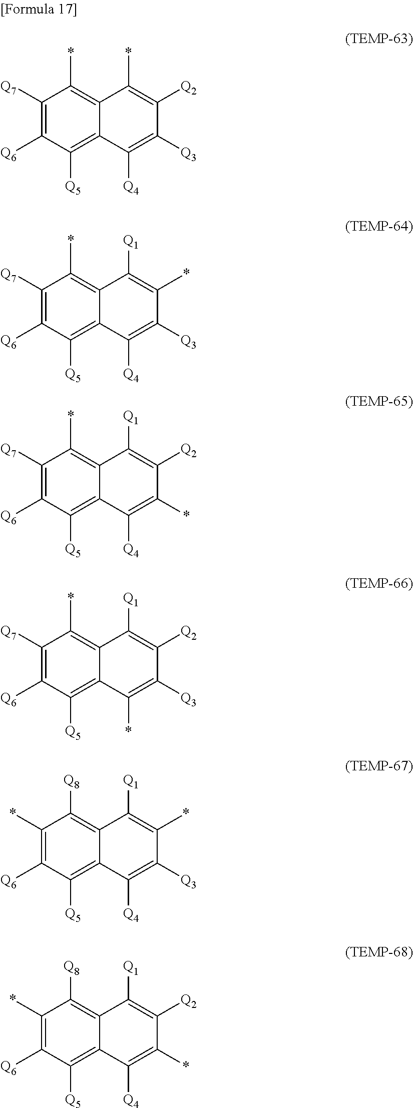

In the formulae (TEMP-63) to (TEMP-68), Q₁ to Q₈ are each independently a hydrogen atom or a substituent.

In the formulae (TEMP-63) to (TEMP-68), each * represents a bonding position.

The substituted or unsubstituted divalent heterocyclic group mentioned herein is, unless otherwise specified herein, preferably a group represented by any one of formulae (TEMP-69) to (TEMP-102) below.

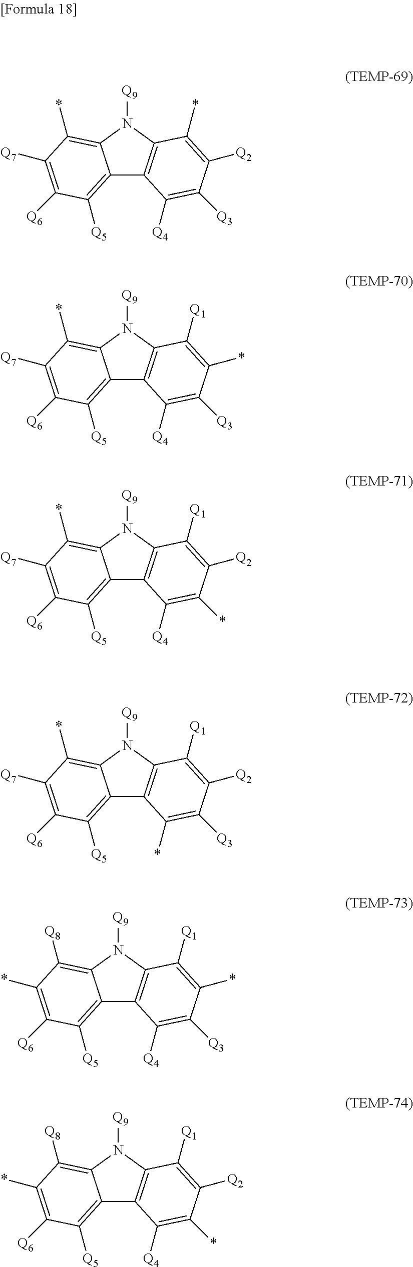

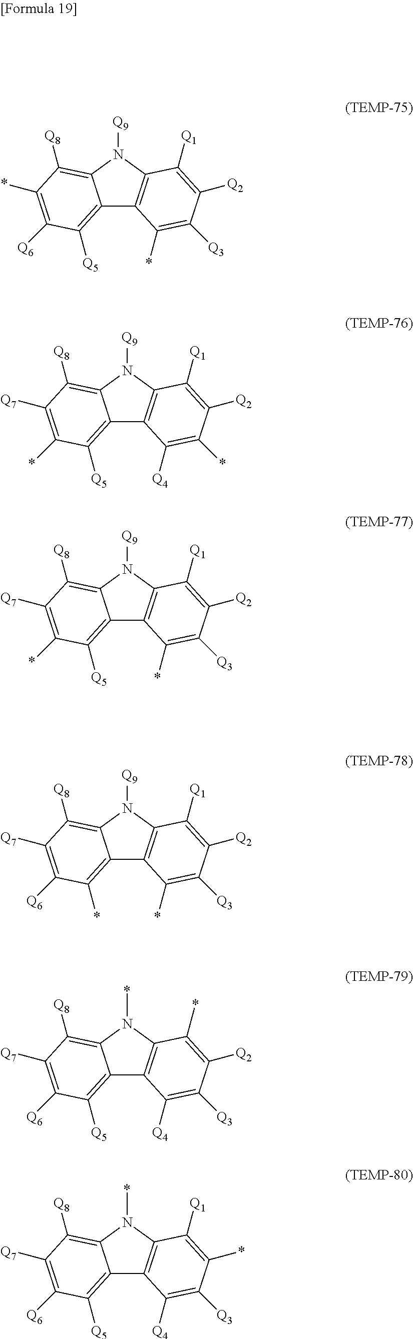

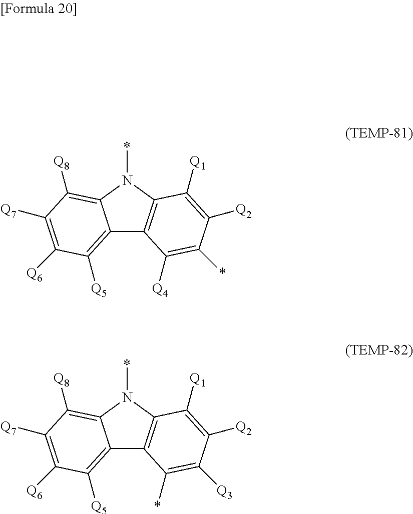

In the formulae (TEMP-69) to (TEMP-82), Q₁ to Q₉ are each independently a hydrogen atom or a substituent.

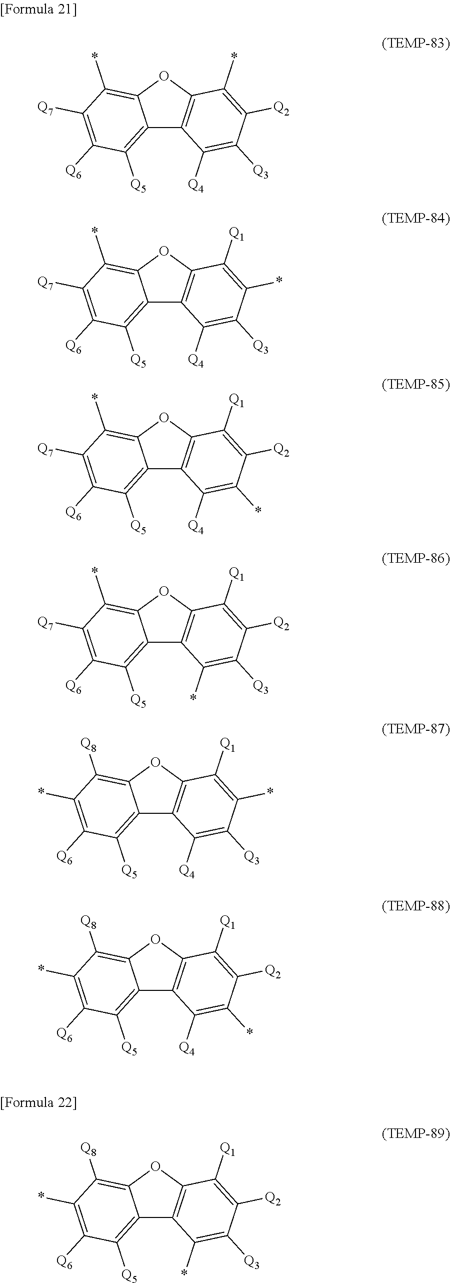

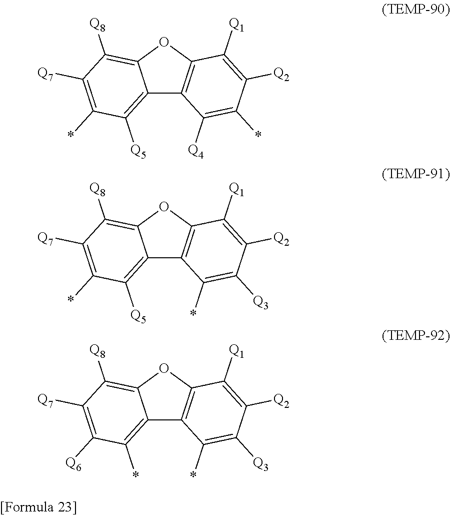

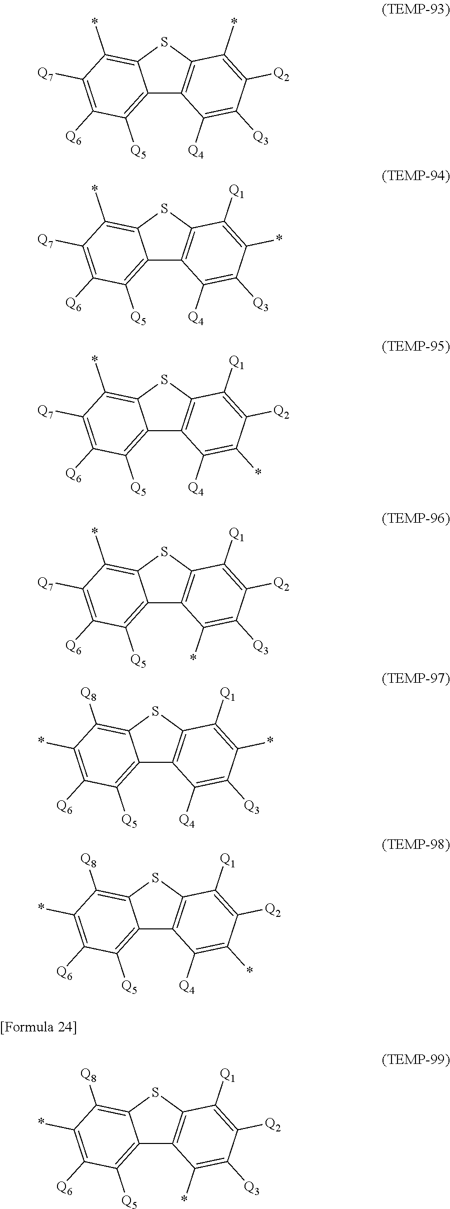

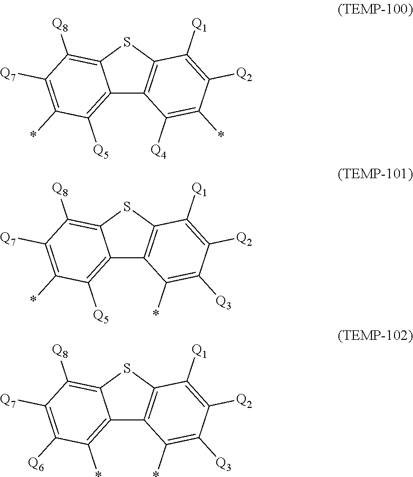

In the formulae (TEMP-83) to (TEMP-102), Q₁ to Q₈ are each independently a hydrogen atom or a substituent.

The substituent mentioned herein has been described above.

### Instance of “Bonded to Form Ring”

Instances where “at least one combination of adjacent two or more (of . . . ) are mutually bonded to form a substituted or unsubstituted monocyclic ring, mutually bonded to form a substituted or unsubstituted fused ring, or not mutually bonded” mentioned herein refer to instances where “at least one combination of adjacent two or more (of . . . ) are mutually bonded to form a substituted or unsubstituted monocyclic ring, “at least one combination of adjacent two or more (of . . . ) are mutually bonded to form a substituted or unsubstituted fused ring,” and “at least one combination of adjacent two or more (of . . . ) are not mutually bonded.”

Instances where “at least one combination of adjacent two or more (of . . . ) are mutually bonded to form a substituted or unsubstituted monocyclic ring” and “at least one combination of adjacent two or more (of . . . ) are mutually bonded to form a substituted or unsubstituted fused ring” mentioned herein (these instances will be sometimes collectively referred to as an instance of “bonded to form a ring” hereinafter) will be described below. An anthracene compound having a basic skeleton in a form of an anthracene ring and represented by a formula (TEMP-103) below will be used as an example for the description.

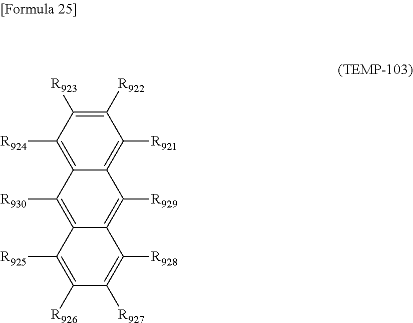

For instance, when “at least one combination of adjacent two or more of R₉₂₁ to R₉₃₀ are mutually bonded to form a ring,” the combination of adjacent ones of R₉₂₁ to R₉₃₀(i.e. the combination at issue) is a combination of R₉₂₁ and R₉₂₂, a combination of R₉₂₂ and R₉₂₃, a combination of R₉₂₃ and R₉₂₄, a combination of R₉₂₄ and R₉₃₀, a combination of R₉₃₀ and R₉₂₅, a combination of R₉₂₅ and R₉₂₆, a combination of R₉₂₆ and R₉₂₇, a combination of R₉₂₇ and R₉₂₈, a combination of R₉₂₈ and R₉₂₉, or a combination of R₉₂₉ and R₉₂₁.

The term “at least one combination” means that two or more of the above combinations of adjacent two or more of R₉₂₁ to R₉₃₀ may concurrently form rings. For instance, when R₉₂₁ and R₉₂₂ are mutually bonded to form a ring Q_{A} and R₉₂₅ and R₉₂₆ are concurrently mutually bonded to form a ring QB, the anthracene compound represented by the formula (TEMP-103) is represented by a formula (TEMP-104) below.

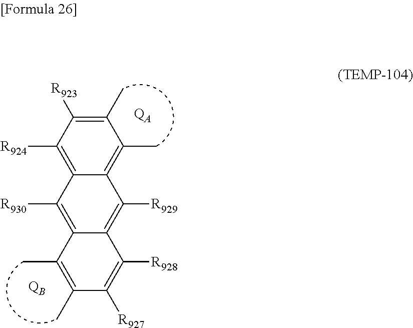

The instance where the “combination of adjacent two or more” form a ring means not only an instance where the “two” adjacent components are bonded but also an instance where adjacent “three or more” are bonded. For instance, R₉₂₁ and R₉₂₂ are mutually bonded to form a ring Q_{A} and R₉₂₂ and R₉₂₃ are mutually bonded to form a ring Q_{c}, and mutually adjacent three components (R₉₂₁, R₉₂₂ and R₉₂₃) are mutually bonded to form a ring fused to the anthracene basic skeleton. In this case, the anthracene compound represented by the formula (TEMP-103) is represented by a formula (TEMP-105) below. In the formula (TEMP-105) below, the ring Q_{A} and the ring Q_{c} share R₉₂₂.

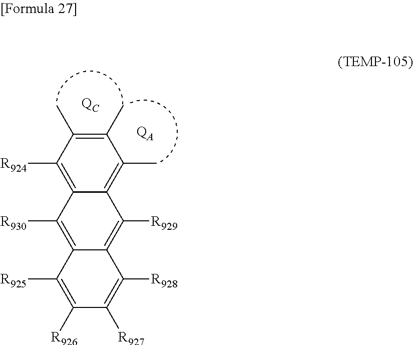

The formed “monocyclic ring” or “fused ring” may be, in terms of the formed ring in itself, a saturated ring or an unsaturated ring. When the “combination of adjacent two” form a “monocyclic ring” or a “fused ring,” the “monocyclic ring” or “fused ring” may be a saturated ring or an unsaturated ring. For instance, the ring Q_{A} and the ring QB formed in the formula (TEMP-104) are each independently a “monocyclic ring” or a “fused ring.” Further, the ring Q_{A} and the ring OC formed in the formula (TEMP-105) are each a “fused ring.” The ring Q_{A} and the ring Q_{C} in the formula (TEMP-105) are fused to form a fused ring. When the ring Q_{A} in the formula (TEMP-104) is a benzene ring, the ring Q_{A} is a monocyclic ring. When the ring Q_{A} in the formula (TEMP-104) is a naphthalene ring, the ring Q_{A} is a fused ring.

The “unsaturated ring” represents an aromatic hydrocarbon ring or an aromatic heterocyclic ring. The “saturated ring” represents an aliphatic hydrocarbon ring or a non-aromatic heterocyclic ring.

Specific examples of the aromatic hydrocarbon ring include a ring formed by terminating a bond of a group in the specific example of the specific example group G1 with a hydrogen atom.

Specific examples of the aromatic heterocyclic ring include a ring formed by terminating a bond of an aromatic heterocyclic group in the specific example of the specific example group G2 with a hydrogen atom.

Specific examples of the aliphatic hydrocarbon ring include a ring formed by terminating a bond of a group in the specific example of the specific example group G6 with a hydrogen atom.

The phrase “to form a ring” herein means that a ring is formed only by a plurality of atoms of a basic skeleton, or by a combination of a plurality of atoms of the basic skeleton and one or more optional atoms. For instance, the ring Q_{A} formed by mutually bonding R₉₂₁ and R₉₂₂ shown in the formula (TEMP-104) is a ring formed by a carbon atom of the anthracene skeleton bonded to R₉₂₁, a carbon atom of the anthracene skeleton bonded to R₉₂₂, and one or more optional atoms. Specifically, when the ring Q_{A} is a monocyclic unsaturated ring formed by R₉₂₁ and R₉₂₂, the ring formed by a carbon atom of the anthracene skeleton bonded to R₉₂₁, a carbon atom of the anthracene skeleton bonded to R₉₂₂, and four carbon atoms is a benzene ring.

The “optional atom” is, unless otherwise specified herein, preferably at least one atom selected from the group consisting of a carbon atom, nitrogen atom, oxygen atom, and sulfur atom. A bond of the optional atom (e.g. a carbon atom and a nitrogen atom) not forming a ring may be terminated by a hydrogen atom or the like or may be substituted by an “optional substituent” described later. When the ring includes an optional element other than carbon atom, the formed ring is a heterocyclic ring.

The number of “one or more optional atoms” forming the monocyclic ring or fused ring is, unless otherwise specified herein, preferably in a range from 2 to 15, more preferably in a range from 3 to 12, further preferably in a range from 3 to 5. Unless otherwise specified herein, the ring, which may be a “monocyclic ring” or “fused ring,” is preferably a “monocyclic ring.”

Unless otherwise specified herein, the ring, which may be a “saturated ring” or “unsaturated ring,” is preferably an “unsaturated ring.”

Unless otherwise specified herein, the “monocyclic ring” is preferably a benzene ring.

Unless otherwise specified herein, the “unsaturated ring” is preferably a benzene ring.

When “at least one combination of adjacent two or more” (of . . . ) are “mutually bonded to form a substituted or unsubstituted monocyclic ring” or “mutually bonded to form a substituted or unsubstituted fused ring,” unless otherwise specified herein, at least one combination of adjacent two or more of components are preferably mutually bonded to form a substituted or unsubstituted “unsaturated ring” formed of a plurality of atoms of the basic skeleton, and 1 to 15 atoms of at least one element selected from the group consisting of carbon, nitrogen, oxygen and sulfur.

When the “monocyclic ring” or the “fused ring” has a substituent, the substituent is the substituent described in later-described “optional substituent.” When the “monocyclic ring” or the “fused ring” has a substituent, specific examples of the substituent are the substituents described in the above under the subtitle “Substituent Mentioned Herein.”

When the “saturated ring” or the “unsaturated ring” has a substituent, the substituent is the substituent described in later-described “optional substituent.” When the “monocyclic ring” or the “fused ring” has a substituent, specific examples of the substituent are the substituents described in the above under the subtitle “Substituent Mentioned Herein.”

The above is the description for the instances where “at least one combination of adjacent two or more (of . . . ) are mutually bonded to form a substituted or unsubstituted monocyclic ring” and “at least one combination of adjacent two or more (of . . . ) are mutually bonded to form a substituted or unsubstituted fused ring” mentioned herein (sometimes referred to as an instance of “bonded to form a ring”).

### Substituent for Substituted or Unsubstituted Group

In an exemplary embodiment herein, the substituent for the substituted or unsubstituted group (sometimes referred to as an “optional substituent” hereinafter), is for instance, a group selected from the group consisting of an unsubstituted alkyl group having 1 to 50 carbon atoms, an unsubstituted alkenyl group having 2 to 50 carbon atoms, an unsubstituted alkynyl group having 2 to 50 carbon atoms, an unsubstituted cycloalkyl group having 3 to 50 ring carbon atoms, —Si(R₉₀₁)(R₉₀₂)(R₉₀₃), —O—(R₉₀₄), —S—(R₉₀₅), —N(R₉₀₆)(R₉₀₇), a halogen atom, a cyano group, a nitro group, an unsubstituted aryl group having 6 to 50 ring carbon atoms, and an unsubstituted heterocyclic group having 5 to 50 ring atoms,
R₉₀₁ to R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when two or more R₉₀₁ are present, the two or more R₉₀₁ are mutually the same or different;
when two or more R₉₀₂ are present, the two or more R₉₀₂ are mutually the same or different;
when two or more R₉₀₃ are present, the two or more R₉₀₃ are mutually the same or different;
when two or more R₉₀₄ are present, the two or more R₉₀₄ are mutually the same or different;
when two or more R₉₀₅ are present, the two or more R₉₀₅ are mutually the same or different;
when two or more R₉₀₆ are present, the two or more R₉₀₆ are mutually the same or different; and when two or more R₉₀₇ are present, the two or more R₉₀₇ are mutually the same or different.

In an exemplary embodiment, the substituent for the substituted or unsubstituted group is a group selected from the group consisting of an alkyl group having 1 to 50 carbon atoms, an aryl group having 6 to 50 ring carbon atoms, and a heterocyclic group having 5 to 50 ring atoms.

In an exemplary embodiment, the substituent for the substituted or unsubstituted group is a group selected from the group consisting of an alkyl group having 1 to 18 carbon atoms, an aryl group having 6 to 18 ring carbon atoms, and a heterocyclic group having 5 to 18 ring atoms.

Specific examples of the above optional substituent are the same as the specific examples of the substituent described in the above under the subtitle “Substituent Mentioned Herein.”

Unless otherwise specified herein, adjacent ones of the optional substituents may form a “saturated ring” or an “unsaturated ring,” preferably a substituted or unsubstituted saturated five-membered ring, a substituted or unsubstituted saturated six-membered ring, a substituted or unsubstituted unsaturated five-membered ring, or a substituted or unsubstituted unsaturated six-membered ring, more preferably a benzene ring.

Unless otherwise specified herein, the optional substituent may further include a substituent. Examples of the substituent for the optional substituent are the same as the examples of the optional substituent.

Herein, numerical ranges represented by “AA to BB” represent a range whose lower limit is the value (AA) recited before “to” and whose upper limit is the value (BB) recited after “to.”

### First Exemplary Embodiment

An arrangement of an organic EL device according to a first exemplary embodiment of the invention will be described below.

The organic EL device includes an anode, a cathode, and an organic layer between the anode and the cathode. The organic layer includes at least one layer formed from an organic compound(s). Alternatively, the organic layer includes a plurality of layers formed from an organic compound(s). The organic layer may further contain an inorganic compound(s). In the organic EL device of the exemplary embodiment, at least one layer of the organic layer is an emitting layer. For instance, the organic layer may be one emitting layer, or may further include a layer(s) usable in the organic EL device. Examples of the layer usable in the organic EL device, which are not particularly limited, include at least one selected from the group consisting of a hole injecting layer, a hole transporting layer, an electron injecting layer, an electron transporting layer, and a blocking layer.

An organic EL device according to the exemplary embodiment includes an anode, a cathode, and an emitting layer provided between the anode and the cathode in which the emitting layer contains a first compound represented by a formula (1) below and a second compound that exhibits delayed fluorescence and is represented by a formula (2) below, and a singlet energy S₁(M1) of the first compound and a singlet energy S₁(M2) of the second compound satisfy a relationship of a numerical formula (Numerical Formula 1) below.

S₁(M2)>S₁(M1) (Numerical Formula 1)

Combining the first compound represented by the formula (1) and the delayed fluorescent second compound represented by the formula (2) reduces recombination in the first compound and improves probability of exciton generation in the second compound. As a result, deterioration of the first compound is inhibitable and therefore a device having a long lifetime is achievable. Further, a luminous efficiency is improved in an exemplary organic EL device of the exemplary embodiment.

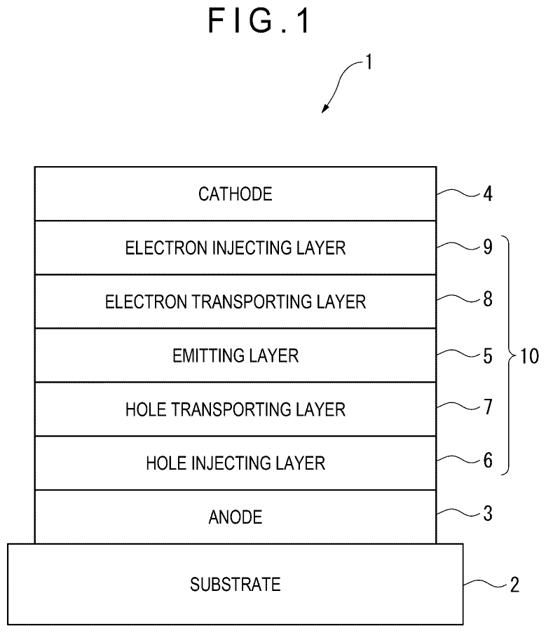

FIG. 1 schematically illustrates an exemplary arrangement of an organic EL device according to the exemplary embodiment.

An organic EL device 1 includes a light-transmissive substrate 2, an anode 3, a cathode 4, and an organic layer 10 provided between the anode 3 and the cathode 4. The organic layer 10 includes a hole injecting layer 6, a hole transporting layer 7, an emitting layer 5, an electron transporting layer 8, and an electron injecting layer 9 that are layered on the anode 3 in this order. The organic EL device of the invention may have any arrangement without being limited to the arrangement of the organic EL device illustrated in FIG. 1.

### Emitting Layer

In the organic EL device according to the exemplary embodiment, the emitting layer contains the first compound and the second compound. In this arrangement, the first compound is preferably a dopant material (also referred to as a guest material, emitter or luminescent material), and the second compound is preferably a host material (also referred to as a matrix material).

In an exemplary arrangement of the exemplary embodiment, the emitting layer may contain a metal complex.

In an exemplary arrangement of the exemplary embodiment, the emitting layer preferably does not contain a phosphorescent material (phosphorescent dopant material).

In an exemplary arrangement of the exemplary embodiment, the emitting layer preferably does not contain a heavy-metal complex and a phosphorescent rare earth metal complex. Examples of the heavy-metal complex herein include iridium complex, osmium complex, and platinum complex.

In an exemplary arrangement of the exemplary embodiment, the emitting layer also preferably does not contain a metal complex.

### First Compound

The first compound is represented by the formula (1) below.

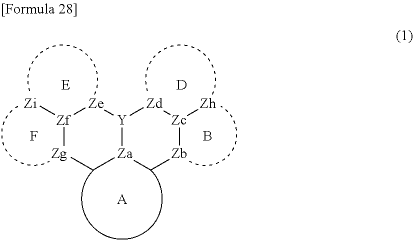

In the formula (1):
a ring A, a ring B, a ring D, a ring E, and a ring F are each independently a cyclic structure selected from the group consisting of a substituted or unsubstituted aryl ring having 6 to 30 ring carbon atoms, and a substituted or unsubstituted heterocyclic ring having 5 to 30 ring atoms,
one of the ring B and the ring D is present or both of the ring B and the ring D are present;
when both of the ring B and the ring D are present, the ring B and the ring D share a bond connecting Zc to Zh;
one of the ring E and the ring F is present or both of the ring E and the ring F are present;
when both of the ring E and the ring F are present, the ring E and the ring F share a bond connecting Zf to Zi;
Za is a nitrogen atom or a carbon atom;
Zb is a nitrogen atom or a carbon atom when the ring B is present, and Zb is an oxygen atom, a sulfur atom, NRb, C(Rb₁)(Rb₂), or Si(Rb₃)(Rb₄) when the ring B is not present;
Zc is a nitrogen atom or a carbon atom;
Zd is a nitrogen atom or a carbon atom when the ring D is present, and Zd is an oxygen atom, a sulfur atom, or NRd when the ring D is not present;
Ze is a nitrogen atom or a carbon atom when the ring E is present, and Ze is an oxygen atom, a sulfur atom, or NRe when the ring E is not present;
Zf is a nitrogen atom or a carbon atom;
Zg is a nitrogen atom or a carbon atom when the ring F is present, and Zg is an oxygen atom, a sulfur atom, NRg, C(Rg₁)(Rg₂), or Si(Rg₃)(Rg₄) when the ring F is not present;
Zh is a nitrogen atom or a carbon atom;
Zi is a nitrogen atom or a carbon atom;
Y is a boron atom, a phosphorus atom, SiRh, P═O, or P═S;
Rb, Rb₁, Rb₂, Rb₃, Rb₄, Rd, Re, Rg, Rg₁, Rg₂, Rg₃, Rg₄, and Rh are each independently a hydrogen atom or a substituent;
Rb, Rb₁, Rb₂, Rb₃, Rb₄, Rd, Re, Rg, Rg₁, Rg₂, Rg₃, Rg₄, and Rh as a substituent are each independently a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted cycloalkyl group having 3 to 30 ring carbon atoms, a group represented by —Si(R₉₁₁)(R₉₁₂)(R₉₁₃), a group represented by —O—(R₉₁₄), a group represented by —S—(R₉₁₅), or a group represented by —N(R₉₁₆)(R₉₁₇); and
a bond between Y and Za, a bond between Y and Zd, and a bond between Y and Ze are each a single bond.

A bond between Y and Za, a bond between Y and Zd, and a bond between Y and Ze are each a single bond and the single bond is not a coordinate bond but a covalent bond.

Herein, a heterocyclic ring is exemplified by a cyclic structure (heterocyclic ring) obtained by removing a bond from a “heterocyclic group” exemplified by the above “Substituent Mentioned Herein.” The heterocyclic ring may be substituted or unsubstituted.

Herein, an aryl ring is exemplified by a cyclic structure (aryl ring) obtained by removing a bond from an “aryl group” exemplified by the above “Substituent Mentioned Herein.” The aryl ring may be substituted or unsubstituted.

In the formula (1), it is also preferable that both of the ring B and the ring D are present and both of the ring E and the ring F are present. With this arrangement, a compound represented by the formula (1) is represented by a formula (1-1) below.

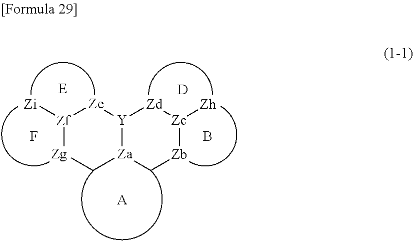

In the formulae (1-1):
a ring A, a ring B, a ring D, a ring E, and a ring F are each independently a cyclic structure selected from the group consisting of a substituted or unsubstituted aryl ring having 6 to 30 ring carbon atoms, and a substituted or unsubstituted heterocyclic ring having 5 to 30 ring atoms;
the ring B and the ring D share a bond connecting Zc to Zh;
the ring E and the ring F share a bond connecting Zf to Zi;
Za, Zb, Zc, Zd, Ze, Zf, Zg, Zh, and Zi are each independently a nitrogen atom or a carbon atom;
Y is a boron atom, a phosphorus atom, SiRh, P═O, or P═S; and
a bond between Y and Za, a bond between Y and Zd, and a bond between Y and Ze are each a single bond.

Y in the formula of the first compound is preferably a boron atom.

Zb and Zg in the formula of the first compound is preferably a nitrogen atom.

In the exemplary embodiment, it is preferable that Rb, Rb₁, Rb₂, Rb₃, Rb₄, Rd, Re, Rg, Rg₁, Rg₂, Rg₃, Rg₄, and Rh are each independently a hydrogen atom, a substituted or unsubstituted aryl group having 6 to 30 ring carbon atoms, or a substituted or unsubstituted heterocyclic group having 5 to 30 ring atoms.

In the exemplary embodiment, it is preferable that Rb, Rb₁, Rb₂, Rb₃, Rb₄, Rd, Re, Rg, Rg₁, Rg₂, Rg₃, Rg₄, and Rh are each independently a hydrogen atom or a substituted or unsubstituted aryl group having 6 to 30 ring carbon atoms.

The first compound of the exemplary embodiment is also preferably a compound represented by a formula (11) below.

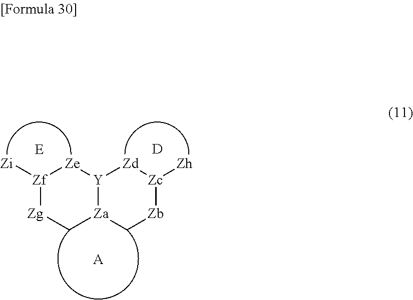

In the formula (11):
a ring A, a ring D, and a ring E are each independently a cyclic structure selected from the group consisting of a substituted or unsubstituted aryl ring having 6 to 30 ring carbon atoms, and a substituted or unsubstituted heterocyclic ring having 5 to 30 ring atoms;
Za is a nitrogen atom or a carbon atom;
Zb is an oxygen atom, a sulfur atom, NRb, C(Rb₁)(Rb₂), or Si(Rb₃)(Rb₄);
Zc is a nitrogen atom or a carbon atom;
Zd is a nitrogen atom or a carbon atom;
Ze is a nitrogen atom or a carbon atom;
Zf is a nitrogen atom or a carbon atom;
Zg is an oxygen atom, a sulfur atom, NRg, C(Rg₁)(Rg₂), or Si(Rg₃)(Rg₄);
Zh is a nitrogen atom or a carbon atom;
Zi is a nitrogen atom or a carbon atom;
Y is a boron atom, a phosphorus atom, SiRh, P═O, or P═S; and
Rb, Rb₁, Rb₂, Rb₃, Rb₄, Rg, Rg₁, Rg₂, Rg₃, Rg₄, and Rh each independently represent the same as Rb, Rb₁, Rb₂, Rb₃, Rb₄, Rg, Rg₁, Rg₂, Rg₃, Rg₄, and Rh in the formula (1).

The first compound of the exemplary embodiment is also preferably a compound represented by a formula (12) below.

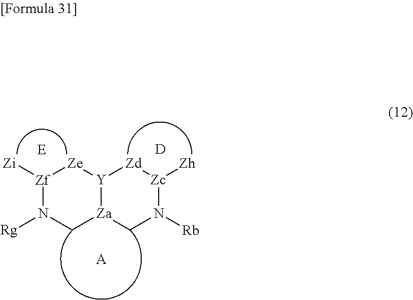

In the formula (12):
a ring A, a ring D, and a ring E are each independently a cyclic structure selected from the group consisting of a substituted or unsubstituted aryl ring having 6 to 30 ring carbon atoms, and a substituted or unsubstituted heterocyclic ring having 5 to 30 ring atoms;
Za, Zc, Zd, Ze, Zf, Zh, and Zi are each independently a nitrogen atom or a carbon atom; and
Rb and Rg each independently represent the same as Rb and Rg in the formula (1).

The first compound of the exemplary embodiment is also preferably a compound represented by a formula (13) below.

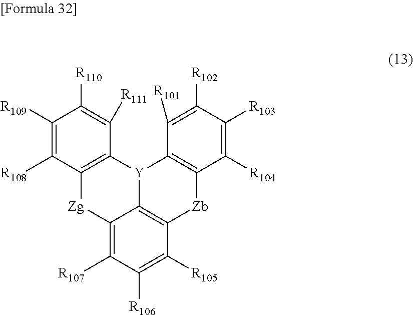

In the formula (13):
Zb is an oxygen atom, a sulfur atom, or NRb;
Zg is an oxygen atom, a sulfur atom, or NRg;
Y is a boron atom, a phosphorus atom, SiRh, P═O, or P═S;
Rb, Rg, and Rh each independently represent the same as Rb, Rg, and Rh in the formula (1);
R₁₀₁ to R₁₁₁ are each independently a hydrogen atom or a substituent; and
R₁₀₁ to R₁₁₁ as a substituent are each independently a group selected from the group consisting of a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted haloalkyl group having 1 to 30 carbon atoms, a substituted or unsubstituted aryloxy group having 6 to 30 ring carbon atoms, a group represented by —Si(R₉₀₁)(R₉₀₂)(R₉₀₃), a group represented by —N(R₉₀₆)(R₉₀₇), a cyano group, and a halogen atom.

In the first compound, R₉₀₁, R₉₀₂, R₉₀₃, R₉₀₆, and R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

The first compound of the exemplary embodiment is also preferably a compound represented by a formula (14) below.

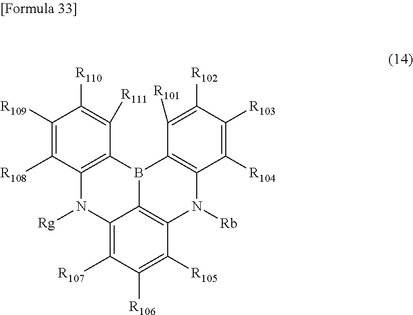

In the formula (14):
R₁₀₁ to R₁₁₁ each independently represent the same as R₁₀₁ to R₁₁₁ in the formula (13); and
Rb and Rg each independently represent the same as Rb and Rg in the formula (1).

In the formulae (12) to (14), Rb and Rg are preferably each independently a substituted or unsubstituted aryl group having 6 to 30 ring carbon atoms, more preferably a substituted or unsubstituted phenyl group.

In the first compound, R₁₀₁ to R₁₁₁ are preferably each independently a hydrogen atom, a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon atoms.

In the first compound, R₁₀₁ to R₁₁₁ are preferably each independently a hydrogen atom or a substituted or unsubstituted aryl group having 6 to 30 ring carbon atoms.

In the first compound, R₁₀₁ to R₁₁₁ are preferably each a hydrogen atom.

The first compound of the exemplary embodiment is also preferably a compound represented by a formula (15) below.

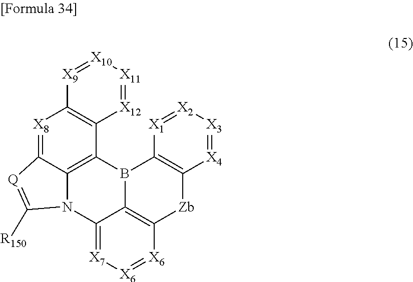

In the formula (15):
Zb is an oxygen atom, a sulfur atom, NRb, C(Rb₁)(Rb₂), or Si(Rb₃)(Rb₄);
X₁ is CR₁₂₁, a nitrogen atom, or a carbon atom bonded to X₁₂ with a single bond;
X₂ is CR₁₂₂ or a nitrogen atom;
X₃ is CR₁₂₃ or a nitrogen atom;
X₄ is CR₁₂₄ or a nitrogen atom;
X₅ is CR₁₂₅ or a nitrogen atom;
X₆ is CR₁₂₆ or a nitrogen atom;
X₇ is CR₁₂₇ or a nitrogen atom;
X₈ is CR₁₂₈ or a nitrogen atom;
X₉ is CR₁₂₉ or a nitrogen atom;
X₁₀ is CR₁₃₀ or a nitrogen atom;
X₁₁ is CR₁₃₁ or a nitrogen atom;
X₁₂ is CR₁₃₂, a nitrogen atom, or a carbon atom bonded to X₁ with a single bond;
Q is CR_{Q} or a nitrogen atom;
at least one combination of adjacent two or more of R₁₂₂ to R₁₃₁ are mutually bonded to form a substituted or unsubstituted monocyclic ring, mutually bonded to form a substituted or unsubstituted fused ring, or not mutually bonded;
at least one combination of adjacent two or more of R₁₂₄, R₁₂₅, Rb, Rb₁, Rb₂, Rb₃, and Rb₄ are mutually bonded to form a substituted or unsubstituted monocyclic ring, mutually bonded to form a substituted or unsubstituted fused ring, or not mutually bonded;
at least one hydrogen atom in a monocyclic ring or a fused ring formed by mutually bonding at least one combination of adjacent two or more of R₁₂₄, R₁₂₅, Rb, Rb₁, Rb₂, Rb₃, and Rb₄ is substituted or not substituted by at least one substituent selected from the group consisting of an alkyl group having 1 to 50 carbon atoms, an aryl group having 6 to 50 ring carbon atoms, a heterocyclic group having 5 to 50 ring atoms, a group represented by —O—(R₁₅₁), and a group represented by —N(R₁₅₂)(R₁₅₃);
at least one hydrogen atom of the substituent is substituted or not substituted by an aryl group having 6 to 50 ring carbon atoms or an alkyl group having 1 to 50 carbon atoms;
R₁₂₁ to R₁₃₂ forming neither the substituted or unsubstituted monocyclic ring nor the substituted or unsubstituted fused ring, R₁₅₀, and R_{Q} are each independently a hydrogen atom, a substituted or unsubstituted alkyl group having 1 to 50 carbon atoms, a substituted or unsubstituted cycloalkyl group having 3 to 50 ring carbon atoms, a group represented by —Si(R₉₅₁)(R₉₅₂)(R₉₅₃), a group represented by —O—(R₉₅₄), a group represented by —S—(R₉₅₅), a group represented by —N(R₉₅₆)(R₉₅₇), a substituted or unsubstituted aralkyl group having 7 to 50 carbon atoms, a group represented by —C(═O)R₉₅₈, a group represented by —COOR₉₅₉, a halogen atom, a cyano group, a nitro group, a substituted or unsubstituted aryl group having 6 to 50 ring carbon atoms, or a substituted or unsubstituted heterocyclic group having 5 to 50 ring atoms;
Rb, Rb₁, Rb₂, Rb₃, and Rb₄ forming neither the substituted or unsubstituted monocyclic ring nor the substituted or unsubstituted fused ring are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
R₁₅₁ to R₁₅₃ and R₉₅₁ to R₉₅₉ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₁₅₁ are present, the plurality of R₁₅₁ are mutually the same or different;
when a plurality of R₁₅₂ are present, the plurality of R₁₅₂ are mutually the same or different;
when a plurality of R₁₅₃ are present, the plurality of R₁₅₃ are mutually the same or different;
when a plurality of R₉₅₁ are present, the plurality of R₉₅₁ are mutually the same or different;
when a plurality of R₉₅₂ are present, the plurality of R₉₅₂ are mutually the same or different;
when a plurality of R₉₅₃ are present, the plurality of R₉₅₃ are mutually the same or different;
when a plurality of R₉₅₄ are present, the plurality of R₉₅₄ are mutually the same or different;
when a plurality of R₉₅₅ are present, the plurality of R₉₅₅ are mutually the same or different;
when a plurality of R₉₅₆ are present, the plurality of R₉₅₆ are mutually the same or different;
when a plurality of R₉₅₇ are present, the plurality of R₉₅₇ are mutually the same or different;
when a plurality of R₉₅₈ are present, the plurality of R₉₅₈ are mutually the same or different; and
when a plurality of R₉₅₉ are present, the plurality of R₉₅₉ are mutually the same or different.

In the first compound represented by the formula (15), when X₁ is a carbon atom bonded to X₁₂ with a single bond and X₁₂ is a carbon atom bonded to X₁ with a single bond, the first compound is represented by a formula (15A) below.

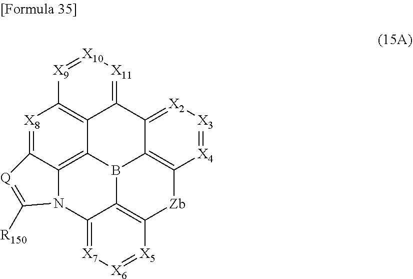

In the formula (15A), Zb, X₂ to X₁₁, R₁₅₀, and Q are each independently as defined in the formula (15).

It is preferable that R₁₂₁ to R₁₃₂, R₁₅₀, and R_{Q} in the first compound represented by the formula (15) are each independently a hydrogen atom, a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon atoms.

It is preferable that R₁₂₁ to R₁₃₂, R₁₅₀, and R_{Q} in the first compound represented by the formula (15) are each independently a hydrogen atom or a substituted or unsubstituted aryl group having 6 to 30 ring carbon atoms.

In the first compound represented by the formula (15), R₁₂₁ to R₁₃₂, R₁₅₀, and R_{Q} are preferably each a hydrogen atom.

The first compound represented by the formula (15) is also preferably a compound represented by a formula (151) below.

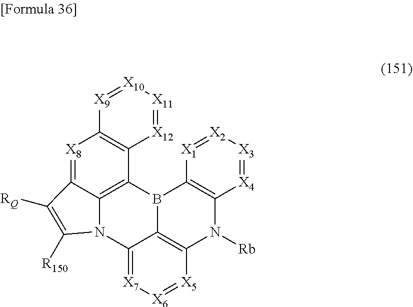

In the formula (151): X₁ to X₁₂ respectively represent the same as X₁ to X₁₂ in the formula (15); and R₁₅₀, R_{Q}, and Rb respectively represent the same as R₁₅₀, R_{Q}, and Rb in the formula (15).

The first compound represented by the formula (15) is also preferably a compound represented by a formula (152) below.

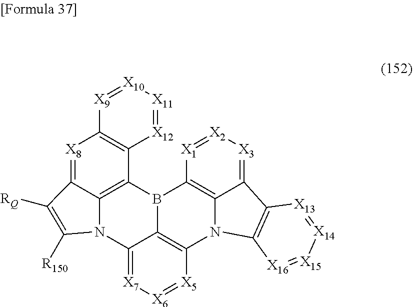

In the formula (152):
X₁ to X₃ and X₅ to X₁₂ respectively represent the same as X₁ to X₃ and X₅ to X₁₂ in the formula (15);
X₁₃ is CR₁₃₃ or a nitrogen atom;
X₁₄ is CR₁₃₄ or a nitrogen atom;
X₁₅ is CR₁₃₅ or a nitrogen atom;
X₁₆ is CR₁₃₆ or a nitrogen atom;
R₁₃₃ to R₁₃₆ are each independently a hydrogen atom, a substituted or unsubstituted alkyl group having 1 to 30 carbon atoms, a substituted or unsubstituted cycloalkyl group having 3 to 50 ring carbon atoms, a group represented by —Si(R₉₅₁)(R₉₅₂)(R₉₅₃), a group represented by —O—(R₉₅₄), a group represented by —S—(R₉₅₅), a group represented by —N(R₉₅₆)(R₉₅₇), a substituted or unsubstituted aralkyl group having 7 to 50 carbon atoms, a group represented by —C(═O)R₉₅₈, a group represented by —COOR₉₅₉, a halogen atom, a cyano group, a nitro group, a substituted or unsubstituted aryl group having 6 to 50 ring carbon atoms, or a substituted or unsubstituted heterocyclic group having 5 to 50 ring atoms; and
R₁₅₀ to R_{Q} respectively represent the same as R₁₅₀ to R_{Q} in the formula (15).

R₁₃₃ to R₁₃₆ in the first compound represented by the formula (15) are preferably each independently a hydrogen atom, a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon atoms.

R₁₃₃ to R₁₃₆ in the first compound represented by the formula (15) are preferably each independently a hydrogen atom or a substituted or unsubstituted aryl group having 6 to 30 ring carbon atoms.

In the first compound, R₁₃₃ to R₁₃₆ are preferably each a hydrogen atom.

The first compound represented by the formula (15) is also preferably a compound represented by a formula (153) below.

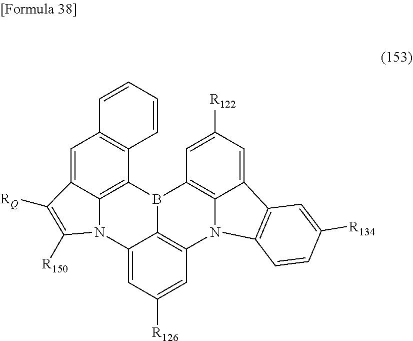

In the formula (153), R₁₂₂, R₁₂₆, R₁₃₄, R₁₅₀, and R_{Q} are each independently a hydrogen atom, a substituted or unsubstituted alkyl group having 1 to 50 carbon atoms, a substituted or unsubstituted cycloalkyl group having 3 to 50 ring carbon atoms, a group represented by —Si(R₉₅₁)(R₉₅₂)(R₉₅₃), a group represented by —O—(R₉₅₄), a group represented by —S—(R₉₅₅), a group represented by —N(R₉₅₆)(R₉₅₇), a substituted or unsubstituted aralkyl group having 7 to 50 carbon atoms, a group represented by —C(═O)R₉₅₈, a group represented by —COOR₉₅₉, a halogen atom, a cyano group, a nitro group, a substituted or unsubstituted aryl group having 6 to 50 ring carbon atoms, or a substituted or unsubstituted heterocyclic group having 5 to 50 ring atoms, R₉₅₁ to R₉₅₉ being defined as in the formula (15).

In the formula (153), it is also preferable that R₁₂₂, R₁₂₆, R₁₃₄, R₁₅₀, and R_{Q} are each independently a hydrogen atom, a substituted or unsubstituted alkyl group having 1 to 10 carbon atoms, a substituted or unsubstituted aryl group having 6 to 12 ring carbon atoms, or a substituted or unsubstituted heterocyclic group having 5 to 18 ring atoms.

In the formula (153), it is also preferable that R₁₂₂ and R₁₃₄ are preferably each independently a hydrogen atom or a substituted or unsubstituted alkyl group having 1 to 10 carbon atoms.

In the formula (153), it is also preferable that R₁₂₆, R₁₅₀, and R_{Q} are each independently a substituted or unsubstituted aryl group having 6 to 12 ring carbon atoms.

The first compound of the exemplary embodiment is also preferably a compound represented by a formula (16) below.

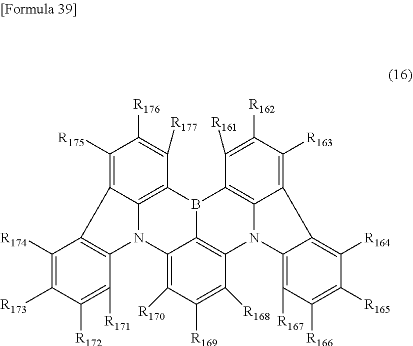

In the formula (16):
at least one combination of adjacent two or more of R₁₆₁ to R₁₇₇ are mutually bonded to form a substituted or unsubstituted monocyclic ring, mutually bonded to form a substituted or unsubstituted fused ring, or not mutually bonded;
R₁₆₁ to R₁₇₇ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aralkyl group having 7 to 50 carbon atoms, a group represented by —Si(R₉₆₁)(R₉₆₂)(R₉₆₃), a group represented by —O—(R₉₆₄), a group represented by —S—(R₉₆₅), a group represented by —N(R₉₆₆)(R₉₆₇), a group represented by —C(═O)R₉₆₈, a group represented by —COOR₉₆₉, a halogen atom, a cyano group, a nitro group, a substituted or unsubstituted aryl group having 6 to 50 ring carbon atoms, or a substituted or unsubstituted heterocyclic group having 5 to 50 ring atoms;
R₉₆₁ to R₉₆₉ are each independently a hydrogen atom, a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms;
when a plurality of R₉₆₁ are present, the plurality of R₉₆₁ are mutually the same or different;
when a plurality of R₉₆₂ are present, the plurality of R₉₆₂ are mutually the same or different;
when a plurality of R₉₆₃ are present, the plurality of R₉₆₃ are mutually the same or different;
when a plurality of R₉₆₄ are present, the plurality of R₉₆₄ are mutually the same or different;
when a plurality of R₉₆₅ are present, the plurality of R₉₆₅ are mutually the same or different;
when a plurality of R₉₆₆ are present, the plurality of R₉₆₆ are mutually the same or different;
when a plurality of R₉₆₇ are present, the plurality of R₉₆₇ are mutually the same or different;
when a plurality of R₉₆₈ are present, the plurality of R₉₆₈ are mutually the same or different; and
when a plurality of R₉₆₉ are present, the plurality of R₉₆₉ are mutually the same or different.

R₁₆₁ to R₁₇₇ in the first compound represented by the formula (16) are preferably each independently a hydrogen atom, a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon atoms.

In the first compound represented by the formula (16), at least one of R₁₆₈ to R₁₇₀ is preferably a substituted or unsubstituted aryl group having 6 to 50 ring carbon atoms or a substituted or unsubstituted heterocyclic group having 5 to 50 ring atoms.

R₁₆₁ to R₁₇₇ in the first compound represented by the formula (16) are preferably each independently a hydrogen atom or a substituted or unsubstituted aryl group having 6 to 30 ring carbon atoms.

In the first compound represented by the formula (16), R₁₆₁ to R₁₇₇ are also preferably each a hydrogen atom.

In the first compound represented by the formula (16), it is also preferable that at least one combination of adjacent two or more of R₁₆₁ to R₁₇₇ are mutually bonded to form a ring represented by a formula (16A) below.

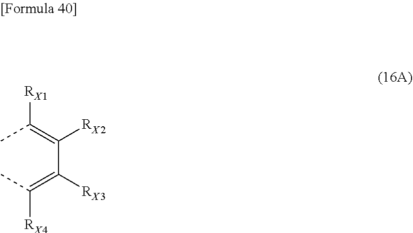

In the formula (16A): a dotted line represents a bonding position; and R_{X1} to R_{X4} are Each Independently a Hydrogen Atom, a Substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aralkyl group having 7 to 50 carbon atoms, a group represented by —Si(R₉₆₁)(R₉₆₂)(R₉₆₃), a group represented by —O—(R₉₆₄), a group represented by —S—(R₉₆₅), a group represented by —N(R₉₆₆)(R₉₆₇), a group represented by —C(═O)Rees, a group represented by —COOR₉₆₉, a halogen atom, a cyano group, a nitro group, a substituted or unsubstituted aryl group having 6 to 50 ring carbon atoms, or a substituted or unsubstituted heterocyclic group having 5 to 50 ring atoms.

When a plurality of R_{X1} are present, the plurality of R_{X1} are mutually the same or different.

When a plurality of R_{X2} are present, the plurality of R_{X2} are mutually the same or different.

When a plurality of R_{X3} are present, the plurality of R_{X3} are mutually the same or different.

When a plurality of R_{X4} are present, the plurality of R_{X4} are mutually the same or different.

In the first compound represented by the formula (16): it is also preferable that at least one combination of a combination of R₁₆₁ and R₁₆₂, a combination of R₁₆₅ and R₁₆₆, a combination of R₁₇₂ and R₁₇₃, or a combination of R₁₇₆ and R₁₇₇ are mutually bonded to form a ring represented by the formula (16A).

In the first compound represented by the formula (16), it is preferable that a combination of R₁₆₁ and R₁₆₂ and a combination of R₁₇₆ and R₁₇₇ do not form a ring represented by the formula (16A) concurrently.

In the first compound represented by the formula (16), it is also preferable that a combination of R₁₆₅ and R₁₆₆ are mutually bonded to form a ring represented by the formula (16A) and a combination of R₁₇₂ and R₁₇₃ are mutually bonded to form a ring represented by the formula (16A). The first compound with this arrangement is represented by a formula (161) below.

The first compound represented by the formula (16) is also preferably a compound represented by the formula (161) below.

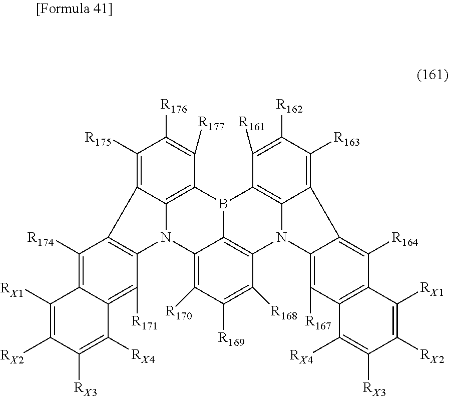

In the formula (161), R₁₆₁ to R₁₆₄, R₁₆₇ to R₁₇₁, R₁₇₄ to R₁₇₇, and R_{X1} to R_{X4} each independently represent the same as R₁₆₁ to R₁₆₄, R₁₆₇ to R₁₇₁, and R₁₇₄ to R₁₇₇ in the formula (16) and R_{X1} to R_{X4} in the formula (16A).

The first compound represented by the formula (16) is also preferably a compound represented by a formula (162) below.

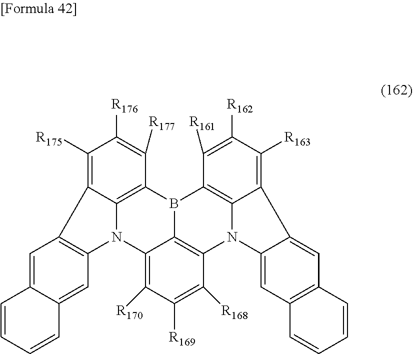

In the formula (162): R₁₆₁ to R₁₆₃, R₁₆₈ to R₁₇₀, and R₁₇₅ to R₁₇₇ each independently represent the same as R₁₆₁ to R₁₆₃, R₁₆₈ to R₁₇₀, and R₁₇₅ to R₁₇₇ in the formula (16).

The first compound represented by the formula (16) is also preferably a compound represented by a formula (163) below.

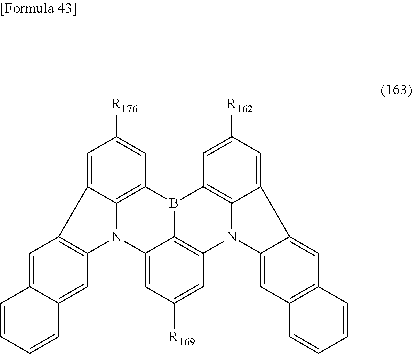

In the formula (163), R₁₆₂, R₁₆₉, and R₁₇₆ each independently represent the same as R₁₆₂, R₁₆₉, and R₁₇₆ in the formula (16).

In the formulae (16) and (161) to (163) representing the first compound, R₁₆₉ is also preferably a substituted or unsubstituted aryl group having 6 to 30 ring carbon atoms.

In the formulae (16) and (161) to (163) representing the first compound, R₁₆₉ is also preferably a substituted or unsubstituted phenyl group.

The first compound of the exemplary embodiment is also preferably a compound represented by a formula (171) or formula (172) below.

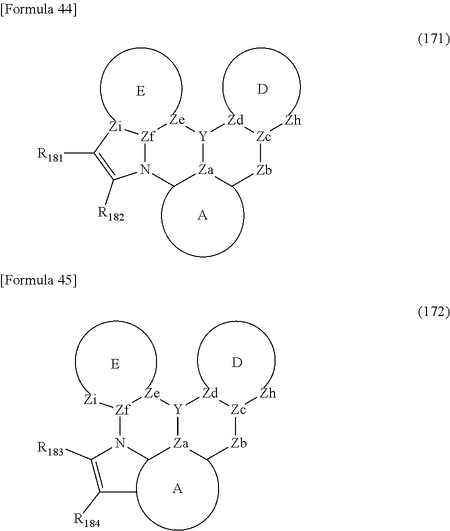

In the formulae (171) and (172):
a ring A, a ring D, and a ring E are each independently a cyclic structure selected from the group consisting of a substituted or unsubstituted aryl ring having 6 to 30 ring carbon atoms, and a substituted or unsubstituted heterocyclic ring having 5 to 30 ring atoms;
Za is a nitrogen atom or a carbon atom;
Zb is an oxygen atom, a sulfur atom, or NRb;
Zc is a nitrogen atom or a carbon atom;
Zd is a carbon atom or a nitrogen atom;
Ze is a carbon atom or a nitrogen atom;
Zf is a nitrogen atom or a carbon atom;
Zh is a nitrogen atom or a carbon atom;
Zi is a nitrogen atom or a carbon atom;
Y is a boron atom, a phosphorus atom, SiRh, P═O, or P═S;
Rb and Rh are each independently a hydrogen atom or a substituent;
Rb and Rh as a substituent are each independently selected from the group consisting of a substituted or unsubstituted aryl group having 6 to 30 ring carbon atoms, a substituted or unsubstituted heterocyclic group having 5 to 30 ring atoms, and a substituted or unsubstituted alkyl group having 1 to 30 carbon atoms;
a combination of R₁₈₁ and R₁₈₂ are mutually bonded to form a substituted or unsubstituted monocyclic ring, mutually bonded to form a substituted or unsubstituted fused ring, or not mutually bonded;
a combination of R₁₈₃ and R₁₈₄ are mutually bonded to form a substituted or unsubstituted monocyclic ring, mutually bonded to form a substituted or unsubstituted fused ring, or not mutually bonded;
R₁₈₁ to R₁₈₄ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aralkyl group having 7 to 50 carbon atoms, a group represented by —Si(R₉₇₁)(R₉₇₂)(R₉₇₃), a group represented by —O—(R₉₇₄), a group represented by —S—(R₉₇₅), a group represented by —N(R₉₇₆)(R₉₇₇), a group represented by —C(═O)R₉₇₈, a group represented by —COOR₉₇₉, a halogen atom, a cyano group, a nitro group, a substituted or unsubstituted aryl group having 6 to 50 ring carbon atoms, or a substituted or unsubstituted heterocyclic group having 5 to 50 ring atoms;
R₉₇₁ to R₉₇₉ are each independently a hydrogen atom, a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms;
when a plurality of R₉₇₁ are present, the plurality of R₉₇₁ are mutually the same or different;
when a plurality of R₉₇₂ are present, the plurality of R₉₇₂ are mutually the same or different;
when a plurality of R₉₇₃ are present, the plurality of R₉₇₃ are mutually the same or different;
when a plurality of R₉₇₄ are present, the plurality of R₉₇₄ are mutually the same or different;
when a plurality of R₉₇₅ are present, the plurality of R₉₇₅ are mutually the same or different;
when a plurality of R₉₇₆ are present, the plurality of R₉₇₆ are mutually the same or different;
when a plurality of R₉₇₇ are present, the plurality of R₉₇₇ are mutually the same or different;
when a plurality of R₉₇₈ are present, the plurality of R₉₇₈ are mutually the same or different; and
when a plurality of R₉₇₉ are present, the plurality of R₉₇₉ are mutually the same or different.

In the first compound represented by the formula (171) or (172), R₁₈₁ to R₁₈₄ are preferably each independently a hydrogen atom, a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon atoms.

In the first compound represented by the formula (171) or (172), R₁₈₁ to R₁₈₄ are preferably each independently a hydrogen atom or a substituted or unsubstituted aryl group having 6 to 30 ring carbon atoms.

In the first compound represented by the formula (171) or (172), R₁₈₁ to R₁₈₄ are each preferably a hydrogen atom.

The first compound of the exemplary embodiment is also preferably a compound represented by a formula (18) below.

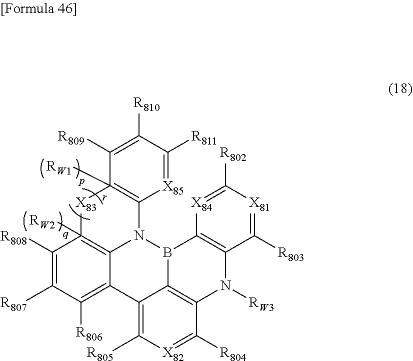

In the formula (18):
r is 0 or 1;
when r is 0, p and q are 1 and R_{W1} and R_{W2} are present;
when r is 1, p and q are 0 and R_{W1} and R_{W2} are not present;
X₈₁ is a nitrogen atom or CR₁₉₁;
X₈₂ is a nitrogen atom or CR₁₉₂;
X₈₃ is a single bond, an oxygen atom, a sulfur atom, Si(R₁₉₃)(R₁₉₄), C(R₁₉₅)(R₁₉₆) or BR₁₉₇;
X₈₄ is R₈₀₁ or a carbon atom bonded to X₈₅ with a single bond;
X₈₅ is R₈₁₂ or a carbon atom bonded to X₈₄ with a single bond;
at least one combination of adjacent two or more of R₁₉₁, R₁₉₂, R₁₉₃, R₁₉₄, R₁₉₅, R₁₉₆, R₁₉₇, R₈₀₁, R₈₀₂, R₈₀₃, R₈₀₄, R₈₀₅, R₈₀₆, R₈₀₇, R₈₀₈, R₈₀₉, R₈₁₀, R₈₁₁, R₈₁₂, R_{W1}, R_{W2}, and R_{W3} are mutually bonded to form a substituted or unsubstituted monocyclic ring, mutually bonded to form a substituted or unsubstituted fused ring, or not mutually bonded;
R_{W3} forming neither the substituted or unsubstituted monocyclic ring nor the substituted or unsubstituted fused ring is a substituted or unsubstituted aryl group having 6 to 50 ring carbon atoms, a substituted or unsubstituted heterocyclic group having 5 to 50 ring atoms, or a substituted or unsubstituted alkyl group having 1 to 5 carbon atoms;
R₁₉₁, R₁₉₂, R₁₉₃, R₁₉₄, R₁₉₅, R₁₉₆, R₁₉₇, R₈₀₁, R₈₀₂, R₈₀₃, R₈₀₄, R₈₀₅, R₈₀₆, R₈₀₇, R_{80 8}, R₈₀₉, R₈₁₀, R₈₁₁, R₈₁₂, R_{W1}, and R_{W2} forming neither the substituted or unsubstituted monocyclic ring nor the substituted or unsubstituted fused ring are each independently a hydrogen atom, a deuterium atom, a group represented by —Si(R₉₈₁)(R₉₈₂)(R₉₈₃), a group represented by —O—(R₉₈₄), a group represented by —S—(R₉₈₅), a group represented by —N(R₉₈₆)(R₉₈₇), a group represented by —B(R₉₈₈)(R₉₈₉), a group represented by —OSO₂(R₉₉₀), a cyano group, a halogen atom, a substituted or unsubstituted alkyl group having 1 to 50 carbon atoms, a substituted or unsubstituted cycloalkyl group having 3 to 50 ring carbon atoms, a substituted or unsubstituted fluoroalkyl group having 1 to 50 carbon atoms, a substituted or unsubstituted thioalkoxy group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted aryl group having 6 to 50 ring carbon atoms, or a substituted or unsubstituted heterocyclic group having 5 to 50 ring atoms;
R₉₈₁ to R₉₉₀ are each independently a hydrogen atom, a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms;
when a plurality of R₉₈₁ are present, the plurality of R₉₈₁ are mutually the same or different;
when a plurality of R₉₈₂ are present, the plurality of R₉₈₂ are mutually the same or different;
when a plurality of R₉₈₃ are present, the plurality of R₉₈₃ are mutually the same or different;
when a plurality of R₉₈₄ are present, the plurality of R₉₈₄ are mutually the same or different;
when a plurality of R₉₈₅ are present, the plurality of R₉₈₅ are mutually the same or different;
when a plurality of R₉₈₆ are present, the plurality of R₉₈₆ are mutually the same or different;
when a plurality of R₉₈₇ are present, the plurality of R₉₈₇ are mutually the same or different;
when a plurality of R₉₈₈ are present, the plurality of R₉₈₈ are mutually the same or different;
when a plurality of R₉₈₉ are present, the plurality of R₉₈₉ are mutually the same or different; and
when a plurality of R₉₉₀ are present, the plurality of R₉₉₀ are mutually the same or different.

The first compound represented by the formula (18) in which X₈₄ is a carbon atom bonded to X₈₅ with a single bond and X₈₅ is a carbon atom bonded to X₈₄ with a single bond is represented by a formula (18A) below.

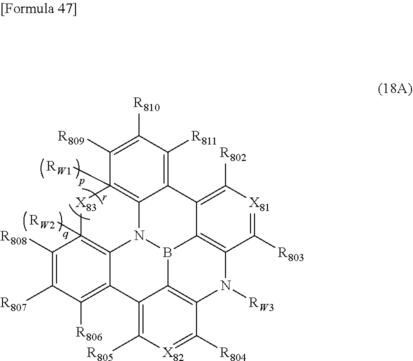

In the formula (18A), r, p, q, R₈₀₂ to R₁₁, R_{W1}, R_{W2}, R_{W3}, X₈₁, X₈₂, and X₈₃ are defined as in the formula (18).

R₁₉₁ to R₁₉₇, R₈₀₁ to R₈₁₂, R_{W1}, and R_{W2} in the first compound represented by the formula (18) are preferably each independently a hydrogen atom, a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon atoms.

R₁₉₁ to R₁₉₇, R₈₀₁ to R₈₁₂, R_{W1}, and R_{W2} in the first compound represented by the formula (18) are preferably each independently a hydrogen atom or a substituted or unsubstituted aryl group having 6 to 30 ring carbon atoms.

In the first compound represented by the formula (18), R₁₉₁ to R₁₉₇, R₈₀₁ to R₈₁₂, R_{W1}, and R_{W2} are preferably each a hydrogen atom.

The first compound represented by the formula (18) is also preferably a compound represented by a formula (181) below.

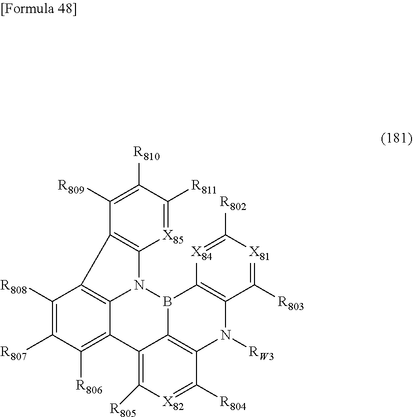

In the formula (181):
X₈₁, X₈₂, X₈₄, and X₈₅ respectively represent the same as X₈₁, X₈₂, X₈₄, and X₈₅ in the formula (18); and
R₈₀₂ to R₈₁₁ and R_{W3} respectively represent the same as R₈₀₂ to R₈₁₁ and R_{W3} in the formula (18).

The first compound represented by the formula (18) is also preferably a compound represented by a formula (182) below.

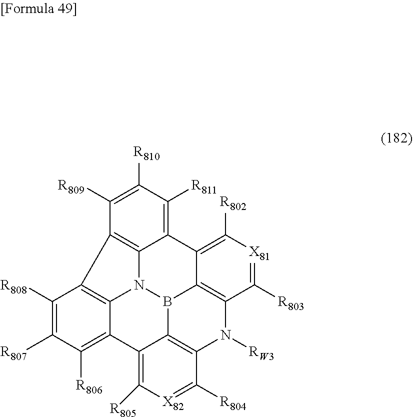

In the formula (182):
X₈₁ and X₈₂ respectively represent the same as X₈₁ and X₈₂ in the formula (18); and
R₈₀₂ to R₈₁₁ and R_{W3} respectively represent the same as R₈₀₂ to R₈₁₁ and R_{W3} in the formula (18).

In the first compound, the substituent for “the substituted or unsubstituted” group is preferably an unsubstituted alkyl group having 1 to 25 carbon atoms, an unsubstituted alkenyl group having 2 to 25 carbon atoms, an unsubstituted alkynyl group having 2 to 25 carbon atoms, an unsubstituted cycloalkyl group having 3 to 25 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), an unsubstituted aralkyl group having 7 to 50 carbon atoms, a group represented by —C(═O)R₉₀₈, a group represented by —COOR₉₀₉, a group represented by —S(═O)₂R₉₄₁, a group represented by —(═O)(R₉₄₂)(R₉₄₃), a group represented by —Ge(R₉₄₄)(R₉₄₅)(R₉₄₆), a halogen atom, a cyano group, a nitro group, an unsubstituted aryl group having 6 to 25 ring carbon atoms, or an unsubstituted heterocyclic group having 5 to 25 ring atoms.

R₉₀₁ to R₉₀₆ and R₉₄₁ to R₉₄₆ are preferably each independently a hydrogen atom, an unsubstituted alkyl group having 1 to 25 carbon atoms, an unsubstituted aryl group having 6 to 25 ring carbon atoms, or an unsubstituted heterocyclic group having 5 to 25 ring atoms.

In the first compound, the substituent for “the substituted or unsubstituted” group is preferably a halogen atom, an unsubstituted alkyl group having 1 to 25 carbon atoms, an unsubstituted aryl group having 6 to 25 ring carbon atoms, or an unsubstituted heterocyclic group having 5 to 25 ring atoms.

In the first compound, the substituent for “the substituted or unsubstituted” group is preferably an unsubstituted alkyl group having 1 to 10 carbon atoms, an unsubstituted aryl group having 6 to 12 ring carbon atoms, or an unsubstituted heterocyclic group having 5 to 12 ring atoms.

In first compound, the groups specified to be “substituted or unsubstituted” are each also preferably an “unsubstituted” group.

The maximum peak wavelength of the first compound is preferably in a range from 500 nm to 560 nm, more preferably in a range from 500 nm to 540 nm, and still more preferably in a range from 500 nm to 530 nm.

Herein, the maximum peak wavelength of the compound means a peak wavelength of a fluorescence spectrum exhibiting a maximum luminous intensity among fluorescence spectra measured in a toluene solution in which a measurement target compound is dissolved at a concentration ranging from 10⁻⁶ mol/l to 10⁻⁵ mol/l. A spectrophotofluorometer (F-7000 manufactured by Hitachi, Ltd.) can be used as a measurement apparatus.

The first compound is preferably a compound that emits green fluorescence.

The first compound is preferably a material exhibiting a high emission quantum efficiency.

### Method of Producing First Compound

The first compound of the exemplary embodiment can be produced by application of known substitution reactions and materials depending on a target compound, according to a synthesis method described in Examples described later or in a similar manner as the synthesis method.

### Specific Examples of First Compound

Specific examples of the first compound in the exemplary embodiment include compounds as follows. However, the invention is by no means limited to the specifically listed compounds.

In the specific examples of the compound herein, D represents a deuterium atom, Me represents a methyl group, tBu represents a tert-butyl group, and a Ph represents a phenyl group.

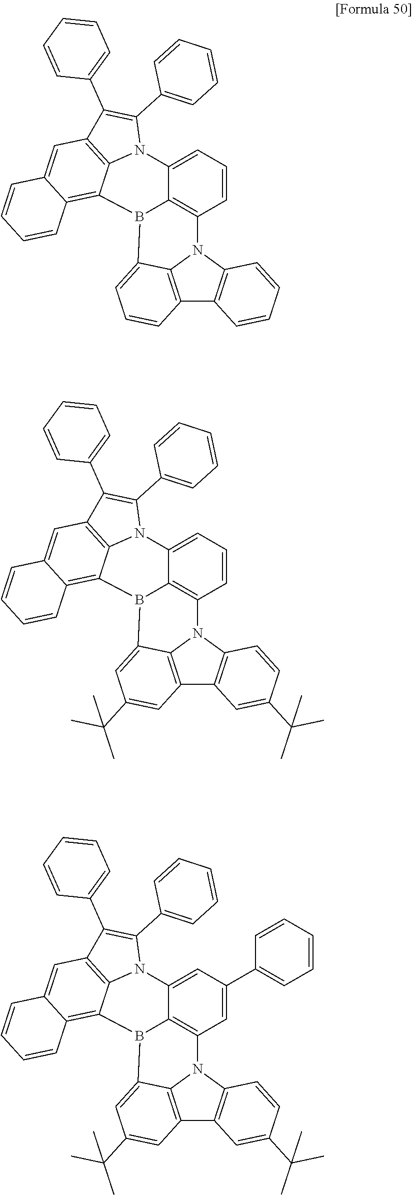 [SMILES: c1ccc(-c2c(-c3ccccc3)n3c4c(c5ccccc5cc24)B2c4c-3cccc4-n3c4ccccc4c4cccc2c43)cc1 ; CC(C)(C)c1ccc2c(c1)c1cc(C(C)(C)C)cc3c1n2-c1cccc2c1B3c1c3ccccc3cc3c(-c4ccccc4)c(-c4ccccc4)n-2c13 ; CC(C)(C)c1ccc2c(c1)c1cc(C(C)(C)C)cc3c1n2-c1cc(-c2ccccc2)cc2c1B3c1c3ccccc3cc3c(-c4ccccc4)c(-c4ccccc4)n-2c13]

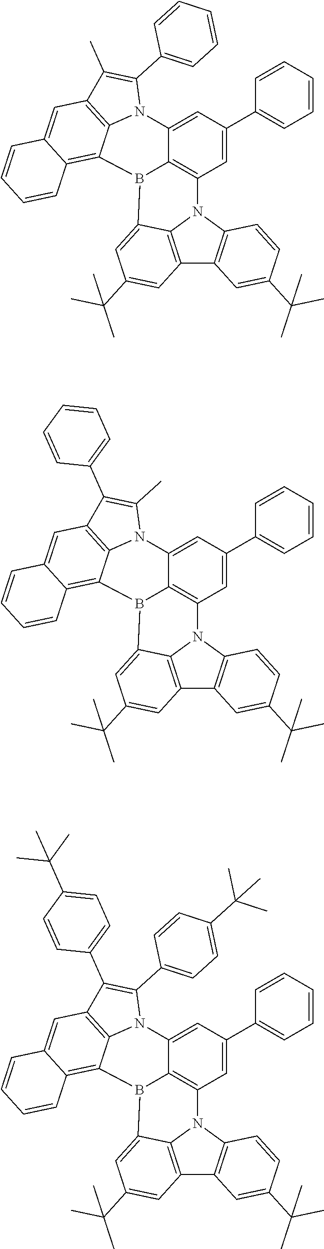 [SMILES: Cc1c(-c2ccccc2)n2c3c(c4ccccc4cc13)B1c3c-2cc(-c2ccccc2)cc3-n2c3ccc(C(C)(C)C)cc3c3cc(C(C)(C)C)cc1c32 ; Cc1c(-c2ccccc2)c2cc3ccccc3c3c2n1-c1cc(-c2ccccc2)cc2c1B3c1cc(C(C)(C)C)cc3c4cc(C(C)(C)C)ccc4n-2c13 ; CC(C)(C)c1ccc(-c2c(-c3ccc(C(C)(C)C)cc3)n3c4c(c5ccccc5cc24)B2c4c-3cc(-c3ccccc3)cc4-n3c4ccc(C(C)(C)C)cc4c4cc(C(C)(C)C)cc2c43)cc1]

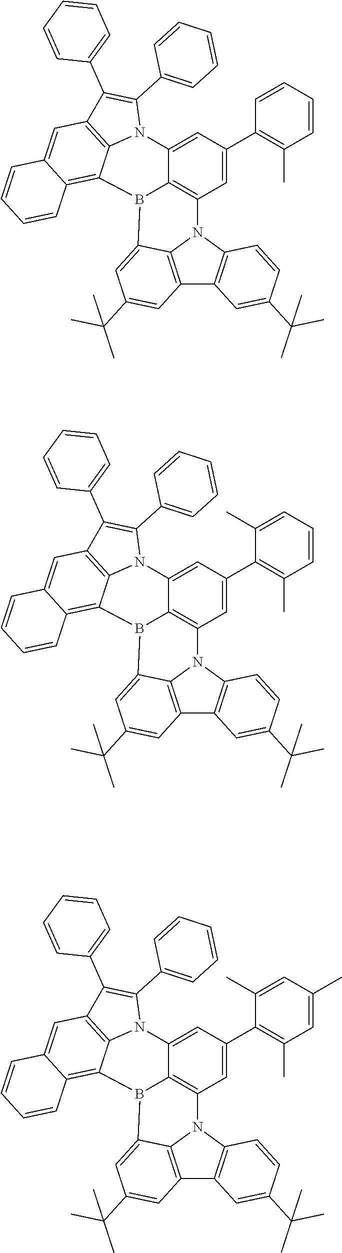 [SMILES: Cc1ccccc1-c1cc2c3c(c1)-n1c4ccc(C(C)(C)C)cc4c4cc(C(C)(C)C)cc(c41)B3c1c3ccccc3cc3c(-c4ccccc4)c(-c4ccccc4)n-2c13 ; Cc1cccc(C)c1-c1cc2c3c(c1)-n1c4ccc(C(C)(C)C)cc4c4cc(C(C)(C)C)cc(c41)B3c1c3ccccc3cc3c(-c4ccccc4)c(-c4ccccc4)n-2c13 ; Cc1cc(C)c(-c2cc3c4c(c2)-n2c5ccc(C(C)(C)C)cc5c5cc(C(C)(C)C)cc(c52)B4c2c4ccccc4cc4c(-c5ccccc5)c(-c5ccccc5)n-3c24)c(C)c1]

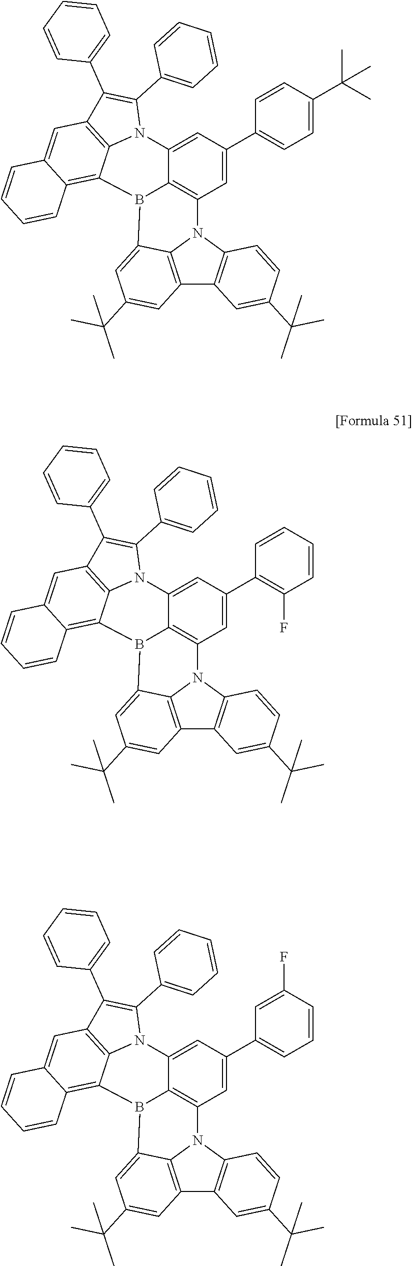 [SMILES: CC(C)(C)c1ccc(-c2cc3c4c(c2)-n2c5ccc(C(C)(C)C)cc5c5cc(C(C)(C)C)cc(c52)B4c2c4ccccc4cc4c(-c5ccccc5)c(-c5ccccc5)n-3c24)cc1 ; CC(C)(C)c1ccc2c(c1)c1cc(C(C)(C)C)cc3c1n2-c1cc(-c2ccccc2F)cc2c1B3c1c3ccccc3cc3c(-c4ccccc4)c(-c4ccccc4)n-2c13 ; CC(C)(C)c1ccc2c(c1)c1cc(C(C)(C)C)cc3c1n2-c1cc(-c2cccc(F)c2)cc2c1B3c1c3ccccc3cc3c(-c4ccccc4)c(-c4ccccc4)n-2c13]

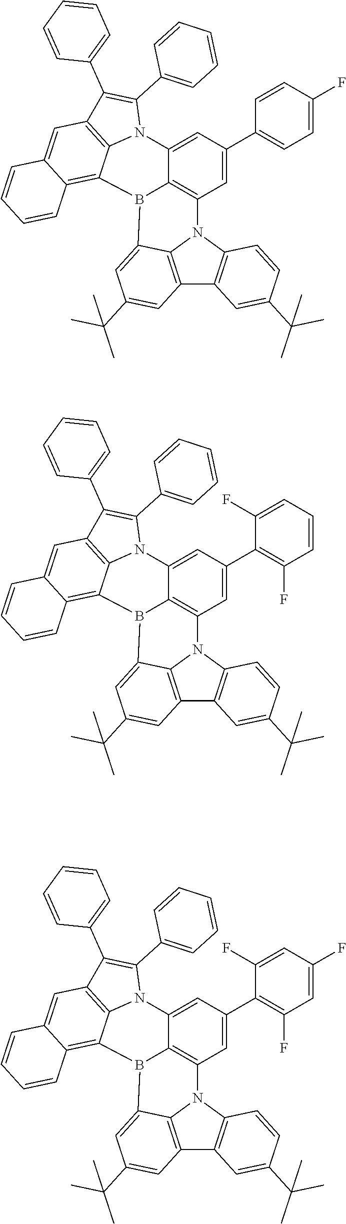 [SMILES: CC(C)(C)c1ccc2c(c1)c1cc(C(C)(C)C)cc3c1n2-c1cc(-c2ccc(F)cc2)cc2c1B3c1c3ccccc3cc3c(-c4ccccc4)c(-c4ccccc4)n-2c13 ; CC(C)(C)c1ccc2c(c1)c1cc(C(C)(C)C)cc3c1n2-c1cc(-c2c(F)cccc2F)cc2c1B3c1c3ccccc3cc3c(-c4ccccc4)c(-c4ccccc4)n-2c13 ; CC(C)(C)c1ccc2c(c1)c1cc(C(C)(C)C)cc3c1n2-c1cc(-c2c(F)cc(F)cc2F)cc2c1B3c1c3ccccc3cc3c(-c4ccccc4)c(-c4ccccc4)n-2c13]

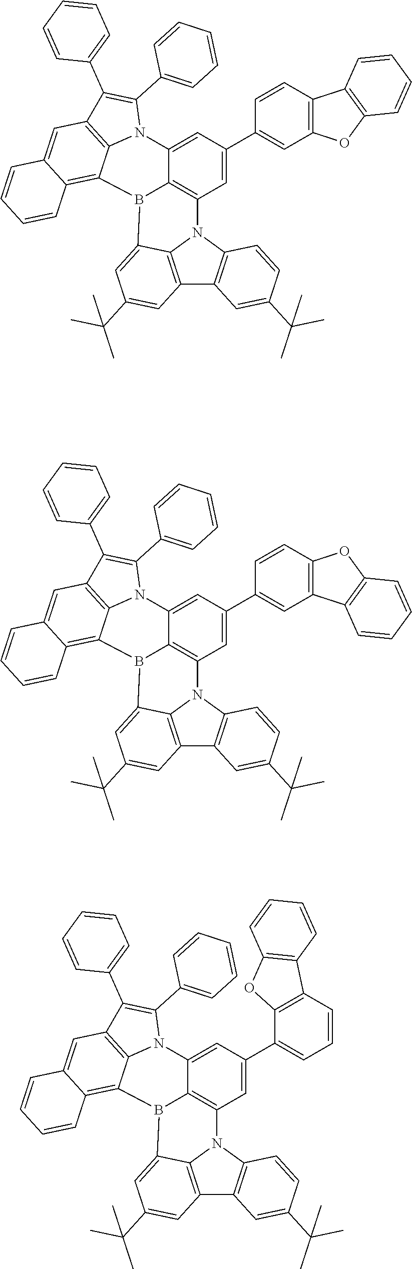 [SMILES: CC(C)(C)c1ccc2c(c1)c1cc(C(C)(C)C)cc3c1n2-c1cc(-c2ccc4c(c2)oc2ccccc24)cc2c1B3c1c3ccccc3cc3c(-c4ccccc4)c(-c4ccccc4)n-2c13 ; CC(C)(C)c1ccc2c(c1)c1cc(C(C)(C)C)cc3c1n2-c1cc(-c2ccc4oc5ccccc5c4c2)cc2c1B3c1c3ccccc3cc3c(-c4ccccc4)c(-c4ccccc4)n-2c13 ; CC(C)(C)c1ccc2c(c1)c1cc(C(C)(C)C)cc3c1n2-c1cc(-c2cccc4c2oc2ccccc24)cc2c1B3c1c3ccccc3cc3c(-c4ccccc4)c(-c4ccccc4)n-2c13]

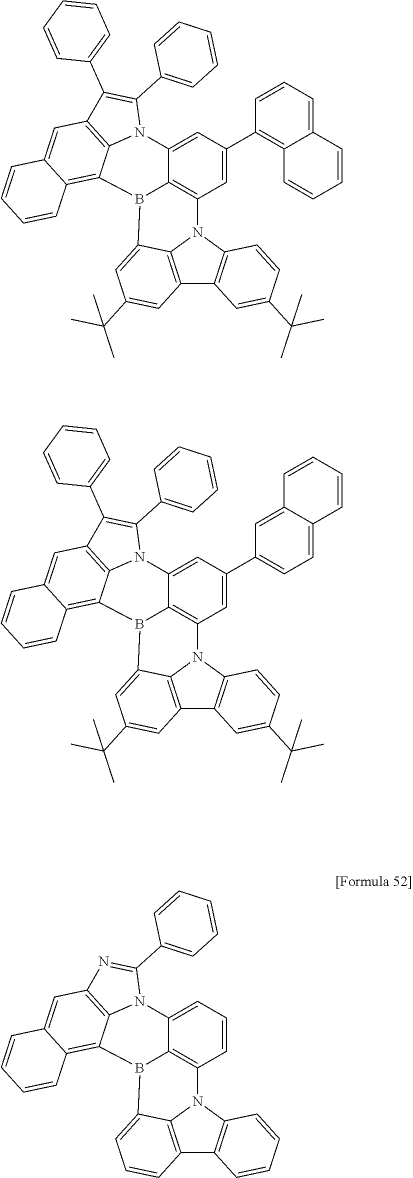 [SMILES: CC(C)(C)c1ccc2c(c1)c1cc(C(C)(C)C)cc3c1n2-c1cc(-c2cccc4ccccc24)cc2c1B3c1c3ccccc3cc3c(-c4ccccc4)c(-c4ccccc4)n-2c13 ; CC(C)(C)c1ccc2c(c1)c1cc(C(C)(C)C)cc3c1n2-c1cc(-c2ccc4ccccc4c2)cc2c1B3c1c3ccccc3cc3c(-c4ccccc4)c(-c4ccccc4)n-2c13 ; c1ccc(-c2nc3cc4ccccc4c4c3n2-c2cccc3c2B4c2cccc4c5ccccc5n-3c24)cc1]

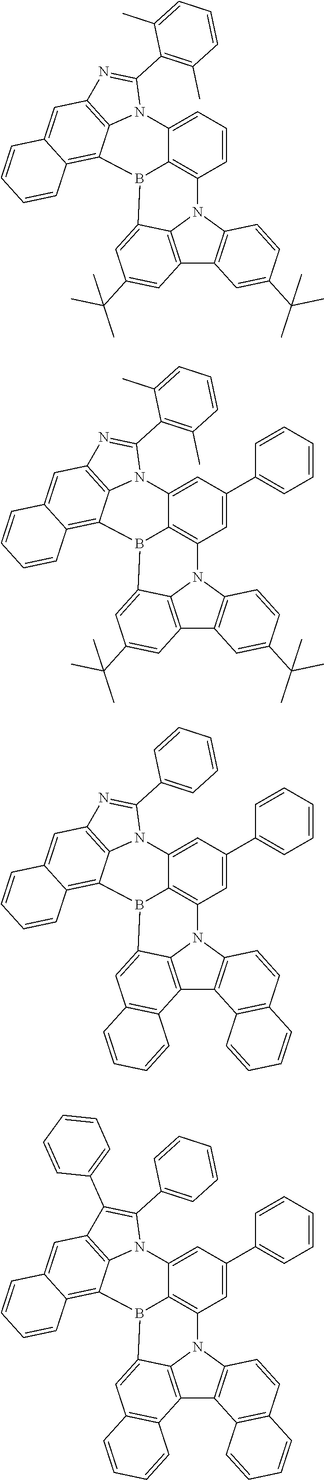

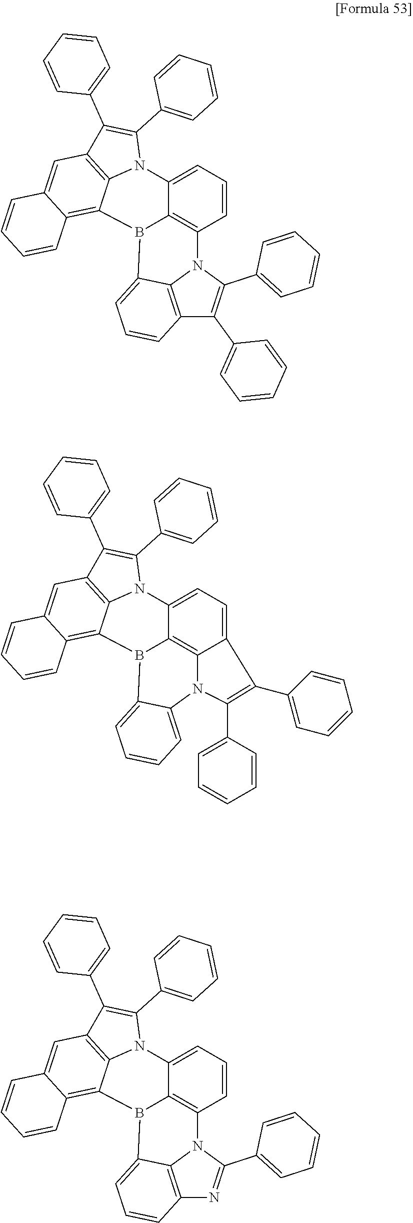 [SMILES: c1ccc(-c2c(-c3ccccc3)n3c4c(cccc24)B2c4c-3cccc4-n3c(-c4ccccc4)c(-c4ccccc4)c4cc5ccccc5c2c43)cc1 ; c1ccc(-c2c(-c3ccccc3)n3c4c5c(ccc24)-n2c(-c4ccccc4)c(-c4ccccc4)c4cc6ccccc6c(c42)B5c2ccccc2-3)cc1 ; c1ccc(-c2c(-c3ccccc3)n3c4c(c5ccccc5cc24)B2c4c(cccc4-3)-n3c(-c4ccccc4)nc4cccc2c43)cc1]

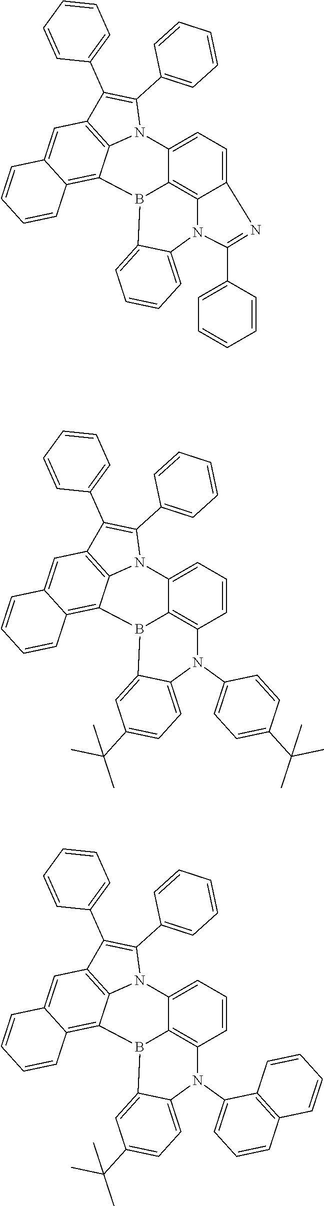 [SMILES: c1ccc(-c2c(-c3ccccc3)n3c4c(c5ccccc5cc24)B2c4ccccc4-n4c(-c5ccccc5)nc5ccc-3c2c54)cc1 ; CC(C)(C)c1ccc(N2c3ccc(C(C)(C)C)cc3B3c4c2cccc4-n2c(-c4ccccc4)c(-c4ccccc4)c4cc5ccccc5c3c42)cc1 ; CC(C)(C)c1ccc2c(c1)B1c3c(cccc3-n3c(-c4ccccc4)c(-c4ccccc4)c4cc5ccccc5c1c43)N2c1cccc2ccccc12]

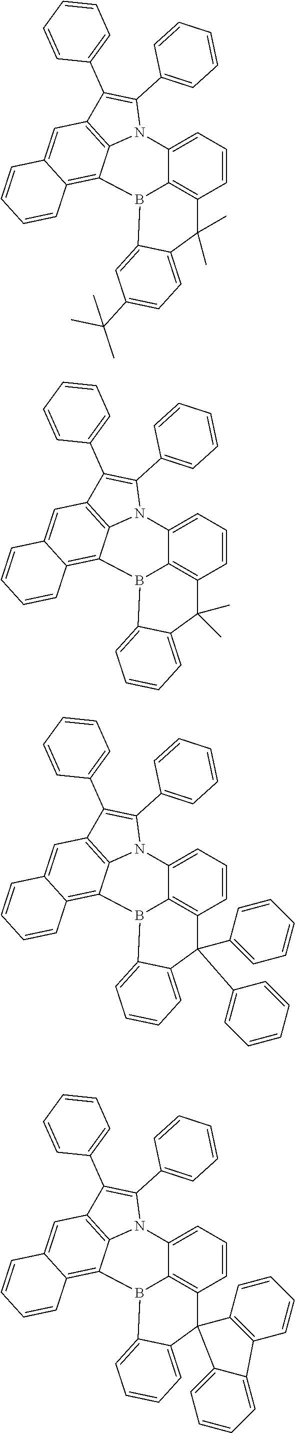

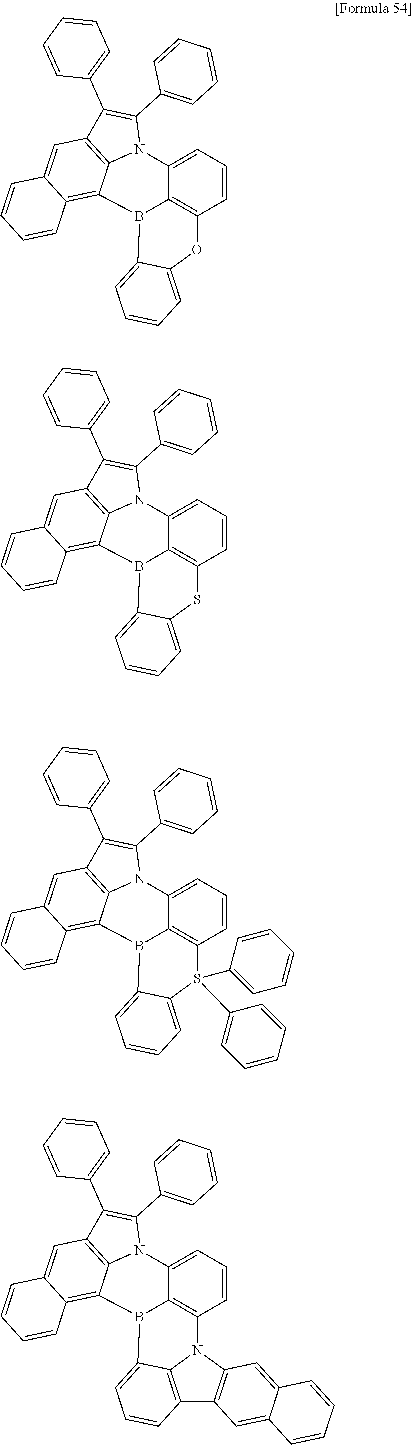 [SMILES: c1ccc(-c2c(-c3ccccc3)n3c4c(c5ccccc5cc24)B2c4ccccc4Oc4cccc-3c42)cc1 ; c1ccc(-c2c(-c3ccccc3)n3c4c(c5ccccc5cc24)B2c4ccccc4Sc4cccc-3c42)cc1 ; c1ccc(-c2c(-c3ccccc3)n3c4c(c5ccccc5cc24)B2c4ccccc4S(c4ccccc4)(c4ccccc4)c4cccc-3c42)cc1 ; c1ccc(-c2c(-c3ccccc3)n3c4c(c5ccccc5cc24)B2c4c-3cccc4-n3c4cc5ccccc5cc4c4cccc2c43)cc1]

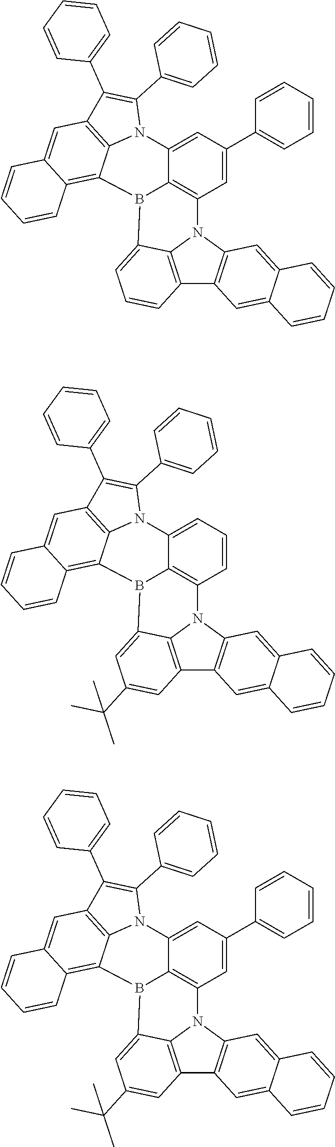 [SMILES: c1ccc(-c2cc3c4c(c2)-n2c5cc6ccccc6cc5c5cccc(c52)B4c2c4ccccc4cc4c(-c5ccccc5)c(-c5ccccc5)n-3c24)cc1 ; CC(C)(C)c1cc2c3c(c1)c1cc4ccccc4cc1n3-c1cccc3c1B2c1c2ccccc2cc2c(-c4ccccc4)c(-c4ccccc4)n-3c12 ; CC(C)(C)c1cc2c3c(c1)c1cc4ccccc4cc1n3-c1cc(-c3ccccc3)cc3c1B2c1c2ccccc2cc2c(-c4ccccc4)c(-c4ccccc4)n-3c12]

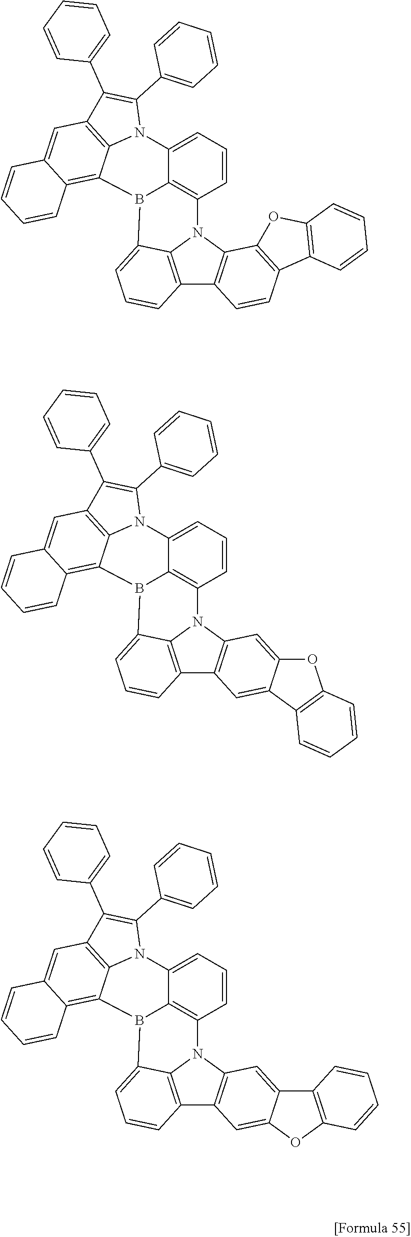 [SMILES: c1ccc(-c2c(-c3ccccc3)n3c4c(c5ccccc5cc24)B2c4c-3cccc4-n3c4c2cccc4c2ccc4c5ccccc5oc4c23)cc1 ; c1ccc(-c2c(-c3ccccc3)n3c4c(c5ccccc5cc24)B2c4c-3cccc4-n3c4cc5oc6ccccc6c5cc4c4cccc2c43)cc1 ; c1ccc(-c2c(-c3ccccc3)n3c4c(c5ccccc5cc24)B2c4c-3cccc4-n3c4cc5c(cc4c4cccc2c43)oc2ccccc25)cc1]

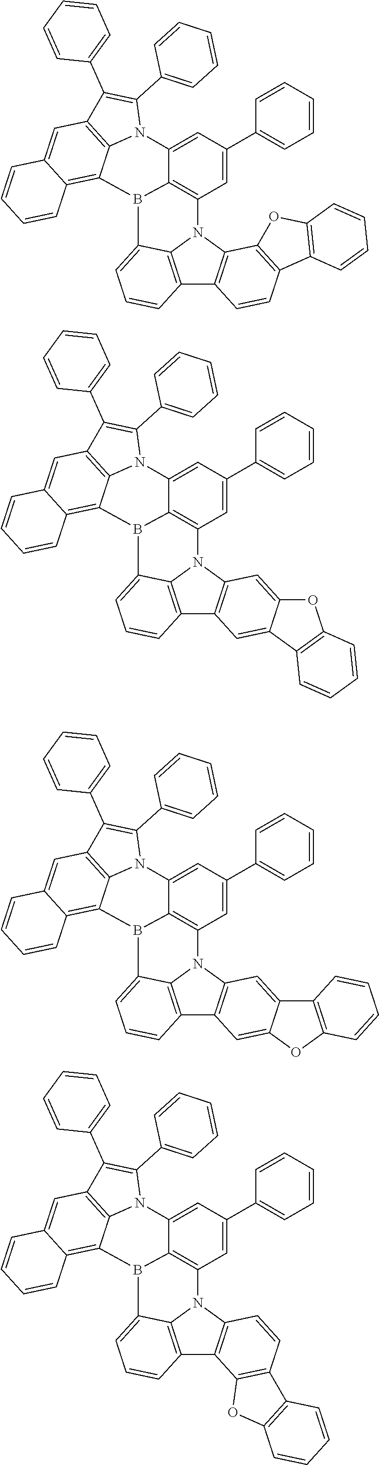

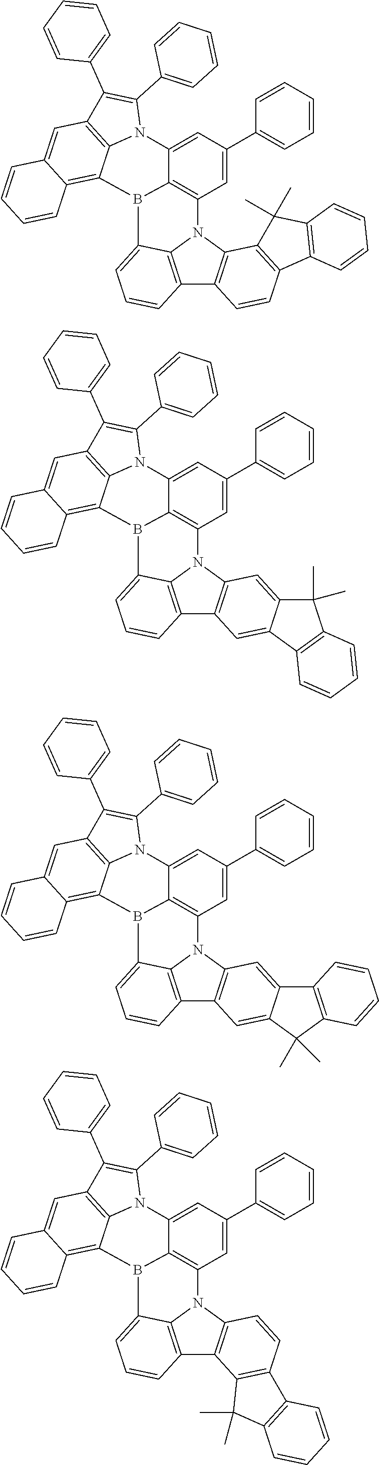

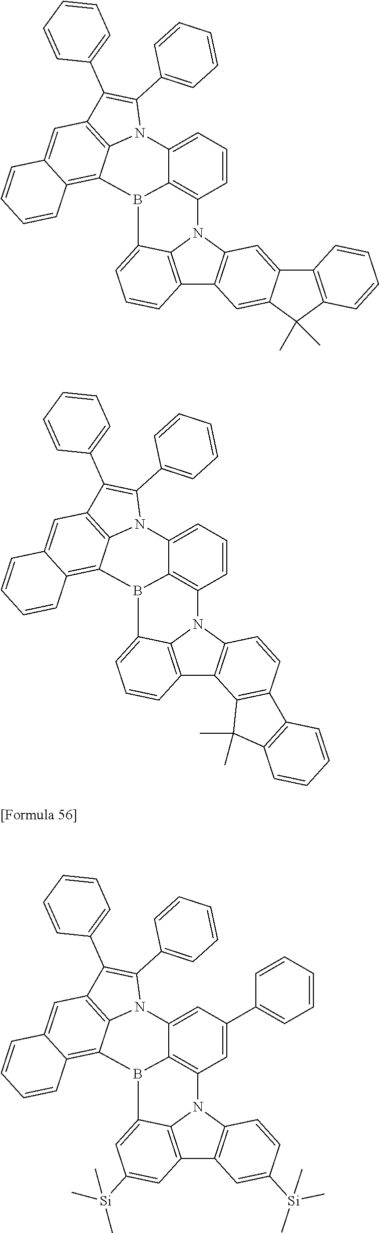 [SMILES: CC1(C)c2ccccc2-c2cc3c(cc21)c1cccc2c1n3-c1cccc3c1B2c1c2ccccc2cc2c(-c4ccccc4)c(-c4ccccc4)n-3c12 ; CC1(C)c2ccccc2-c2ccc3c(c21)c1cccc2c1n3-c1cccc3c1B2c1c2ccccc2cc2c(-c4ccccc4)c(-c4ccccc4)n-3c12 ; C[Si](C)(C)c1ccc2c(c1)c1cc([Si](C)(C)C)cc3c1n2-c1cc(-c2ccccc2)cc2c1B3c1c3ccccc3cc3c(-c4ccccc4)c(-c4ccccc4)n-2c13]

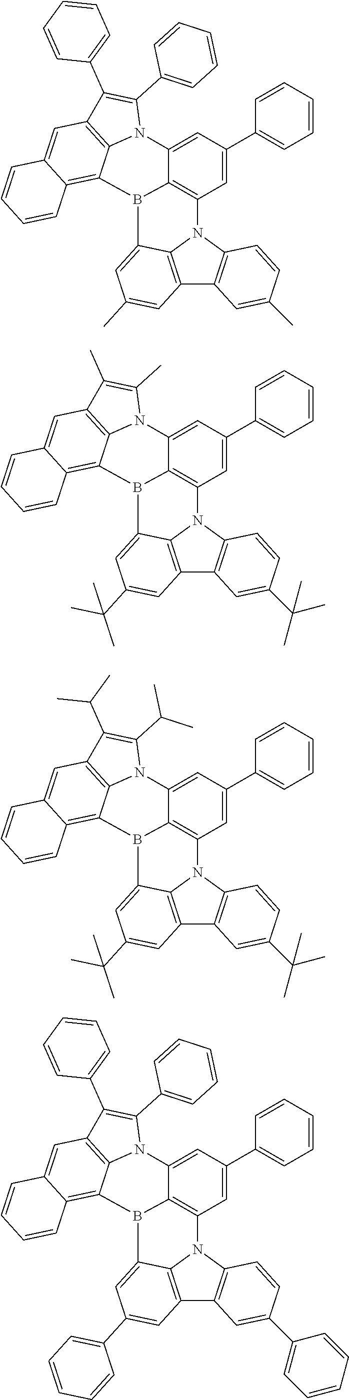

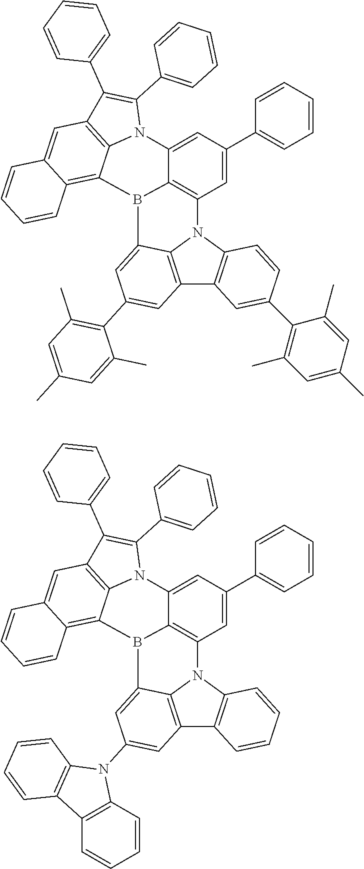 [SMILES: Cc1cc(C)c(-c2ccc3c(c2)c2cc(-c4c(C)cc(C)cc4C)cc4c2n3-c2cc(-c3ccccc3)cc3c2B4c2c4ccccc4cc4c(-c5ccccc5)c(-c5ccccc5)n-3c24)c(C)c1 ; c1ccc(-c2cc3c4c(c2)-n2c5ccccc5c5cc(-n6c7ccccc7c7ccccc76)cc(c52)B4c2c4ccccc4cc4c(-c5ccccc5)c(-c5ccccc5)n-3c24)cc1]

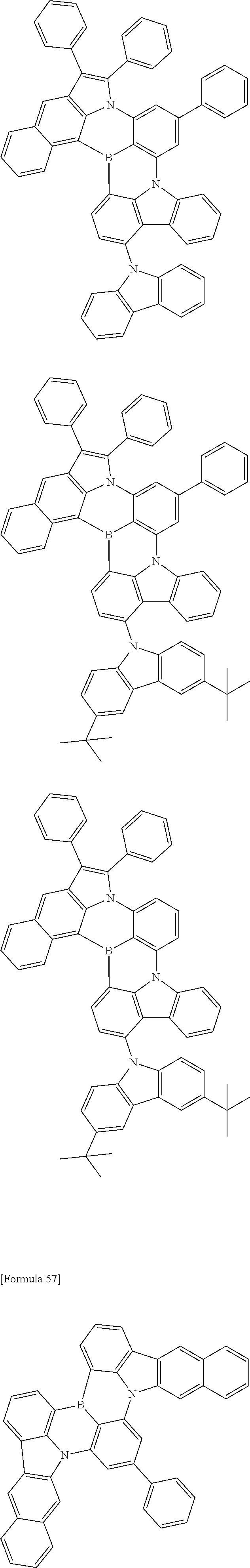 [SMILES: c1ccc(-c2cc3c4c(c2)-n2c5ccccc5c5c(-n6c7ccccc7c7ccccc76)ccc(c52)B4c2c4ccccc4cc4c(-c5ccccc5)c(-c5ccccc5)n-3c24)cc1 ; CC(C)(C)c1ccc2c(c1)c1cc(C(C)(C)C)ccc1n2-c1ccc2c3c1c1ccccc1n3-c1cc(-c3ccccc3)cc3c1B2c1c2ccccc2cc2c(-c4ccccc4)c(-c4ccccc4)n-3c12 ; CC(C)(C)c1ccc2c(c1)c1cc(C(C)(C)C)ccc1n2-c1ccc2c3c1c1ccccc1n3-c1cccc3c1B2c1c2ccccc2cc2c(-c4ccccc4)c(-c4ccccc4)n-3c12 ; c1ccc(-c2cc3c4c(c2)-n2c5cc6ccccc6cc5c5cccc(c52)B4c2cccc4c5cc6ccccc6cc5n-3c24)cc1]

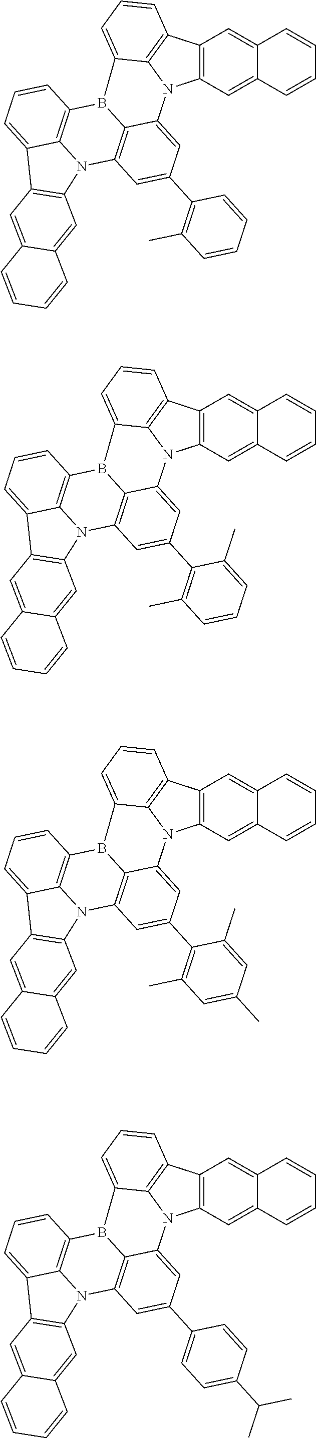 [SMILES: Cc1ccccc1-c1cc2c3c(c1)-n1c4cc5ccccc5cc4c4cccc(c41)B3c1cccc3c4cc5ccccc5cc4n-2c13 ; Cc1cccc(C)c1-c1cc2c3c(c1)-n1c4cc5ccccc5cc4c4cccc(c41)B3c1cccc3c4cc5ccccc5cc4n-2c13 ; Cc1cc(C)c(-c2cc3c4c(c2)-n2c5cc6ccccc6cc5c5cccc(c52)B4c2cccc4c5cc6ccccc6cc5n-3c24)c(C)c1 ; CC(C)c1ccc(-c2cc3c4c(c2)-n2c5cc6ccccc6cc5c5cccc(c52)B4c2cccc4c5cc6ccccc6cc5n-3c24)cc1]

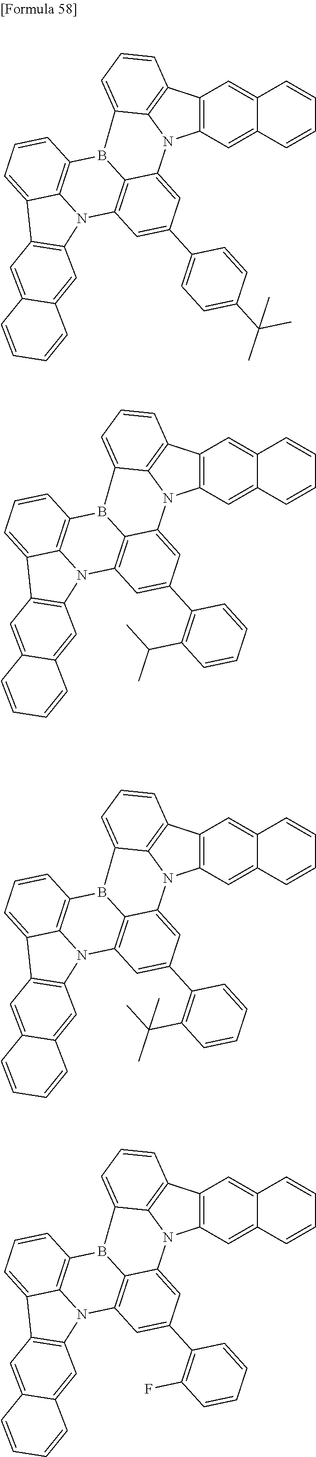 [SMILES: CC(C)(C)c1ccc(-c2cc3c4c(c2)-n2c5cc6ccccc6cc5c5cccc(c52)B4c2cccc4c5cc6ccccc6cc5n-3c24)cc1 ; CC(C)c1ccccc1-c1cc2c3c(c1)-n1c4cc5ccccc5cc4c4cccc(c41)B3c1cccc3c4cc5ccccc5cc4n-2c13 ; CC(C)(C)c1ccccc1-c1cc2c3c(c1)-n1c4cc5ccccc5cc4c4cccc(c41)B3c1cccc3c4cc5ccccc5cc4n-2c13 ; Fc1ccccc1-c1cc2c3c(c1)-n1c4cc5ccccc5cc4c4cccc(c41)B3c1cccc3c4cc5ccccc5cc4n-2c13]

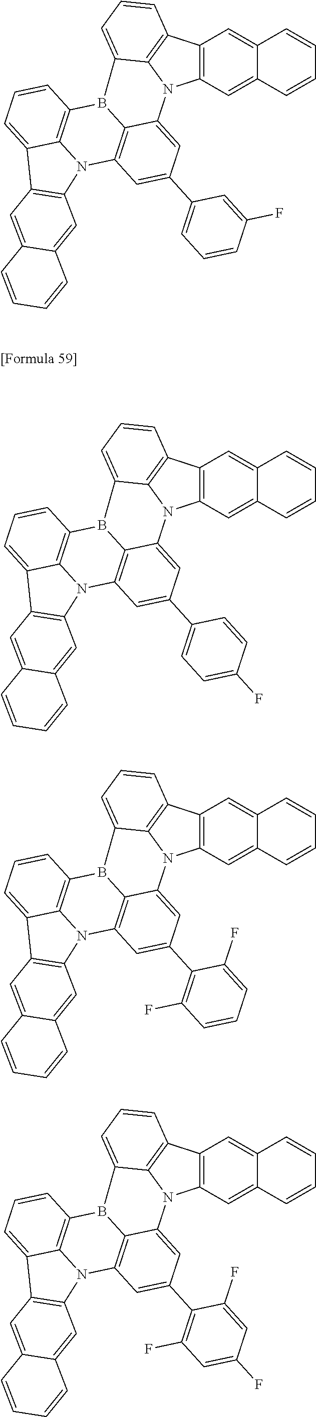 [SMILES: Fc1cccc(-c2cc3c4c(c2)-n2c5cc6ccccc6cc5c5cccc(c52)B4c2cccc4c5cc6ccccc6cc5n-3c24)c1 ; Fc1ccc(-c2cc3c4c(c2)-n2c5cc6ccccc6cc5c5cccc(c52)B4c2cccc4c5cc6ccccc6cc5n-3c24)cc1 ; Fc1cccc(F)c1-c1cc2c3c(c1)-n1c4cc5ccccc5cc4c4cccc(c41)B3c1cccc3c4cc5ccccc5cc4n-2c13 ; Fc1cc(F)c(-c2cc3c4c(c2)-n2c5cc6ccccc6cc5c5cccc(c52)B4c2cccc4c5cc6ccccc6cc5n-3c24)c(F)c1]

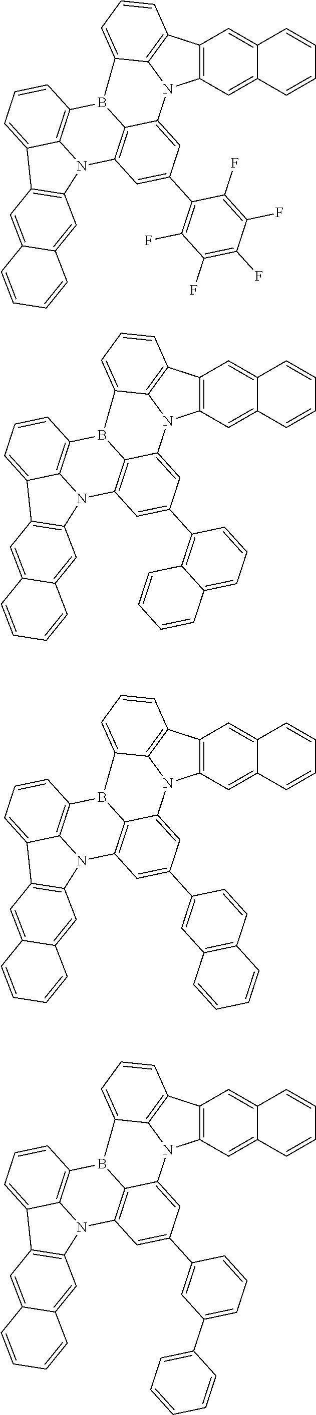 [SMILES: Fc1c(F)c(F)c(-c2cc3c4c(c2)-n2c5cc6ccccc6cc5c5cccc(c52)B4c2cccc4c5cc6ccccc6cc5n-3c24)c(F)c1F ; c1ccc2cc3c(cc2c1)c1cccc2c1n3-c1cc(-c3cccc4ccccc34)cc3c1B2c1cccc2c4cc5ccccc5cc4n-3c12 ; c1ccc2cc(-c3cc4c5c(c3)-n3c6cc7ccccc7cc6c6cccc(c63)B5c3cccc5c6cc7ccccc7cc6n-4c35)ccc2c1 ; c1ccc(-c2cccc(-c3cc4c5c(c3)-n3c6cc7ccccc7cc6c6cccc(c63)B5c3cccc5c6cc7ccccc7cc6n-4c35)c2)cc1]

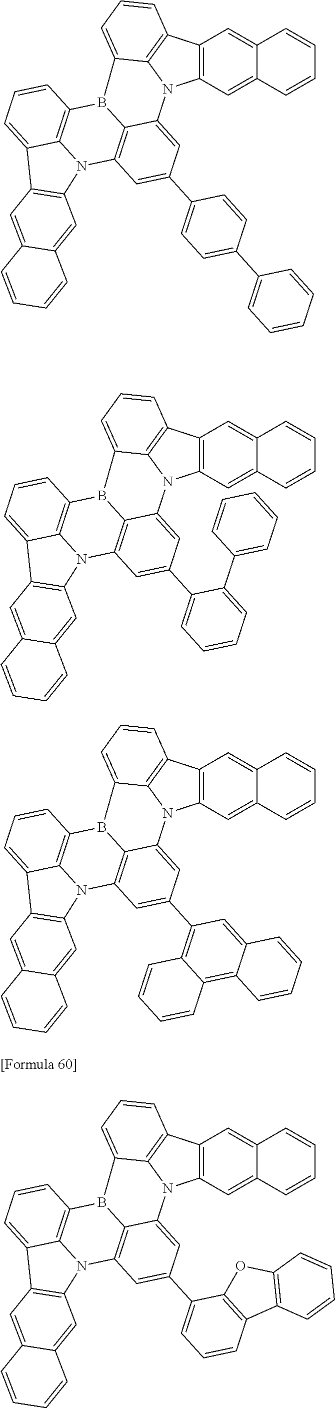 [SMILES: c1ccc(-c2ccc(-c3cc4c5c(c3)-n3c6cc7ccccc7cc6c6cccc(c63)B5c3cccc5c6cc7ccccc7cc6n-4c35)cc2)cc1 ; c1ccc(-c2ccccc2-c2cc3c4c(c2)-n2c5cc6ccccc6cc5c5cccc(c52)B4c2cccc4c5cc6ccccc6cc5n-3c24)cc1 ; c1ccc2cc3c(cc2c1)c1cccc2c1n3-c1cc(-c3cc4ccccc4c4ccccc34)cc3c1B2c1cccc2c4cc5ccccc5cc4n-3c12 ; c1ccc2cc3c(cc2c1)c1cccc2c1n3-c1cc(-c3cccc4c3oc3ccccc34)cc3c1B2c1cccc2c4cc5ccccc5cc4n-3c12]

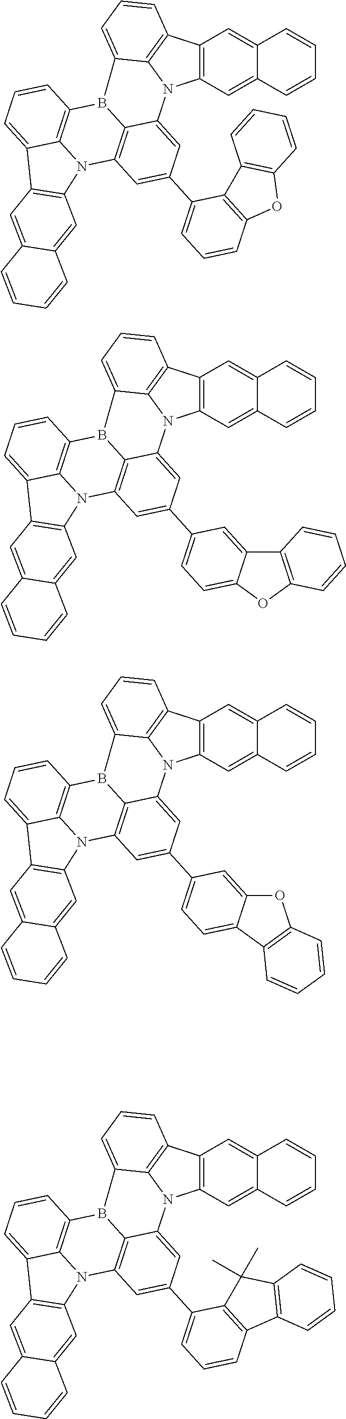 [SMILES: c1ccc2cc3c(cc2c1)c1cccc2c1n3-c1cc(-c3cccc4oc5ccccc5c34)cc3c1B2c1cccc2c4cc5ccccc5cc4n-3c12 ; c1ccc2cc3c(cc2c1)c1cccc2c1n3-c1cc(-c3ccc4oc5ccccc5c4c3)cc3c1B2c1cccc2c4cc5ccccc5cc4n-3c12 ; c1ccc2cc3c(cc2c1)c1cccc2c1n3-c1cc(-c3ccc4c(c3)oc3ccccc34)cc3c1B2c1cccc2c4cc5ccccc5cc4n-3c12 ; CC1(C)c2ccccc2-c2cccc(-c3cc4c5c(c3)-n3c6cc7ccccc7cc6c6cccc(c63)B5c3cccc5c6cc7ccccc7cc6n-4c35)c21]

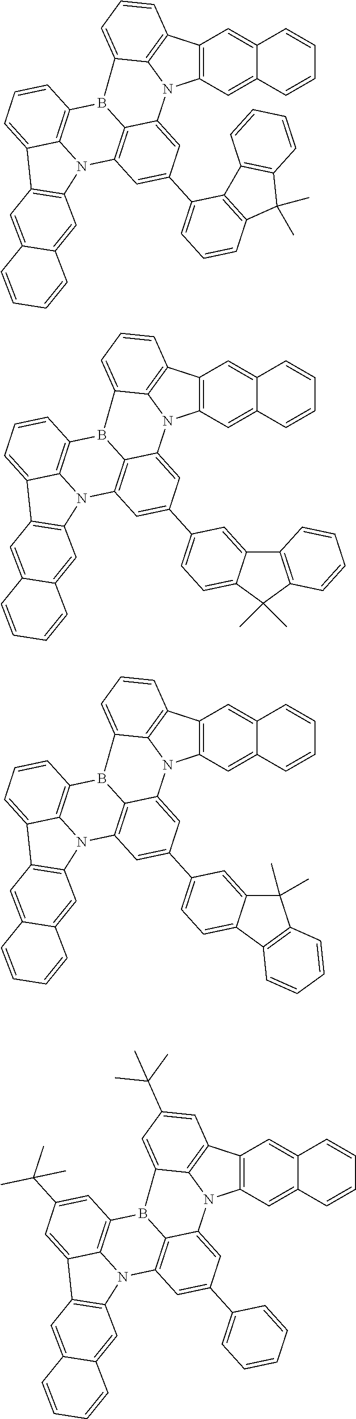 [SMILES: CC1(C)c2ccccc2-c2c(-c3cc4c5c(c3)-n3c6cc7ccccc7cc6c6cccc(c63)B5c3cccc5c6cc7ccccc7cc6n-4c35)cccc21 ; CC1(C)c2ccccc2-c2cc(-c3cc4c5c(c3)-n3c6cc7ccccc7cc6c6cccc(c63)B5c3cccc5c6cc7ccccc7cc6n-4c35)ccc21 ; CC1(C)c2ccccc2-c2ccc(-c3cc4c5c(c3)-n3c6cc7ccccc7cc6c6cccc(c63)B5c3cccc5c6cc7ccccc7cc6n-4c35)cc21 ; CC(C)(C)c1cc2c3c(c1)c1cc4ccccc4cc1n3-c1cc(-c3ccccc3)cc3c1B2c1cc(C(C)(C)C)cc2c4cc5ccccc5cc4n-3c12]

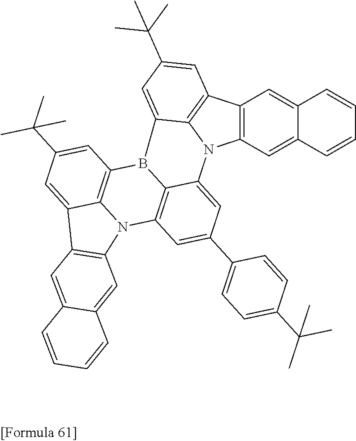 [SMILES: CC(C)(C)c1ccc(-c2cc3c4c(c2)-n2c5cc6ccccc6cc5c5cc(C(C)(C)C)cc(c52)B4c2cc(C(C)(C)C)cc4c5cc6ccccc6cc5n-3c24)cc1]

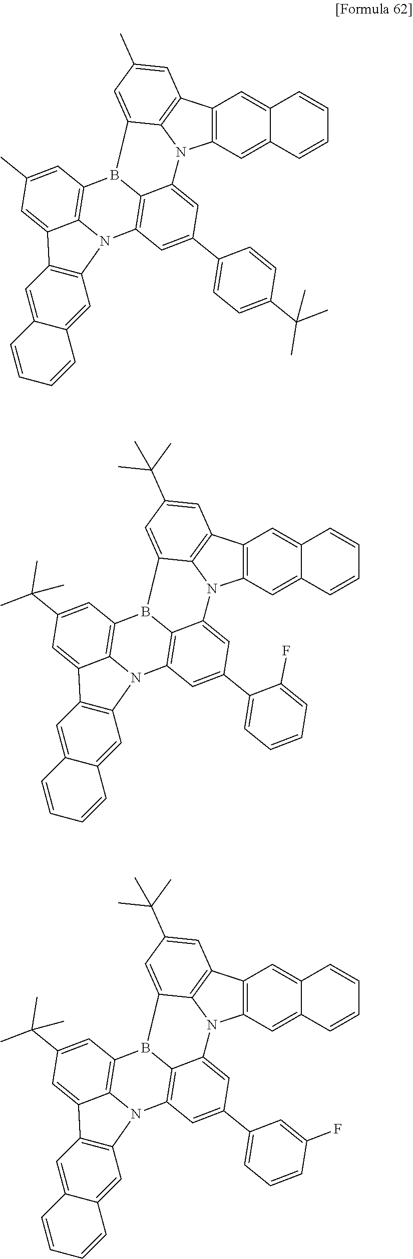 [SMILES: Cc1cc2c3c(c1)c1cc4ccccc4cc1n3-c1cc(-c3ccc(C(C)(C)C)cc3)cc3c1B2c1cc(C)cc2c4cc5ccccc5cc4n-3c12 ; CC(C)(C)c1cc2c3c(c1)c1cc4ccccc4cc1n3-c1cc(-c3ccccc3F)cc3c1B2c1cc(C(C)(C)C)cc2c4cc5ccccc5cc4n-3c12 ; CC(C)(C)c1cc2c3c(c1)c1cc4ccccc4cc1n3-c1cc(-c3cccc(F)c3)cc3c1B2c1cc(C(C)(C)C)cc2c4cc5ccccc5cc4n-3c12]

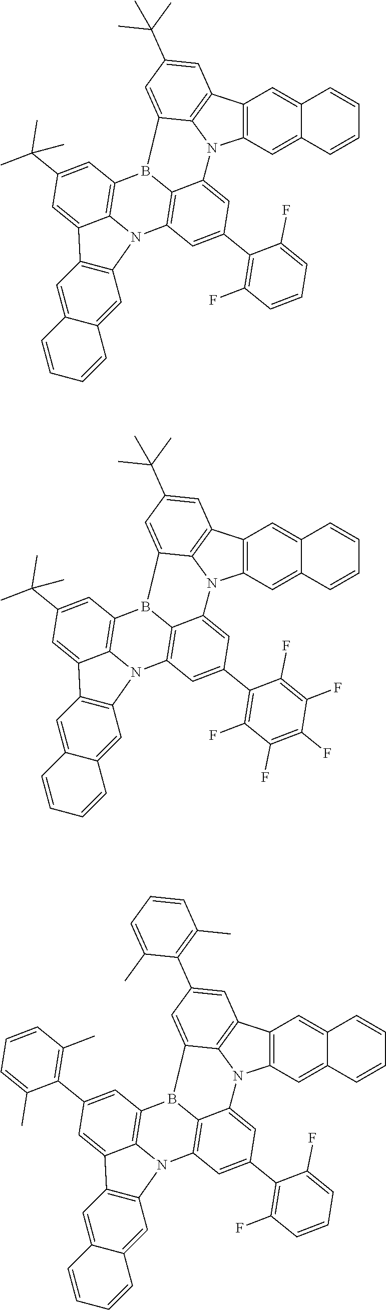 [SMILES: CC(C)(C)c1cc2c3c(c1)c1cc4ccccc4cc1n3-c1cc(-c3c(F)cccc3F)cc3c1B2c1cc(C(C)(C)C)cc2c4cc5ccccc5cc4n-3c12 ; CC(C)(C)c1cc2c3c(c1)c1cc4ccccc4cc1n3-c1cc(-c3c(F)c(F)c(F)c(F)c3F)cc3c1B2c1cc(C(C)(C)C)cc2c4cc5ccccc5cc4n-3c12 ; Cc1cccc(C)c1-c1cc2c3c(c1)c1cc4ccccc4cc1n3-c1cc(-c3c(F)cccc3F)cc3c1B2c1cc(-c2c(C)cccc2C)cc2c4cc5ccccc5cc4n-3c12]

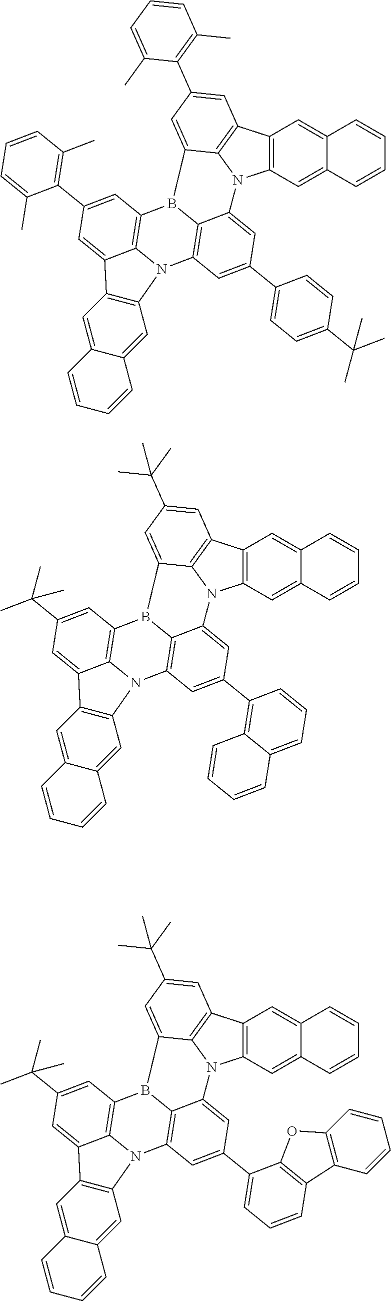

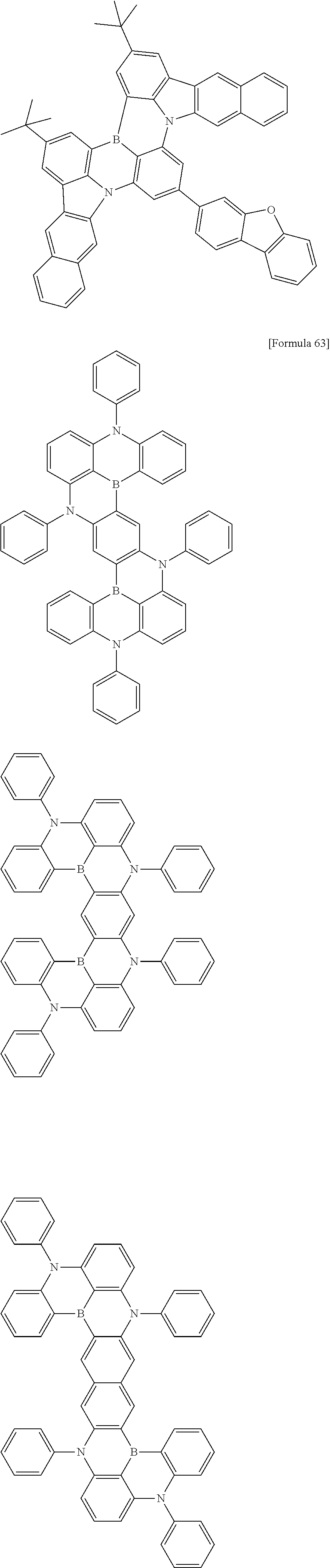 [SMILES: CC(C)(C)c1cc2c3c(c1)c1cc4ccccc4cc1n3-c1cc(-c3ccc4c(c3)oc3ccccc34)cc3c1B2c1cc(C(C)(C)C)cc2c4cc5ccccc5cc4n-3c12 ; c1ccc(N2c3ccccc3B3c4cc5c(cc4N(c4ccccc4)c4cccc2c43)B2c3ccccc3N(c3ccccc3)c3cccc(c32)N5c2ccccc2)cc1 ; c1ccc(N2c3ccccc3B3c4cc5c(cc4N(c4ccccc4)c4cccc2c43)N(c2ccccc2)c2cccc3c2B5c2ccccc2N3c2ccccc2)cc1 ; c1ccc(N2c3ccccc3B3c4cc5cc6c(cc5cc4N(c4ccccc4)c4cccc2c43)B2c3ccccc3N(c3ccccc3)c3cccc(c32)N6c2ccccc2)cc1]

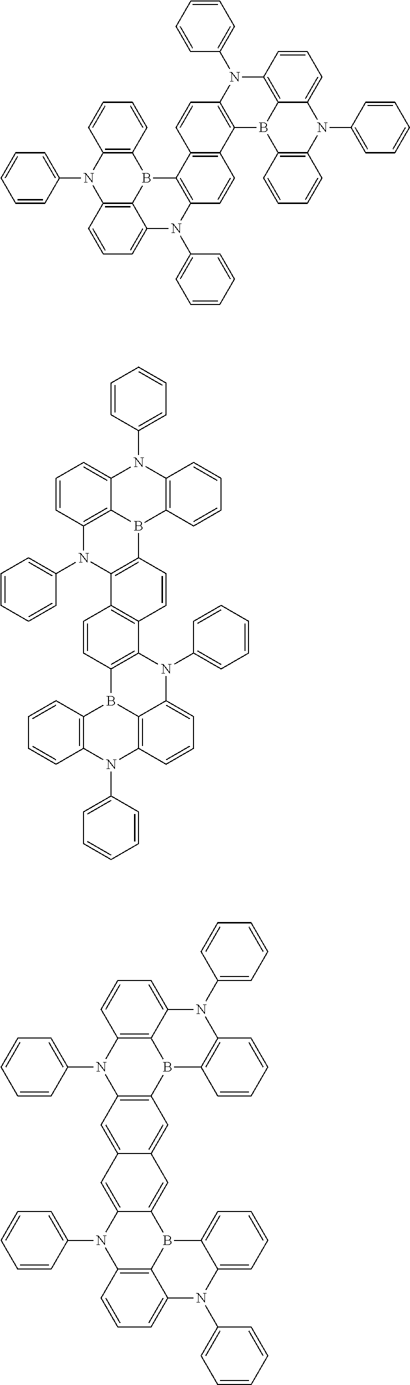 [SMILES: c1ccc(N2c3ccccc3B3c4c2cccc4N(c2ccccc2)c2ccc4c5c(ccc4c23)N(c2ccccc2)c2cccc3c2B5c2ccccc2N3c2ccccc2)cc1 ; c1ccc(N2c3ccccc3B3c4ccc5c6c(ccc5c4N(c4ccccc4)c4cccc2c43)B2c3ccccc3N(c3ccccc3)c3cccc(c32)N6c2ccccc2)cc1 ; c1ccc(N2c3ccccc3B3c4cc5cc6c(cc5cc4N(c4ccccc4)c4cccc2c43)N(c2ccccc2)c2cccc3c2B6c2ccccc2N3c2ccccc2)cc1]

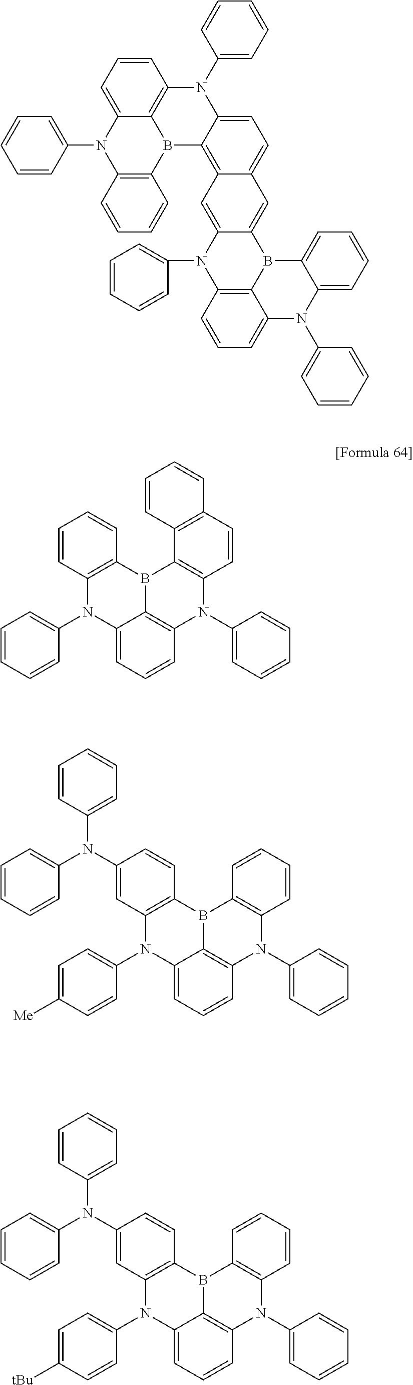 [SMILES: c1ccc(N2c3ccccc3B3c4cc5ccc6c(c5cc4N(c4ccccc4)c4cccc2c43)B2c3ccccc3N(c3ccccc3)c3cccc(c32)N6c2ccccc2)cc1 ; c1ccc(N2c3ccccc3B3c4c2cccc4N(c2ccccc2)c2ccc4ccccc4c23)cc1 ; Cc1ccc(N2c3cc(N(c4ccccc4)c4ccccc4)ccc3B3c4ccccc4N(c4ccccc4)c4cccc2c43)cc1 ; CC(C)(C)c1ccc(N2c3cc(N(c4ccccc4)c4ccccc4)ccc3B3c4ccccc4N(c4ccccc4)c4cccc2c43)cc1]

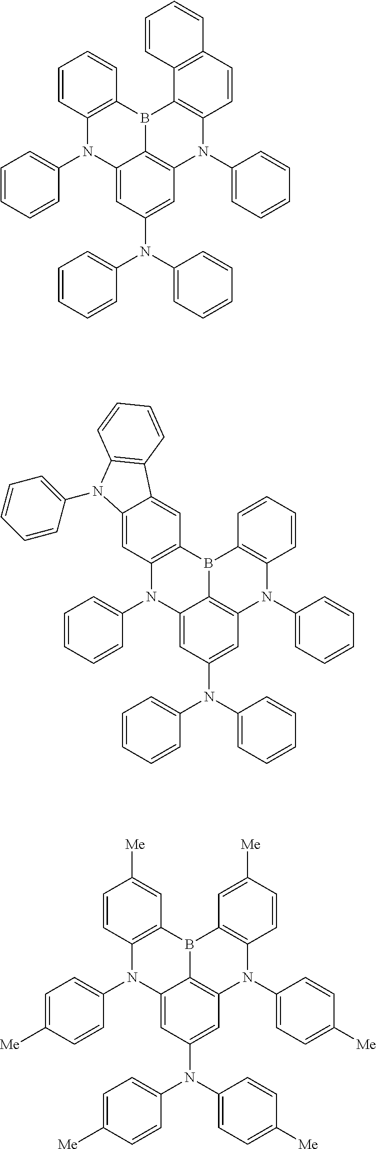 [SMILES: c1ccc(N(c2ccccc2)c2cc3c4c(c2)N(c2ccccc2)c2ccc5ccccc5c2B4c2ccccc2N3c2ccccc2)cc1 ; c1ccc(N(c2ccccc2)c2cc3c4c(c2)N(c2ccccc2)c2cc5c(cc2B4c2ccccc2N3c2ccccc2)c2ccccc2n5-c2ccccc2)cc1 ; Cc1ccc(N(c2ccc(C)cc2)c2cc3c4c(c2)N(c2ccc(C)cc2)c2ccc(C)cc2B4c2cc(C)ccc2N3c2ccc(C)cc2)cc1]

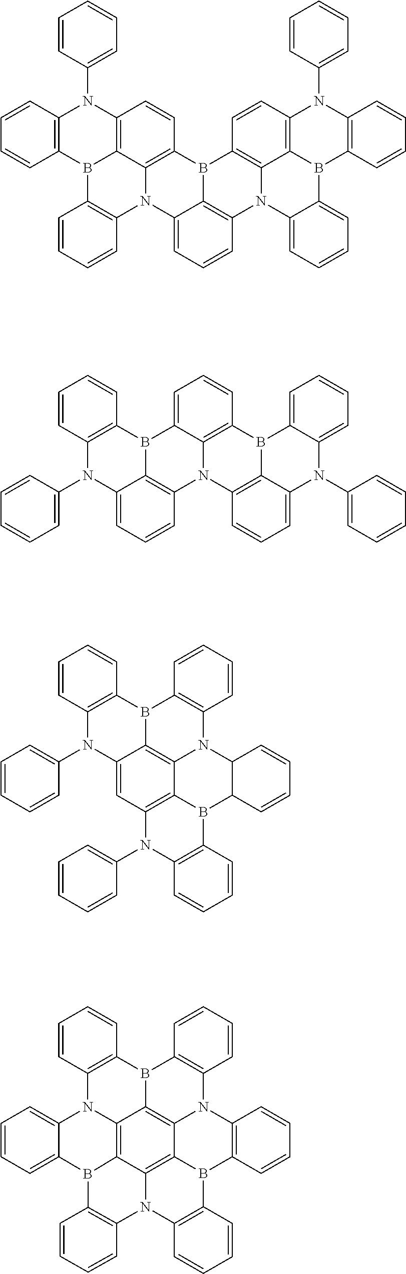 [SMILES: c1ccc(N2c3ccccc3B3c4ccccc4N4c5cccc6c5B(c5ccc2c3c54)c2ccc3c4c2N6c2ccccc2B4c2ccccc2N3c2ccccc2)cc1 ; c1ccc(N2c3ccccc3B3c4cccc5c4N(c4cccc2c43)c2cccc3c2B5c2ccccc2N3c2ccccc2)cc1 ; C1=CC2B3c4ccccc4N(c4ccccc4)c4cc5c6c(c43)N(c3ccccc3B6c3ccccc3N5c3ccccc3)C2C=C1 ; c1ccc2c(c1)B1c3ccccc3N3c4ccccc4B4c5ccccc5N5c6ccccc6B6c7ccccc7N2c2c1c3c4c5c26]

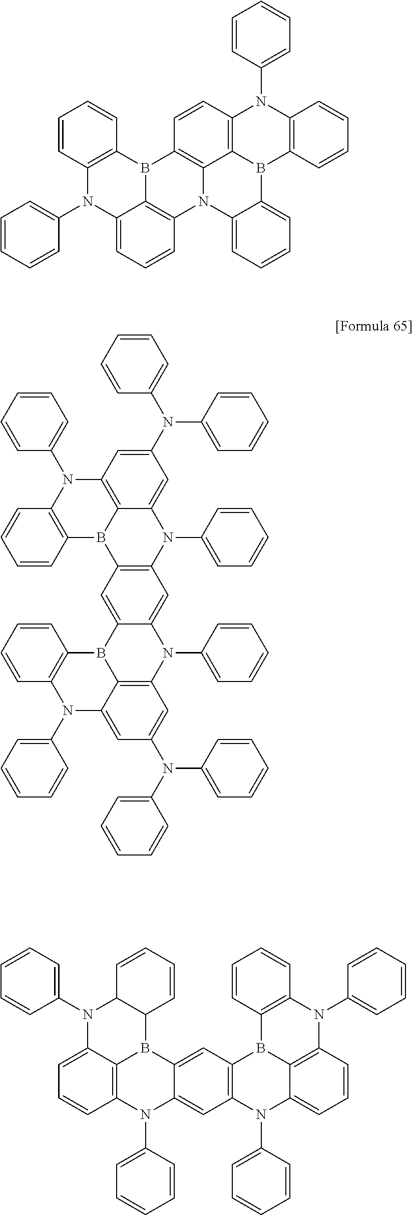 [SMILES: c1ccc(N2c3ccccc3B3c4ccc5c6c4N(c4ccccc4B6c4ccccc4N5c4ccccc4)c4cccc2c43)cc1 ; c1ccc(N(c2ccccc2)c2cc3c4c(c2)N(c2ccccc2)c2cc5c(cc2B4c2ccccc2N3c2ccccc2)B2c3ccccc3N(c3ccccc3)c3cc(N(c4ccccc4)c4ccccc4)cc(c32)N5c2ccccc2)cc1 ; C1=CC2B3c4cc5c(cc4N(c4ccccc4)c4cccc(c43)N(c3ccccc3)C2C=C1)N(c1ccccc1)c1cccc2c1B5c1ccccc1N2c1ccccc1]

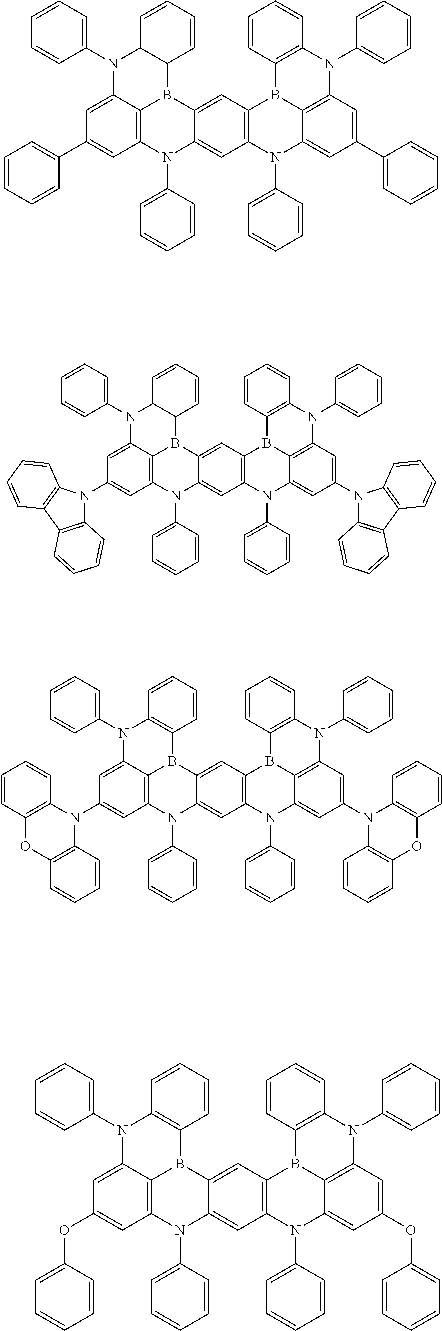

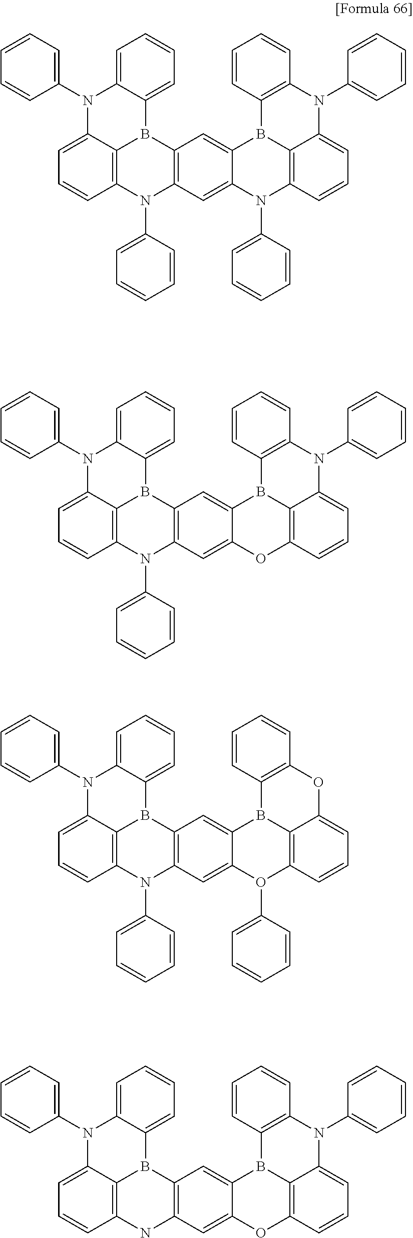

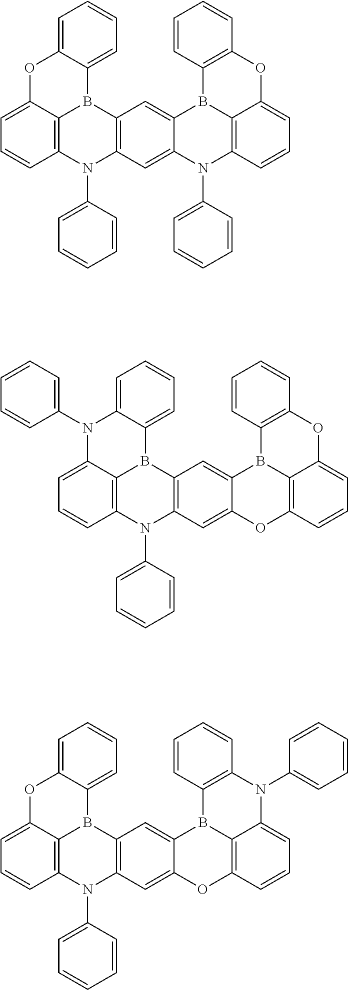 [SMILES: c1ccc(N2c3cc4c(cc3B3c5ccccc5Oc5cccc2c53)B2c3ccccc3Oc3cccc(c32)N4c2ccccc2)cc1 ; c1ccc(N2c3ccccc3B3c4cc5c(cc4N(c4ccccc4)c4cccc2c43)Oc2cccc3c2B5c2ccccc2O3)cc1 ; c1ccc(N2c3ccccc3B3c4cc5c(cc4Oc4cccc2c43)N(c2ccccc2)c2cccc3c2B5c2ccccc2O3)cc1]

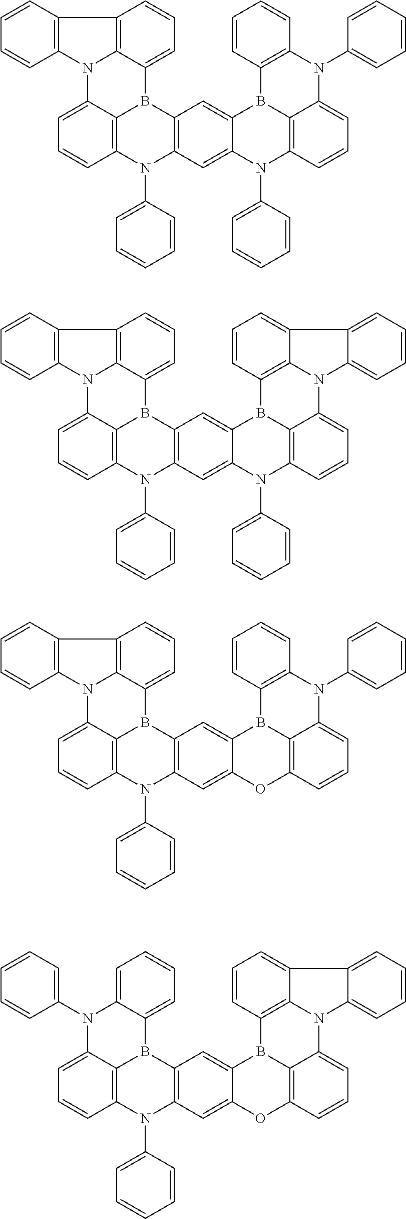

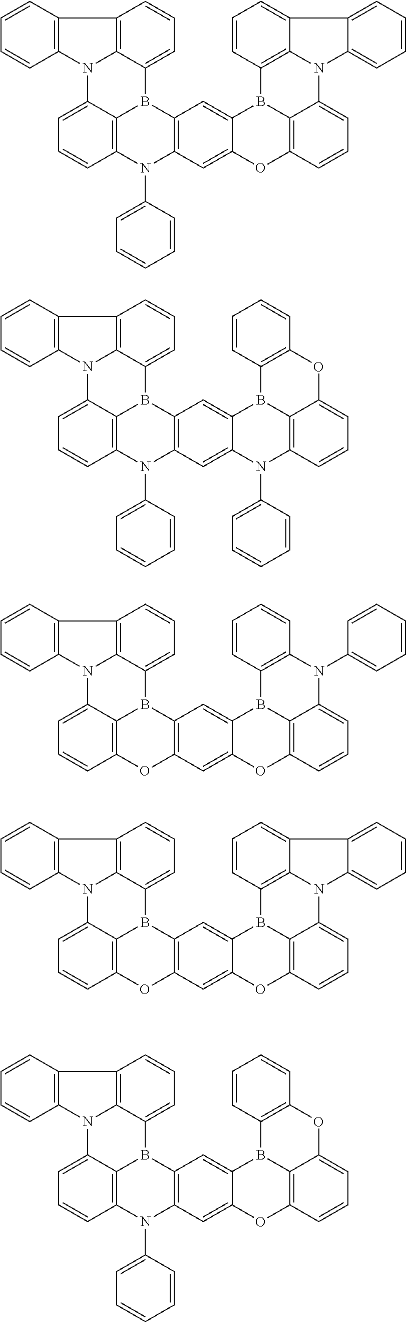

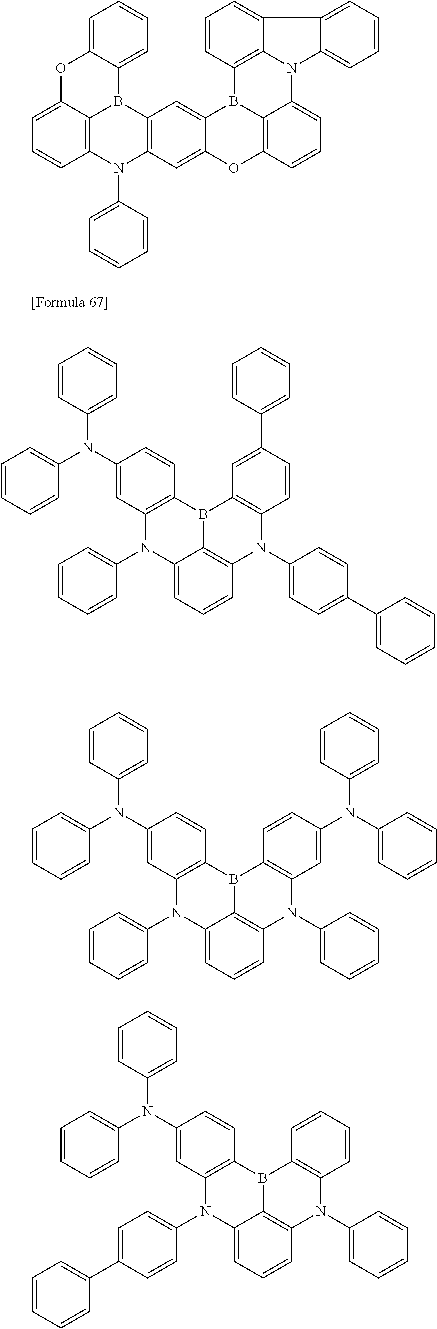

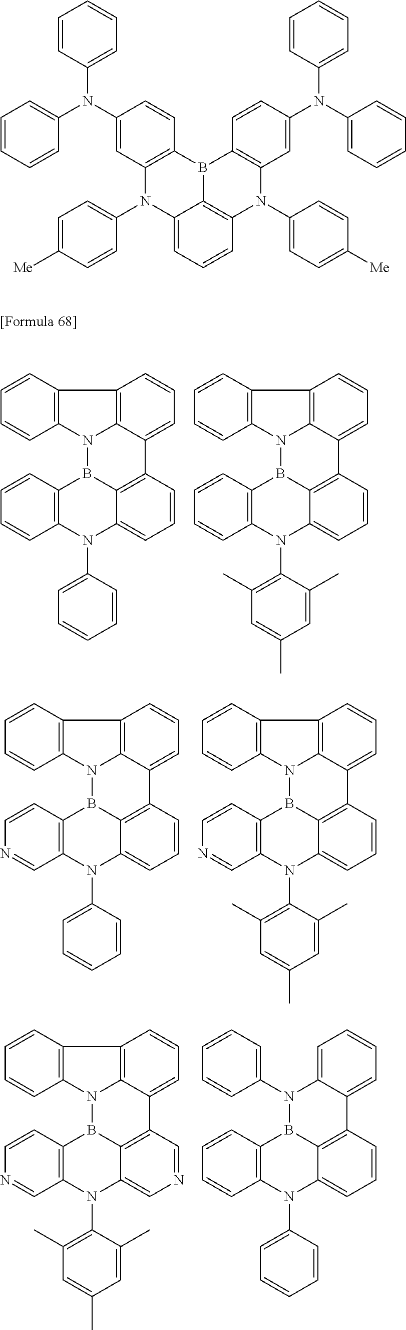

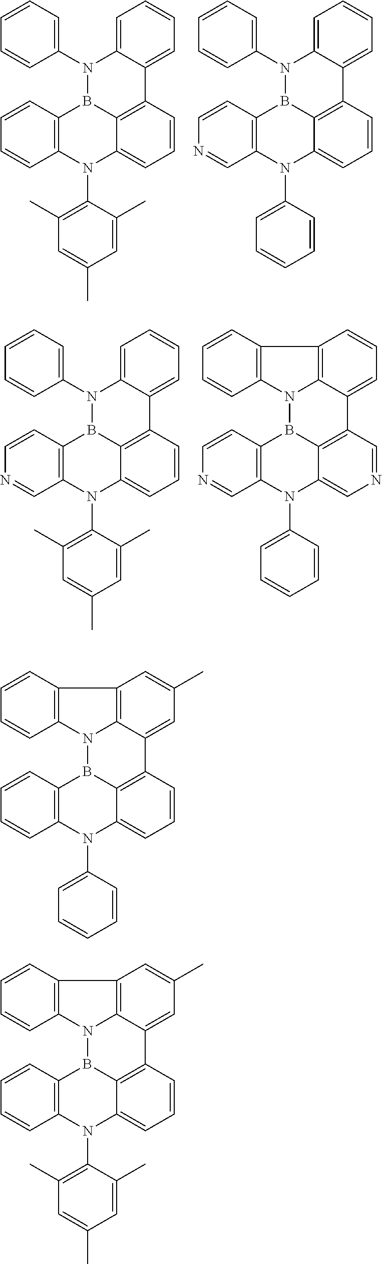

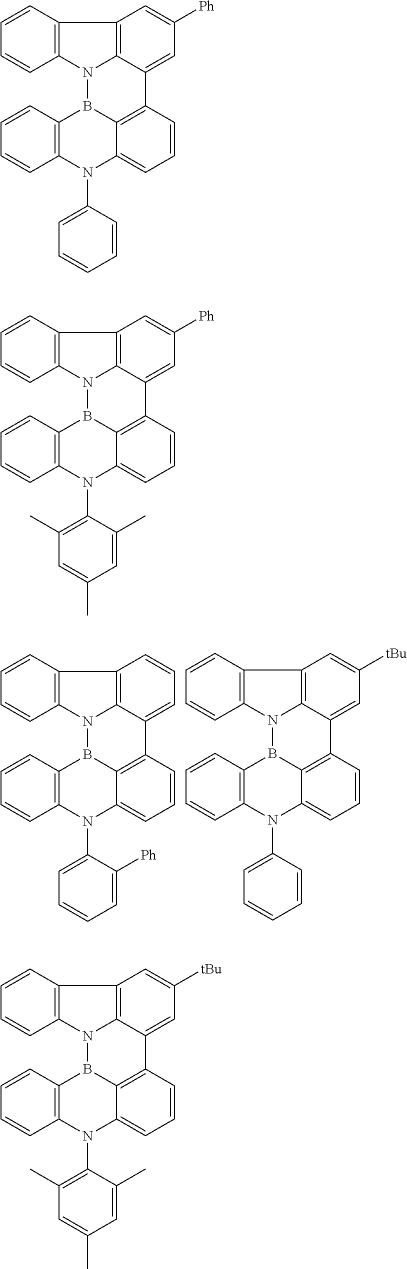

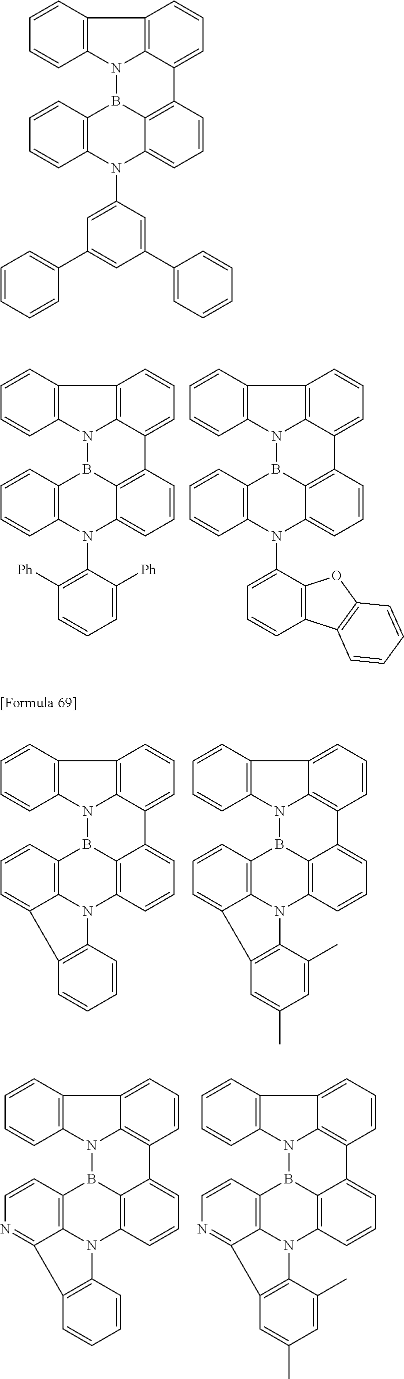

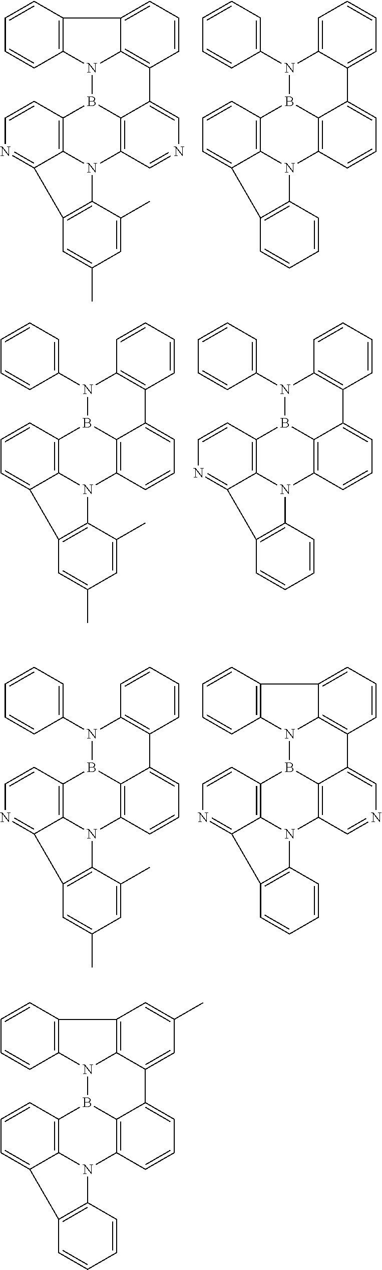

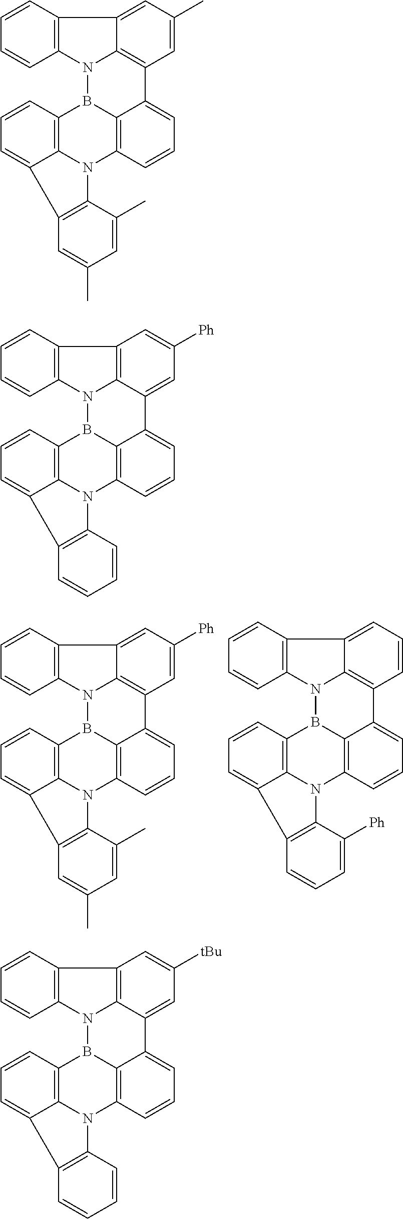

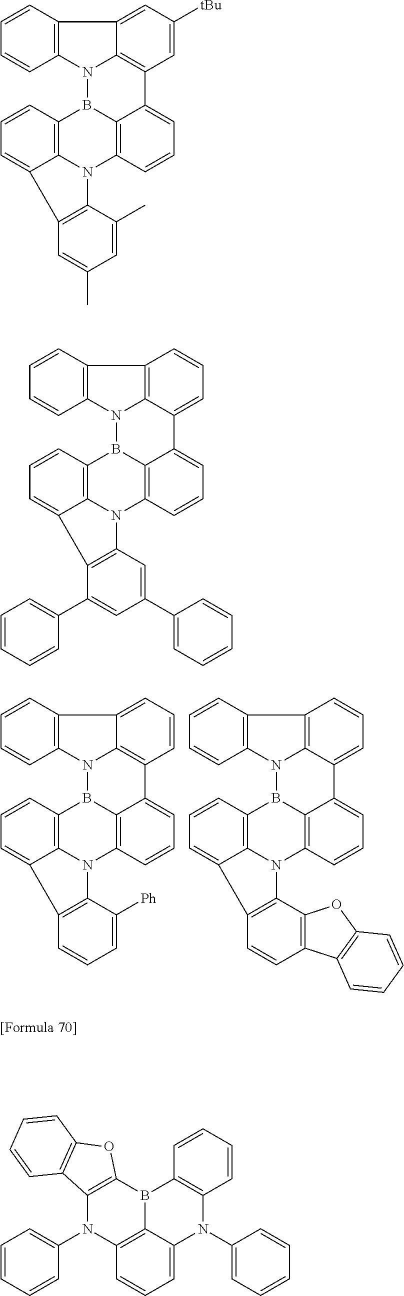

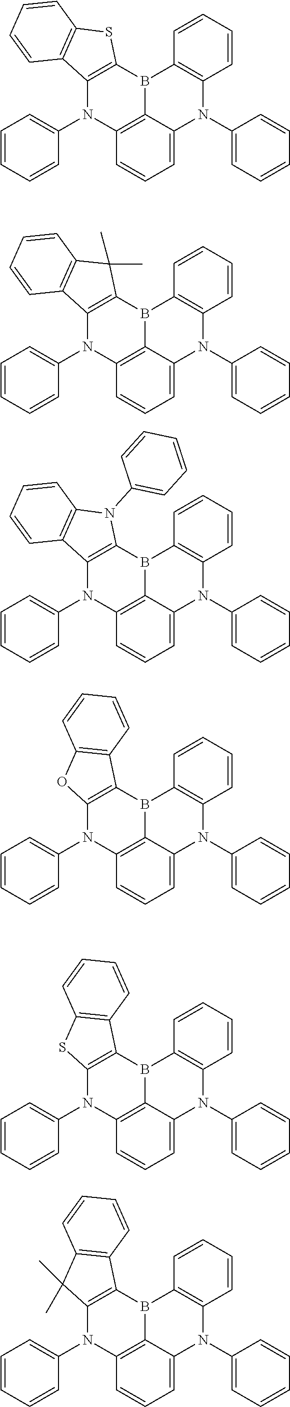

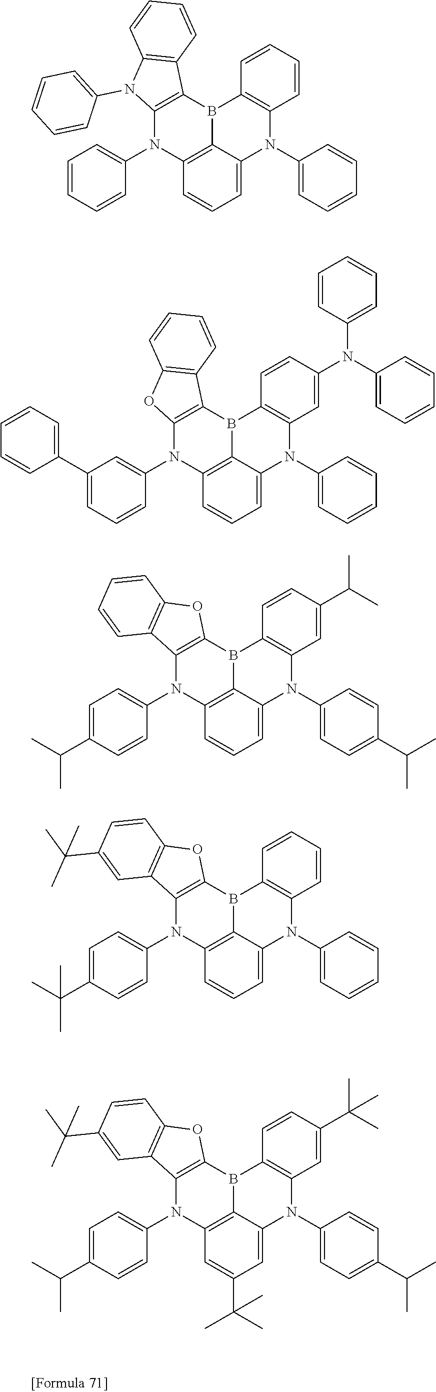

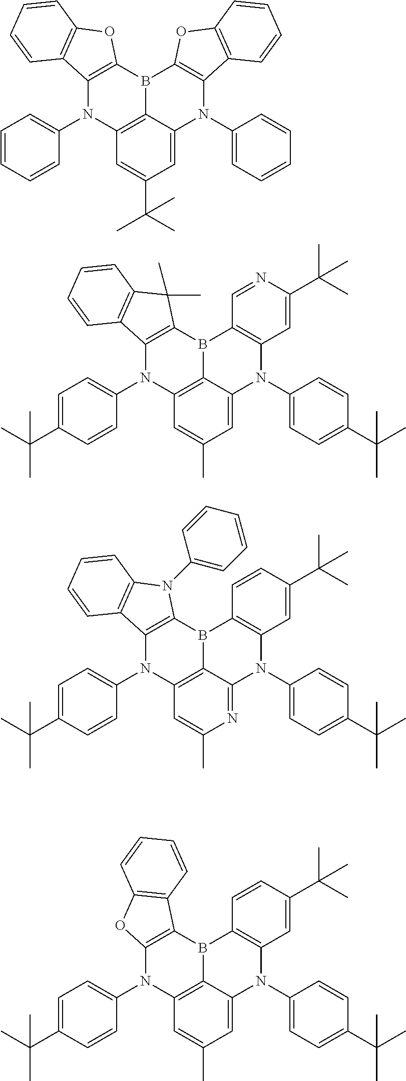

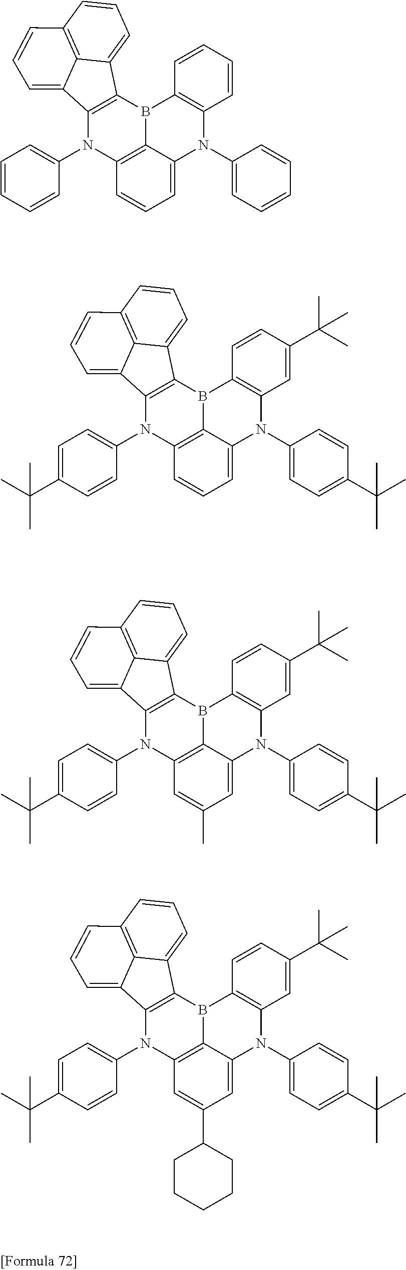

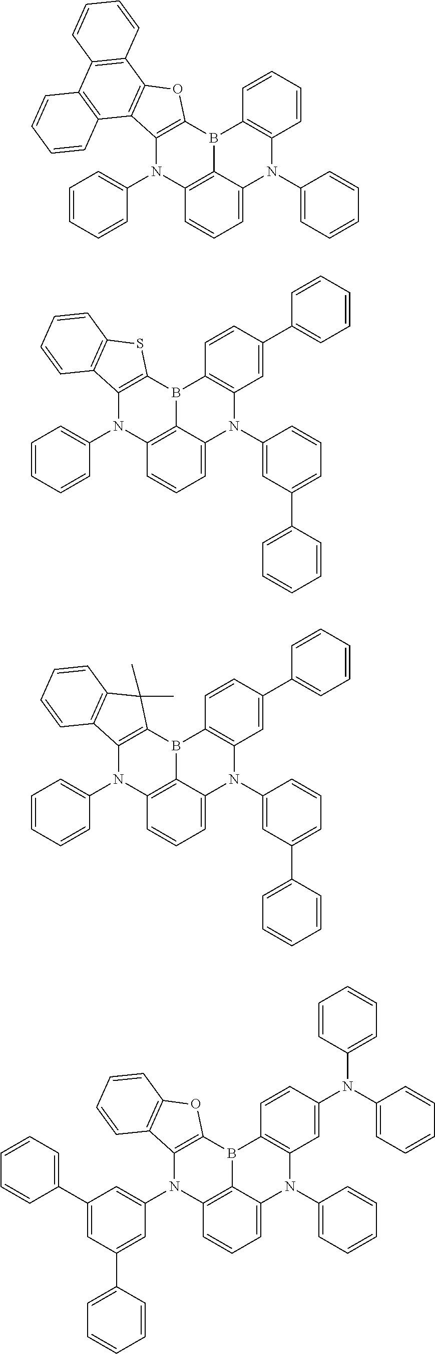

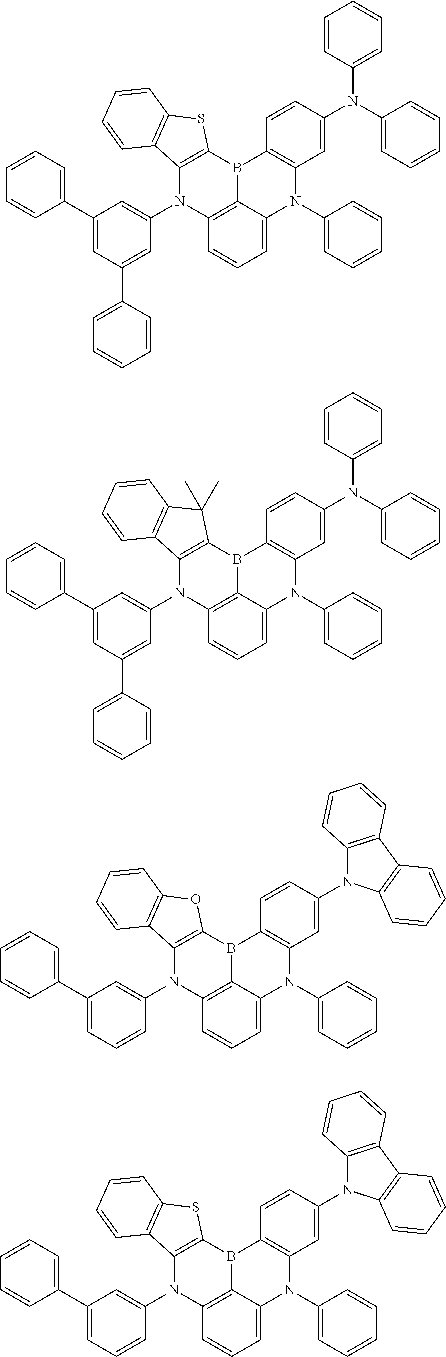

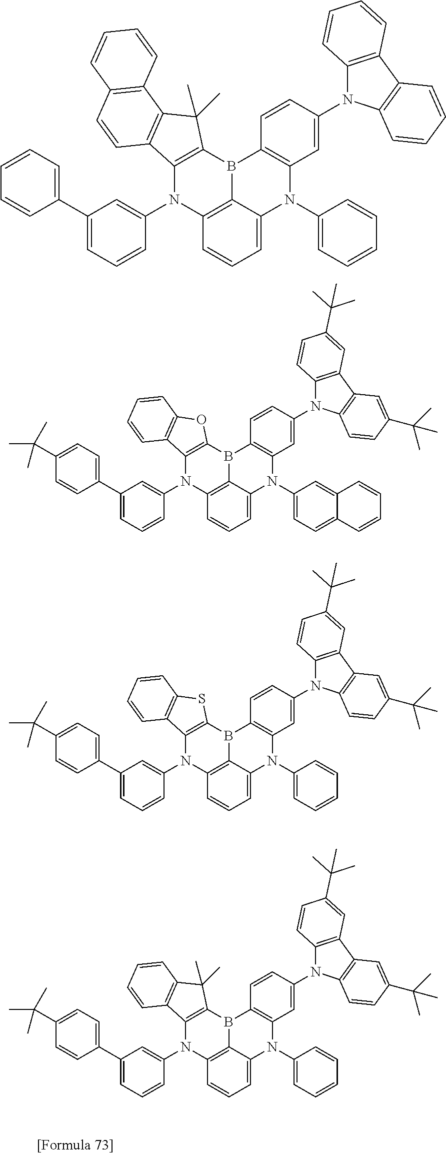

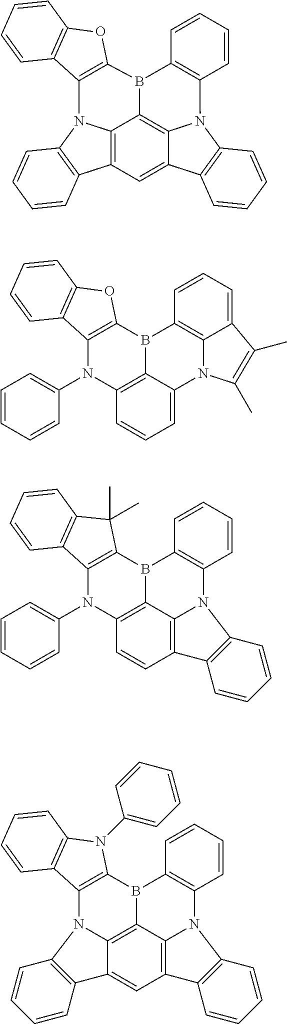 [SMILES: c1ccc2c(c1)B1c3oc4ccccc4c3-n3c4ccccc4c4cc5c6ccccc6n-2c5c1c43 ; Cc1c(C)n2c3c(cccc13)B1c3oc4ccccc4c3N(c3ccccc3)c3cccc-2c31 ; CC1(C)C2=C(c3ccccc31)N(c1ccccc1)c1ccc3c4ccccc4n4c3c1B2c1ccccc1-4 ; c1ccc(-n2c3c(c4ccccc42)-n2c4ccccc4c4cc5c6ccccc6n6c5c(c42)B3c2ccccc2-6)cc1]

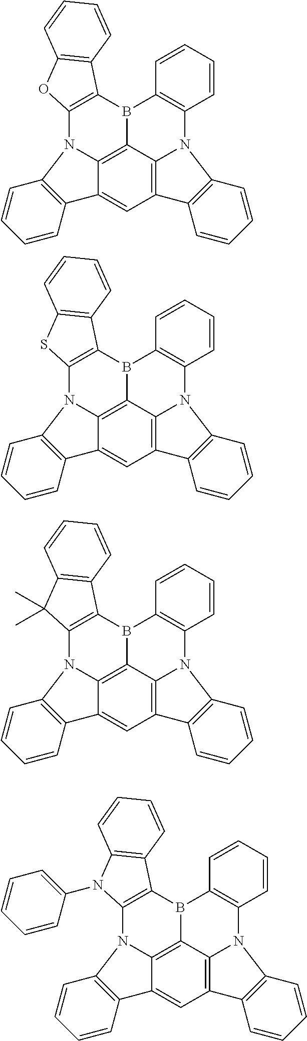

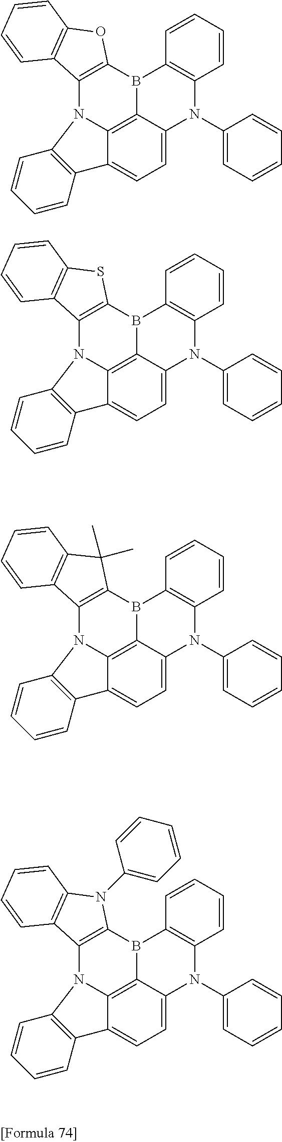

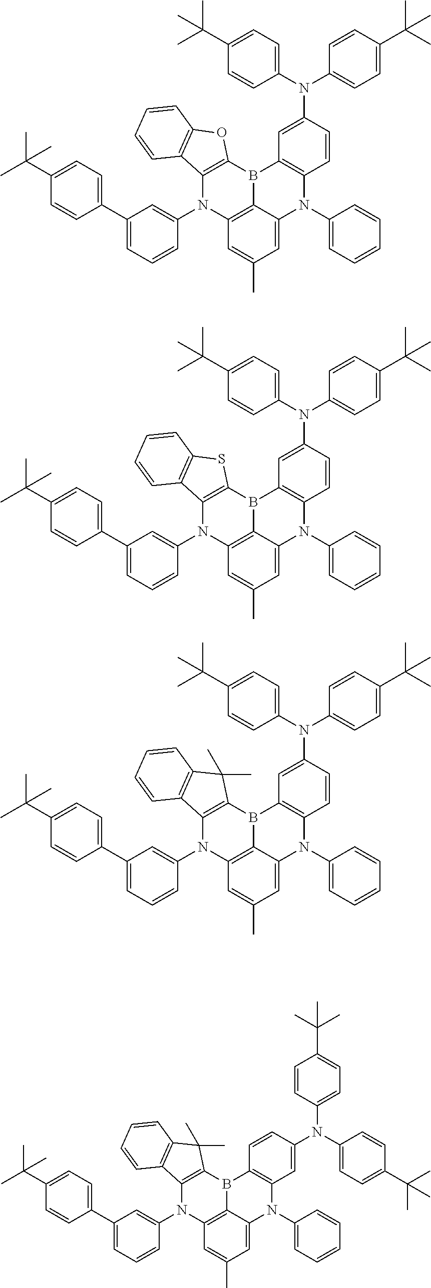

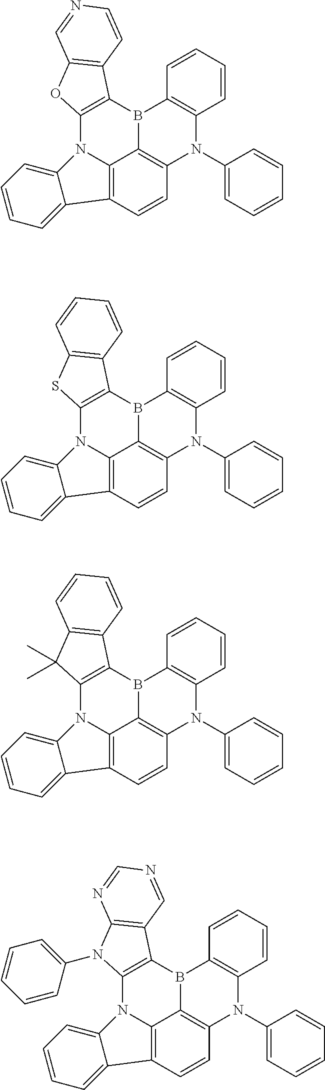 [SMILES: c1ccc(N2c3ccccc3B3c4c(oc5cnccc45)-n4c5ccccc5c5ccc2c3c54)cc1 ; c1ccc(N2c3ccccc3B3c4c(sc5ccccc45)-n4c5ccccc5c5ccc2c3c54)cc1 ; CC1(C)C2=C(B3c4ccccc4N(c4ccccc4)c4ccc5c6ccccc6n2c5c43)c2ccccc21 ; c1ccc(N2c3ccccc3B3c4c(n(-c5ccccc5)c5ncncc45)-n4c5ccccc5c5ccc2c3c54)cc1]

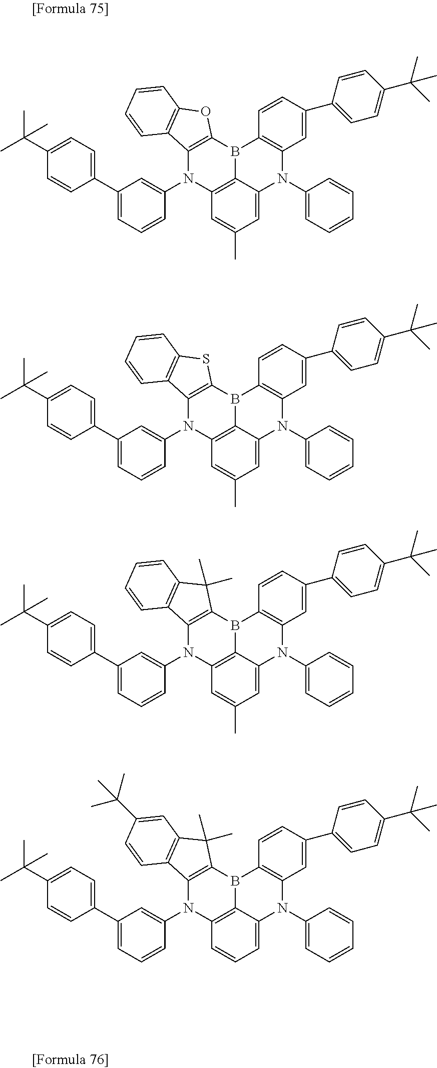

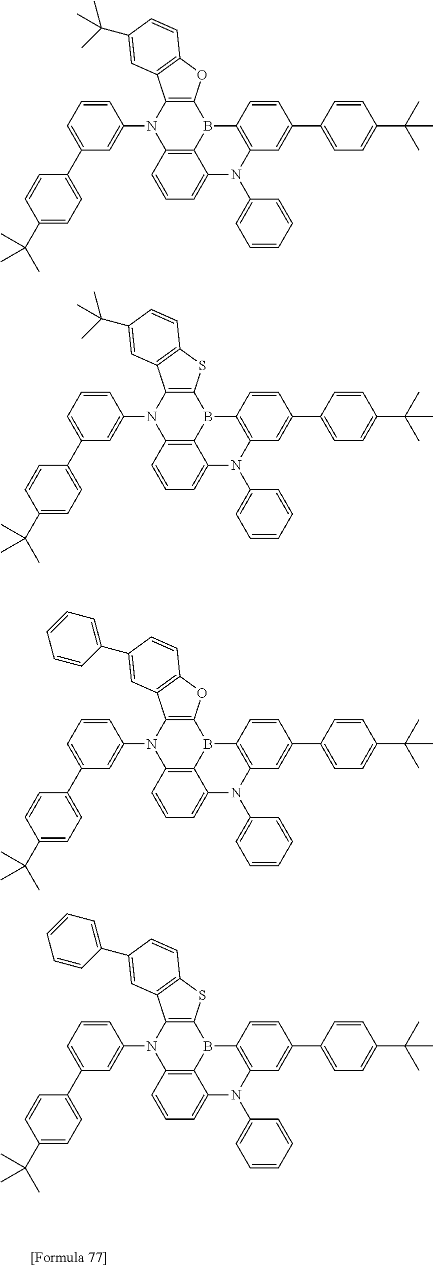

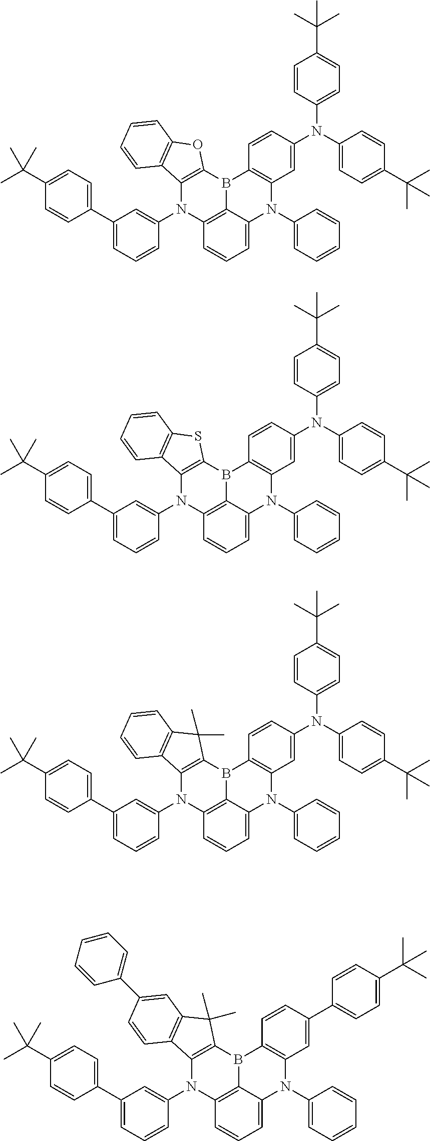

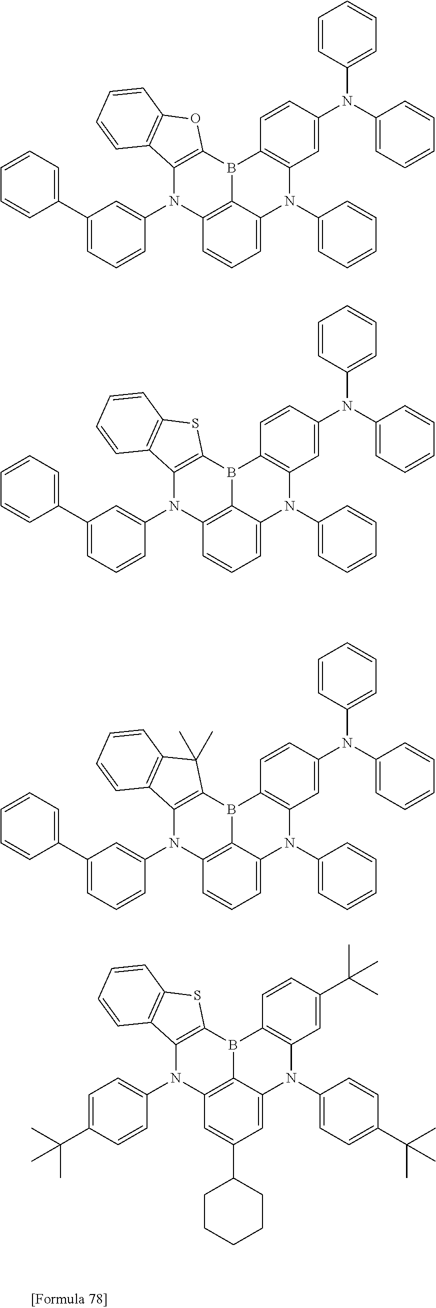

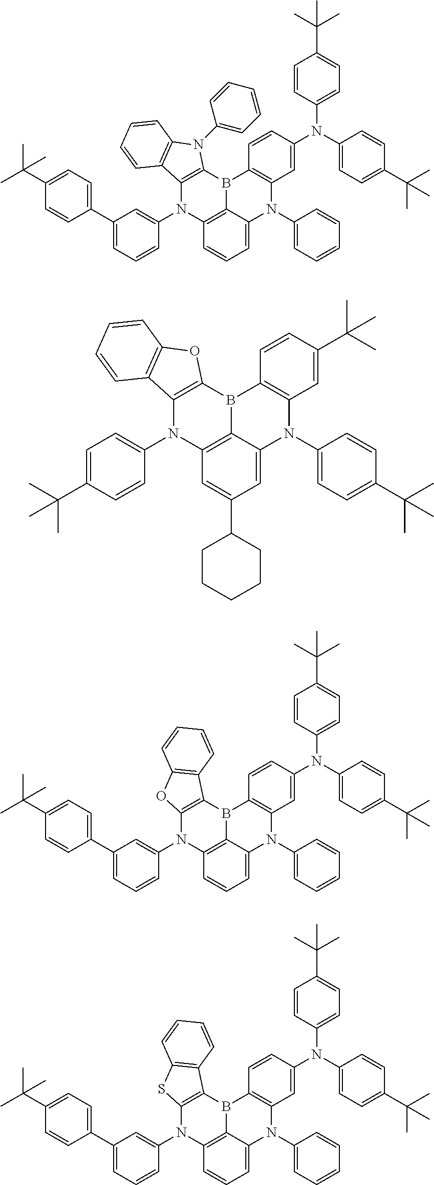

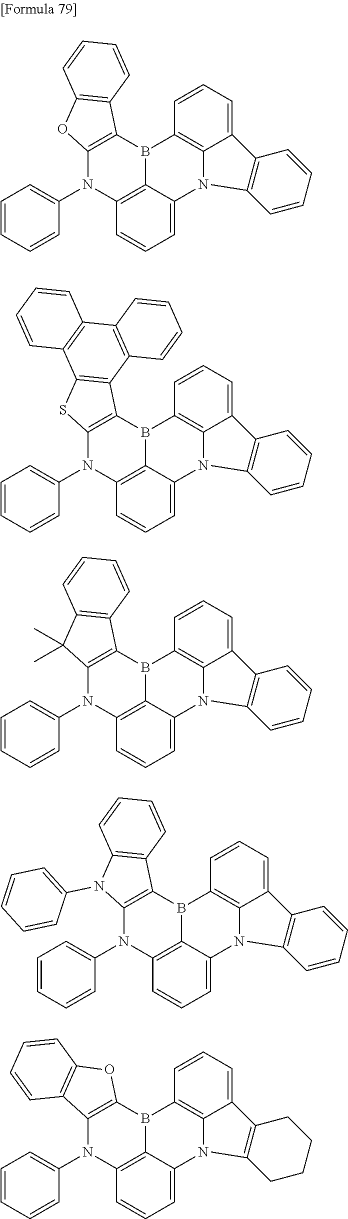

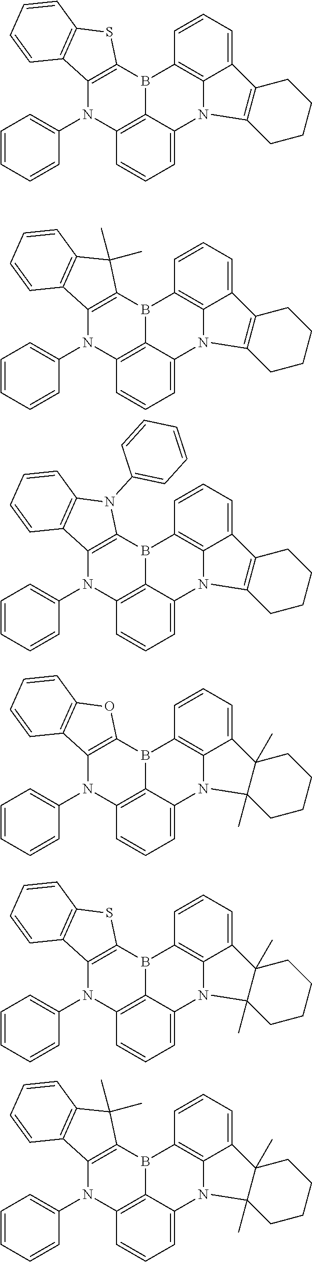

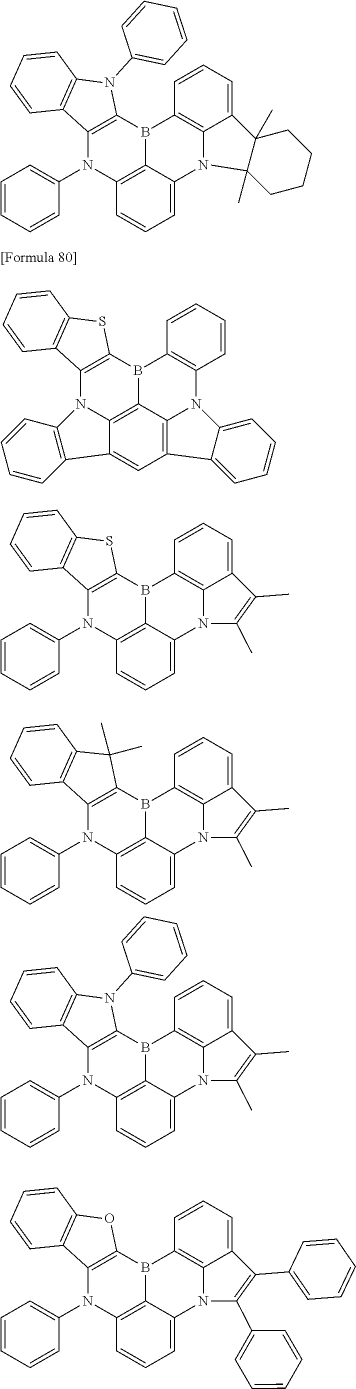

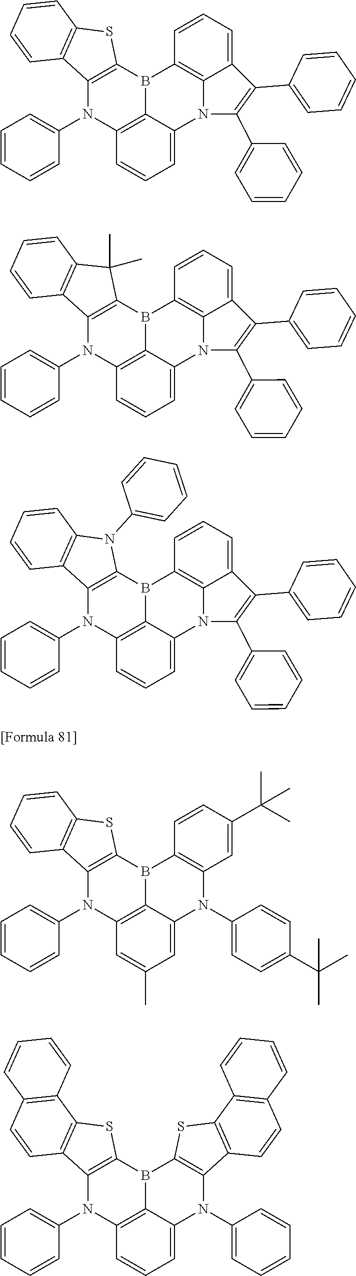

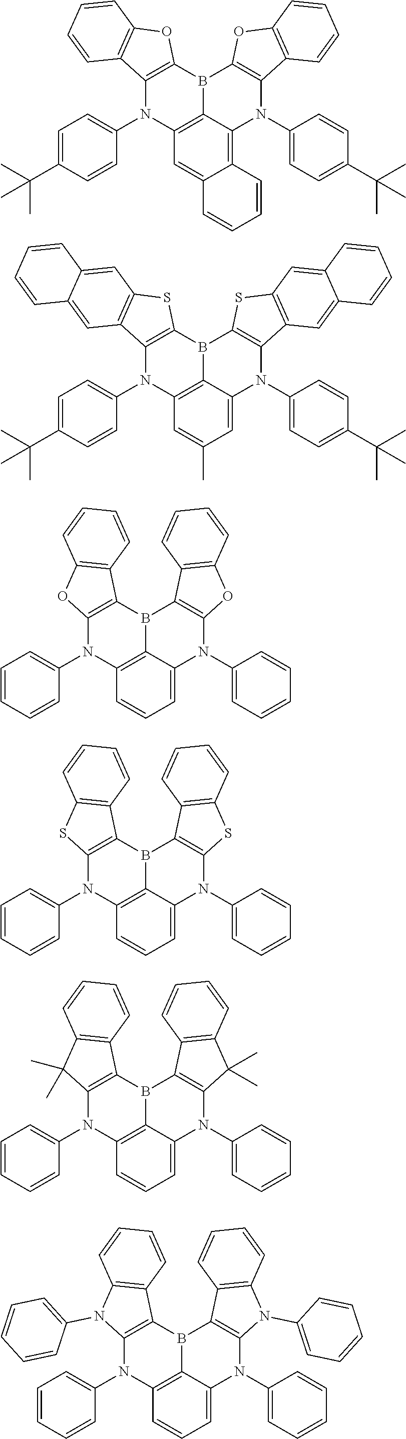

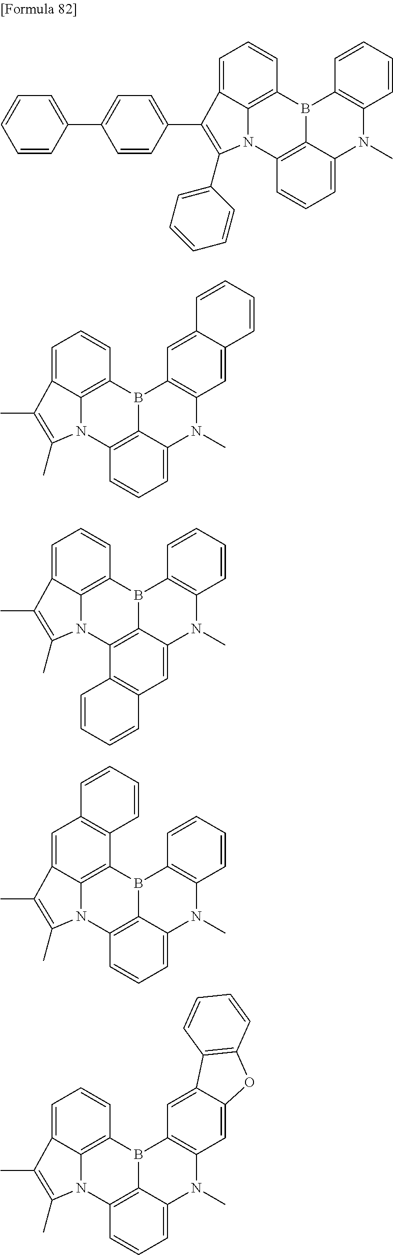

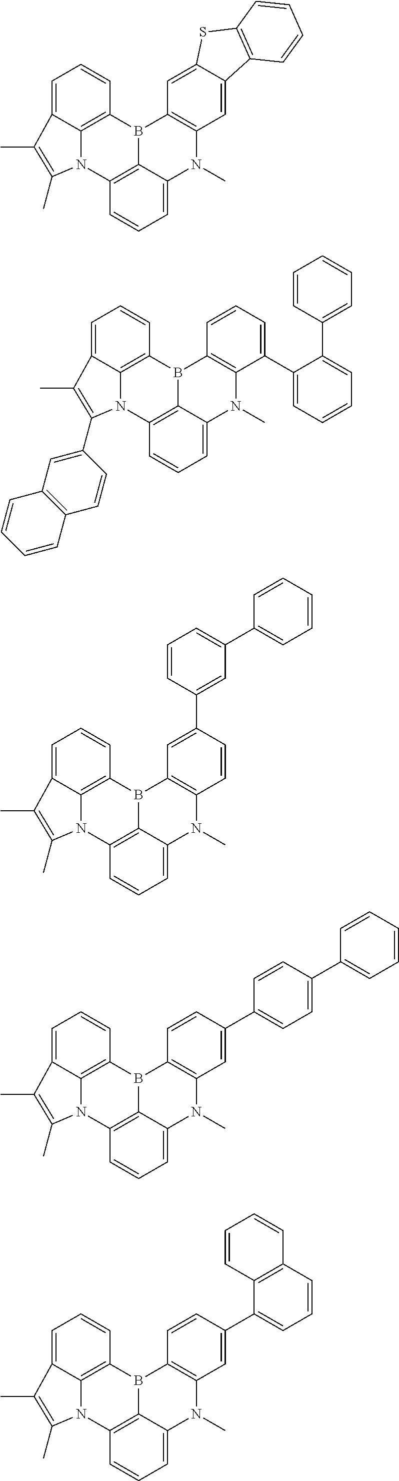 [SMILES: Cc1c(C)n2c3c(cccc13)B1c3cc4sc5ccccc5c4cc3N(C)c3cccc-2c31 ; Cc1c(-c2ccc3ccccc3c2)n2c3c(cccc13)B1c3cccc(-c4ccccc4-c4ccccc4)c3N(C)c3cccc-2c31 ; Cc1c(C)n2c3c(cccc13)B1c3cc(-c4cccc(-c5ccccc5)c4)ccc3N(C)c3cccc-2c31 ; Cc1c(C)n2c3c(cccc13)B1c3ccc(-c4ccc(-c5ccccc5)cc4)cc3N(C)c3cccc-2c31 ; Cc1c(C)n2c3c(cccc13)B1c3ccc(-c4cccc5ccccc45)cc3N(C)c3cccc-2c31]

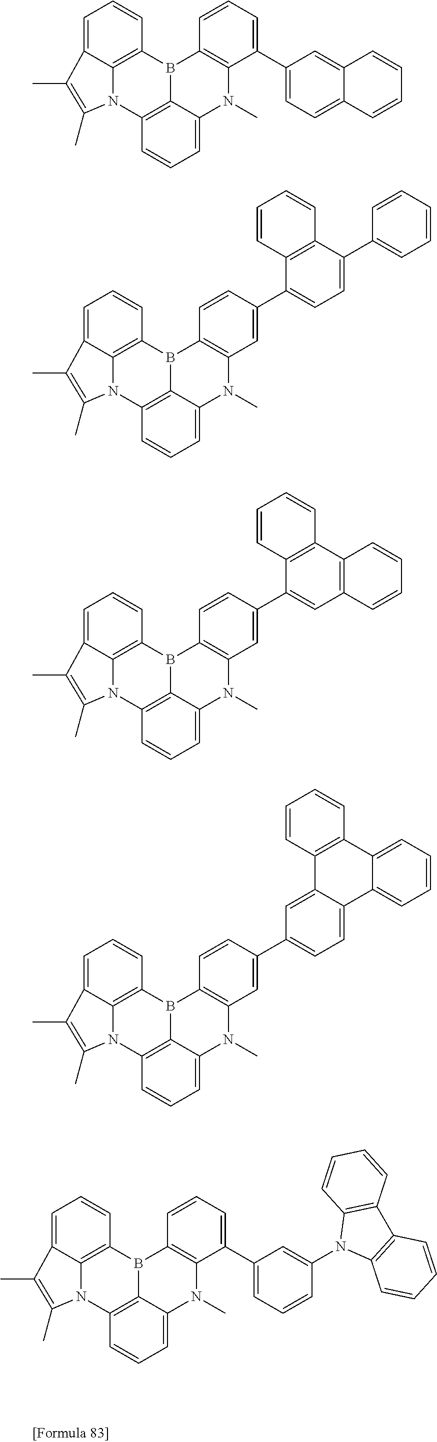 [SMILES: Cc1c(C)n2c3c(cccc13)B1c3cccc(-c4ccc5ccccc5c4)c3N(C)c3cccc-2c31 ; Cc1c(C)n2c3c(cccc13)B1c3ccc(-c4ccc(-c5ccccc5)c5ccccc45)cc3N(C)c3cccc-2c31 ; Cc1c(C)n2c3c(cccc13)B1c3ccc(-c4cc5ccccc5c5ccccc45)cc3N(C)c3cccc-2c31 ; Cc1c(C)n2c3c(cccc13)B1c3ccc(-c4ccc5c6ccccc6c6ccccc6c5c4)cc3N(C)c3cccc-2c31 ; Cc1c(C)n2c3c(cccc13)B1c3cccc(-c4cccc(-n5c6ccccc6c6ccccc65)c4)c3N(C)c3cccc-2c31]

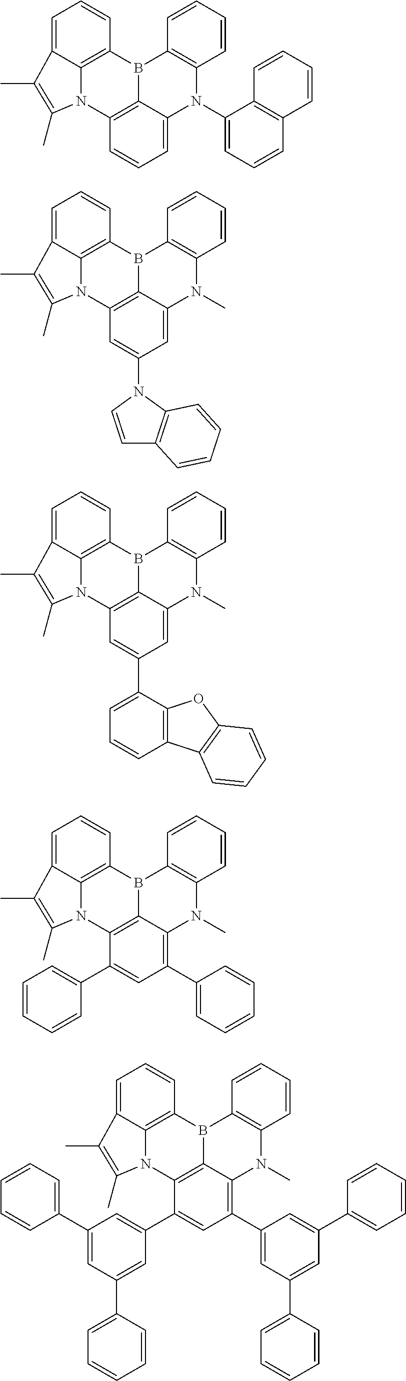

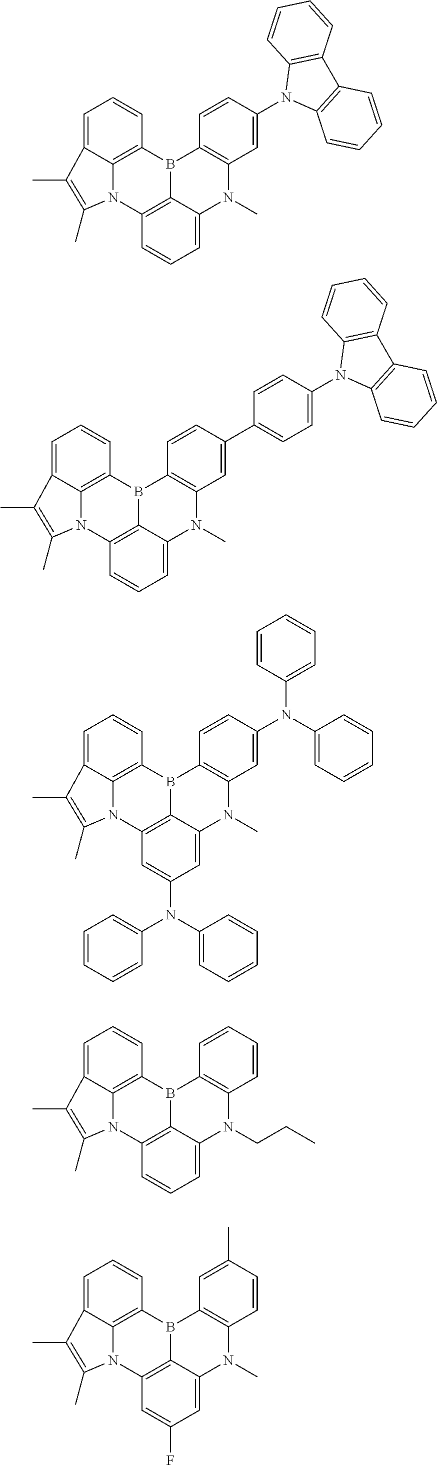

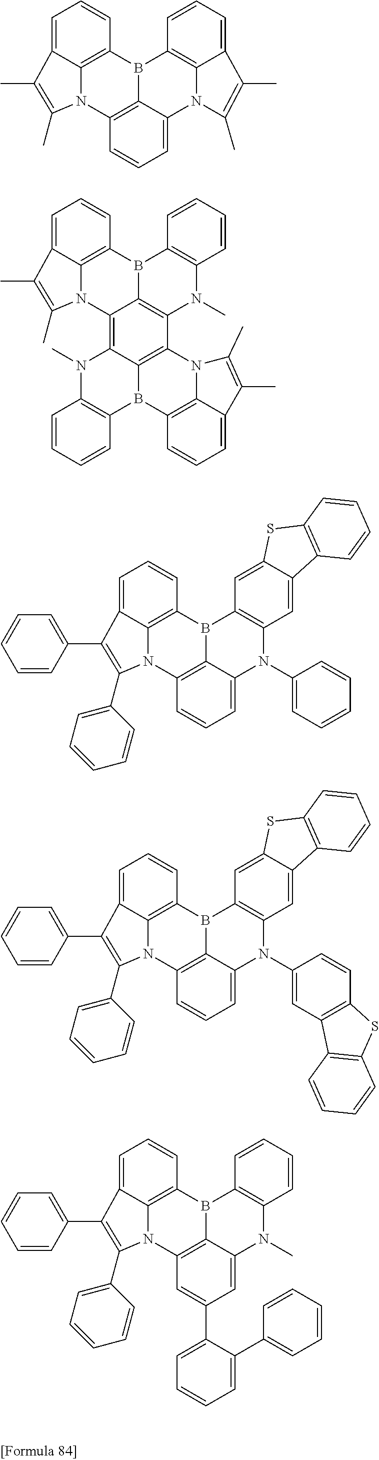

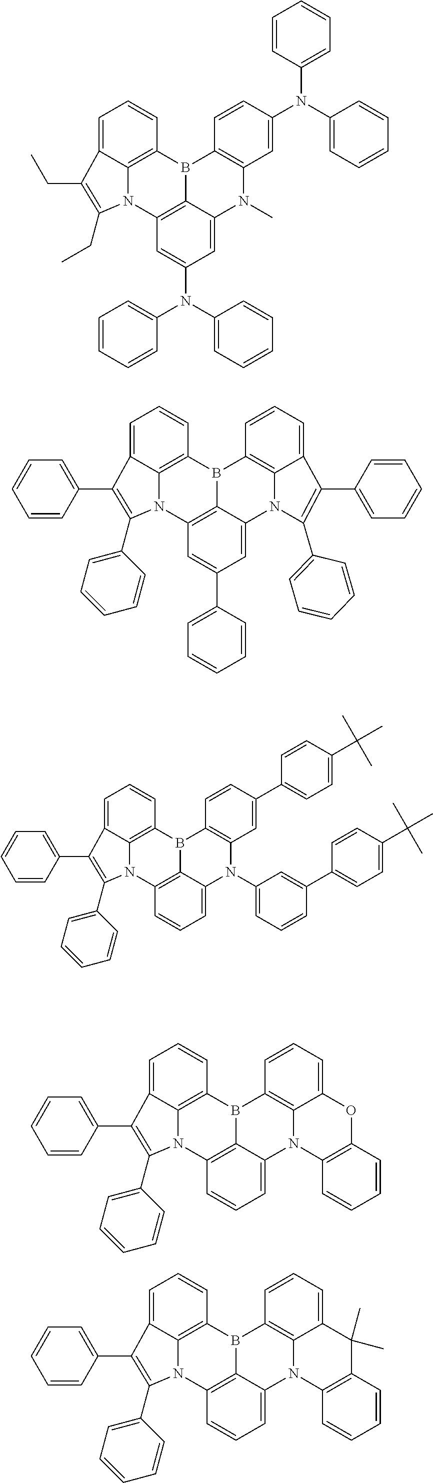

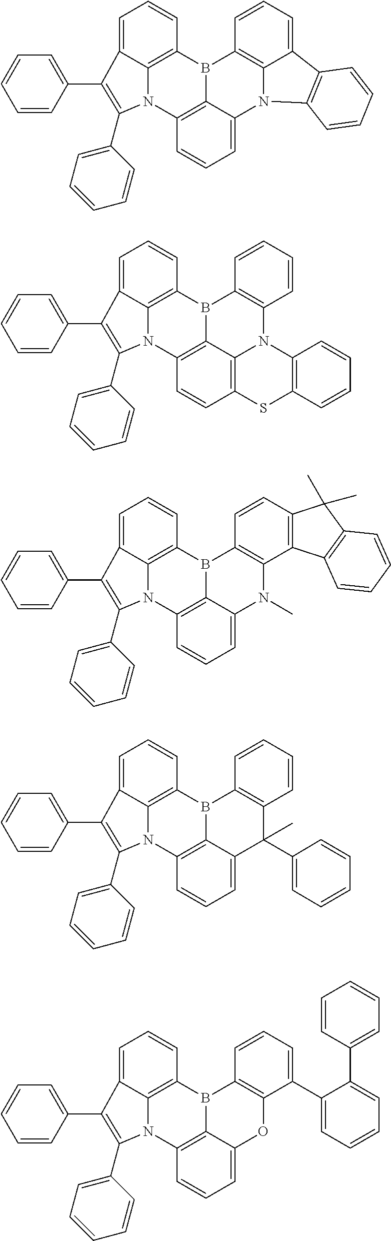

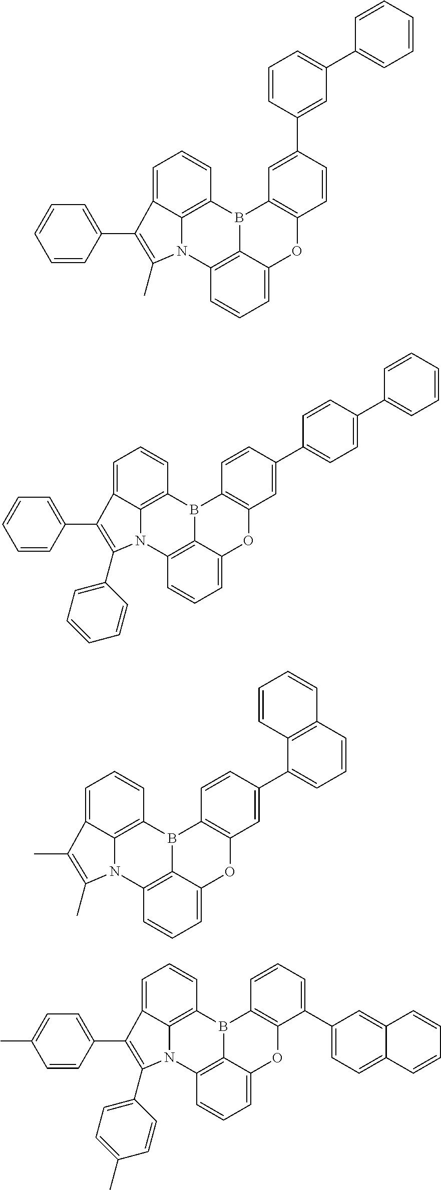

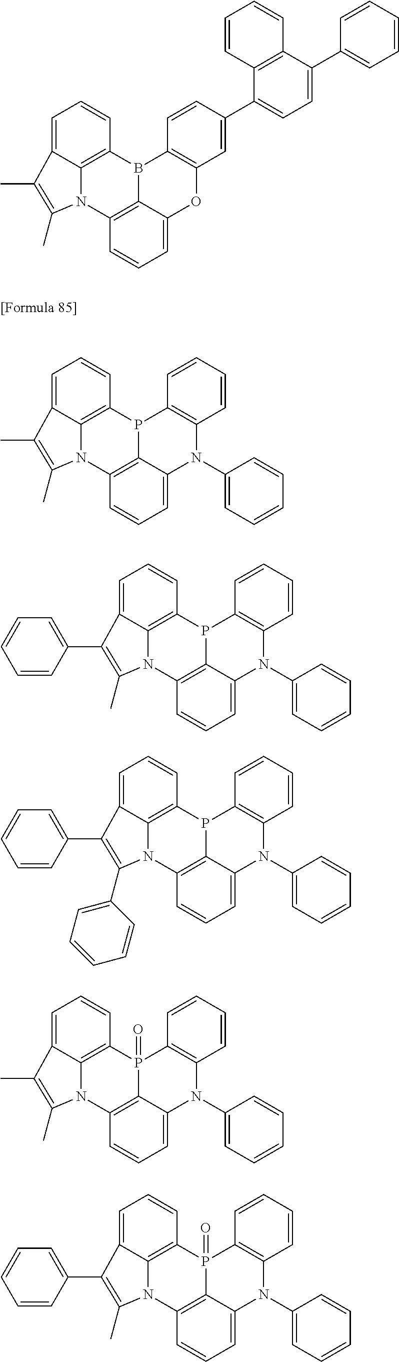

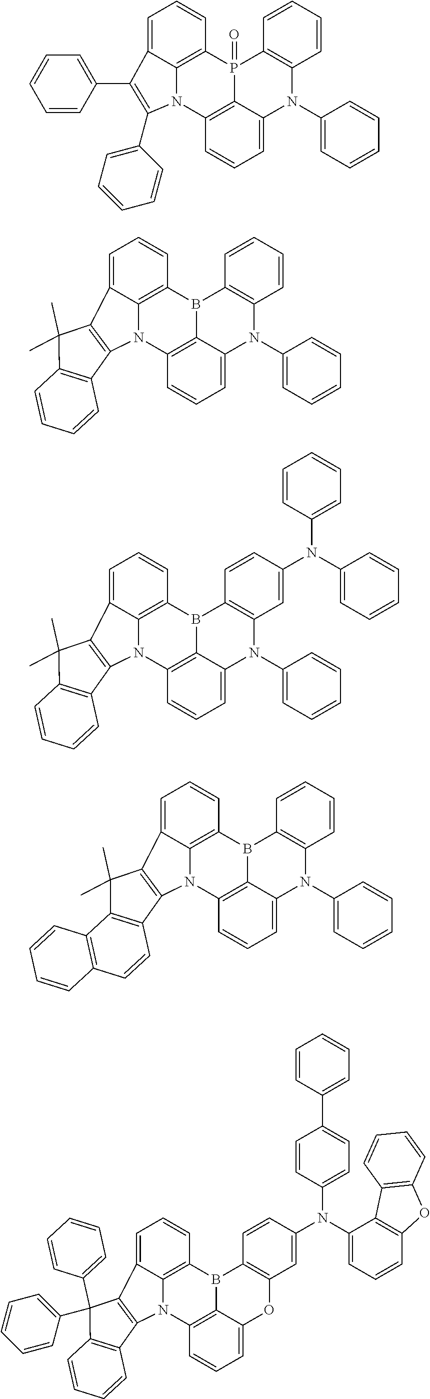

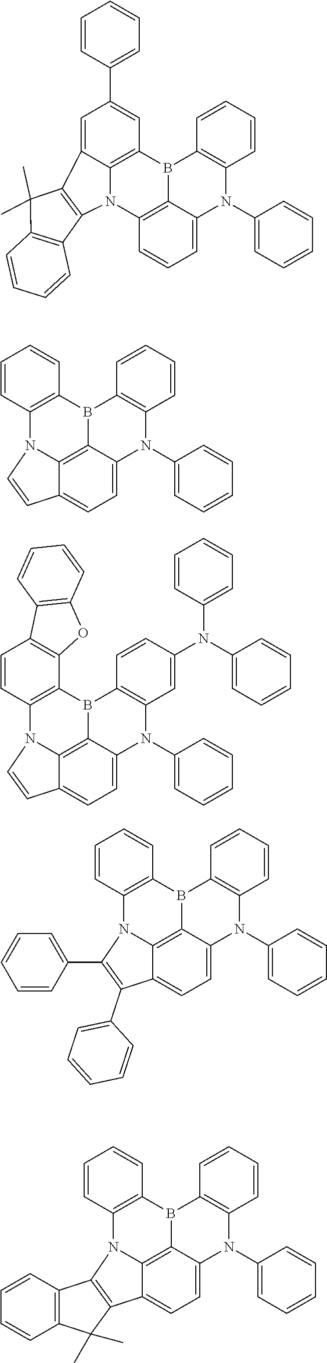

### Second Compound

The second compound according to the exemplary embodiment is a thermally activated delayed fluorescent compound.

The second compound is represented by a formula (2) below.

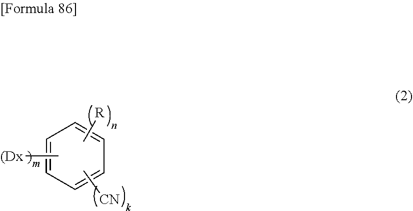

In the formula (2):
k is 1, 2, 3, 4, or 5;
Dx is a group represented by a formula (21), a formula (22), or a formula (23) below;
m is 1, 2, 3, or 4;
when m is 2, 3, or 4, a plurality of Dx are mutually the same or different;
n is 0, 1, 2, or 3;
when n is 2 or 3, a plurality of R are mutually the same or different;
a sum of m and n is 2 or more;
each R is independently a halogen atom or a substituent; and
each R as a substituent is independently a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted cycloalkyl group having 3 to 30 ring carbon atoms, a group represented by —Si(R₉₁₁)(R₉₁₂)(R₉₁₃), a group represented by —O—(R₉₁₄), a group represented by —S—(R₉₁₅), or a group represented by —N(R₉₁₆)(R₉₁₇).

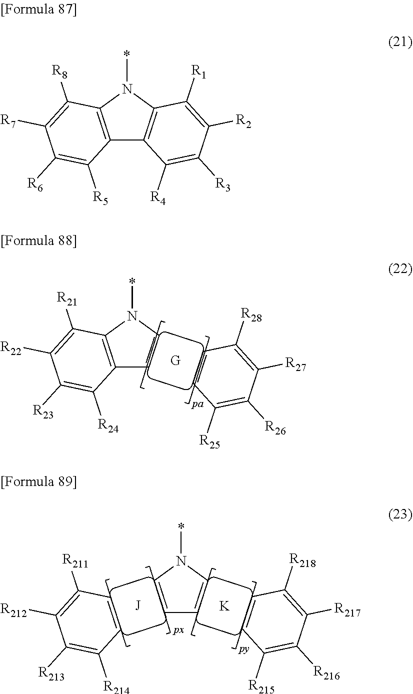

R₁ to R₈ in the formula (21) are each independently a hydrogen atom, a halogen atom, or a substituent.

R₂₁ to R₂₈ in the formula (22) are each independently a hydrogen atom, a halogen atom, or a substituent, or at least one combination of a combination of R₂₁ and R₂₂, a combination of R₂₂ and R₂₃, a combination of R₂₃ and R₂₄, a combination of R₂s and R₂s, a combination of R₂₆ and R₂₇, or a combination of R₂₇ and R₂₅ are mutually bonded to form a ring.

R₂₁₁ to R₂₁₈ in the formula (23) are each independently a hydrogen atom, a halogen atom, or a substituent, or at least one combination of a combination of R₂₁₁ and R₂₁₂, a combination of R₂₁₂ and R₂₁₃, a combination of R₂₁₃ and R₂₁₄, a combination of R₂₁₅ and R₂₁₆, a combination of R₂₁₆ and R₂₁₇, or a combination of R₂₁₇ and R₂₁₈ are mutually bonded to form a ring.

R₁ to R₈ as a substituent, R₂₁ to R₂₅ as a substituent, and R₂₁₁ to R₂₁₈ as a substituent are each independently a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted cycloalkyl group having 3 to 30 ring carbon atoms, a group represented by —Si(R₉₁₁)(R₉₁₂)(R₉₁₃), a group represented by —O—(R₉₁₄), a group represented by —S—(R₉₁₅), or a group represented by —N(R₉₁₆)(R₉₁₇).

In the formulae (22) and (23):
a ring G, a ring J, and a ring K are each independently any cyclic structure selected from the group consisting of cyclic structures represented by formulae (24), (25), and (26) below;
the ring G, the ring J, and the ring K are fused with adjacent rings at any positions;
pa, px and py are each independently 1, 2, 3, or 4;
when pa is 2, 3, or 4, a plurality of rings G are mutually the same or different;
when px is 2, 3, or 4, a plurality of rings J are mutually the same or different;
when py is 2, 3, or 4, a plurality of rings K are mutually the same or different; and
each * in the formulae (21) to (23) represents a bonding position to a benzene ring in the formula (2).

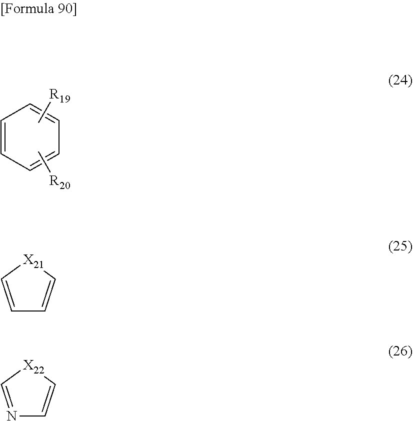

In the formula (24): R₁₉ and R₂₀ are each independently a hydrogen atom, a halogen atom, or a substituent, or a combination of R₁₉ and R₂₀ are mutually bonded to form a ring.

In the formulae (25) and (26):
X₂₁ and X₂₂ are each independently a sulfur atom, an oxygen atom, NR₂₂₀,
CR₂₂₁R₂₂₂, or SiR₂₂₃R₂₂₄;
R₂₂₀ is a hydrogen atom, a halogen atom, or a substituent;
R₂₂₁ and R₂₂₂ are each independently a hydrogen atom, a halogen atom, or a substituent, or a combination of R₂₂₁ and R₂₂₂ are mutually bonded to form a ring;
R₂₂₃ and R₂₂₄ are each independently a hydrogen atom, a halogen atom, or a substituent, or a combination of R₂₂₃ and R₂₂₄ are mutually bonded to form a ring; and
R₁₉, R₂₀, R₂₂₀, R₂₂₁, R₂₂₂, R₂₂₃, and R₂₂₄ as a substituent each independently represent the same as R₁ to R₈ as a substituent.

In the first compound and the second compound, R₉₁₁ to R₉₁₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₁₁ are present, the plurality of R₁₁ are mutually the same or different;
when a plurality of R₉₁₂ are present, the plurality of R₉₁₂ are mutually the same or different;
when a plurality of R₉₁₃ are present, the plurality of R₉₁₃ are mutually the same or different;
when a plurality of R₉₁₄ are present, the plurality of R₉₁₄ are mutually the same or different;
when a plurality of R₉₁₅ are present, the plurality of R₉₁₅ are mutually the same or different;
when a plurality of R₉₁₆ are present, the plurality of R₉₁₆ are mutually the same or different; and
when a plurality of R₉₁₇ are present, the plurality of R₉₁₇ are mutually the same or different.

In an exemplary organic EL device (first example of the organic EL device) of the exemplary embodiment, at least one Dx in the formula (2) representing the second compound is a group represented by the formula (22) or (23). In the first example of the organic EL device, at least one Dx is a group represented by the formula (22) in which pa is 2, 3, or 4 and each ring G has any cyclic structure selected from the group consisting of cyclic structures represented by formulae (25) and (26) below, or at least one Dx is a group represented by the formula (23) in which at least one of px or py is 2, 3, or 4 and at least one ring J, at least one ring K, or both of at least one ring J and at least one ring K have any cyclic structure selected from the group consisting of cyclic structures represented by the formulae (25) and (26). In the first example of the organic EL device, the emitting layer contains the second compound having at least one group represented by the formula (22) or (23) in combination with any one of the above-listed compounds as the first compound.

In an exemplary organic EL device (second example of the organic EL device) of the exemplary embodiment, the second compound is a compound represented by the formula (2). In the second example of the organic EL device, the emitting layer contains the compound represented by the formula (2) in combination with the first compound represented by one of the formulae (15), (16), (171), (172), and (18).

In an exemplary organic EL device (third example of the organic EL device) of the exemplary embodiment, the second compound is a compound that is represented by the formula (2) and does not contain a group represented by the formula (22) or (23). In the third example of the organic EL device, the emitting layer contains the second compound not having a group represented by the formula (22) or (23) in combination with the first compound represented by one of the formulae (15), (16), (171), (172), and (18).

In the exemplary embodiment, m in the formula (2) is preferably 2.

In the exemplary embodiment, a compound represented by the formula (2) is preferably represented by a formula (210), (220) or (230) below.

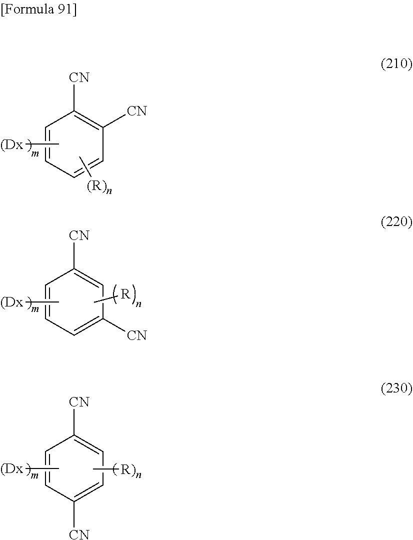

In the formulae (210), (220), and (230), Dx, m, R, and n respectively represent the same as Dx, m, R, and n in the formula (2).

In the exemplary embodiment, a compound represented by the formula (2) is also preferably any compound selected from the group consisting of compounds represented by formulae (211) to (218) below.

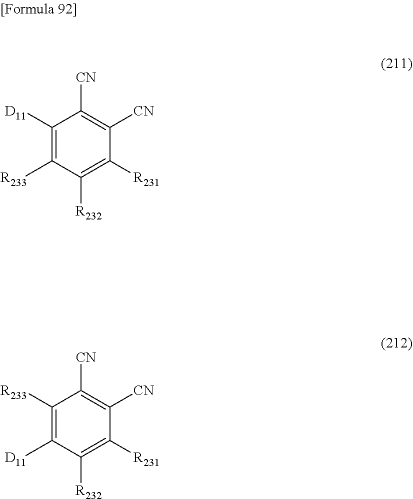

In the formulae (211) and (212):
D₁₁ is a group represented by the formula (22) or (23); and
R₂₃₁ to R₂₃₃ each independently represent the same as R in the formula (2), at least one of R₂₃₁ to R₂₃₃ being a substituent, R₂₃₁ to R₂₃₃ as a substituent representing the same as R as a substituent in the formula (2).

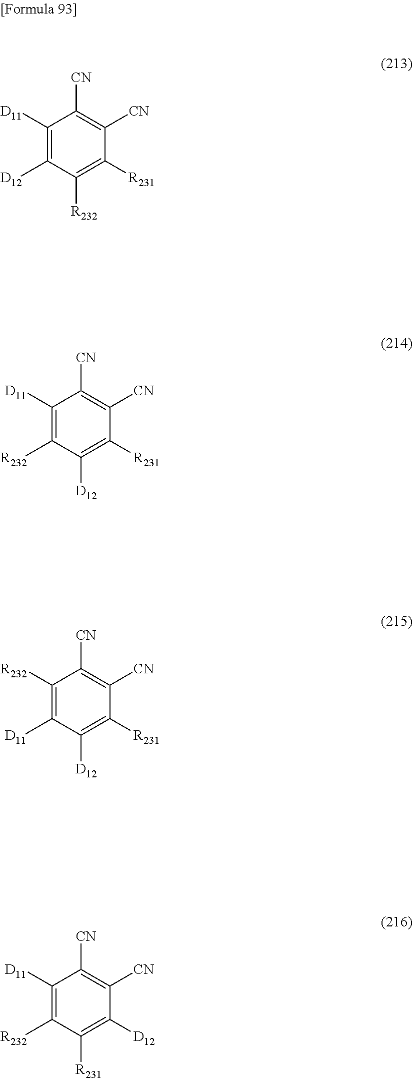

In the formulae (213) to (216):
D₁₁ and D₁₂ each independently represent the same as Dx in the formula (2), at least one of D₁₁ or D₁₂ being a group represented by the formula (22) or (23); and
R₂₃₁ and R₂₃₂ each independently represent the same as R in the formula (2), at least one of R₂₃₁ or R₂₃₂ being a substituent, R₂₃₁ and R₂₃₂ as a substituent representing the same as R as a substituent in the formula (2).

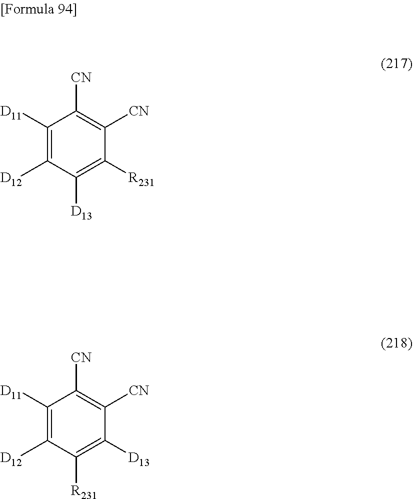

In the formulae (217) and (218):
D₁₁ to D₁₃ each independently represent the same as Dx in the formula (2), at least one of D₁₁ to D₁₃ being a group represented by the formula (22) or (23); and
R₂₃₁ is a substituent and R₂₃₁ as a substituent represents the same as R as a substituent in the formula (2).

In the exemplary embodiment, a compound represented by the formula (2) is also preferably any compound selected from the group consisting of compounds represented by formulae (221) to (229) below.

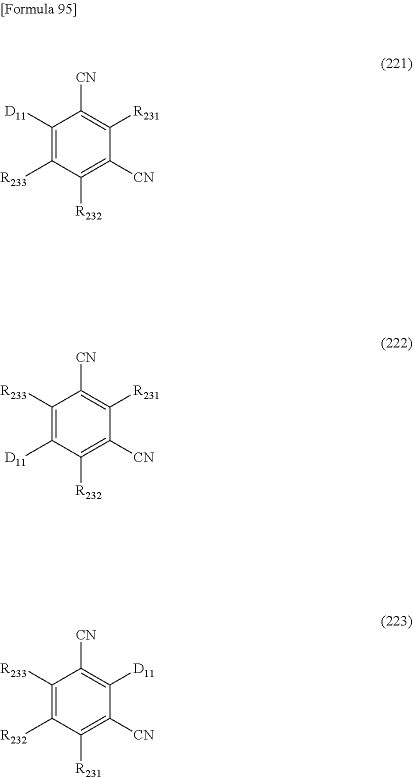

In the formulae (221) to (223):
D₁₁ is a group represented by the formula (22) or (23); and
R₂₃₁ to R₂₃₃ each independently represent the same as R in the formula (2), at least one of R₂₃₁ to R₂₃₃ being a substituent, R₂₃₁ to R₂₃₃ as a substituent representing the same as R as a substituent in the formula (2).

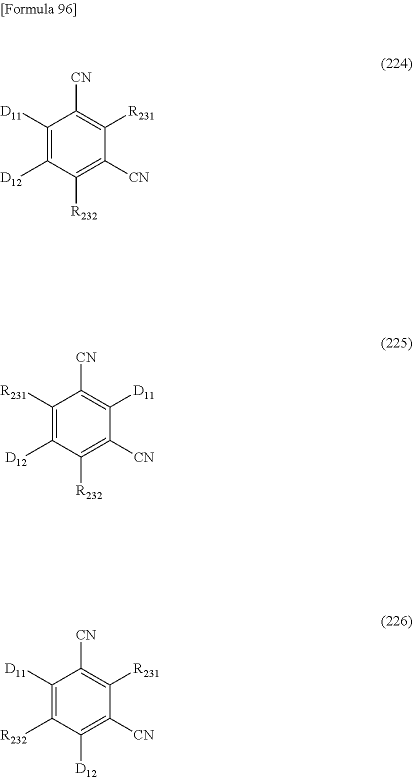

In the formulae (224) to (226):
D₁₁ and D₁₂ each independently represent the same as Dx in the formula (2), at least one of D₁₁ or D₁₂ being a group represented by the formula (22) or (23); and
R₂₃₁ and R₂₃₂ each independently represent the same as R in the formula (2), at least one of R₂₃₁ or R₂₃₂ being a substituent, R₂₃₁ and R₂₃₂ as a substituent representing the same as R as a substituent in the formula (2).

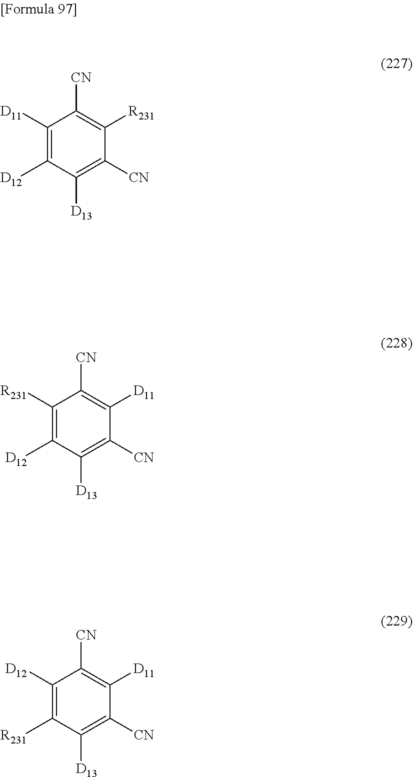

In the formulae (227) to (229):
D₁₁ to D₁₃ each independently represent the same as Dx in the formula (2), at least one of D₁₁ to D₁₃ being a group represented by the formula (22) or (23); and
R₂₃₁ is a substituent and R₂₃₁ as a substituent represents the same as R as a substituent in the formula (2).

In the exemplary embodiment, a compound represented by the formula (2) is also preferably a compound represented by the formula (226).

In the exemplary embodiment, a compound represented by the formula (2) is also preferably any compound selected from the group consisting of compounds represented by formulae (231) to (235) below.

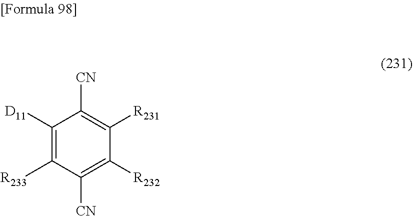

In the formula (231):
D₁₁ is a group represented by the formula (22) or (23); and
R₂₃₁ to R₂₃₃ each independently represent the same as R in the formula (2), at least one of R₂₃₁ to R₂₃₃ being a substituent, R₂₃₁ to R₂₃₃ as a substituent representing the same as R as a substituent in the formula (2).

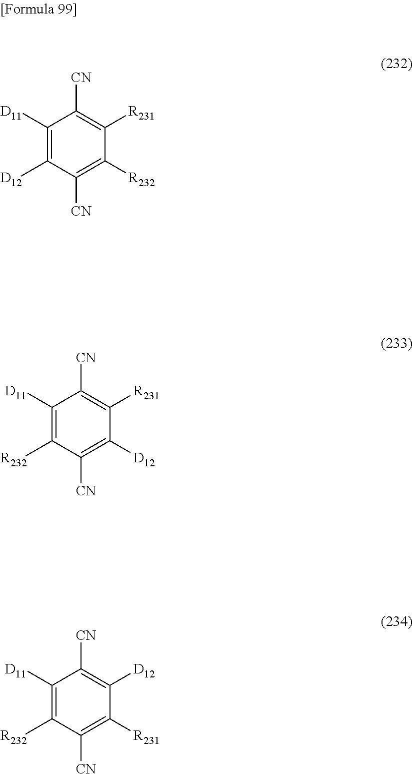

In the formulae (232) to (234):
D₁₁ and D₁₂ each independently represent the same as Dx in the formula (2), at least one of D₁₁ or D₁₂ being a group represented by the formula (22) or (23); and
R₂₃₁ and R₂₃₂ each independently represent the same as R in the formula (2), at least one of R₂₃₁ or R₂₃₂ being a substituent, R₂₃₁ and R₂₃₂ as a substituent representing the same as R as a substituent in the formula (2).

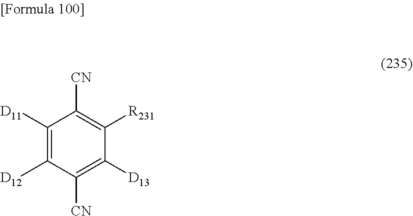

In the formula (235):
D₁₁ to D₁₃ each independently represent the same as Dx in the formula (2), at least one of D₁₁ to D₁₃ being a group represented by the formula (22) or (23); and
R₂₃₁ is a substituent and R₂₃₁ as a substituent represents the same as R as a substituent in the formula (2).

In the exemplary embodiment, a compound represented by the formula (2) is also preferably a compound represented by the formula (234).

In the exemplary embodiment, a compound represented by the formula (2) is also preferably a compound represented by a formula (236) below.

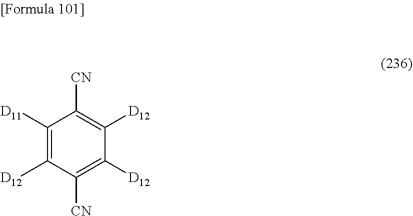

In the formula (236):
D₁₁ is a group represented by a formula (22A), (22B), (22C), (22D), (22E) or (22F) below;
three D₁₂ are each independently a group represented by the formula (21); and
D₁₁ and D₁₂ are mutually different. D₁₂ are

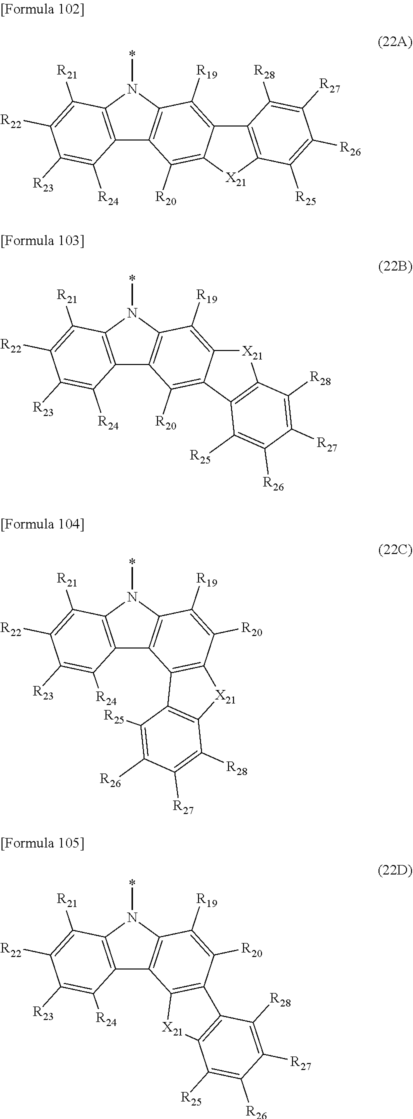

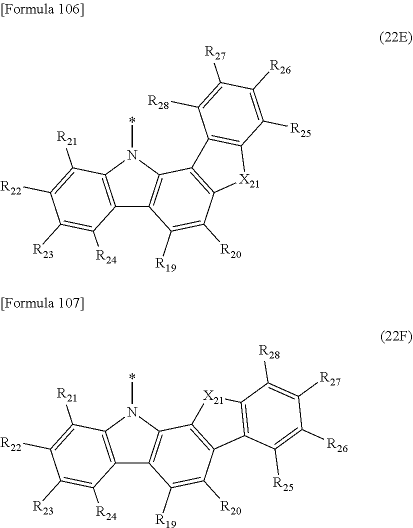

In the formulae (22A), (22B), (22C), (22D), (22E), and (22F):
R₂₁ to R₂₈ each independently represent the same as R₂₁ to R₂₈ in the formula (22);
R₁₉ and R₂₀ each independently represent the same as R₁₉ and R₂₀ in the formula (24);
X₂₁ represents the same as X₂₁ in the formula (25); and
each * in the formulae (22A), (22B), (22C), (22D), (22E), and (22F) represents a bonding position to a benzene ring in the formula (2).

In the formulae (22A), (22B), (22C), (22D), (22E), and (22F), it is preferable that none of a combination of R₂₁ and R₂₂, a combination of R₂₂ and R₂₃, a combination of R₂₃ and R₂₄, a combination of R₂₅ and R₂₆, a combination of R₂₆ and R₂₇, a combination of R₂₇ and R₂₅, and a combination of R₁₉ and R₂₀ are mutually bonded.

In the second compound, a group represented by the formula (22) is preferably a group selected from the group consisting of groups represented by the formulae (22A), (22D), and (22F).

In the second compound, X₂₁ is preferably an oxygen atom or a sulfur atom.

The compound according to the exemplary embodiment preferably has, as Dx in the formula (2), at least one group selected from the group consisting of groups represented by the formulae (22A), (22B), (22C), (22D), (22E), and (22F).

More preferably, the compound according to the exemplary embodiment has, as Dx in the formula (2), at least one group selected from the group consisting of groups represented by the formulae (22A), (22B), (22C), (22D), (22E), and (22F), in which X₂₁ is an oxygen atom or a sulfur atom.

Each Dx in the formulae (210), (220), and (230) is preferably independently any group selected from the group consisting of groups represented by the formulae (22A), (22B), (22C), (22D), (22E), and (22F).

D₁₁, D₁₂, and D₁₃ in the formulae (211) to (218), (221) to (229), (231) to (235) are each independently any group selected from the group consisting of groups represented by the formulae (22A), (22B), (22C), (22D), (22E), and (22F).

In the exemplary embodiment, each R as a substituent of the second compound is preferably independently a substituted or unsubstituted aryl group having 6 to 30 ring carbon atoms, a substituted or unsubstituted heterocyclic group having 5 to 30 ring atoms, or a substituted or unsubstituted alkyl group having 1 to 6 carbon atoms.

In the exemplary embodiment, R₁ to R₈, R₂₁ to R₂₈, and R₂₁₁ to R₂₁₈ of the second compound are preferably each independently a hydrogen atom, a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon atoms.

In the exemplary embodiment, R₁ to R₈, R₂₁ to R₂₈, and R₂₁₁ to R₂₁₈ of the second compound are preferably each independently a hydrogen atom or a substituted or unsubstituted aryl group having 6 to 30 ring carbon atoms.

In the second compound of the exemplary embodiment, R₁ to R₈, R₂₁ to R₂₈, and R₂₁₁ to R₂₁₈ are preferably each a hydrogen atom.

In the second compound, the substituent for “the substituted or unsubstituted” group is preferably an unsubstituted alkyl group having 1 to 25 carbon atoms, an unsubstituted alkenyl group having 2 to 25 carbon atoms, an unsubstituted alkynyl group having 2 to 25 carbon atoms, an unsubstituted cycloalkyl group having 3 to 25 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), an unsubstituted aralkyl group having 7 to 50 carbon atoms, a group represented by —C(═O)R₉₀₀, a group represented by —COOR₉₀₉, a group represented by —S(═O)₂R₉₄₁, a group represented by —(═O)(R₉₄₂)(R₉₄₃), a group represented by —Ge(R₉₄₄)(R₉₄₅)(R₉₄₆), a halogen atom, a cyano group, a nitro group, an unsubstituted aryl group having 6 to 25 ring carbon atoms, or an unsubstituted heterocyclic group having 5 to 25 ring atoms.
R₉₀₁ to R₉₀₉ and R₉₄₁ to R₉₄₆ are preferably each independently a hydrogen atom, an unsubstituted alkyl group having 1 to 25 carbon atoms, an unsubstituted aryl group having 6 to 25 ring carbon atoms, or an unsubstituted heterocyclic group having 5 to 25 ring atoms.

In the second compound, the substituent for “the substituted or unsubstituted” group is preferably a halogen atom, an unsubstituted alkyl group having 1 to 25 carbon atoms, an unsubstituted aryl group having 6 to 25 ring carbon atoms, or an unsubstituted heterocyclic group having 5 to 25 ring atoms.

In the second compound, the substituent for “the substituted or unsubstituted” group is preferably an unsubstituted alkyl group having 1 to 10 carbon atoms, an unsubstituted aryl group having 6 to 12 ring carbon atoms, or an unsubstituted heterocyclic group having 5 to 12 ring atoms.

In the second compound, the groups specified to be “substituted or unsubstituted” are each also preferably an “unsubstituted” group.

In the organic EL device according to the exemplary embodiment, it is preferable that a delayed fluorescent material as the second compound is the host material.

In the organic EL device according to the exemplary embodiment, it is preferable that a delayed fluorescent material as the second compound is the host material and a compound according to the first exemplary embodiment as the first compound is the dopant material.

Delayed Fluorescence Delayed fluorescence is explained in “Yuki Hando-tai no Debaisu Bussei (Device Physics of Organic Semiconductors)” (edited by ADACHI, Chihaya, published by Kodansha, on pages 261-268). This document describes that, if an energy difference ΔE₁₃ of a fluorescent material between a singlet state and a triplet state is reducible, a reverse energy transfer from the triplet state to the singlet state, which usually occurs at a low transition probability, would occur at a high efficiency to express thermally activated delayed fluorescence (TADF). Further, a generation mechanism of delayed fluorescence is explained in FIG. 10.38 in the document. The delayed fluorescent material of the exemplary embodiment is preferably a compound exhibiting thermally activated delayed fluorescence generated by such a mechanism.

In general, emission of delayed fluorescence can be checked by measuring the transient PL (Photo Luminescence).

The behavior of delayed fluorescence can also be analyzed based on the decay curve obtained from the transient PL measurement. The transient PL measurement is a method of irradiating a sample with a pulse laser to excite the sample, and measuring the decay behavior (transient characteristics) of PL emission after the irradiation is stopped. PL emission in TADF materials is classified into a light emission component from a singlet exciton generated by the first PL excitation and a light emission component from a singlet exciton generated via a triplet exciton. The lifetime of the singlet exciton generated by the first PL excitation is on the order of nanoseconds and is very short. Therefore, light emission from the singlet exciton rapidly attenuates after irradiation with the pulse laser.

On the other hand, the delayed fluorescence is gradually attenuated due to light emission from a singlet exciton generated via a triplet exciton having a long lifetime. As described above, there is a large temporal difference between the light emission from the singlet exciton generated by the first PL excitation and the light emission from the singlet exciton generated via the triplet exciton. Therefore, the luminous intensity derived from delayed fluorescence can be determined.

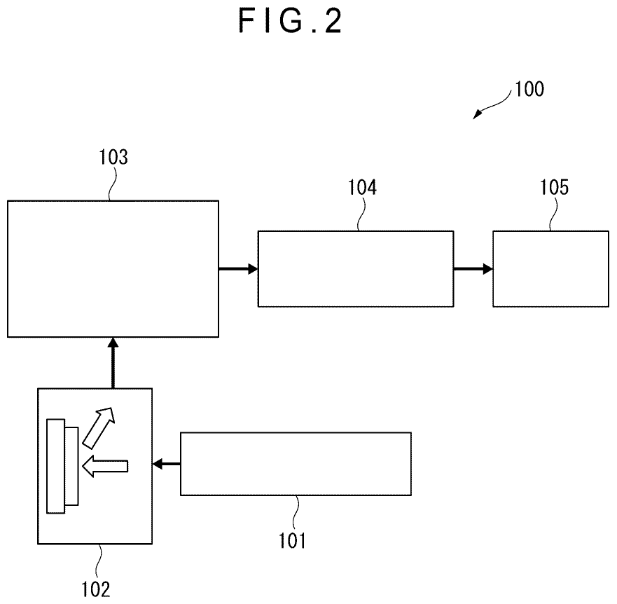

FIG. 2 is a schematic diagram of an exemplary apparatus for measuring the transient PL. An example of a method of measuring the transient PL as illustrated in FIG. 2 and an example of behavior analysis of delayed fluorescence will be described.

A transient PL measuring apparatus 100 in FIG. 2 includes: a pulse laser 101 capable of radiating a light having a predetermined wavelength; a sample chamber 102 configured to house a measurement sample; a spectrometer 103 configured to divide a light radiated from the measurement sample; a streak camera 104 configured to provide a two-dimensional image; and a personal computer 105 configured to import and analyze the two-dimensional image. An apparatus for measuring the transient PL is not limited to the apparatus illustrated in FIG. 2

The sample housed in the sample chamber 102 is obtained by forming a thin film, in which a matrix material is doped with a doping material at a concentration of 12 mass %, on a quartz substrate.

The thin film sample housed in the sample chamber 102 is irradiated with the pulse laser from the pulse laser 101 to excite the doping material. Emission is extracted in a direction of 90 degrees with respect to a radiation direction of the excited light. The extracted emission is divided by the spectrometer 103 to form a two-dimensional image in the streak camera 104. As a result, the two-dimensional image is obtainable in which the ordinate axis represents a time, the abscissa axis represents a wavelength, and a bright spot represents a luminous intensity. When this two-dimensional image is taken out at a predetermined time axis, an emission spectrum in which the ordinate axis represents the luminous intensity and the abscissa axis represents the wavelength is obtainable. Moreover, when this two-dimensional image is taken out at the wavelength axis, a decay curve (transient PL) in which the ordinate axis represents a logarithm of the luminous intensity and the abscissa axis represents the time is obtainable.

For instance, a thin film sample A was prepared as described above from a reference compound H1 below as the matrix material and a reference compound D1 below as the doping material and was measured in terms of the transient PL.

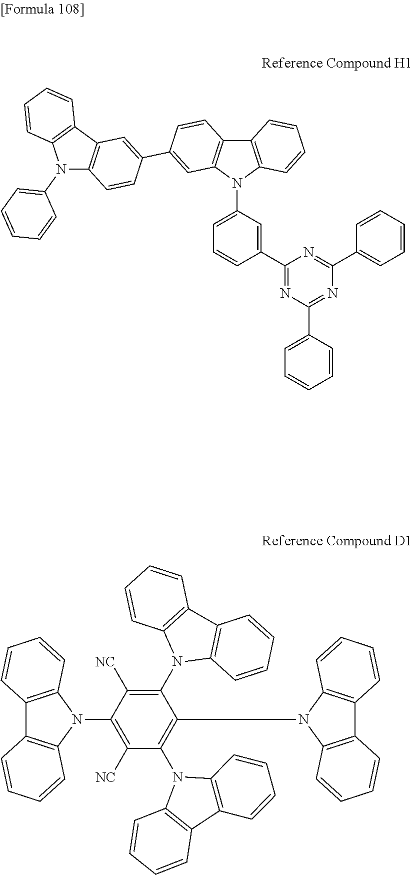 [SMILES: c1ccc(-c2nc(-c3ccccc3)nc(-c3cccc(-n4c5ccccc5c5ccc(-c6ccc7c(c6)c6ccccc6n7-c6ccccc6)cc54)c3)n2)cc1 ; N#Cc1c(-n2c3ccccc3c3ccccc32)c(C#N)c(-n2c3ccccc3c3ccccc32)c(-n2c3ccccc3c3ccccc32)c1-n1c2ccccc2c2ccccc21]

The decay curve was analyzed with respect to the above thin film sample A and a thin film sample B. The thin film sample B was produced in the same manner as described above from a reference compound H2 below as the matrix material and the reference compound D1 as the doping material.

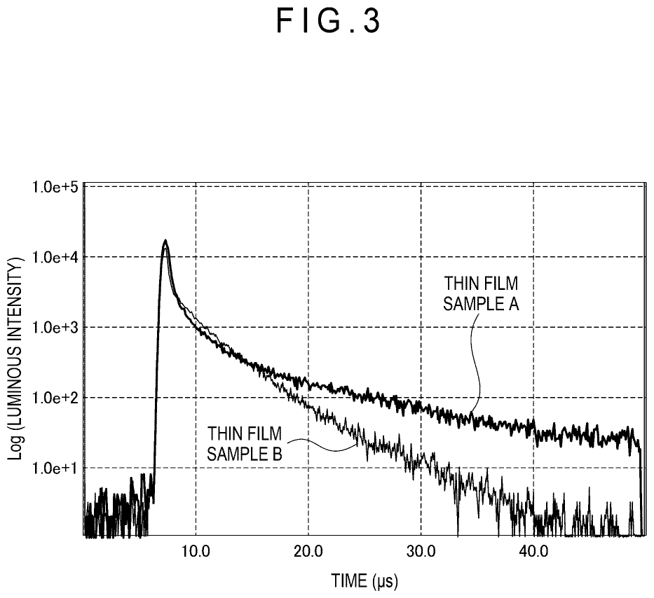

FIG. 3 illustrates decay curves obtained from transient PL obtained by measuring the thin film samples A and B.

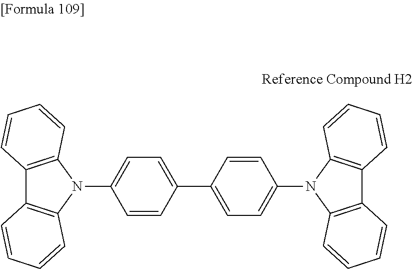 [SMILES: c1ccc2c(c1)c1ccccc1n2-c1ccc(-c2ccc(-n3c4ccccc4c4ccccc43)cc2)cc1]

As described above, an emission decay curve in which the ordinate axis represents the luminous intensity and the abscissa axis represents the time can be obtained by the transient PL measurement. Based on the emission decay curve, a fluorescence intensity ratio between fluorescence emitted from a singlet state generated by photo-excitation and delayed fluorescence emitted from a singlet state generated by reverse energy transfer via a triplet state can be estimated. In a delayed fluorescent material, a ratio of the intensity of the slowly decaying delayed fluorescence to the intensity of the promptly decaying fluorescence is relatively large.

Specifically, Prompt emission and Delay emission are present as emission from the delayed fluorescent material. Prompt emission is observed promptly when the excited state is achieved by exciting the compound of the exemplary embodiment with a pulse beam (i.e., a beam emitted from a pulse laser) having a wavelength absorbable by the delayed fluorescent material. Delay emission is observed not promptly when the excited state is achieved but after the excited state is achieved.

Herein, a sample produced by the following method is used for measuring delayed fluorescence of the delayed fluorescent material. For instance, the delayed fluorescent material is dissolved in toluene to prepare a dilute solution with an absorbance of 0.05 or less at the excitation wavelength to eliminate the contribution of self-absorption. In order to prevent quenching due to oxygen, the sample solution is frozen and degassed and then sealed in a cell with a lid under an argon atmosphere to obtain an oxygen-free sample solution saturated with argon.

The fluorescence spectrum of the sample solution is measured with a spectrofluorometer FP-8600 (produced by JASCO Corporation), and the fluorescence spectrum of a 9,10-diphenylanthracene ethanol solution is measured under the same conditions. Using the fluorescence area intensities of both spectra, the total fluorescence quantum yield is calculated by an equation (1) in Morris et al. J. Phys. Chem. 80 (1976) 969.

An amount of Prompt emission, an amount of Delay emission and a ratio between the amounts thereof can be obtained according to the method as described in “Nature 492, 234-238, 2012” (Reference Document 1). The amount of Prompt emission and the amount of Delay emission may be calculated using an apparatus different from one described in Reference Document 1 or one shown in FIG. 2

In the exemplary embodiment, provided that an amount of Prompt emission of a measurement target compound (delayed fluorescent material) is denoted by X_{P} and an amount of Delay emission is denoted by X_{D}, a value of X_{D}/X_{P} is preferably 0.05 or more.

The amounts of Prompt emission and Delay emission and a ratio of the amounts thereof in compounds other than the delayed fluorescent material herein are measured in the same manner as those of the delayed fluorescent material.

### ΔST

In the exemplary embodiment, a difference (S₁−T_{77K}) between a lowest singlet energy S₁ and an energy gap T_{77K} at 77K is defined as ΔST.

A difference ΔST(H) between a lowest singlet energy S₁(H) of the delayed fluorescent material and an energy gap T_{77K}(H) at 77K of the delayed fluorescent material is preferably less than 0.3 eV, more preferably less than 0.2 eV, still more preferably less than 0.1 eV, and still further more preferably less than 0.01 eV. In other words, ΔST(H) preferably satisfies a relationship of a numerical formula (Numerical Formula 10, Numerical Formula 11, Numerical Formula 12, or Numerical Formula 13) below.

ΔST(H)=S₁(H)−T_{77K}(H)<0.3 eV (Numerical Formula 10)

ΔST(H)=S₁(H)−T_{77K}(H)<0.2 eV (Numerical Formula 11)

ΔST(H)=S₁(H)−T_{77K}(H)<0.1 eV (Numerical Formula 12)

ΔST(H)=S₁(H)−T_{77K}(H)<0.01 eV (Numerical Formula 13)

Relationship between Triplet Energy and Energy Gap at 77K

Here, a relationship between a triplet energy and an energy gap at 77K will be described. In the exemplary embodiment, the energy gap at 77K is different from a typical triplet energy in some aspects.

The triplet energy is measured as follows. First, a solution in which a compound (measurement target) is dissolved in an appropriate solvent is encapsulated in a quartz glass tube to prepare a sample. A phosphorescence spectrum (ordinate axis: phosphorescent luminous intensity, abscissa axis: wavelength) of the sample is measured at a low temperature (77K). A tangent is drawn to the rise of the phosphorescence spectrum close to the short-wavelength region. The triplet energy is calculated by a predetermined conversion equation based on a wavelength value at an intersection of the tangent and the abscissa axis.

Herein, of the compounds of the exemplary embodiment, the thermally activated delayed fluorescent compound is preferably a compound having a small ΔST. When ΔST is small, intersystem crossing and inverse intersystem crossing are likely to occur even at a low temperature (77K), so that the singlet state and the triplet state coexist.

As a result, the spectrum to be measured in the same manner as the above includes emission from both the singlet state and the triplet state. Although it is difficult to distinguish from which state, the singlet state or the triplet state, light is emitted, the value of the triplet energy is basically considered dominant.

Accordingly, in the exemplary embodiment, the triplet energy is measured by the same method as a typical triplet energy T, but a value measured in the following manner is referred to as an energy gap T_{77K} in order to differentiate the measured energy from the typical triplet energy in a strict meaning. The measurement target compound is dissolved in EPA (diethylether:isopentane:ethanol=5:5:2 in volume ratio) at a concentration of 10 μmol/L, and the obtained solution is put in a quartz cell to provide a measurement sample. A phosphorescence spectrum (ordinate axis: phosphorescent luminous intensity, abscissa axis: wavelength) of the sample is measured at a low temperature (77K). A tangent is drawn to the rise of the phosphorescence spectrum close to the short-wavelength region. An energy amount is calculated by a conversion equation (F1) below based on a wavelength value λ_{edge} [nm] at an intersection of the tangent and the abscissa axis and is defined as an energy gap T_{77K} at 77K.

Conversion Equation (F1): T_{77K} [eV]=1239.85/λ_{edge}

The tangent to the rise of the phosphorescence spectrum close to the short-wavelength region is drawn as follows. While moving on a curve of the phosphorescence spectrum from the short-wavelength region to the local maximum value closest to the short-wavelength region among the local maximum values of the phosphorescence spectrum, a tangent is checked at each point on the curve toward the long-wavelength of the phosphorescence spectrum. An inclination of the tangent is increased along the rise of the curve (i.e., a value of the ordinate axis is increased). A tangent drawn at a point of the local maximum inclination (i.e., a tangent at an inflection point) is defined as the tangent to the rise of the phosphorescence spectrum close to the short-wavelength region.

A local maximum point where a peak intensity is 15% or less of the maximum peak intensity of the spectrum is not counted as the above-mentioned local maximum peak intensity closest to the short-wavelength region. The tangent drawn at a point that is closest to the local maximum peak intensity closest to the short-wavelength region and where the inclination of the curve is the local maximum is defined as a tangent to the rise of the phosphorescence spectrum close to the short-wavelength region.

For phosphorescence measurement, a spectrophotofluorometer body F-4500 (manufactured by Hitachi High-Technologies Corporation) is usable. Any apparatus for phosphorescence measurement is usable. A combination of a cooling unit, a low temperature container, an excitation light source and a light-receiving unit may be used for phosphorescence measurement.

### Lowest Singlet Energy S₁

A method of measuring the lowest singlet energy S₁ with use of a solution (occasionally referred to as a solution method) is exemplified by a method below.

A toluene solution of a measurement target compound at a concentration of 10 μmol/L is prepared and put in a quartz cell. An absorption spectrum (ordinate axis: absorption intensity, abscissa axis: wavelength) of the thus-obtained sample is measured at a normal temperature (300K). A tangent is drawn to the fall of the absorption spectrum close to the long-wavelength region, and a wavelength value λ_{edge} (nm) at an intersection of the tangent and the abscissa axis is assigned to a conversion equation (F2) below to calculate the lowest singlet energy.

Conversion Equation (F2): S₁[eV]=1239.85/λ_{edge}

Any apparatus for measuring absorption spectrum is usable. For instance, a spectrophotometer (U3310 manufactured by Hitachi, Ltd.) is usable.

The tangent to the fall of the absorption spectrum close to the long-wavelength region is drawn as follows. While moving on a curve of the absorption spectrum from the local maximum value closest to the long-wavelength region, among the local maximum values of the absorption spectrum, in a long-wavelength direction, a tangent at each point on the curve is checked. An inclination of the tangent is decreased and increased in a repeated manner as the curve falls (i.e., a value of the ordinate axis is decreased). A tangent drawn at a point where the inclination of the curve is the local minimum closest to the long-wavelength region (except when absorbance is 0.1 or less) is defined as the tangent to the fall of the absorption spectrum close to the long-wavelength region.

The local maximum absorbance of 0.2 or less is not counted as the above-mentioned local maximum absorbance closest to the long-wavelength region.

### Method of Producing Second Compound

The second compound according to the exemplary embodiment can be produced by application of known substitution reactions and materials depending on a target compound, according to a synthesis method described in Examples described later or in a similar manner as the synthesis method.

### Specific Examples of Second Compound

Specific examples of the second compound in the exemplary embodiment include compounds as follows. However, the invention is by no means limited to the specifically listed compounds.

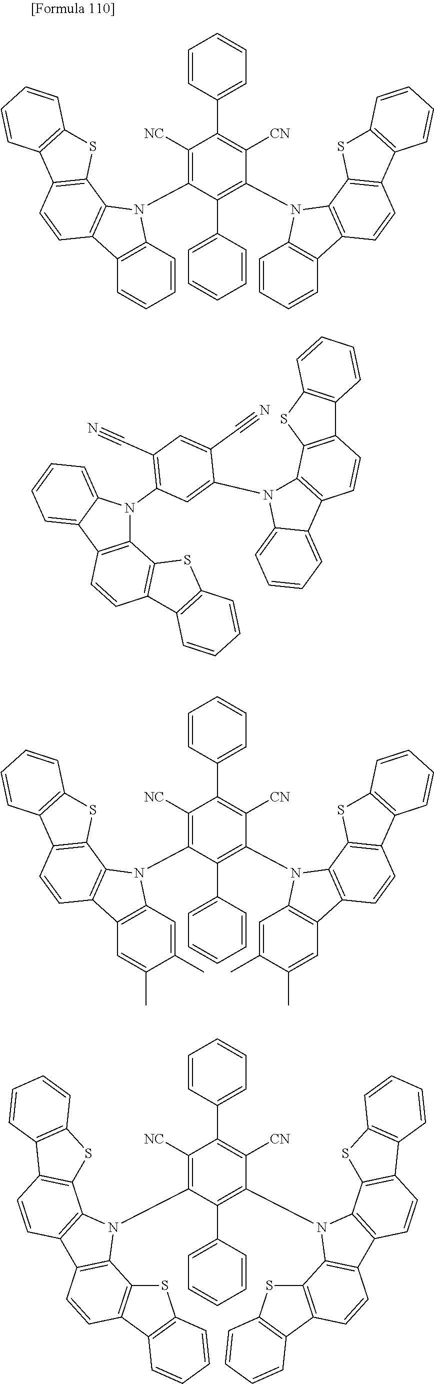

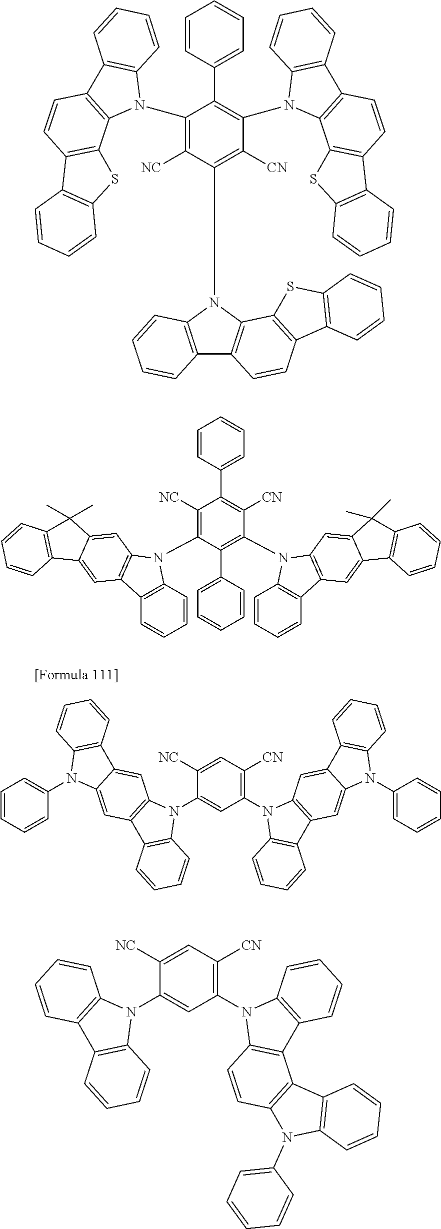 [SMILES: N#Cc1c(-n2c3ccccc3c3ccc4c5ccccc5sc4c32)c(C#N)c(-n2c3ccccc3c3ccc4c5ccccc5sc4c32)c(-c2ccccc2)c1-n1c2ccccc2c2ccc3c4ccccc4sc3c21 ; CC1(C)c2ccccc2-c2cc3c4ccccc4n(-c4c(C#N)c(-c5ccccc5)c(C#N)c(-n5c6ccccc6c6cc7c(cc65)C(C)(C)c5ccccc5-7)c4-c4ccccc4)c3cc21 ; N#Cc1cc(C#N)c(-n2c3ccccc3c3cc4c(cc32)c2ccccc2n4-c2ccccc2)cc1-n1c2ccccc2c2cc3c(cc21)c1ccccc1n3-c1ccccc1 ; N#Cc1cc(C#N)c(-n2c3ccccc3c3c4c5ccccc5n(-c5ccccc5)c4ccc32)cc1-n1c2ccccc2c2ccccc21]

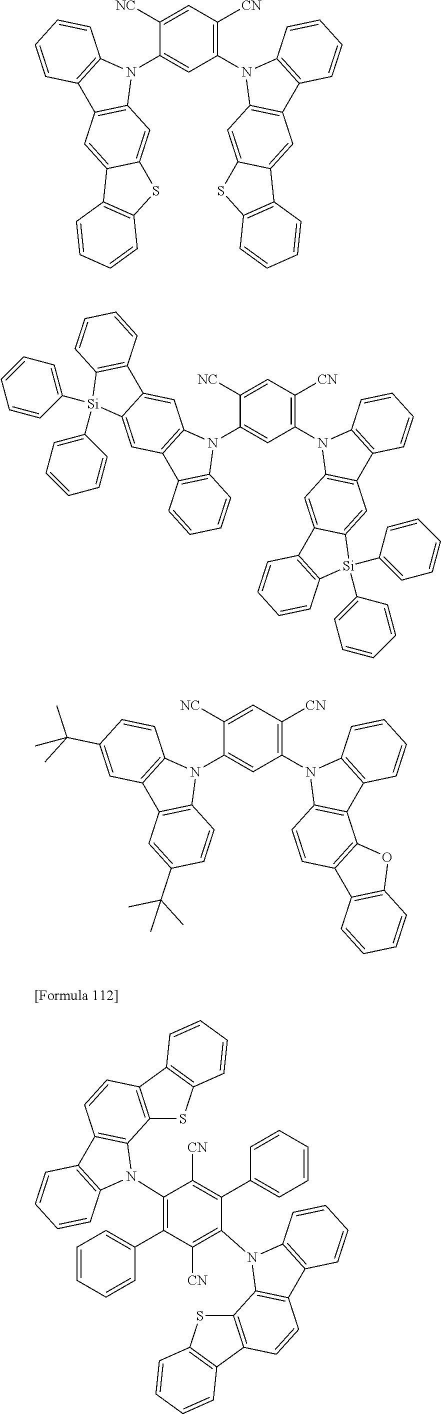

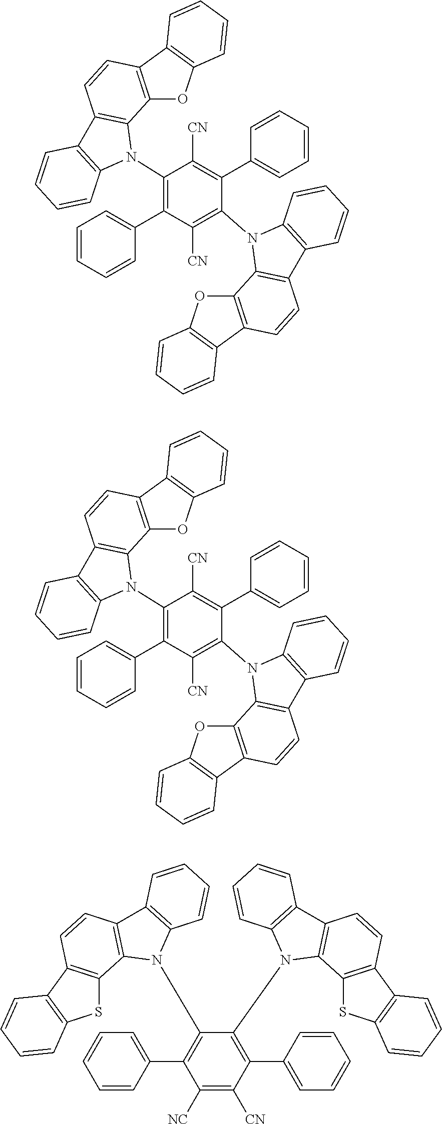

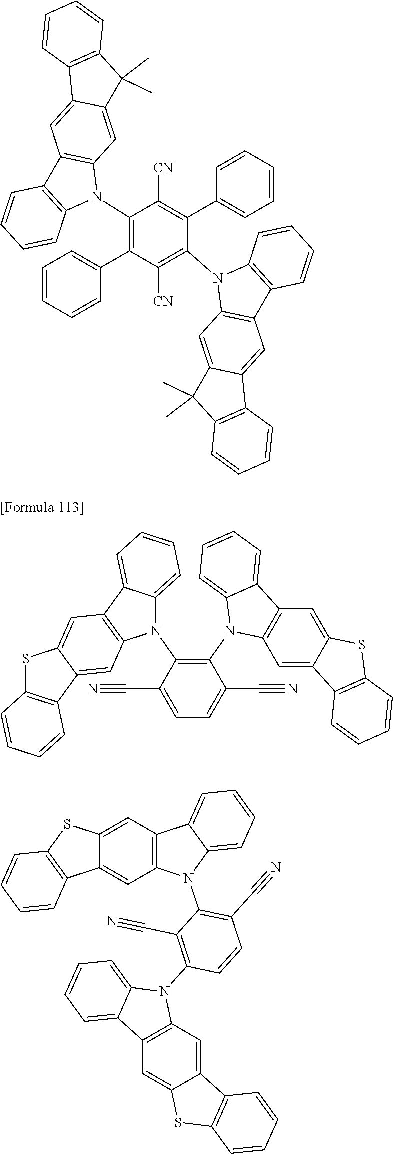 [SMILES: CC1(C)c2ccccc2-c2cc3c4ccccc4n(-c4c(C#N)c(-c5ccccc5)c(-n5c6ccccc6c6cc7c(cc65)C(C)(C)c5ccccc5-7)c(C#N)c4-c4ccccc4)c3cc21 ; N#Cc1ccc(C#N)c(-n2c3ccccc3c3cc4sc5ccccc5c4cc32)c1-n1c2ccccc2c2cc3sc4ccccc4c3cc21 ; N#Cc1ccc(-n2c3ccccc3c3cc4sc5ccccc5c4cc32)c(C#N)c1-n1c2ccccc2c2cc3sc4ccccc4c3cc21]

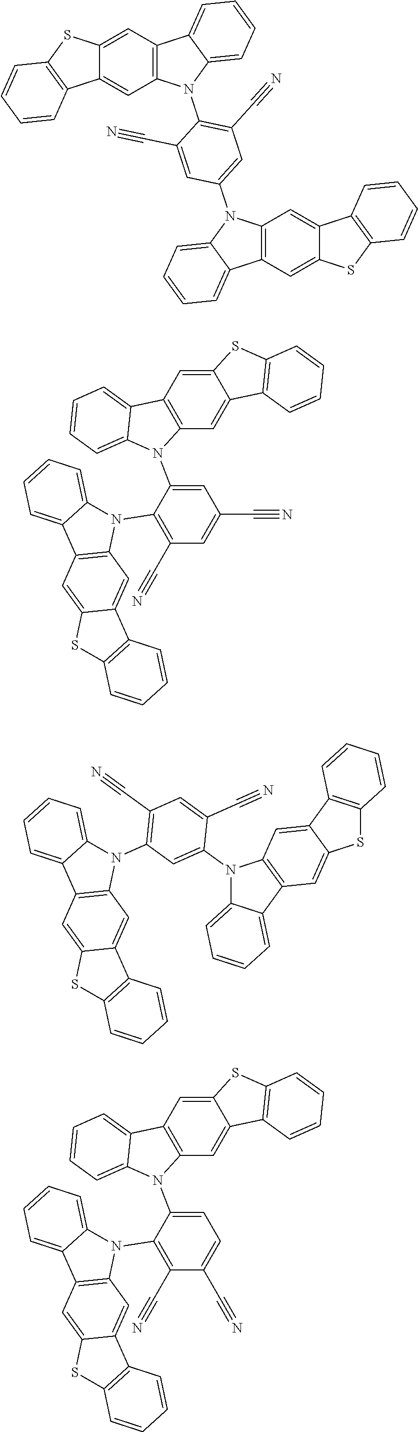 [SMILES: N#Cc1cc(-n2c3ccccc3c3cc4sc5ccccc5c4cc32)cc(C#N)c1-n1c2ccccc2c2cc3sc4ccccc4c3cc21 ; N#Cc1cc(C#N)c(-n2c3ccccc3c3cc4sc5ccccc5c4cc32)c(-n2c3ccccc3c3cc4sc5ccccc5c4cc32)c1 ; N#Cc1cc(C#N)c(-n2c3ccccc3c3cc4sc5ccccc5c4cc32)cc1-n1c2ccccc2c2cc3sc4ccccc4c3cc21 ; N#Cc1ccc(-n2c3ccccc3c3cc4sc5ccccc5c4cc32)c(-n2c3ccccc3c3cc4sc5ccccc5c4cc32)c1C#N]

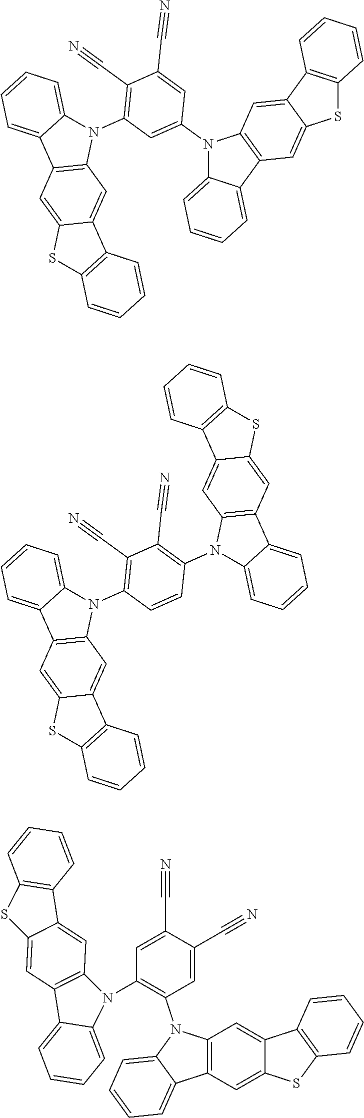 [SMILES: N#Cc1cc(-n2c3ccccc3c3cc4sc5ccccc5c4cc32)cc(-n2c3ccccc3c3cc4sc5ccccc5c4cc32)c1C#N ; N#Cc1c(-n2c3ccccc3c3cc4sc5ccccc5c4cc32)ccc(-n2c3ccccc3c3cc4sc5ccccc5c4cc32)c1C#N ; N#Cc1cc(-n2c3ccccc3c3cc4sc5ccccc5c4cc32)c(-n2c3ccccc3c3cc4sc5ccccc5c4cc32)cc1C#N]

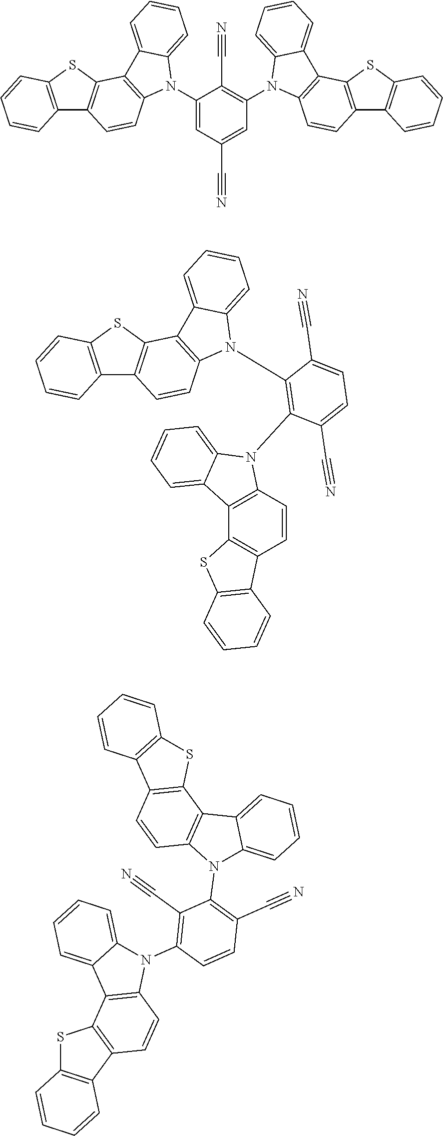 [SMILES: N#Cc1cc(-n2c3ccccc3c3c4sc5ccccc5c4ccc32)c(C#N)c(-n2c3ccccc3c3c4sc5ccccc5c4ccc32)c1 ; N#Cc1ccc(C#N)c(-n2c3ccccc3c3c4sc5ccccc5c4ccc32)c1-n1c2ccccc2c2c3sc4ccccc4c3ccc21 ; N#Cc1ccc(-n2c3ccccc3c3c4sc5ccccc5c4ccc32)c(C#N)c1-n1c2ccccc2c2c3sc4ccccc4c3ccc21]

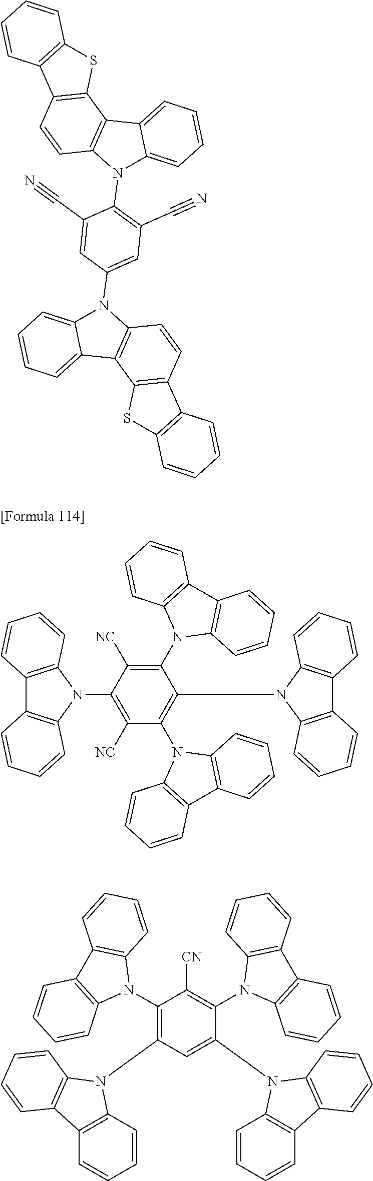 [SMILES: N#Cc1cc(-n2c3ccccc3c3c4sc5ccccc5c4ccc32)cc(C#N)c1-n1c2ccccc2c2c3sc4ccccc4c3ccc21 ; N#Cc1c(-n2c3ccccc3c3ccccc32)c(C#N)c(-n2c3ccccc3c3ccccc32)c(-n2c3ccccc3c3ccccc32)c1-n1c2ccccc2c2ccccc21 ; N#Cc1c(-n2c3ccccc3c3ccccc32)c(-n2c3ccccc3c3ccccc32)cc(-n2c3ccccc3c3ccccc32)c1-n1c2ccccc2c2ccccc21]

Relationship between First Compound and Second Compound in Emitting Layer

In the organic EL device according to the exemplary embodiment, a lowest singlet energy S₁(M1) of the first compound and a lowest singlet energy S₁(M2) of the second compound satisfy a relationship of a numerical formula (Numerical Formula 1) below.

An energy gap T_{77K}(M2) at 77K of the second compound is preferably larger than an energy gap T_{77K}(M1) at 77K of the first compound. In other words, a relationship of a numerical formula (Numerical Formula 5) below is preferably satisfied.

T_{77K}(M2)>T_{77K}(M1) (Numerical Formula 5)

When the organic EL device according to the exemplary embodiment emits light, it is preferable that the first compound mainly emits light in the emitting layer.

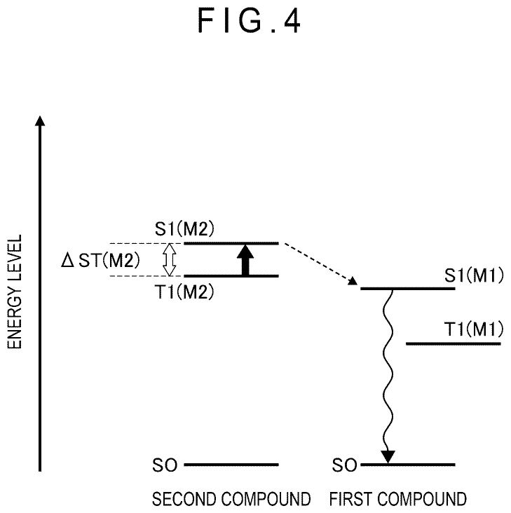

TADF Mechanism FIG. 4 illustrates an example of a relationship between energy levels of the first compound and the second compound in the emitting layer. In FIG. 4, S0 represents a ground state. S₁(M2) represents a lowest singlet state of the second compound. T1 (M2) represents a lowest triplet state of the second compound. S₁ (M1) represents a lowest singlet state of the first compound. T1 (M1) represents a lowest triplet state of the first compound.

A dashed arrow directed from S1 (M2) to S1 (M1) in FIG. 4 represents Förster energy transfer from the lowest singlet state of the second compound to the first compound.

As illustrated in FIG. 4, when a compound having a small ΔST(M2) is used as the second compound, inverse intersystem crossing from the lowest triplet state T1(M2) to the lowest singlet state S1(M2) can be caused by a heat energy. Subsequently, Förster energy transfer from the lowest singlet state S1 (M2) of the second compound to the first compound occurs to generate the lowest singlet state S1 (M1). Consequently, fluorescence from the lowest singlet state S1 (M1) of the first compound can be observed. It is inferred that the internal quantum efficiency can be theoretically raised up to 100% also by using delayed fluorescence by the TADF mechanism.

The organic EL device according to the exemplary embodiment preferably emits green light. When the organic EL device according to the exemplary embodiment emits a green light, the maximum peak wavelength of the light emitted from the organic EL device is preferably in a range from 500 nm to 560 nm.

The maximum peak wavelength of the light emitted from the organic EL device is measured as follows.

Voltage is applied on the organic EL device such that a current density becomes 10 mA/cm², where spectral radiance spectrum is measured by a spectroradiometer CS-2000 (manufactured by Konica Minolta, Inc.).

A peak wavelength of an emission spectrum, at which the luminous intensity of the resultant spectral radiance spectrum is at the maximum, is measured and defined as the maximum peak wavelength (unit: nm).

### Film Thickness of Emitting Layer

A film thickness of the emitting layer of the organic EL device according to the exemplary embodiment is preferably in a range from 5 nm to 50 nm, more preferably in a range from 7 nm to 50 nm, and still more preferably in a range from 10 nm to 50 nm. When the film thickness of the emitting layer is 5 nm or more, the formation of the emitting layer and the adjustment of the chromaticity are likely to be facilitated. When the film thickness of the emitting layer is 50 nm or less, an increase in the drive voltage is likely to be inhibited.

### Content Ratios of Compounds in Emitting Layer

For instance, content ratios of the first compound and the second compound in the emitting layer preferably fall within ranges shown below.

The content ratio of the second compound is preferably in a range from 10 mass % to 80 mass %, more preferably in a range from 10 mass % to 60 mass %, and still more preferably in a range from 20 mass % to 60 mass %. The content ratio of the second compound may be in a range from 90 mass % to 99.9 mass %, may be in a range from 95 mass % to 99.9 mass %, and may be in a range from 99 mass % to 99.9 mass %.

The content ratio of the first compound is preferably in a range from 0.01 mass % to 10 mass %, more preferably in a range from 0.01 mass % to 5 mass %, and still more preferably in a range from 0.01 mass % to 1 mass %.

It should be noted that the emitting layer of the exemplary embodiment may contain a material other than the first compound and the second compound.

The emitting layer may contain a single type of the first compound or may contain two or more types of the first compound.

The emitting layer may contain a single type of the second compound or may contain two or more types of the second compound.

### Substrate

The substrate is used as a support for the organic EL device. For instance, glass, quartz, plastics and the like are usable for the substrate. A flexible substrate is also usable. The flexible substrate is a bendable substrate. Examples of the flexible substrate include a plastic substrate made using polycarbonate, polyarylate, polyethersulfone, polypropylene, polyester, polyvinyl fluoride, and polyvinyl chloride. Moreover, an inorganic vapor deposition film is also usable.

### Anode

Metal, an alloy, an electrically conductive compound, a mixture thereof, or the like having a large work function (specifically, 4.0 eV or more) is preferably used as the anode formed on the substrate. Specific examples of the material include ITO (Indium Tin Oxide), indium oxide-tin oxide containing silicon or silicon oxide, indium oxide-zinc oxide, indium oxide containing tungsten oxide and zinc oxide, and graphene. In addition, gold (Au), platinum (Pt), nickel (Ni), tungsten (W), chrome (Cr), molybdenum (Mo), iron (Fe), cobalt (Co), copper (Cu), palladium (Pd), titanium (Ti), and nitrides of a metal material (e.g., titanium nitride) are usable.

The material is typically formed into a film by a sputtering method. For instance, the indium oxide-zinc oxide can be formed into a film by the sputtering method using a target in which zinc oxide in a range from 1 mass % to 10 mass % is added to indium oxide. Moreover, for instance, the indium oxide containing tungsten oxide and zinc oxide can be formed by the sputtering method using a target in which tungsten oxide in a range from 0.5 mass % to 5 mass % and zinc oxide in a range from 0.1 mass % to 1 mass % are added to indium oxide. In addition, the anode may be formed by a vacuum deposition method, a coating method, an inkjet method, a spin coating method or the like.

Among the organic layers formed on the anode, since the hole injecting layer adjacent to the anode is formed of a composite material into which holes are easily injectable irrespective of the work function of the anode, a material usable as an electrode material (e.g., metal, an alloy, an electroconductive compound, a mixture thereof, and the elements belonging to the group 1 or 2 of the periodic table) is also usable for the anode.

A material having a small work function such as elements belonging to Groups 1 and 2 in the periodic table of the elements, specifically, an alkali metal such as lithium (Li) and cesium (Cs), an alkaline earth metal such as magnesium (Mg), calcium (Ca) and strontium (Sr), alloys (e.g., MgAg and AILi) including the alkali metal or the alkaline earth metal, a rare earth metal such as europium (Eu) and ytterbium (Yb), alloys including the rare earth metal are also usable for the anode. It should be noted that the vacuum deposition method and the sputtering method are usable for forming the anode using the alkali metal, alkaline earth metal and the alloy thereof. Further, when a silver paste is used for the anode, the coating method and the inkjet method are usable.

### Cathode

It is preferable to use metal, an alloy, an electroconductive compound, a mixture thereof, or the like having a small work function (specifically, 3.8 eV or less) for the cathode. Examples of the material for the cathode include elements belonging to Groups 1 and 2 in the periodic table of the elements, specifically, an alkali metal such as lithium (Li) and cesium (Cs), an alkaline earth metal such as magnesium (Mg), calcium (Ca) and strontium (Sr), alloys (e.g., MgAg and AILi) including the alkali metal or the alkaline earth metal, a rare earth metal such as europium (Eu) and ytterbium (Yb), and alloys including the rare earth metal.

It should be noted that the vacuum deposition method and the sputtering method are usable for forming the cathode using the alkali metal, alkaline earth metal and the alloy thereof. Further, when a silver paste is used for the cathode, the coating method and the inkjet method are usable.

By providing the electron injecting layer, various conductive materials such as Al, Ag, ITO, graphene, and indium oxide-tin oxide containing silicon or silicon oxide may be used for forming the cathode regardless of the work function. The conductive materials can be formed into a film using the sputtering method, inkjet method, spin coating method and the like.

### Hole Injecting Layer

The hole injecting layer is a layer containing a substance exhibiting a high hole injectability. Examples of the substance exhibiting a high hole injectability include molybdenum oxide, titanium oxide, vanadium oxide, rhenium oxide, ruthenium oxide, chrome oxide, zirconium oxide, hafnium oxide, tantalum oxide, silver oxide, tungsten oxide, and manganese oxide.

In addition, the examples of the highly hole-injectable substance include: an aromatic amine compound, which is a low-molecule organic compound, such that 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(4-diphenylaminophenyl)-N-phenylamino]biphenyl (abbreviation: DPAB), 4,4′-bis(N-{4-[N′-(3-methylphenyl)-N′-phenylamino]phenyl}-N-phenylamino)biphenyl (abbreviation: DNTPD), 1,3,5-tris[N-(4-diphenylaminophenyl)-N-phenylamino]benzene (abbreviation: DPA3B), 3-[N-(9-phenylcarbazole-3-yl)-N-phenylamino]-9-phenylcarbazole (abbreviation: PCzPCA1), 3,6-bis[N-(9-phenylcarbazole-3-yl)-N-phenylamino]-9-phenylcarbazole (abbreviation: PCzPCA2), and 3-[N-(1-naphthyl)-N-(9-phenylcarbazole-3-yl)amino]-9-phenylcarbazole (abbreviation: PCzPCN1).

In addition, a high polymer compound (e.g., oligomer, dendrimer and polymer) is usable as the substance exhibiting a high hole injectability. Examples of the high-molecule compound include poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N′-phenylamino}phenyl)methacrylamide](abbreviation: PTPDMA), and poly[N,N′-bis(4-butylphenyl)-N,N′-bis(phenyl)benzidine] (abbreviation: Poly-TPD). Moreover, an acid-added high polymer compound such as poly(3,4-ethylenedioxythiophene)/poly(styrene sulfonic acid) (PEDOT/PSS) and polyaniline/poly(styrene sulfonic acid) (PAni/PSS) are also usable.

### Hole Transporting Layer

The hole transporting layer is a layer containing a highly hole-transporting substance. An aromatic amine compound, carbazole derivative, anthracene derivative and the like are usable for the hole transporting layer. Specific examples of a material for the hole transporting layer include 4,4′-bis[N-(1-naphthyl)-N-phenylamino]biphenyl (abbreviation: NPB), N,N′-bis(3-methylphenyl)-N,N′-diphenyl-[1,1′-biphenyl]-4,4′-diamine (abbreviation: TPD), 4-phenyl-4′-(9-phenylfluorene-9-yl)triphenylamine (abbreviation: BAFLP), 4,4′-bis[N-(9,9-dimethylfluorene-2-yl)-N-phenylamino]biphenyl (abbreviation: DFLDPBi), 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), and 4,4′-bis[N-(spiro-9,9′-bifluorene-2-yl)-N-phenylamino]biphenyl (abbreviation: BSPB). The above-described substances mostly have a hole mobility of 10⁻⁶ cm²/(Vs) or more.

For the hole transporting layer, a carbazole derivative such as CBP, CzPA, and PCzPA and an anthracene derivative such as t-BuDNA, DNA, and DPAnth may be used. A high polymer compound such as poly(N-vinylcarbazole) (abbreviation: PVK) and poly(4-vinyltriphenylamine) (abbreviation: PVTPA) is also usable.

However, in addition to the above substances, any substance exhibiting a higher hole transportability than an electron transportability may be used. It should be noted that the layer containing the substance exhibiting a high hole transportability may be not only a single layer but also a laminate of two or more layers formed of the above substance(s).

### Electron Transporting Layer

The electron transporting layer is a layer containing a highly electron-transporting substance. For the electron transporting layer, 1) a metal complex such as an aluminum complex, beryllium complex, and zinc complex, 2) a hetero aromatic compound such as imidazole derivative, benzimidazole derivative, azine derivative, carbazole derivative, and phenanthroline derivative, and 3) a high polymer compound are usable. Specifically, as a low-molecule organic compound, a metal complex such as Alq, tris(4-methyl-8-quinolinato)aluminum (abbreviation: Almq3), bis(10-hydroxybenzo[h]quinolinato)beryllium (abbreviation: BeBq2), BAIq, Znq, ZnPBO and ZnBTZ is usable. In addition to the metal complex, a heteroaromatic compound such as 2-(4-biphenylyl)-5-(4-tert-butylphenyl)-1,3,4-oxadiazole (abbreviation: PBD), 1,3-bis[5-(ptert-butylphenyl)-1,3,4-oxadiazole-2-yl]benzene (abbreviation: OXD-7), 3-(4-tert-butylphenyl)-4-phenyl-5-(4-biphenylyl)-1,2,4-triazole (abbreviation: TAZ), 3-(4-tert-butylphenyl)-4-(4-ethylphenyl)-5-(4-biphenylyl)-1,2,4-triazole (abbreviation: p-EtTAZ), bathophenanthroline (abbreviation: BPhen), bathocuproine (abbreviation: BCP), and 4,4′-bis(5-methylbenzoxazole-2-yl)stilbene (abbreviation: BzOs) is usable. The above-described substances mostly have an electron mobility of 10⁻⁶ cm²/(Vs) or more. It should be noted that any substance other than the above substance may be used for the electron transporting layer as long as the substance exhibits a higher electron transportability than the hole transportability. It should be noted that the electron transporting layer may be not only a single layer but also a laminate of two or more layers formed of the above substance(s).

Further, a high polymer compound is usable for the electron transporting layer. For instance, poly[(9,9-dihexylfluorene-2,7-diyl)-co-(pyridine-3,5-diyl)](abbreviation: PF-Py), poly[(9,9-dioctylfluorene-2,7-diyl)-co-(2,2′-bipyridine-6,6′-diyl)](abbreviation: PF-BPy) and the like are usable.

### Electron Injecting Layer

The electron injecting layer is a layer containing a highly electron-injectable substance. Examples of a material for the electron injecting layer include an alkali metal, alkaline earth metal and a compound thereof, examples of which include lithium (Li), cesium (Cs), calcium (Ca), lithium fluoride (LiF), cesium fluoride (CsF), calcium fluoride (CaF₂), and lithium oxide (LiOx). In addition, the alkali metal, alkaline earth metal or the compound thereof may be added to the substance exhibiting the electron transportability in use. Specifically, for instance, magnesium (Mg) added to Alq may be used. In this case, the electrons can be more efficiently injected from the cathode.

Alternatively, the electron injecting layer may be provided by a composite material in a form of a mixture of the organic compound and the electron donor. Such a composite material exhibits excellent electron injectability and electron transportability since electrons are generated in the organic compound by the electron donor. In this case, the organic compound is preferably a material excellent in transporting the generated electrons. Specifically, the above examples (e.g., the metal complex and the hetero aromatic compound) of the substance forming the electron transporting layer are usable. As the electron donor, any substance exhibiting electron donating property to the organic compound is usable. Specifically, the electron donor is preferably alkali metal, alkaline earth metal and rare earth metal such as lithium, cesium, magnesium, calcium, erbium and ytterbium. The electron donor is also preferably alkali metal oxide and alkaline earth metal oxide such as lithium oxide, calcium oxide, and barium oxide. Moreover, a Lewis base such as magnesium oxide is usable. Further, the organic compound such as tetrathiafulvalene (abbreviation: TTF) is usable.

### Layer Formation Method

A method for forming each layer of the organic EL device in the exemplary embodiment is subject to no limitation except for the above particular description. However, known methods of dry film-forming such as vacuum deposition, sputtering, plasma or ion plating and wet film-forming such as spin coating, dipping, flow coating or ink-jet are applicable.

### Film Thickness

A thickness of each of the organic layers in the organic EL device according to the exemplary embodiment is not limited except for the above particular description. In general, the thickness preferably ranges from several nanometers to 1 μm because excessively small film thickness is likely to cause defects (e.g. pin holes) and excessively large thickness leads to the necessity of applying high voltage and consequent reduction in efficiency.

According to the exemplary embodiment, a high-performance organic EL device can be provided. The performance of the organic EL device is evaluable in terms of, for instance, luminance, emission wavelength, chromaticity, luminous efficiency, drive voltage, and lifetime. According to an example of the exemplary embodiment, an organic EL device having a long lifetime can be provided. Further, a luminous efficiency is improved in an exemplary organic EL device of the exemplary embodiment.

The organic EL device according to the exemplary embodiment is usable in an electronic device such as a display device and a light-emitting unit.

### Second Exemplary Embodiment

### Organic Electroluminescence Device

An arrangement of an organic EL device according to a second exemplary embodiment will be described below. In the description of the second exemplary embodiment, the same components as those in the first exemplary embodiment are denoted by the same reference signs and names to simplify or omit an explanation of the components. In the second exemplary embodiment, the same materials and compounds as described in the first exemplary embodiment are usable, unless otherwise specified.

The organic EL device according to the second exemplary embodiment is different from the organic EL device according to the first exemplary embodiment in that the emitting layer further includes a third compound. The second exemplary embodiment is the same as the first exemplary embodiment in other respects.

In the second exemplary embodiment, the emitting layer preferably contains the first compound, the second compound, and the third compound. In this arrangement, it is preferable that the first compound is a dopant material and the second compound is a host material. The third compound is preferably not a dopant material. For instance, the emitting layer of the second exemplary embodiment may contain the second compound and the third compound in total at 50 mass % or more, 60 mass % or more, 70 mass % or more, 80 mass % or more, 90 mass % or more, or 95 mass % or more, with respect to the total mass of the emitting layer.

### Third Compound

The third compound of the exemplary embodiment may be a thermally activated delayed fluorescent compound or a compound exhibiting no thermally activated delayed fluorescence. However, the third compound is preferably a compound exhibiting no thermally activated delayed fluorescence.

A singlet energy S₁(M3) of the third compound and the singlet energy S₁(M2) of the second compound satisfy a relationship of a numerical formula (Numerical Formula 2) below.

S₁(M3)>S₁(M2) (Numerical Formula 2)

The third compound, which is not particularly limited, is preferably a compound other than an amine compound. In other words, the third compound preferably does not have a substituted or unsubstituted amino group. As the third compound, for instance, a carbazole derivative, dibenzofuran derivative, and a dibenzothiophen derivative are usable. However, the third compound is not limited to these derivatives.

It is also preferable that the third compound is a compound having, in a molecule, at least one partial structure of a partial structure represented by a formula (31) below, a partial structure represented by a formula (32) below, a partial structure represented by a formula (33A) below, or a partial structure represented by a formula (34A) below.

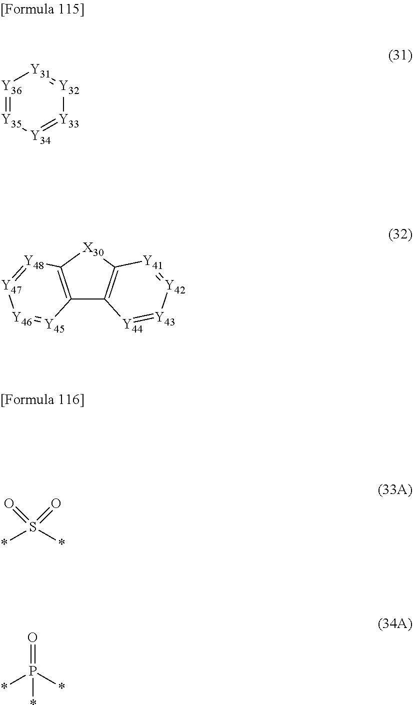

In the formula (31):
Y₃₁ to Y₃₆ are each independently a nitrogen atom or a carbon atom bonded to another atom in a molecule of the third compound; and
at least one of Y₃₁ to Y₃₆ is a carbon atom bonded to another atom in a molecule of the third compound.

In the formula (32):
Y₄₁ to Y₄₈ are each independently a nitrogen atom or a carbon atom bonded to another atom in a molecule of the third compound; and
at least one of Y₄₁ to Y₄₈ is a carbon atom bonded to another atom in a molecule of the third compound.
X₃₀ is a nitrogen atom bonded to another atom in a molecule of the third compound, an oxygen atom, or a sulfur atom.

In the formulae (33A) and (34A), each * independently represents a bonding position to another atom or another structure in a molecule of the third compound.

The third compound preferably has, in a molecule, the partial structure represented by the formula (31) and the partial structure represented by the formula (32) in total in a range from two to ten, more preferably in a range from four to eight.

In the formula (32), it is also preferable that at least two of Y₄₁ to Y₄₈ are each a carbon atom bonded to another atom in a molecule of the third compound to form a cyclic structure containing the carbon atoms.

For instance, the partial structure represented by the formula (32) is preferably any partial structure selected from the group consisting of partial structures represented by formulae (321), (322), (323), (324), (325), and (326) below.

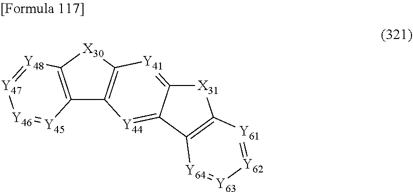

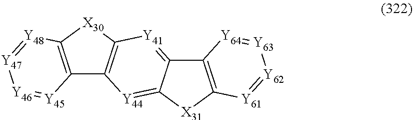

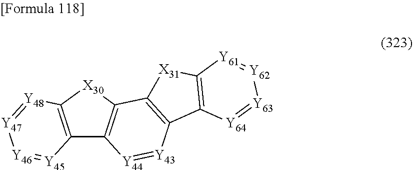

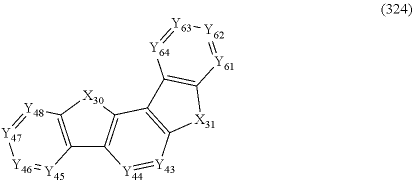

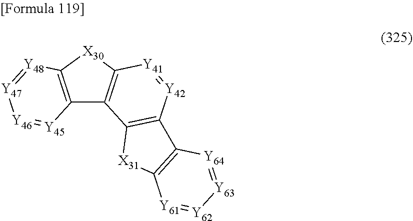

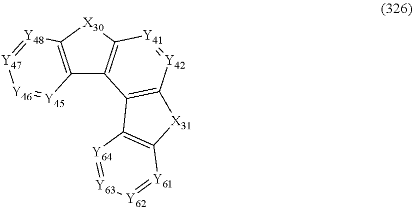

In the formulae (321) to (326):
each X₃₀ is independently a nitrogen atom bonded to another atom in a molecule of the third compound, an oxygen atom, or a sulfur atom;
Y₄₁ to Y₄₅ are each independently a nitrogen atom or a carbon atom bonded to another atom in a molecule of the third compound;
each X₃₁ is independently a nitrogen atom bonded to another atom in a molecule of the third compound, an oxygen atom, a sulfur atom, or a carbon atom bonded to another atom in a molecule of the third compound; and
Y₆₁ to Y₆₄ are each independently a nitrogen atom or a carbon atom bonded to another atom in a molecule of the third compound.

The third compound of the exemplary embodiment preferably has a partial structure represented by the formula (323) of the formulae (321) to (326).

The partial structure represented by the formula (31) is preferably contained in the third compound as at least one group selected from the group consisting of groups represented by formulae (33) and (34) below.

The third compound also preferably has at least one of a partial structure represented by the formula (33) or a partial structure represented by the formula (34). Since the bonding positions are at a meta position as shown in the partial structures represented by the formulae (33) and (34), an energy gap T_{77K}(M3) at 77K of the third compound can be kept high.

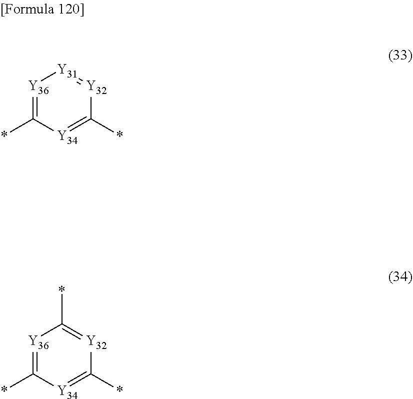

In the formula (33), Y₃₁ Y₃₂ Y₃₄, and Y₃₆ are each independently a nitrogen atom or CR₃₁.

In the formula (34), Y₃₂, Y₃₄, and Y₃₆ are each independently a nitrogen atom or CR₃₁.

In the formulae (33), and (34):
each R₃₁ is independently a hydrogen atom or a substituent;
each R₃₁ as a substituent is independently a group selected from the group consisting of a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a group represented by —Si(R₉₀₁)(R₉₀₂)(R₉₀₃), a substituted germanium group, a substituted phosphine oxide group, a halogen atom, a cyano group, a nitro group, and a substituted or unsubstituted carboxy group.

However, the substituted or unsubstituted aryl group having 6 to 30 ring carbon atoms for R₃₁ is preferably a non-fused ring.

In the formulae (33) and (34), each * independently represents a bonding position to another atom or another structure in a molecule of the third compound.

In the third compound, R₉₀₁ to R₉₀₃ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

In the formula (33), Y₃₁ Y₃₂ Y₃₄, and Y₃₆ are preferably each independently CR₃₁. A plurality of R₃₁ are mutually the same or different.

In the formula (34), Y₃₂, Y₃₄, and Y₃₆ are preferably each independently CR₃₁. A plurality of R₃₁ are mutually the same or different.

A substituted germanium group is preferably represented by —Ge(R₃₀₁)₃. Each R₃₀₁ is independently a substituent. A substituent R₃₀₁ is preferably a substituted or unsubstituted alkyl group having 1 to 30 carbon atoms or a substituted or unsubstituted aryl group having 6 to 30 ring carbon atoms. A plurality of R₃₀₁ are mutually the same or different.

The partial structure represented by the formula (32) is preferably contained in the third compound as at least one group selected from the group consisting of groups represented by formulae (35) to (39) and (30a) below.

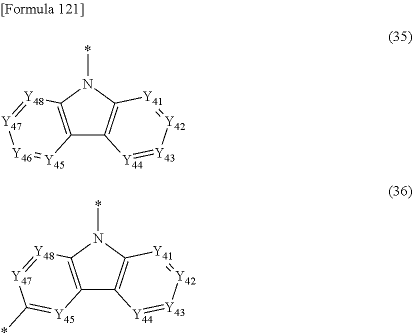

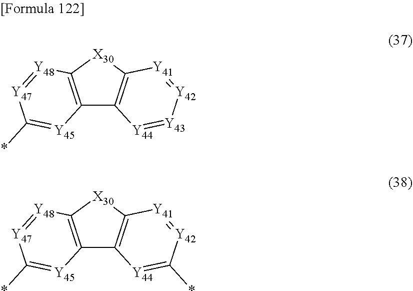

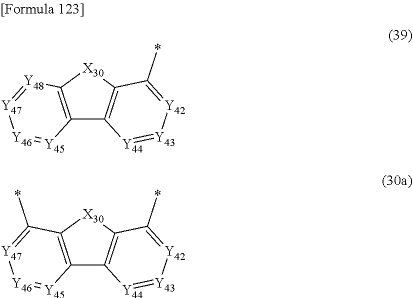

In the formula (35), Y₄₁ to Y₄₅ are each independently a nitrogen atom or CR₃₂.

In the formulae (36) and (37), Y₄₁ to Y₄₅ Y₄₇, and Y₄₅ are each independently a nitrogen atom or CR₃₂.

In the formula (38), Y₄₁, Y₄₂, Y₄₄, Y₄₅, Y₄₇, and Y₄₅ are each independently a nitrogen atom or CR₃₂.

In the formula (39), Y₄₂ to Y₄₅ are each independently a nitrogen atom or CR₃₂.

In the formula (30a), Y₄₂ to Y₄₇ are each independently a nitrogen atom or CR₃₂.

In the formulae (35) to (39) and (30a):
each R₃₂ is independently a hydrogen atom or a substituent;
each R₃₂ as a substituent is independently a group selected from the group consisting of a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a group represented by —Si(R₉₀₁)(R₉₀₂)(R₉₀₃), a substituted germanium group, a substituted phosphine oxide group, a halogen atom, a cyano group, a nitro group, and a substituted or unsubstituted carboxy group; and
a plurality of R₃₂ are mutually the same or different.

In the formulae (37) to (39) and (30a):
X₃₀ is NR₃₃, an oxygen atom, or a sulfur atom;
R₃₃ is a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a group represented by —Si(R₉₀₁)(R₉₀₂)(R₉₀₃), a substituted germanium group, a substituted phosphine oxide group, a fluorine atom, a cyano group, a nitro group, and a substituted or unsubstituted carboxy group; and
a plurality of R₃₃ are mutually the same or different.

However, the substituted or unsubstituted aryl group having 6 to 30 ring carbon atoms for R₃₃ is preferably a non-fused ring.

In the formulae (35) to (39) and (30a), each * independently represents a bonding position to another atom or another structure in a molecule of the third compound.

In the formula (35), Y₄₁ to Y₄₅ are preferably each independently CR₃₂. In the formulae (36) and (37), Y₄₁ to Y₄₅, Y₄₇, and Y₄₅ are preferably each independently CR₃₂. In the formula (38), Y₄₁, Y₄₂, Y₄₄, Y₄₅, Y₄₇, and Y₄₅ are preferably each independently CR₃₂. In the formula (39), Y₄₂ to Y₄₅ are preferably each independently CR₃₂. In the formula (30a), Y₄₂ to Y₄₇ are preferably each independently CR₃₂. A plurality of R₃₂ are mutually the same or different.

In the third compound, X₃₀ is preferably an oxygen atom or a sulfur atom.

In the third compound, R₃₁ and R₃₂ are preferably each independently a hydrogen atom or a substituent. It is preferable that R₃₁ as a substituent and R₃₂ as a substituent are each independently any group selected from the group consisting of a fluorine atom, a cyano group, a substituted or unsubstituted alkyl group having 1 to 30 carbon atoms, a substituted or unsubstituted aryl group having 6 to 30 ring carbon atoms, and a substituted or unsubstituted heterocyclic group having 5 to 30 ring atoms. It is more preferable that R₃₁ and R₃₂ are a hydrogen atom, a cyano group, a substituted or unsubstituted aryl group having 6 to 30 ring carbon atoms, or a substituted or unsubstituted heterocyclic group having 5 to 30 ring atoms. However, when R₃₁ and R₃₂ each as a substituent are a substituted or unsubstituted aryl group having 6 to 30 ring carbon atoms, the aryl group is preferably a non-fused ring.

The third compound is also preferably an aromatic hydrocarbon compound or an aromatic heterocyclic compound.

A substituted phosphine oxide group is also preferably a substituted or unsubstituted diaryl phosphine oxide group.

Specific examples of a substituted or unsubstited diaryl phosphine oxide group include a diphenyl phosphine oxide group and a ditolyl phosphine oxide group.

A substituted carboxy group is exemplified by a benzoyloxy group.

### Method of Producing Third Compound

The third compound is producible according to a method described in, for instance, International Publication Nos. WO2012/153780 and WO2013/038650. The third compound is also producible, for instance, through a known alternative reaction using a known material(s) tailored for the target compound.

### Specific Examples of Third Compound

Specific examples of the third compound in the exemplary embodiment are shown below. However, the third compound in the invention is by no means limited to the specific examples.

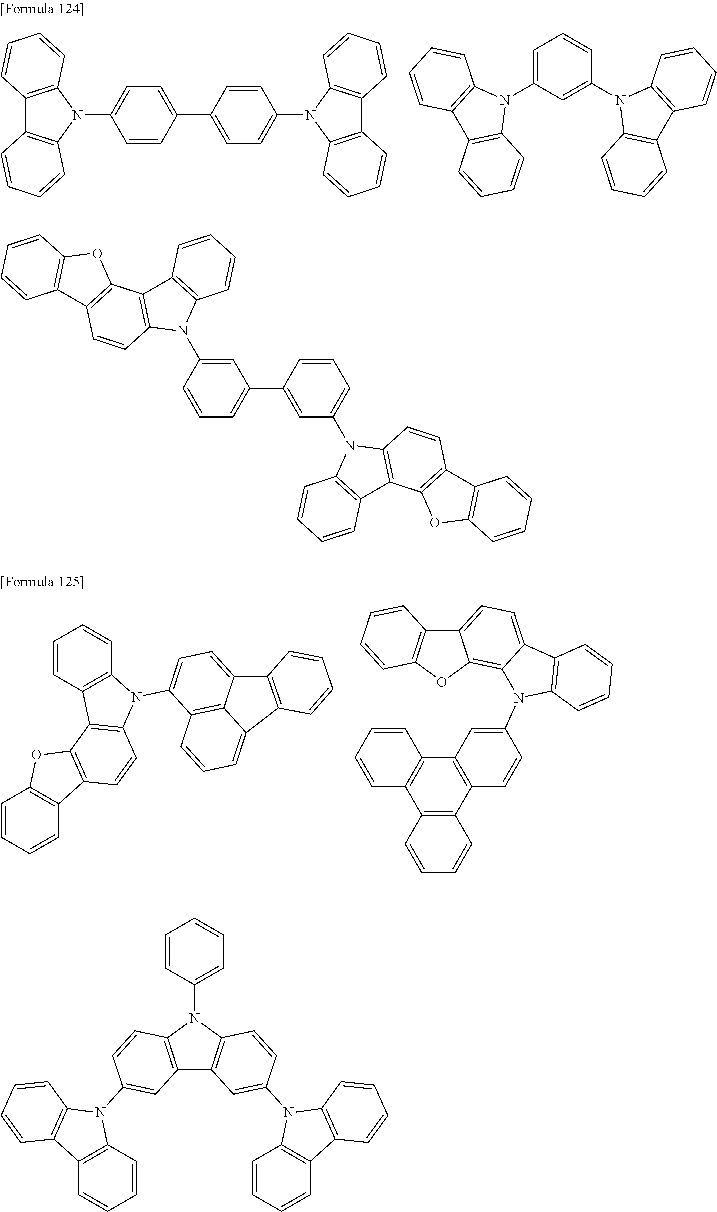

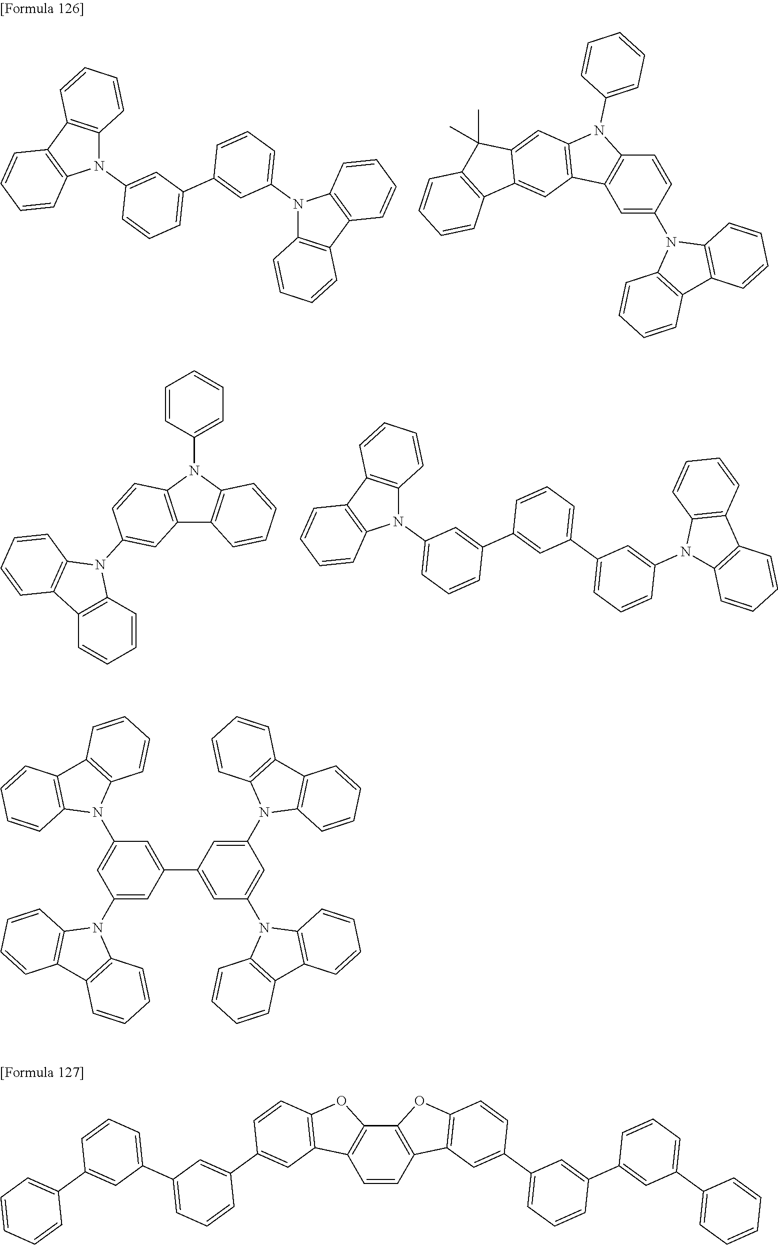

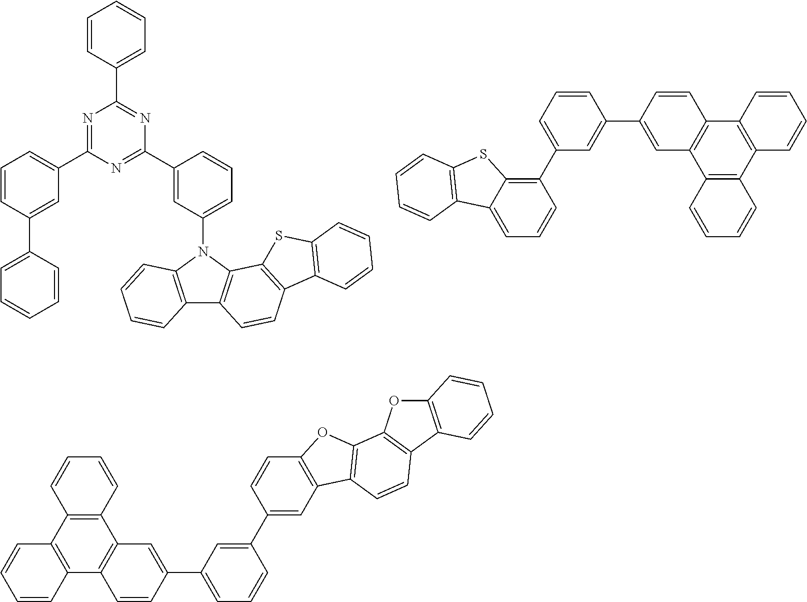

Relationship between First Compound, Second Compound, and Third Compound in Emitting Layer

In the organic EL device according to the exemplary embodiment, the lowest singlet energy S₁(M2) of the second compound and the lowest singlet energy S₁(M3) of the third compound satisfy a relationship of the numerical formula (Numerical Formula 2) below.

The energy gap T_{77K}(M3) at 77K of the third compound is preferably larger than the energy gap T_{77K}(M1) at 77K of the first compound.

The energy gap T_{77K}(M3) at 77K of the third compound is preferably larger than the energy gap T_{77K}(M2) at 77K of the second compound.

The lowest singlet energy S₁(M1) of the first compound, the lowest singlet energy S₁(M2) of the second compound, and the lowest singlet energy S₁(M3) of the third compound preferably satisfy a relationship of a numerical formula (Numerical Formula 2A).

S₁(M3)>S₁(M2)>S₁(M1) (Numerical Formula 2A)

The energy gap T_{77K}(M1) at 77K of the first compound, the energy gap T_{77K}(M2) at 77K of the second compound, and the energy gap T_{77K}(M3) at 77K of the third compound preferably satisfy a relationship of a numerical formula (Numerical Formula 2B) below.

T_{77K}(M3)>T_{77K}(M2)>T_{77K}(M1) (Numerical Formula 2B)

When the organic EL device according to the exemplary embodiment emits light, it is preferable that the first compound emits light in the emitting layer.

The organic EL device according to the exemplary embodiment preferably emits a green light similar to the organic EL device of the first exemplary embodiment.

When the organic EL device according to the exemplary embodiment emits a green light, the maximum peak wavelength of the light emitted from the organic EL device is preferably in a range from 500 nm to 560 nm.

The maximum peak wavelength of the light emitted from the organic EL device can be measured according to the same method as in the organic EL device of the first exemplary embodiment.

### Content Ratios of Compounds in Emitting Layer

When the emitting layer contains the first compound, the second compound, and the third compound, content ratios of the first compound, the second compound, and the third compound in the emitting layer preferably fall within ranges below.

The content ratio of the first compound is preferably in a range from 0.01 mass % to 10 mass %, more preferably in a range from 0.01 mass % to 5 mass %, and still more preferably in a range from 0.01 mass % to 2 mass %.

The content ratio of the second compound is preferably in a range from 10 mass % to 80 mass %, more preferably in a range from 10 mass % to 60 mass %, and still more preferably in a range from 20 mass % to 60 mass %.

The content ratio of the third compound is preferably in a range from 10 mass % to 80 mass %.

The upper limit of a total of the content ratios of the first compound, the second compound, and the third compound in the emitting layer is 100 mass %. It should be noted that the emitting layer of the exemplary embodiment may contain a material other than the first compound, the second compound, and the third compound.

The emitting layer may contain a single type of the first compound or may contain two or more types of the first compound. The emitting layer may contain a single type of the second compound or may contain two or more types of the second compound. The emitting layer may contain a single type of the third compound or may contain two or more types of the third compound.

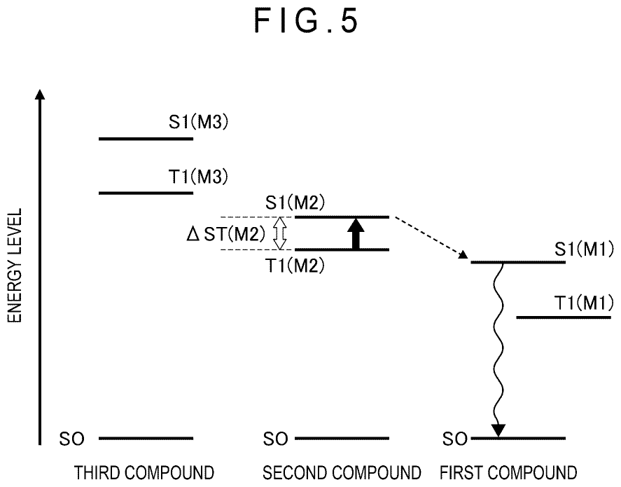

FIG. 5 illustrates an example of a relationship between energy levels of the first compound, the second compound, and the third compound in the emitting layer. In FIG. 5, S0 represents a ground state. S1(M1) represents the lowest singlet state of the first compound. T1(M1) represents the lowest triplet state of the first compound. S1(M2) represents the lowest singlet state of the second compound. T1(M2) represents the lowest triplet state of the second compound. S1(M3) represents the lowest singlet state of the third compound. T1 (M3) represents the lowest triplet state of the third compound. A dashed arrow directed from S1(M2) to S1(M1) in FIG. 5 represents Förster energy transfer from the lowest singlet state of the second compound to the first compound.

As illustrated in FIG. 5, when a compound having a small ΔST(M2) (delayed fluorescent material) is used as the second compound, inverse intersystem crossing from the lowest triplet state T1 (M2) to the lowest singlet state S1 (M2) can be caused by a heat energy. Subsequently, Förster energy transfer from the lowest singlet state S1 (M2) of the second compound to the first compound occurs to generate the lowest singlet state S1(M1). Consequently, fluorescence from the lowest singlet state S1 (M1) of the first compound can be observed. It is inferred that the internal quantum efficiency can be theoretically raised up to 100% also by using delayed fluorescence using the TADF mechanism.

According to the exemplary embodiment, a high-performance organic EL device can be provided. The performance of the organic EL device is evaluable in terms of, for instance, luminance, emission wavelength, chromaticity, luminous efficiency, drive voltage, and lifetime. According to an example of the exemplary embodiment, an organic EL device having a long lifetime can be provided. Further, a luminous efficiency is improved in an exemplary organic EL device of the exemplary embodiment.

The organic EL device according to the exemplary embodiment is usable in an electronic device such as a display device and a light-emitting unit.

### Third Exemplary Embodiment

### Electronic Device

An electronic device according to a third exemplary embodiment is installed with any one of the organic EL devices according to the above exemplary embodiments. Examples of the electronic device include a display device and a light-emitting unit. Examples of the display device include a display component (e.g., an organic EL panel module), TV, mobile phone, tablet and personal computer. Examples of the light-emitting unit include an illuminator and a vehicle light.

### Modification of Embodiment(s)

The scope of the invention is not limited by the above-described exemplary embodiments but includes any modification and improvement as long as such modification and improvement are compatible with the invention.

For instance, the emitting layer is not limited to a single layer, but may be provided by layering a plurality of emitting layers. When the organic EL device has a plurality of emitting layers, it is only required that at least one of the organic layers satisfies the condition(s) described in the above exemplary embodiments. At least one of the emitting layers preferably contains the compound(s) according to the first exemplary embodiment. When one of the emitting layers contains the compounds according to the first exemplary embodiment, for instance, the rest of the emitting layers may be a fluorescent emitting layer(s) or a phosphorescent emitting layer(s) with use of emission caused by electron transfer from the triplet state directly to the ground state.

When the organic EL device includes a plurality of emitting layers, these emitting layers may be mutually adjacently provided, or may form a so-called tandem organic EL device, in which a plurality of emitting units are layered via an intermediate layer.

For instance, a blocking layer may be provided adjacent to at least one of a side of the emitting layer close to the anode or a side of the emitting layer close to the cathode. The blocking layer is preferably provided in contact with the emitting layer to block at least any of holes, electrons, or excitons.

For instance, when the blocking layer is provided in contact with the side of the emitting layer close to the cathode, the blocking layer permits transport of electrons and blocks holes from reaching a layer provided closer than the blocking layer to the cathode (e.g., the electron transporting layer) beyond the blocking layer. When the organic EL device includes the electron transporting layer, the blocking layer is preferably interposed between the emitting layer and the electron transporting layer.

When the blocking layer is provided in contact with the side of the emitting layer close to the anode, the blocking layer permits transport of holes and blocks electrons from reaching a layer provided closer than the blocking layer to the anode (e.g., the hole transporting layer) beyond the blocking layer. When the organic EL device includes the hole transporting layer, the blocking layer is preferably interposed between the emitting layer and the hole transporting layer.

Alternatively, the blocking layer may be provided adjacent to the emitting layer so that the excitation energy does not leak out from the emitting layer toward neighboring layer(s). The blocking layer blocks excitons generated in the emitting layer from being transferred to a layer(s) (e.g., the electron transporting layer and the hole transporting layer) closer than the blocking layer to the electrode(s) beyond the blocking layer.

The emitting layer is preferably bonded with the blocking layer.

Specific structure, form and the like of the components in the invention may be designed in any manner as long as an object of the invention can be achieved.

### EXAMPLES

Examples of the invention will be described below. However, the invention is by no means limited to these Examples.

### Compounds

Structures of a compound represented by the formula (1) and used for producing organic EL devices in Examples 1, 2, and 2-1 to 2-8 are shown below.

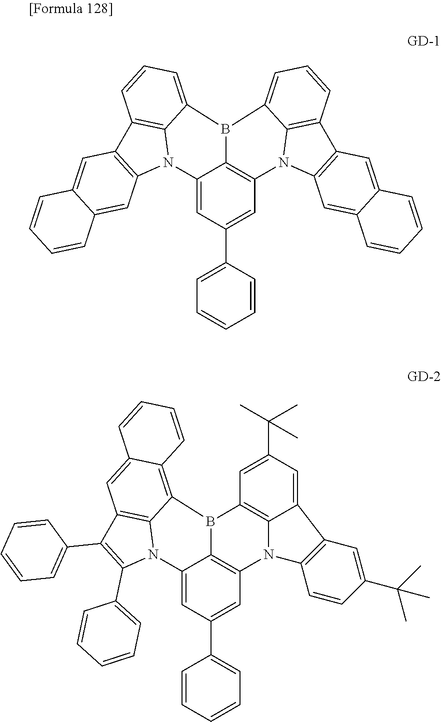 [SMILES: c1ccc(-c2cc3c4c(c2)-n2c5cc6ccccc6cc5c5cccc(c52)B4c2cccc4c5cc6ccccc6cc5n-3c24)cc1 ; CC(C)(C)c1ccc2c(c1)c1cc(C(C)(C)C)cc3c1n2-c1cc(-c2ccccc2)cc2c1B3c1c3ccccc3cc3c(-c4ccccc4)c(-c4ccccc4)n-2c13]

Structures of comparative compounds used for producing organic EL devices in Comparatives 1-3 and 2-1 are shown below.

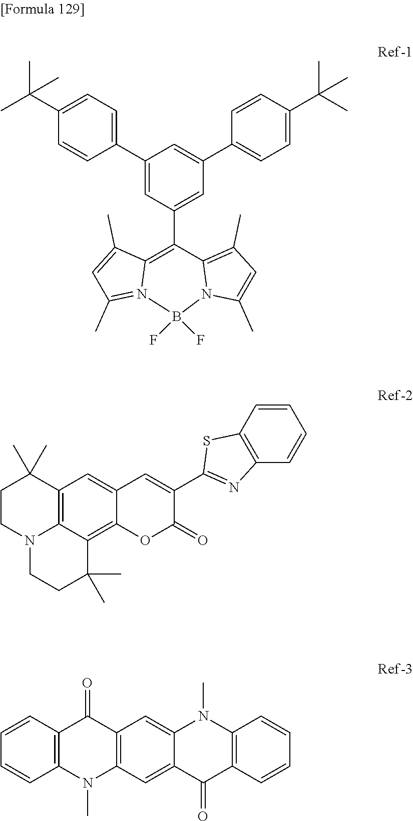

Structures of a compound represented by the formula (2) and used for producing organic EL devices in Examples 1, 2, and 2-1 to 2-8 are shown.

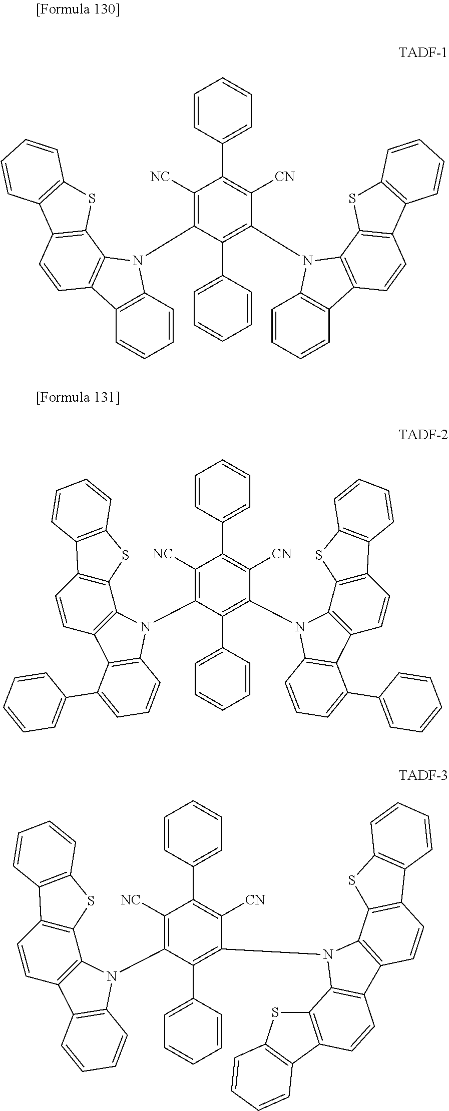 [SMILES: N#Cc1c(-c2ccccc2)c(C#N)c(-n2c3ccccc3c3ccc4c5ccccc5sc4c32)c(-c2ccccc2)c1-n1c2ccccc2c2ccc3c4ccccc4sc3c21 ; N#Cc1c(-c2ccccc2)c(C#N)c(-n2c3cccc(-c4ccccc4)c3c3ccc4c5ccccc5sc4c32)c(-c2ccccc2)c1-n1c2cccc(-c3ccccc3)c2c2ccc3c4ccccc4sc3c21 ; N#Cc1c(-c2ccccc2)c(C#N)c(-n2c3c(ccc4c5ccccc5sc43)c3ccc4c5ccccc5sc4c32)c(-c2ccccc2)c1-n1c2ccccc2c2ccc3c4ccccc4sc3c21]

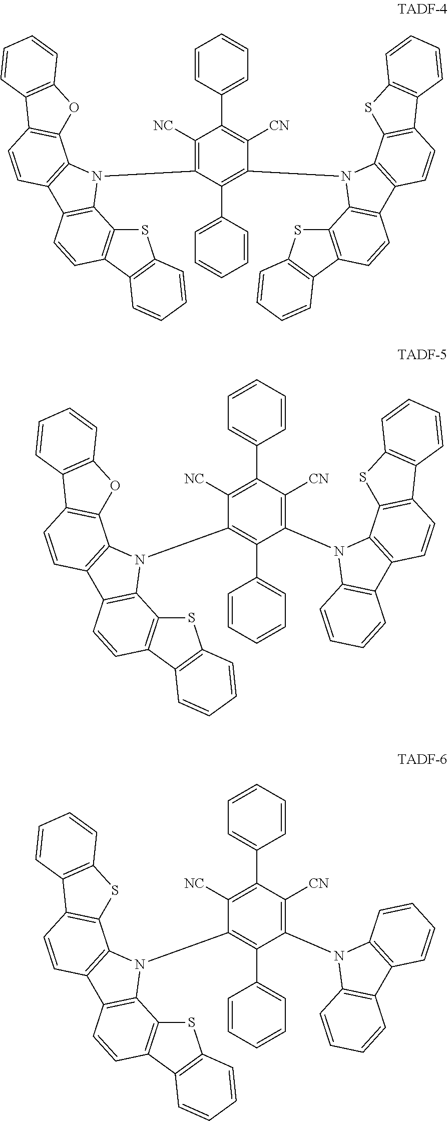 [SMILES: N#Cc1c(-c2ccccc2)c(C#N)c(-n2c3c(ccc4c5ccccc5sc43)c3ccc4c5ccccc5sc4c32)c(-c2ccccc2)c1-n1c2c(ccc3c4ccccc4oc32)c2ccc3c4ccccc4sc3c21 ; N#Cc1c(-c2ccccc2)c(C#N)c(-n2c3c(ccc4c5ccccc5oc43)c3ccc4c5ccccc5sc4c32)c(-c2ccccc2)c1-n1c2ccccc2c2ccc3c4ccccc4sc3c21 ; N#Cc1c(-c2ccccc2)c(C#N)c(-n2c3c(ccc4c5ccccc5sc43)c3ccc4c5ccccc5sc4c32)c(-c2ccccc2)c1-n1c2ccccc2c2ccccc21]

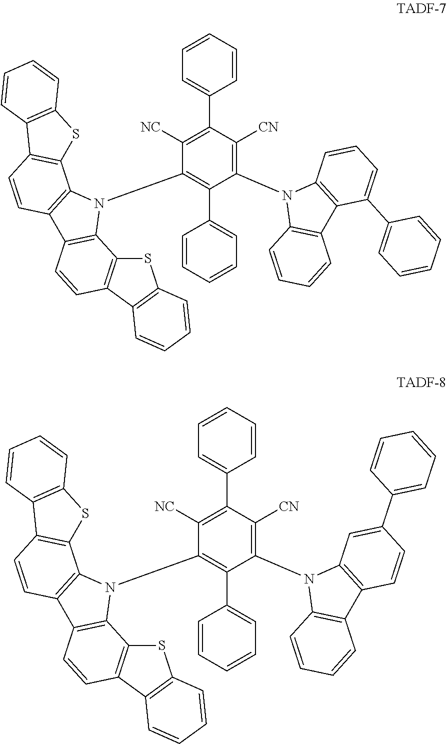 [SMILES: N#Cc1c(-c2ccccc2)c(C#N)c(-n2c3c(ccc4c5ccccc5sc43)c3ccc4c5ccccc5sc4c32)c(-c2ccccc2)c1-n1c2ccccc2c2c(-c3ccccc3)cccc21 ; N#Cc1c(-c2ccccc2)c(C#N)c(-n2c3c(ccc4c5ccccc5sc43)c3ccc4c5ccccc5sc4c32)c(-c2ccccc2)c1-n1c2ccccc2c2ccc(-c3ccccc3)cc21]

Structures of other compounds used for producing organic EL devices in Examples 1, 2, and 2-1 to 2-8 and Comparatives1-3 and 2-1 are shown below.

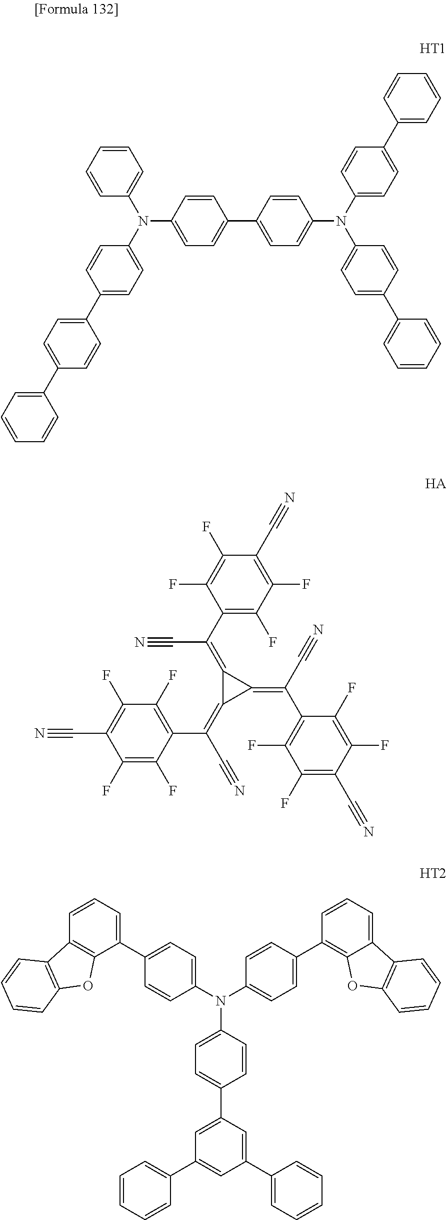 [SMILES: c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; c1ccc(-c2cc(-c3ccccc3)cc(-c3ccc(N(c4ccc(-c5cccc6c5oc5ccccc56)cc4)c4ccc(-c5cccc6c5oc5ccccc56)cc4)cc3)c2)cc1]

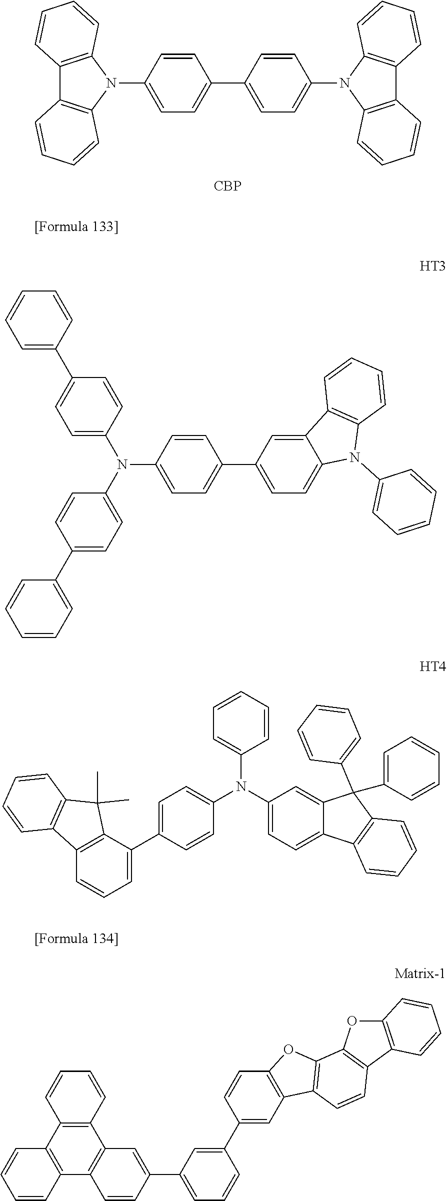 [SMILES: c1ccc2c(c1)c1ccccc1n2-c1ccc(-c2ccc(-n3c4ccccc4c4ccccc43)cc2)cc1 ; "CBP" ; c1ccc(-c2ccc(N(c3ccc(-c4ccccc4)cc3)c3ccc(-c4ccc5c(c4)c4ccccc4n5-c4ccccc4)cc3)cc2)cc1 ; CC1(C)c2ccccc2-c2cccc(-c3ccc(N(c4ccccc4)c4ccc5c(c4)C(c4ccccc4)(c4ccccc4)c4ccccc4-5)cc3)c21 ; c1cc(-c2ccc3oc4c(ccc5c6ccccc6oc54)c3c2)cc(-c2ccc3c4ccccc4c4ccccc4c3c2)c1]

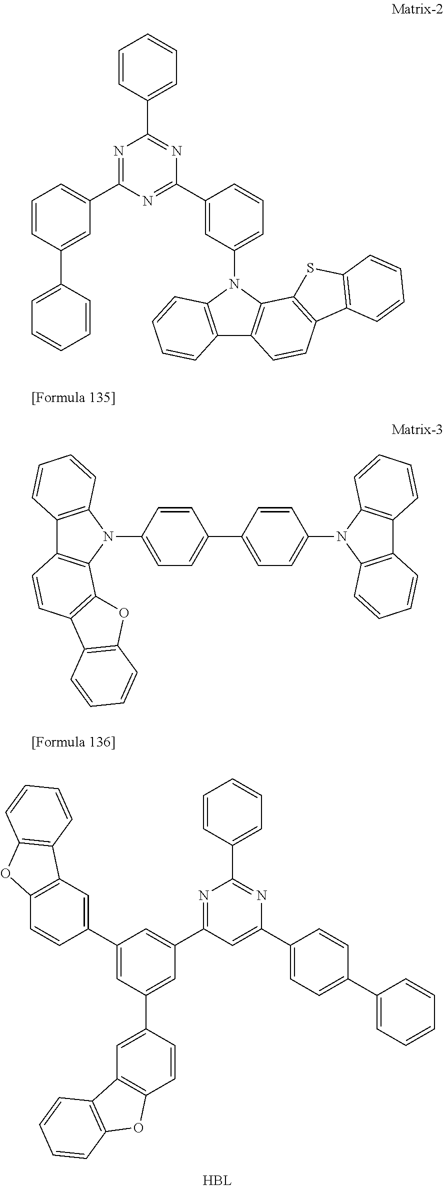 [SMILES: c1ccc(-c2cccc(-c3nc(-c4ccccc4)nc(-c4cccc(-n5c6ccccc6c6ccc7c8ccccc8sc7c65)c4)n3)c2)cc1 ; c1ccc2c(c1)oc1c2ccc2c3ccccc3n(-c3ccc(-c4ccc(-n5c6ccccc6c6ccccc65)cc4)cc3)c21 ; c1ccc(-c2ccc(-c3cc(-c4cc(-c5ccc6oc7ccccc7c6c5)cc(-c5ccc6oc7ccccc7c6c5)c4)nc(-c4ccccc4)n3)cc2)cc1]

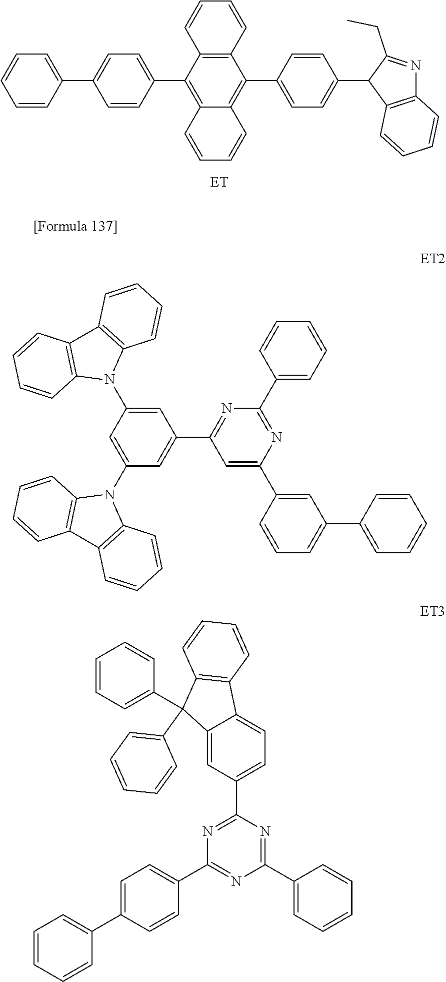 [SMILES: CCC1=Nc2ccccc2C1c1ccc(-c2c3ccccc3c(-c3ccc(-c4ccccc4)cc3)c3ccccc23)cc1 ; c1ccc(-c2cccc(-c3cc(-c4cc(-n5c6ccccc6c6ccccc65)cc(-n5c6ccccc6c6ccccc65)c4)nc(-c4ccccc4)n3)c2)cc1 ; c1ccc(-c2ccc(-c3nc(-c4ccccc4)nc(-c4ccc5c(c4)C(c4ccccc4)(c4ccccc4)c4ccccc4-5)n3)cc2)cc1]

### Production of Organic EL Devices

The organic EL devices were produced and evaluated as follows.

### Example 1

A glass substrate (size: 25 mm×75 mm×1.1 mm thick, produced by Geomatec Co., Ltd.) having an ITO transparent electrode (anode) was ultrasonic-cleaned in isopropyl alcohol for five minutes, and then UV-ozone-cleaned for one minute. The film thickness of ITO was 130 nm.

After the glass substrate having the transparent electrode line was cleaned, the glass substrate was mounted on a substrate holder of a vacuum deposition apparatus. Firstly, a compound HT1 and a compound HA were co-deposited on a surface of the glass substrate where the transparent electrode line was provided in a manner to cover the transparent electrode, thereby forming a 10-nm-thick hole injecting layer. The concentrations of the compound HT1 and the compound HA in the hole injecting layer were 97 mass % and 3 mass %, respectively.

Next, the compound HT1 was vapor-deposited on the hole injecting layer to form a 110-nm-thick first hole transporting layer.

A compound HT2 was then vapor-deposited on the first hole transporting layer to form a 5-nm-thick second hole transporting layer.

A compound CBP was then vapor-deposited on the second hole transporting layer to form a 5-nm-thick electron blocking layer.

Next, a compound Matrix-1 and a compound Matrix-2 each as the third compound, a compound TADF-1 as the second compound, and a compound GD-1 as the first compound were co-deposited on the electron blocking layer to form a 25-nm-thick emitting layer. The concentrations of the compound Matrix-1, the compound Matrix-2, the compound TADF-1, and the compound GD-1 in the emitting layer were 24 mass %, 25 mass %, 50 mass %, and 1 mass %, respectively.

A compound HBL was then vapor-deposited on the emitting layer to form a 5-nm-thick hole blocking layer.

A compound ET was then vapor-deposited on the hole blocking layer to form a 50-nm-thick electron transporting layer.

Next, lithium fluoride (LiF) was vapor-deposited on the electron transporting layer to form a 1-nm-thick electron injectable electrode (cathode).

Subsequently, metal aluminum (Al) was vapor-deposited on the electron injectable electrode to form an 80-nm-thick metal Al cathode.

A device arrangement of the organic EL device in Example 1 is roughly shown as follows.

ITO(130)/HT1:HA(10,97%:3%)/HT1(110)/HT2(5)/CBP(5)/Matrix-1:Matrix-2:TADF-1:GD-1(25,24%:25%:50%:1%)/HBL(5)/ET(50)/LiF(1)/Al(80)

Numerals in parentheses represent a film thickness (unit: nm).

The numerals (97%:3%) represented by percentage in the same parentheses indicate a ratio (mass %) between the compound HT1 and the compound HA in the hole injecting layer. The numerals (24%:24%:25%:1%) represented by percentage in the same parentheses indicate a ratio (mass %) between the compound Matrix-1, the compound Matrix-2, the compound TADF-1, and the compound GD-1 in the emitting layer. Similar notations apply to the description below.

### Example 2

An organic EL device in Example 2 was produced in the same manner as in Example 1 except that the compound GD-1 in the emitting layer in Example 1 was replaced by the first compound shown in Table 1.

### Comparatives 1 to 3

The organic EL devices in Comparatives 1 to 3 were produced in the same manner as in Example 1 except that the compound GD-1 in the emitting layer in Example 1 was replaced by the first compound shown in Table 1.

### Example 2-1

A glass substrate (size: 25 mm×75 mm×1.1 mm thick, manufactured by Geomatec Co., Ltd.) having an ITO transparent electrode (anode) was ultrasonic-cleaned in isopropyl alcohol for five minutes, and then UV-ozone-cleaned for one minute. The film thickness of ITO was 130 nm.

After the glass substrate having the transparent electrode line was cleaned, the glass substrate was mounted on a substrate holder of a vacuum deposition apparatus. Firstly, a compound HT3 and the compound HA were co-deposited on a surface of the glass substrate where the transparent electrode line was provided in a manner to cover the transparent electrode, thereby forming a 10-nm-thick hole injecting layer. The concentrations of the compound HT3 and the compound HA in the hole injecting layer were 97 mass % and 3 mass %, respectively.

Next, the compound HT3 was vapor-deposited on the hole injecting layer to form a 90-nm-thick hole transporting layer.

A compound HT4 was then vapor-deposited on the hole transporting layer to form a 30-nm-thick electron blocking layer.

Next, a compound Matrix-3 as the third compound, the compound TADF-1 as the second compound, and a compound GD-2 as the first compound were co-deposited on the electron blocking layer to form a 25-nm-thick emitting layer. The concentrations of the compound Matrix-3, the compound TADF-1, and the compound GD-2 in the emitting layer were 71.2 mass %, 28 mass %, and 0.8 mass %, respectively.

Next, a compound ET2 was vapor-deposited on the emitting layer to form a 5-nm-thick hole blocking layer.

Next, a compound ET3 and Liq were co-deposited on the hole blocking layer to form a 50-nm-thick electron transporting layer. The concentrations of the compound ET3 and Liq in the electron transporting layer were 50 mass % and 50 mass %, respectively. Liq is an abbreviation of (8-quinolinolato)lithium ((8-Quinolinolato)lithium).

Next, ytterbium (Yb) was vapor-deposited on the electron transporting layer to form a 1-nm-thick electron injectable electrode (cathode).

Subsequently, metal aluminum (Al) was vapor-deposited on the electron injectable electrode to form an 80-nm-thick metal Al cathode.

A device arrangement of the organic EL device in Example 2-1 is roughly shown as follows.

ITO(130)/HT3:HA(10,97%:3%)/HT3(90)/HT4(30)/Matrix-3:TADF-1:GD-2(25,71.2%:28%:0.8%)/ET2(5)/ET3:Liq(50,50%:50%)/Yb(1)/Al(80)

### Examples 2-2 to 2-8

Organic EL devices in Example 2-2 to 2-8 were produced in the same manner as in Example 2-1 except that the compound TADF-1 in the emitting layer in Example 2-1 was replaced by the second compound shown in Table 2.

### Comparatives 2-1

An organic EL device in Comparative 2-1 was produced in the same manner as in Example 2-5 except that the compound GD-2 in the emitting layer in Example 2-5 was replaced by the first compound shown in Table 2.

### Evaluation of Organic EL Devices

The organic EL devices produced were evaluated as follows. Tables 1 and 2 show evaluation results. Tables 1 and 2 also show the singlet energies S1 of the first compound, the second compound, and the third compound used in the emitting layer of each Example.

### Maximum Peak Wavelength λₚ and Emission Full Width at Half Maximum FWHM

Voltage was applied on the produced organic EL device such that a current density was 10 mA/cm², where spectral radiance spectrum was measured by a spectroradiometer CS-2000 (manufactured by Konica Minolta, Inc.). Maximum peak wavelength λₚ (unit: nm) and emission full width at half maximum FWHM (unit: nm) were calculated based on the obtained spectral-radiance spectra.

### Lifetime LT95

Voltage was applied to the organic EL device produced in each Example so that a current density was 50 mA/cm², where a time (LT95 (unit: h)) elapsed before a luminance intensity was reduced to 95% of the initial luminance intensity was measured as a lifetime. The luminance intensity was measured by a spectroradiometer CS-2000 (manufactured by Konica Minolta, Inc.).

“LT95 (relative values)” in Table 1 are calculated from measurement values of LT95 in respective Example (Examples 1, 2, and Comparatives 1 to 3) according to a numerical formula (Numerical Formula 1X) below.

LT95(relative value)=(LT95 of each Example/LT95 of Comparative 1)×100 (Numerical Formula 1X)

“LT95 (relative values)” in Table 2 are calculated from measurement values of LT95 in respective Example (Examples 2-1 to 2-8 and Comparatives 2-1) according to a numerical formula (Numerical Formula 2X) below.

LT95(relative value)=(LT95 of each Example/LT95 of Comparative 2-1)×100 (Numerical Formula 2X)

### External Quantum Efficiency EQE

Voltage was applied on the produced organic EL device such that a current density was 10 mA/cm², where spectral radiance spectrum was measured by a spectroradiometer CS-2000 (manufactured by Konica Minolta, Inc.). The external quantum efficiency EQE (unit: %) was calculated from the obtained spectral radiance spectra, assuming that the spectra was provided under a Lambertian radiation. Table 2 shows “EQE” (unit: %) as the relative values.

“EQE (relative values)” in Table 2 are calculated from measurement values of EQE in respective Example (Examples 2-1 to 2-8 and Comparatives 2-1) according to a numerical formula (Numerical Formula 3X) below.

EQE(relative value)=(EQE of each Example/EQE of Comparative 2-1)×100 (Numerical Formula 3X)

### CIE1931 Chromaticity

Voltage was applied on the produced organic EL device such that a current density was 10 mA/cm², where CIE1931 chromaticity coordinates (x, y) were measured by a spectroradiometer CS-2000 (manufactured by Konica Minolta, Inc.).

### Drive Voltage

A voltage (unit: V) was measured when current was applied between the anode and the cathode of the organic EL device such that a current density was 10 mA/cm².

TABLE 1

| | | | | | | | | | | | Device evaluation | |
|---|---|---|---|---|---|---|---|---|---|---|---|---|
| | Emitting layer | | | | | | | | | | LT95 | |
| | Third compound | | | | Second compound | | | First compound | | | (Relative | |
| | | S₁ | | S₁ | | S₁ | ΔST | | S₁ | λ | value) | λp |
| | Name | [eV] | Name | [eV] | Name | [eV] | [eV] | Name | [eV] | [nm] | [%] | [nm] |
| Ex. 1 | Matrix-1 | 3.68 | Matrix-2 | 3.08 | TADF-1 | 2.66 | <0.01 | GD-1 | 2.44 | 504 | 750 | 516 |
| Ex. 2 | Matrix-1 | 3.68 | Matrix-2 | 3.08 | TADF-1 | 2.66 | <0.01 | GD-2 | 2.39 | 512 | 1250 | 522 |
| Comp. 1 | Matrix-1 | 3.68 | Matrix-2 | 3.08 | TADF-1 | 2.66 | <0.01 | Ref-1 | 2.39 | 515 | 100 | 526 |
| Comp. 2 | Matrix-1 | 3.68 | Matrix-2 | 3.08 | TADF-1 | 2.66 | <0.01 | Ref-2 | 2.51 | 500 | 150 | 521 |
| Comp. 3 | Matrix-1 | 3.68 | Matrix-2 | 3.08 | TADF-1 | 2.66 | <0.01 | Ref-3 | 2.35 | 522 | 200 | 538 |

TABLE 2

| | Emitting layer | | | | | | | | Device evaluation | | | | | | |
|---|---|---|---|---|---|---|---|---|---|---|---|---|---|---|---|
| | Third | | | | | | | | | | | EQE | LT95 | | |
| | compound | | Second compound | | | | | First | Drive | | | (Relative | (Relative | | |
| | | S₁ | | λ | | S₁ | ΔST | compound | voltage | | | value) | value) | λp | FWHM |
| | Name | [eV] | Name | [nm] | PLQY | [eV] | [eV] | Name | [V] | CIEx | CIEy | [%] | [%] | [nm] | [nm] |
| Ex. 2-1 | Matrix-3 | 3.42 | TADF-1 | 493 | 0.88 | 2.66 | <0.01 | GD-2 | 3.99 | 0.245 | 0.691 | 168 | 550 | 520 | 29 |
| Ex. 2-2 | Matrix-3 | 3.42 | TADF-2 | 499 | 0.85 | 2.61 | <0.01 | GD-2 | 3.89 | 0.253 | 0.675 | 204 | 825 | 519 | 30 |
| Ex. 2-3 | Matrix-3 | 3.42 | TADF-3 | 498 | 0.90 | 2.58 | <0.01 | GD-2 | 3.96 | 0.300 | 0.649 | 172 | 950 | 520 | 38 |
| Ex. 2-4 | Matrix-3 | 3.42 | TADF-4 | 493 | 0.99 | 2.52 | <0.01 | GD-2 | 3.81 | 0.249 | 0.690 | 184 | 550 | 521 | 29 |
| Ex. 2-5 | Matrix-3 | 3.42 | TADF-5 | 491 | 0.94 | 2.67 | <0.01 | GD-2 | 3.90 | 0.267 | 0.662 | 152 | 775 | 518 | 33 |
| Ex. 2-6 | Matrix-3 | 3.42 | TADF-6 | 494 | 0.90 | 2.65 | <0.01 | GD-2 | 3.99 | 0.240 | 0.693 | 165 | 525 | 520 | 29 |
| Ex. 2-7 | Matrix-3 | 3.42 | TADF-7 | 494 | 0.91 | 2.65 | <0.01 | GD-2 | 3.97 | 0.228 | 0.701 | 177 | 350 | 520 | 28 |
| Ex. 2-8 | Matrix-3 | 3.42 | TADF-8 | 496 | 0.88 | 2.66 | <0.01 | GD-2 | 3.91 | 0.240 | 0.694 | 157 | 550 | 520 | 29 |
| Comp. | Matrix-3 | 3.42 | TADF-5 | 491 | 0.94 | 2.67 | <0.01 | Ref-1 | 3.87 | 0.256 | 0.693 | 100 | 100 | 524 | 34 |
| 2-1 | | | | | | | | | | | | | | | |

### Evaluation of Compounds

### Thermally Activated Delayed Fluorescence

### Delayed Fluorescence of Compound TADF-1

Delayed fluorescence was checked by measuring transient PL using an apparatus depicted in FIG. 2. The compound TADF-1 was dissolved in toluene to prepare a dilute solution with an absorbance of 0.05 or less at the excitation wavelength to eliminate contribution of self-absorption. In order to prevent quenching due to oxygen, the sample solution was frozen and degassed and then sealed in a cell with a lid under an argon atmosphere to obtain an oxygen-free sample solution saturated with argon.

The fluorescence spectrum of the sample solution was measured with a spectrofluorometer FP-8600 (manufactured by JASCO Corporation), and the fluorescence spectrum of a 9,10-diphenylanthracene ethanol solution was measured under the same conditions. Using the fluorescence area intensities of both spectra, the total fluorescence quantum yield was calculated by an equation (1) in Morris et al. J. Phys. Chem. 80 (1976) 969.

Prompt emission was observed immediately when the excited state was achieved by exciting the compound TADF-1 with a pulse beam (i.e., a beam emitted from a pulse laser) having a wavelength to be absorbed by the compound TADF-1, and Delay emission was observed not immediately when the excited state was achieved but after the excited state was achieved. The delayed fluorescence in Examples means that an amount of Delay emission is 5% or more with respect to an amount of Prompt emission. Specifically, provided that the amount of Prompt emission is denoted by X_{P} and the amount of Delay emission is denoted by X_{D}, the delayed fluorescence means that a value of X_{D}/X_{P} is 0.05 or more.

An amount of Prompt emission, an amount of Delay emission and a ratio between the amounts thereof can be obtained according to the method as described in “Nature 492, 234-238, 2012” (Reference Document 1). The amount of Prompt emission and the amount of Delay emission may be calculated using an apparatus different from one described in Reference Document 1 or one shown in FIG. 2

It was confirmed that the amount of Delay emission was 5% or more with respect to the amount of Prompt emission in the compound TADF-1.

Specifically, it was confirmed that the value of X_{D}/X_{P} was 0.05 or more in the compound TADF-1.

Compounds TADF-2 to TADF-8 were also measured in the same manner as the compound TADF-1, and the value of X_{D}/X_{P} in each of the compounds TADF-2 to TADF-8 was 0.05 or more.

### Singlet Energy S₁

A singlet energy S₁ of each of the compound Matrix-1, the compound Matrix-2, the compounds TADF-1 to TADF-8, the compound GD-1, the compound GD-2, a comparative compound Ref-1, a comparative compound Ref-2, and a comparative compound Ref-3 was measured according to the above-described solution method. Table 1 or 2 shows measurement results.

### ΔST

T_{77K} of each of the compounds TADF-1 to TADF-8 was measured. T_{77K} of each of the compounds TADF-1 to TADF-8 was measured according to the measurement method of the energy gap T_{77K} described in the above “Relationship between Triplet Energy and Energy Gap at 77K.”

ΔST of each of the compounds TADF-1 to TADF-8 was calculated based on the measured lowest singlet energy S₁ and the measured energy gap T_{77K} at 77K. Table 1 or 2 shows values of ΔST of the compound TADF-1. In Tables, “<0.01” indicates that ΔST is less than 0.01 eV.

### Maximum Peak Wavelength of Compounds A

A toluene solution of a measurement target compound at a concentration of 5 μmol/L was prepared and put in a quartz cell. A fluorescent spectrum (ordinate axis: fluorescent luminous intensity, abscissa axis: wavelength) of the thus-obtained sample was measured at a normal temperature (300K). In Examples, the fluorescence spectrum was measured using a spectrophotometer manufactured by Hitachi, Ltd. (apparatus name: F-7000). It should be noted that the apparatus for measuring the fluorescence spectrum is not limited to the apparatus used herein. A peak wavelength of the fluorescence spectrum exhibiting the maximum luminous intensity was defined as the maximum peak wavelength A.

### Synthesis of Compounds

### Synthesis Example 1: Synthesis of Compound TADF-1

A synthetic method of the compound TADF-1 will be described below.

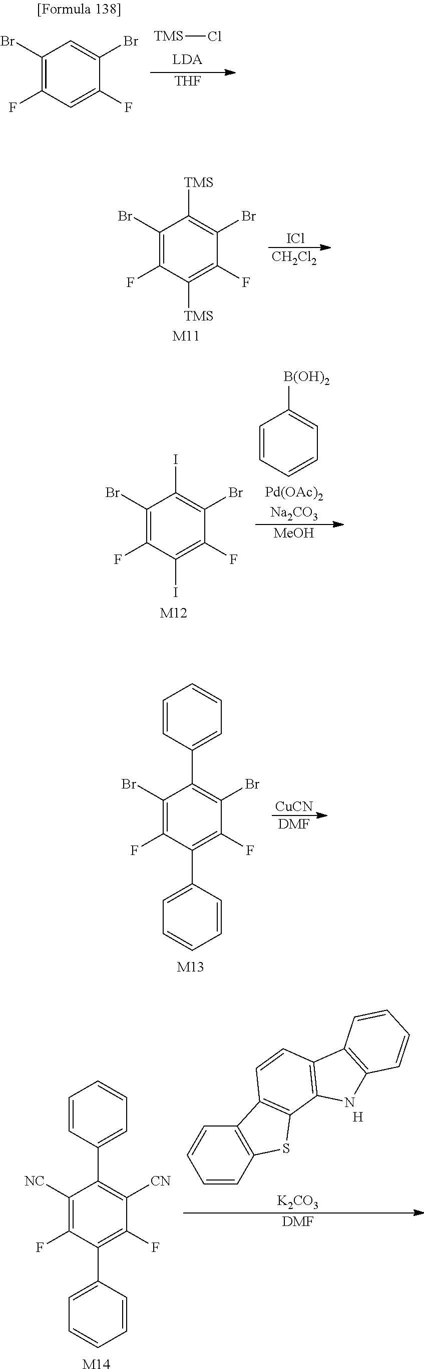

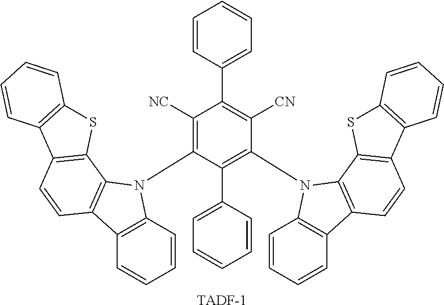

Under a nitrogen atmosphere, 1,5-dibromo-2,4-difluorobenzene (50 g, 184 mmol), chlorotrimethylsilane (60 g, 552 mmol), and THF (200 mL) were put into a 1000-mL three-necked flask. The material in the three-necked flask was cooled to −78 degrees C. in a dry ice/acetone bath. Subsequently, 230 mL of lithium diisopropyl amide (2M, THF solution) was added dropwise to the flask. The material was stirred at −78 degrees C. for two hours, then returned to the room temperature, and further stirred for two hours. After stirring, water (200 mL) was added into the three-necked flask. Subsequently, an organic layer was extracted with acetic ether. The extracted organic layer was washed with water and a saline solution and dried with magnesium sulfate. Then, a solvent was removed by a rotary evaporator under reduced pressure. The obtained intermediate M11 (73 g, 175 mmol, a yield of 95%) was not purified and used for a next reaction. Chlorotrimethylsilane is sometimes abbreviated as TMS-CI. TMS in a formula representing the intermediate M11 stands for a trimethylsilyl group. LDA is an abbreviation for lithium diisopropyl amide.

Under a nitrogen atmosphere, the intermediate M11 (73 g, 175 mmol) and dichloromethane (200 mL) were put into a 1000-mL eggplant flask. Iodine monochloride (85 g, 525 mmol) was dissolved in dichloromethane (200 mL) and added dropwise at 0 degrees C. to the flask. Subsequently, the mixture was stirred at 40 degrees C. for four hours. After stirring, the mixture was returned to the room temperature and added with a saturated aqueous solution of sodium hydrogen sulfite (100 mL). Then, an organic layer was extracted with dichloromethane. The extracted organic layer was washed with water and a saline solution. The washed organic layer was dried with magnesium sulfate. The dried organic layer was condensed by a rotary evaporator. A compound obtained through condensation was purified by silica-gel column chromatography to obtain an intermediate M12 (65 g, 124 mmol, a yield of 71%).

Under a nitrogen atmosphere, the intermediate M12 (22 g, 42 mmol), phenylboronic acid (12.8 g, 105 mmol), palladium acetate (0.47 g, 2.1 mmol), sodium carbonate (22 g, 210 mmol), and methanol (150 mL) were put into a 500-mL three-necked flask and stirred for four hours at 80 degrees C. After stirring, the reaction solution was left to be cooled to the room temperature. Subsequently, an organic layer was extracted with acetic ether. The extracted organic layer was washed with water and a saline solution. The washed organic layer was condensed by a rotary evaporator. A compound obtained through condensation was purified by silica-gel column chromatography to obtain an intermediate M13 (10 g, 24 mmol, a yield of 56%). The structure of the purified compound was identified by ASAP/MS. ASAP/MS is an abbreviation for Atmospheric Pressure Solid Analysis Probe Mass Spectrometry.

Under a nitrogen atmosphere, the intermediate M13 (10 g, 24 mmol), copper cyanide (10.6 g, 118 mmol), and DMF (15 mL) were put into a 200-mL three-necked flask and heated at 150 degrees C. for eight hours with stirring. After stirring, the reaction solution was cooled to the room temperature and then poured into ammonia water (10 mL). Next, an organic layer was extracted with methylene chloride. The extracted organic layer was washed with water and a saline solution. The washed organic layer was dried with magnesium sulfate. After drying, a solvent was removed by a rotary evaporator under reduced pressure. A compound obtained through removal under reduced pressure was purified by silica-gel column chromatography to obtain an intermediate M14 (5.8 g, 18.34 mmol, a yield of 78%). DMF is an abbreviation for N,N-dimethylformamide.

Under a nitrogen atmosphere, the intermediate M14 (1.0 g, 3.2 mmol), 12H-[1]Benzothieno[2,3-a]carbazole (1.9 g, 7 mmol), potassium carbonate (1.3 g, 9.50 mmol), and DMF (30 mL) were put into a 100-mL three-necked flask and stirred at 120 degrees C. for six hours. After stirring, the deposited solid was collected by filtration and purified by silica-gel column chromatography to obtain a compound TADF-1 (1.8 g, 2.2 mmol, a yield of 69%). The obtained compound was identified as the compound TADF-1 by analysis according to ASAP-MS.

### Synthesis Example 2: Synthesis of Compound GD-1

A synthetic method of the compound GD-1 will be described below.

### Production of Intermediate M21

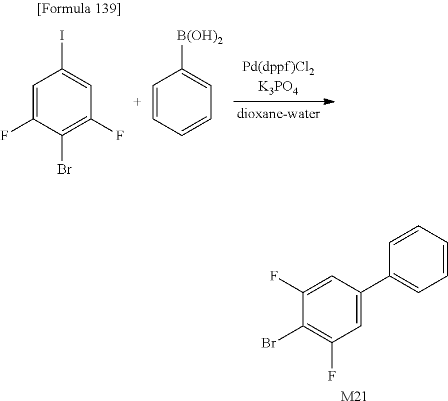

Under an argon atmosphere, a mixture of 2-bromo-1,3-difluoro-5-iodobenzene (47.8 g), phenylboronic acid (18.29 g), tripotassium phosphate (39.8 g), [1,1-bis(diphenyl phosphino) ferrocene]palladium(II) dichloride (1.09 g), 1,4-dioxane (250 mL), and water (125 mL) was stirred at the room temperature for four hours. Toluene (250 mL) and water (200 mL) were added to the obtained mixture to extract an aqueous layer with toluene. An organic layer was washed with a saturated saline solution and subsequently dried with magnesium sulfate, and a solvent was distilled under reduced pressure. The obtained residue was purified by silica-gel column chromatography to obtain an intermediate M21 (35.1 g, 87%). In the reaction scheme Pd(dppf)Cl₂ represents [1,1-bis(diphenylphosphino) ferrocene]palladium(II) dichloride.

### Production of Intermediate M22

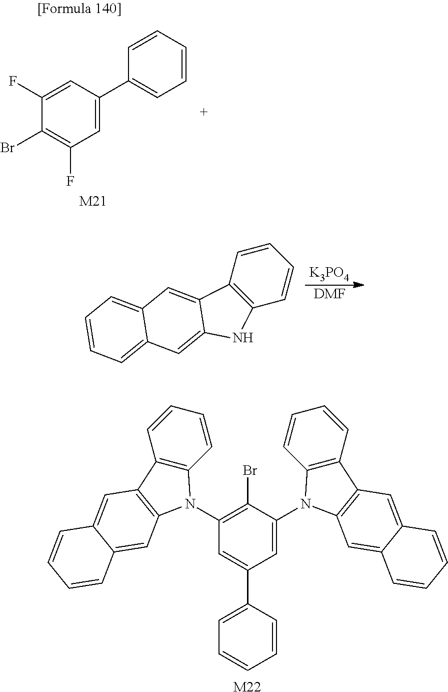

Under a nitrogen atmosphere, a mixture of the intermediate M21 (2.69 g), benzo[b]carbazole (4.34 g), tripotassium phosphate (12.7 g), and dimethylformamide (50 mL) was stirred at 140 degrees C. for 3.5 hours. After cooled to the room temperature, the mixture was added to water (200 mL) to deposit a solid. The deposited solid was collected by filtration and washed with water. The obtained solid was dissolved in dichloromethane, then absorbed onto silica gel, and purified by silica-gel column chromatography to obtain an intermediate M22 (3.16 g, 47%). In the reaction scheme, DMF stands for dimethylformamide.

### Production of Compound GD-1

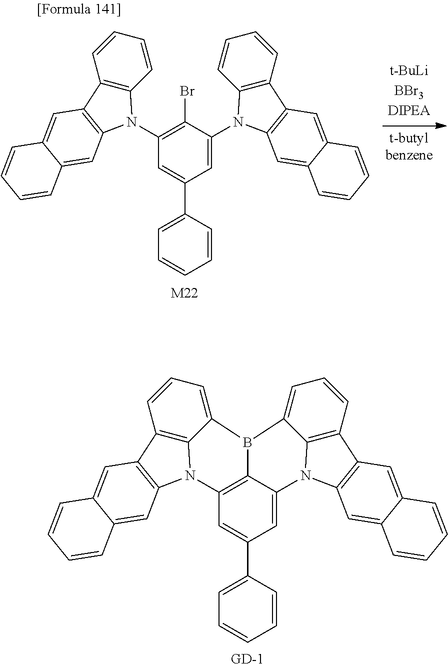

Under an argon atmosphere, the intermediate M22 (1.2 g) was added to t-butylbenzene (18 mL), cooled to 0 degrees C., and then, to which 1.9 M t-butyllithium pentane solution (1.9 mL) was added dropwise. After the dropwise addition, the obtained mixture was heated to 45 degrees C. and stirred for 15 minutes. Subsequently, the reaction mixture was cooled to −55 degrees C., added with boron tribromide (0.43 mL), heated to the room temperature and stirred for one hour. Subsequently, the reaction mixture was cooled to 0 degrees C., added with N,N-diisopropylethylamine (0.79 mL), stirred at the room temperature until the heat generation subsided, then heated to 145 degrees C., and stirred for 2.5 hours. After cooled to the room temperature, the reaction mixture was added with 1 N aqueous potassium acetate solution to deposit a solid. The deposited solid was collected by filtration and washed with water and ethanol. The solid collected by filtration was suspended in methylene chloride and the obtained solid was collected by filtration. Subsequently, the solid was further washed with methylene chloride to obtain 527 mg (a yield of 49%) of an orange solid. As a result of mass spectrometry, this orange solid was a target substance (compound GD-1) and had 593.3 [M+H]⁺ while a molecular weight was 592.51. In the reaction scheme, t-BuLi stands for tert-butyllithium and DIPEA stands for N,N-diisopropylethylamine.

### Synthesis Example 3: Synthesis of Compound GD-2

A synthetic method of the compound GD-2 will be described below.

### Production of Intermediate M23

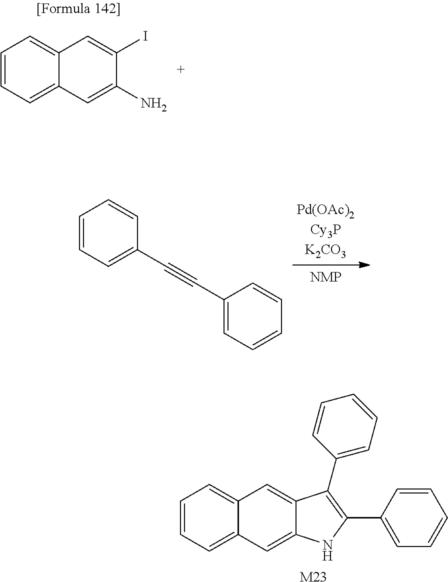

Under an argon atmosphere, a mixture of 2-amino-3-iodonaphthalene (4.28 g), 1,2-diphenylacetylene (3.40 g), palladium(II) acetate (178 mg), tricyclohexylphosphine (446 mg), potassium carbonate (5.49 mg), and N-methylpyrrolidone (360 mL) was stirred at 110 degrees C. for five hours. The obtained mixture was cooled to the room temperature. A portion of N-methylpyrrolidone was distilled away under reduced pressure, then diluted with t-butyl methyl ether, and added to water. An aqueous layer was extracted with t-butyl methyl ether. An organic layer was washed with a saturated saline solution and subsequently dried with magnesium sulfate, and a solvent was distilled away under reduced pressure. The obtained residue was purified by silica-gel column chromatography to obtain an intermediate M23 (2.78 g, 55%). In the reaction scheme, Pd(OAc)₂ represents palladium(II) acetate, Cy₃P represents tricyclohexylphosphine, and NMP represents N-methylpyrrolidone.

### Production of Intermediate M24

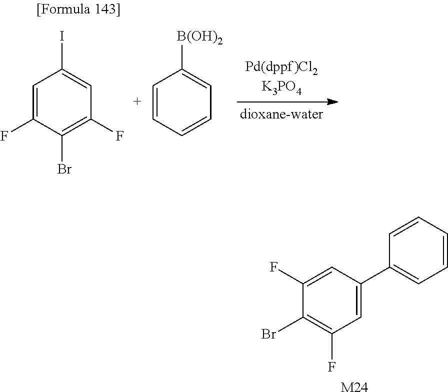

Under an argon atmosphere, a mixture of 2-bromo-1,3-difluoro-5-iodobenzene (47.8 g), phenylboronic acid (18.29 g), tripotassium phosphate (39.8 g), [1,1-bis(diphenyl phosphino) ferrocene]palladium(II) dichloride (1.09 g), 1,4-dioxane (250 mL), and water (125 mL) was stirred at the room temperature for four hours. Toluene (250 mL) and water (200 mL) were added to the obtained mixture to extract an aqueous layer with toluene. An organic layer was washed with a saturated saline solution and subsequently dried with magnesium sulfate, and a solvent was distilled under reduced pressure. The obtained residue was purified by silica-gel column chromatography to obtain an intermediate M24 (35.1 g, 87%). In the reaction scheme, Pd(dppf)Cl₂ represents [1,1-bis(diphenylphosphino) ferrocene]palladium(II) dichloride.

### Production of Intermediate M25

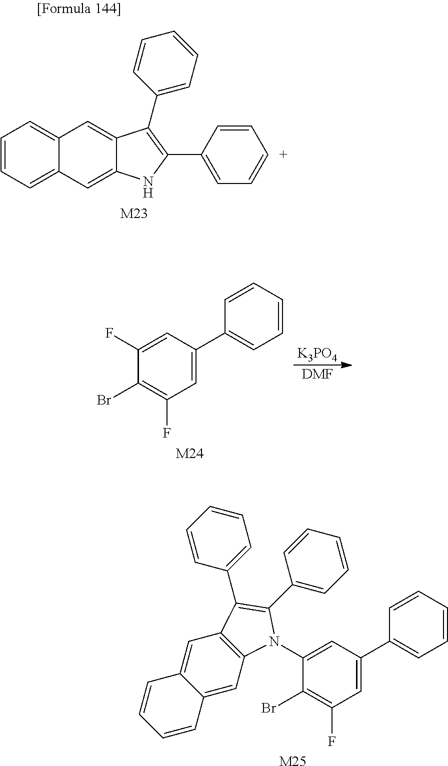

Under an argon atmosphere, a mixture of the intermediate M23 (6.39 g), the intermediate M24 (10.76 g), tripotassium phosphate (21.23 g), and dimethylformamide (140 mL) was stirred at 105 degrees C. for 48 hours. A portion of dimethylformamide was distilled away under reduced pressure, subsequently added to water, and extracted with t-butyl methyl ether. An organic layer was washed with a saturated saline solution and subsequently dried with magnesium sulfate, and a solvent was distilled away under reduced pressure. The obtained residue was purified by silica-gel column chromatography to obtain an intermediate M25 (6.2 g, 55%). In the reaction scheme, DMF stands for dimethylformamide.

### Production of Intermediate M26

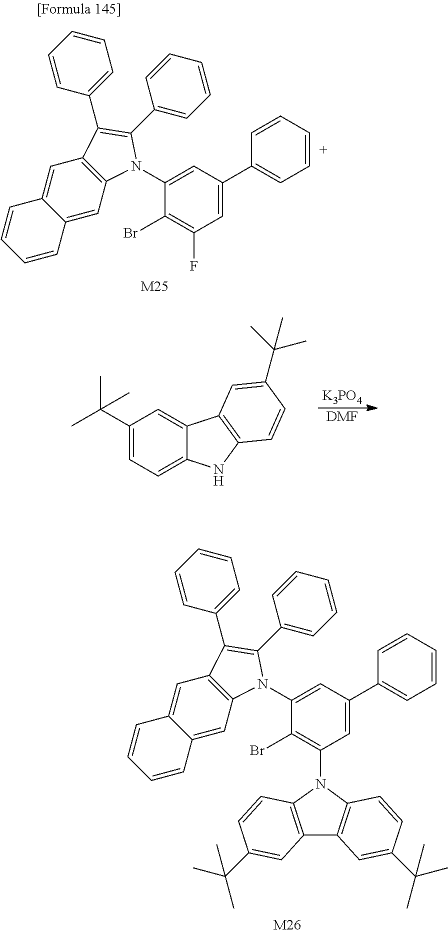

Under an argon atmosphere, a mixture of the intermediate M25 (6.14 g), 3,6-di-tert-butyl-9H-carbazole (3.32 g), tripotassium phosphate (6.88 g), and dimethylformamide (96 mL) was stirred at 105 degrees C. for 20 hours. A portion of dimethylformamide was distilled away under reduced pressure. The obtained mixture was added to 150 mL of water and stirred. The deposited solid was collected by filtration, washed with water, and then dried under reduced pressure. Further, the obtained solid was suspended in 220 mL of ethanol, heated under reflux for one hour. Subsequently, the obtained solid was collected by filtration to obtain 7.31 g (82%) of an intermediate M26.

### Production of Compound GD-2

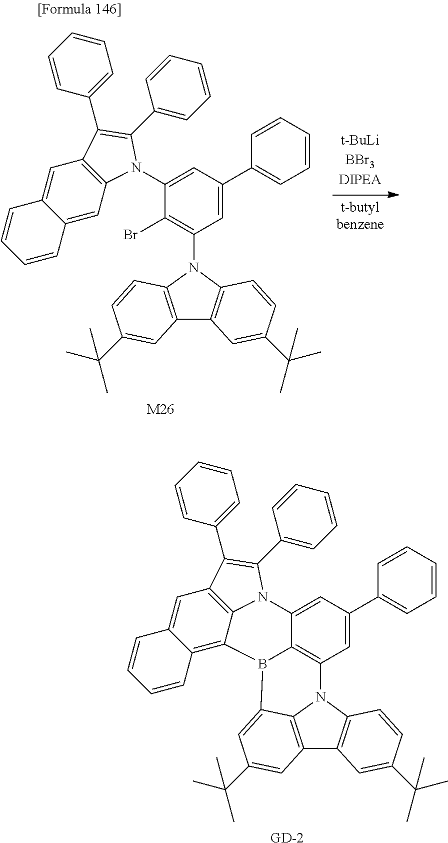

Under an argon atmosphere, the intermediate M26 (2.23 g) was added to t-butylbenzene (33 mL), cooled to −20 degrees C., and then, to which 1.9 M tert-butyllithium pentane solution (2.8 mL) was added dropwise. After the dropwise addition, the obtained mixture was heated to 70 degrees C. and stirred for 30 minutes. Subsequently, a component having a boiling point lower than that of tert-butylbenzene was distilled away under reduced pressure. The obtained mixture was cooled to −55 degrees C., added with boron tribromide (0.57 mL), heated to the room temperature, and stirred for one hour. Subsequently, the reaction mixture was cooled to 0 degrees C., added with N,N-diisopropylethylamine (1.19 mL), stirred at the room temperature until the heat generation subsided, then heated to 130 degrees C., and stirred overnight. After tert-butylbenzene was distilled away under reduced pressure, the residue was purified by flash chromatography to obtain 350 mg of an orange compound. As a result of mass spectrometry, this orange compound was a target substance (compound GD-2) and had 757.4 [M+H]⁺ while a molecular weight was 756.8. In the reaction scheme, t-BuLi stands for tert-butyllithium and DIPEA stands for N,N-diisopropylethylamine.

### EXPLANATION OF CODES

1 . . . organic EL device, 2 . . . substrate, 3 . . . anode, 4 . . . cathode, 5 . . . emitting layer, 6 . . . hole injecting layer, 7 . . . hole transporting layer, 8 . . . electron transporting layer, 9 . . . electron injecting layer

1. An organic electroluminescence device comprising:
an anode;
a cathode; and
an emitting layer provided between the anode and the cathode,
wherein the emitting layer comprises a first compound represented by a formula (I) below and a second compound that exhibits delayed fluorescence and is represented by a formula (2) below, and
a singlet energy S1(M1) of the first compound and a singlet energy S1(M2) of the second compound satisfy a relationship of a numerical formula (Numerical Formula 1) below, S1(M2)>S1(M1) 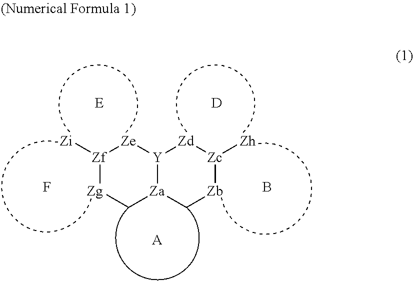
where:
a ring A, a ring B a ring D, a ring E, and a ring F are each independently a cyclic structure selected from the group consisting of a substituted or unsubstituted aryl ring having 6 to 30 ring carbon atoms, and a substituted or unsubstituted heterocyclic ring having 5 to 30 ring atoms:
one of the ring B and the ring D is present or both of the ring B and the ring D are present;
when both of the ring B and the ring D are present, the ring B and the ring D share a bond connecting Ze to Zh;
one of the ring E and the ring F is present or both of the ring E and the ring F are present;
when both of the ring E and the ring F are present, the ring F and the ring F share a bond connecting Zf to Zi;
Za is a nitrogen atom or a carbon atom;
Zb is a nitrogen atom or a carbon atom when the ring B is present, and Zb is an oxygen atom, a sulfur atom, N Rb, C(Rb₁)(Rb₂), or Si(Rb₃)(Rb₄) when the ring B is not present;
Zc is a nitrogen atom or a carbon atom;
Zd is a nitrogen atom or a carbon atom when the ring D is present, and Zd is an oxygen atom, a sulfur atom, or NRd when the ring D is not present;
Ze is a nitrogen atom or a carbon atom when the ring E is present, and Ze is an oxygen atom, a sulfur atom, or NRe when the ring E is not present;
Zf is a nitrogen atom or a carbon atom;
Zg is a nitrogen atom or a carbon atom when the ring F is present, and Zg is an oxygen atom, a sulfur atom, NRg, C(Rg₁)(Rg₂) or Si(Rg₃)(Rg₄) when the ring F is not present;
Zh is a nitrogen atom or a carbon atom;
Zi is a nitrogen atom or a carbon atom;
Y is a boron atom, a phosphorus atom, SiRh, P═O, or P═S;
Rb, Rb₁, Rb₂, Rb₃, Rb₄, Rd, Re, Rg, Rg₁, Rg₂, Rg₃, Rg₄ and Rh are each independently a hydrogen atom or a substituent:
Rb, Rb₁, Rb₂, Rb₃, Rb₄, Rd, Re, Rg, Rg₁, Rg₂, Rg₃, Rg₄, and Rh as a substituent are each independently a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted cycloalkyl group having 3 to 30 ring carbon atoms, a group represented by —Si(R₉₁₁)(R₉₁₃)(R₉₁₃), a group represented by —O—(R₉₁₄), a group represented by —S—(R₉₁₅), or a group represented by —N(R₉₁₆)(R₉₁₇); and
a bond between Y and Za, a bond between Y and Zd, and a bond between Y and Ze are each a single bond, 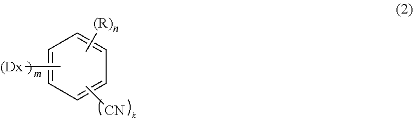
where:
k is 1, 2, 3, 4, or 5;
Dx is a group represented by a formula (21), (22), or (23) below, at least one Dx being a group represented by the formula (22) or (23);
m is 1, 2, 3, or 4;
when m is 2, 3, or 4, a plurality of Dx are mutually the same or different;
n is 0, 1, 2, or 3;
when n is 2 or 3, a plurality of R are mutually the same or different;
a sum of in and n is 2 or more;
each R is independently a halogen atom or a substituent; and
each R as a substituent is independently a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted cycloalkyl group having 3 to 30 ring carbon atoms, a group represented by —Si(R₉₁₁)(R₉₁₂)(R₉₁₃), a group represented by —O—(R₉₁₄), a group represented by —S—(R₉₁₅), or a group represented by —N(R₉₁₆)(R₉₁₇), 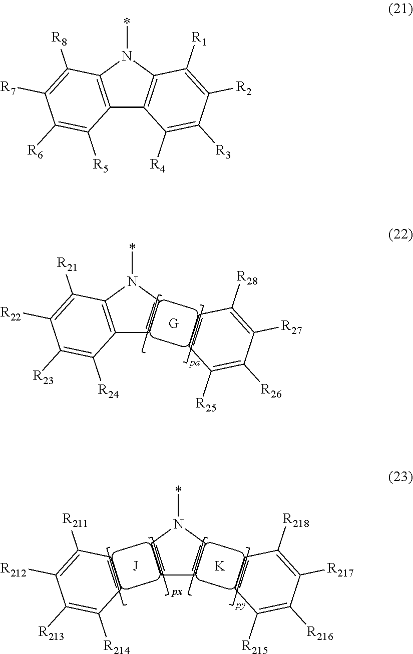
where:
R₁ to R₈ in the formula (21) are each independently a hydrogen atom, a halogen atom, or a substituent;
R₂₁ to R₂₈ in the formula (22) are each independently a hydrogen atom, a halogen atom, or a substituent, or at least one combination of a combination of R₂₁ and R₂₂, a combination of R₂₂ and R₂₃, a combination of R₂₃ and R₂₄, a combination of R₂₅ and R₂₆, a combination of R₂₆ and R₂₇, or a combination of R₂₇ and R₂₈ are mutually bonded to form a ring;
R₂₁₁ to R₂₁₈ in the formula (23) are each independently a hydrogen atom, a halogen atom, or a substituent, or at least one combination of a combination of R₂₁₁ and R₂₁₂, a combination of R₂₁₂ and R₂₁₃, a combination of R₂₁₃ and R₂₁₄, a combination of R₂₁₅ and R₂₁₆, a combination of R₂₁₆ and R₂₁₇, or a combination of R₂₁₇ and R₂₁₈ are mutually bonded to form a ring;
R₁ to R₈ as a substituent, R₂₁ to R₂₈ as a substituent, and R₂₁₁ to R₂₁₈ as a substituent are each independently a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted cycloalkyl group having 3 to 30 ring carbon atoms, a group represented by —Si(R₉₁₁)(R₉₁₂)(R₉₁₃), a group represented by —O—(R₉₁₄), a group represented by —S—(R₉₁₅), or a group represented by —N(R₉₁₆)(R₉₁₇); and
in the formulae (22) and (23):
a ring G, a ring J, and a ring K are each independently any cyclic structure selected from the group consisting of cyclic structures represented by formulae (24), (25), and (26) below;
the ring G, the ring J, and the ring K are fused with adjacent rings at any positions;
pa, px and py are each independently 1, 2, 3, or 4;
when pa is 2, 3, or 4, a plurality of rings G are mutually the same or different;
when px is 2, 3, or 4, a plurality of rings J are mutually the same or different;
when y is 2, 3, or 4, a plurality of rings K are mutually the same or different;
at least one Dx is a group represented by the formula (22) in which pa is 2, 3, or 4 and each ring G has any cyclic structure selected from the group consisting of cyclic structures represented by formulae (25) and (26) below, or at least one Dx is a group represented by the formula (23) in which at least one of px or py is 2, 3, or 4 and at least one ring J, at least one ring K, or both of at least one ring J and at least one ring K have any cyclic structure selected from the group consisting of cyclic structures represented by the formulae (25) and (26); and
each * in the formulae (21) to (23) represents a bonding position to a benzene ring in the formula (2), 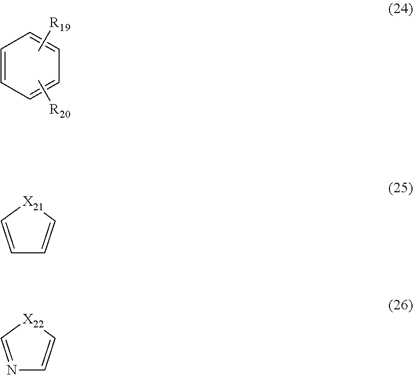
where: in the formula (24):
R₁₉ and R₂₀ are each independently a hydrogen atom, a halogen atom, or a substituent, or a combination of R₁₉ and R₂₀ are mutually bonded to form a ring,
in the formulae (25) and (26):
X₂₁ and X₂₂ are each independently a sulfur atom, an oxygen atom, NR₂₂₀, CR₂₂₁R₂₂₂, or SiR₂₂₃R₂₂₄;
R₂₂₀ is a hydrogen atom, a halogen atom, or a substituent;
R₂₂₁ and R₂₂₂ are each independently a hydrogen atom, a halogen atom, or a substituent, or a combination of R₂₂₁ and R₂₂₂ are mutually bonded to form a ring;
R₂₂₃ and R₂₂₄ are each independently a hydrogen atom, a halogen atom, or a substituent, or a combination of R₂₂₃ and R₂₂₄ are mutually bonded to form a ring; and
R₁₉, R₂₀, R₂₂₀, R₂₂₁, R₂₂₂, R₂₂₃ and R₂₂₄ as a substituent each independently represent the same as R₁ to R₈ as a substituent, and
in the first compound and the second compound:
R₉₁₁ to R₉₁₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₁₁ are present, the plurality of R₉₁₁ are mutually the same or different;
when a plurality of R₉₁₂ are present, the plurality of R₉₁₂ are mutually the same or different;
when a plurality of R₉₁₃ are present, the plurality of R₉₁₃ are mutually the same or different;
when a plurality of R₉₁₄ are present, the plurality of R₉₁₄ are mutually the same or different;
when a plurality of R₉₁₅ are present, the plurality of R₉₁₅ are mutually the same or different;
when a plurality of R₉₁₆ are present, the plurality of R₉₁₆ are mutually the same or different; and
when a plurality of R₉₁₇ are present, the plurality of R₉₁₇ are mutually the same or different.

2. The organic electroluminescence device according to claim 1,
wherein Rb, Rb₁, Rb₂, Rb₃, Rb₄, Rd, Re, Rg, Rg₁, Rg₂, Rg₃, Rg₄, and Rh are each independently a hydrogen atom, a substituted or unsubstituted aryl group having 6 to 30 ring carbon atoms or a substituted or unsubstituted heterocyclic group having 5 to 30 ring atoms.

3. The organic electroluminescence device according to claim 1, wherein Rb, Rb₁, Rb₂, Rb₃, Rb₄, Rd, Re, Rg, Rg₁, Rg₂, Rg₃, Rg₄, and Rh are each independently a hydrogen atom or a substituted or unsubstituted aryl group having 6 to 30 ring carbon atoms.

4. The organic electroluminescence device according to claim 1, wherein the first compound is a compound represented by a formula (11) below, 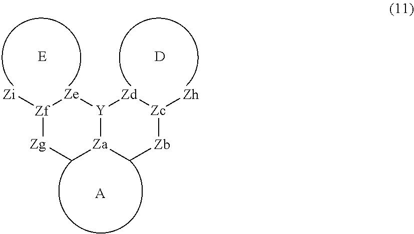
where:
a ring A, a ring D, and a ring E are each independently a cyclic structure selected from the group consisting of a substituted or unsubstituted aryl ring having 6 to 30 ring carbon atoms, and a substituted or unsubstituted heterocyclic ring having 5 to 30 ring atoms;
Za is a nitrogen atom or a carbon atom;
Zb is an oxygen atom, a sulfur atom, NRb, C(Rb₁)(Rb₂), or Si(Rb₃)(Rb₄);
Zc is a nitrogen atom or a carbon atom;
Zd is a nitrogen atom or a carbon atom;
Ze is a nitrogen atom or a carbon atom;
Zf is a nitrogen atom or a carbon atom;
Zg is an oxygen atom, a sulfur atom, NRg, C(Rg₁)(Rg₂), or Si(Rg₃)(Rg₄);
Zh is a nitrogen atom or a carbon atom;
Zi is a nitrogen atom or a carbon atom;
Y is a boron atom, a phosphorus atom, SiRh, P═O, or P═S; and
Rb, Rb₁, Rb₂, Rb₃, Rb₄, Rg, Rg₁, Rg₂, Rg₃, Rg₄, and Rh each independently represent the same as Rb, Rb₁, Rb₂, Rb₃, Rb₄, Rg, Rg₁, Rg₂, Rg₃, Rg₄, and Rh in the formula (1).

5. The organic electroluminescence device according to claim 4, wherein the first compound is a compound represented by a formula (12) below, 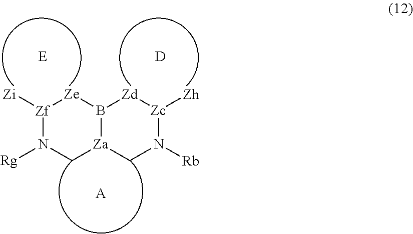
where:
a ring A, a ring D, and a ring E are each independently a cyclic structure selected from the group consisting of a substituted or unsubstituted aryl ring having 6 to 30 ring carbon atoms, and a substituted or unsubstituted heterocyclic ring having 5 to 30 ring atoms;
Za, Zc, Zd, Ze, Lf, Zh, and Zi are each independently a nitrogen atom or a carbon atom; and
Rb and Rg each independently represent the same as Rb and Rg in the formula (1).

6. The organic electroluminescence device according to claim 1, wherein the first compound is a compound represented by a formula (13) below, 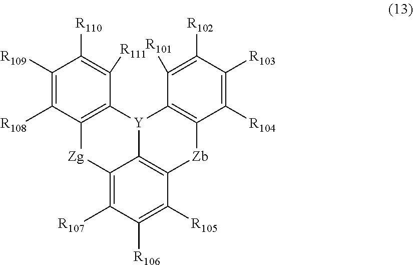
where:
Zb is an oxygen atom, a sulfur atom, or NRb
Zg is an oxygen atom, a sulfur atom, or NRg;
Y is a boron atom, a phosphorus atom, SiRh, P═O, or P═S;
Rb, Rg, and Rh each independently represent the same as Rb, Rg, and Rh in the formula (1);
R₁₀₁ to R₁₁₁ are each independently a hydrogen atom or a substituent;
R₁₀₁ to R₁₁₁ as a substituent are each independently a group selected from the group consisting of a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted haloalkyl group having 1 to 30 carbon atoms, a substituted or unsubstituted aryloxy group having 6 to 30 ring carbon atoms, a group represented by —Si(R₉₀₁)(R₉₀₂)(R₉₀₃), a group represented by —N(R₉₀₆)(R₉₀₇), a cyano group, and a halogen atom, and
in the first compound, R₉₀₁, R₉₀₂, R₉₀₃, R₉₀₆ and R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

7. The organic electroluminescence device according to claim 6, wherein the first compound is a compound represented by a formula (14) below, 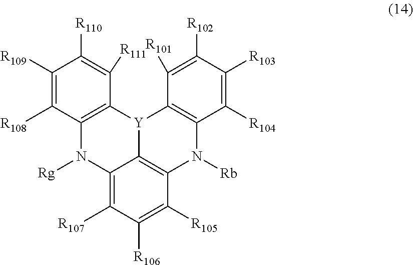
where:
R₁₀₁ to R₁₁₁ each independently represent the same as R₁₀₁ to R₁₁₁ in the formula (13); and
Rb and Rg each independently represent the same as Rb and Rg in the formula (1).

8. The organic electroluminescence device according to claim 6, wherein R₁₀₁ to R₁₁₁ are each independently a hydrogen atom, a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon atoms.

9. The organic electroluminescence device according to claim 6, wherein R₁₀₁ to R₁₁₁ are each independently a hydrogen atom or a substituted or unsubstituted aryl group having 6 to 30 ring carbon atoms.

10. The organic electroluminescence device according to claim 6, wherein R₁₀₁ to R₁₁₁ are each a hydrogen atom.

11. The organic electroluminescence device according to claim 1, wherein the first compound is a compound represented by a formula (15) below, 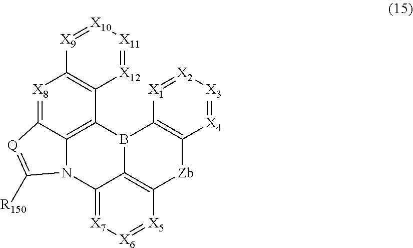
where:
Zb is an oxygen atom a sulfur atom, NRb, C(Rb₁)(Rb₂), or Si(Rb₃)(Rb₄);
X₁ is CR₁₂₁ a nitrogen atom, or a carbon atom bonded to X₁₂ with a single bond;
X₂ is CR₁₂₂ or a nitrogen atom;
X₃ is CR₁₂₃ or a nitrogen atom;
X₄ is CR₁₂₄ or a nitrogen atom;
X₅ is CR₁₂₅ or a nitrogen atom:
X₆ is CR₁₂₆ or a nitrogen atom;
X₇ is CR₁₂₇ or a nitrogen atom;
X₈ is CR₁₂₈ or a nitrogen atom;
X₉ is CR₁₂₉ or a nitrogen atom:
X₁₀ is CR₁₃₀ or a nitrogen atom;
X₁₁ is CR₁₃₁ or a nitrogen atom;
X₁₂ is CR₁₃₂, a nitrogen atom, or a carbon atom bonded to X₁, with a single bond;
Q is CR_{Q} or a nitrogen atom;
at least one combination of adjacent two or more of R₁₂₂ to R₁₃₁ are mutually bonded to form a substituted or unsubstituted monocyclic ring, mutually bonded to form a substituted or unsubstituted fused ring, or not mutually bonded;
at least one combination of adjacent two or more of R₁₂₄, R₁₂₅, Rb, Rb₁, Rb₂, Rb₃, and Rb₄ are mutually bonded to form a substituted or unsubstituted monocyclic ring, mutually bonded to form a substituted or unsubstituted fused ring, or not mutually bonded:
at least one hydrogen atom in a monocycle ring or a fused ring formed by mutually bonding at least one combination of adjacent two or more of R₁₂₄, R₁₂₅, Rb, Rb₁, Rb₂, Rb₃, and Rb₄ is substituted or not substituted by at least one substituent selected from the group consisting of an alkyl group having 1 to 50 carbon atoms, an aryl group having 6 to 50 ring carbon atoms, a heterocyclic group having 5 to 50 ring atoms, a group represented by —O—(R₁₅₁), and a group represented by —N(R₁₅₂)(R₁₅₃);
at least one hydrogen atom of the substituent is substituted or not substituted by an aryl group having 6 to 50 ring carbon atoms or an alkyl group having 1 to 50 carbon atoms;
R₁₂₁ to R₁₃₂ forming neither the substituted or unsubstituted monocyclic ring nor the substituted or unsubstituted fused ring, R₁₅₀, and R_{Q} are each independently a hydrogen atom, a substituted or unsubstituted alkyl group having 1 to 50 carbon atoms, a substituted or unsubstituted cycloalkyl group having 3 to 50 ring carbon atoms, a group represented by —Si(R₉₅₁)(R₉₅₂)(R₉₅₃), a group represented by —O—(R₉₅₄), a group represented by —S—(R₉₅₅), a group represented by —N(R₉₅₆)(R₉₅₇), a substituted or unsubstituted aralkyl group having 7 to 50 carbon atoms, a group represented by —C(═O)R₉₅₈, a group represented by —COOR₉₅₉, a halogen atom, a cyano group, a nitro group, a substituted or unsubstituted aryl group having 6 to 50 ring carbon atoms, or a substituted or unsubstituted heterocyclic group having 5 to 50 ring atoms;
Rb, Rb₁, Rb₂, Rb₃, and Rb₄ forming neither the substituted or unsubstituted monocyclic ring nor the substituted or unsubstituted fused ring are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
R₁₅₁ to R₁₅₃ and R₉₅₁ to R₉₅₉, are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms:
when a plurality of R₁₅₁ are present, the plurality of R₁₅₁ are mutually the same or different;
when a plurality of R₁₅₂ are present, the plurality of R₁₅₂ are mutually the same or different;
when a plurality of R₁₅₃ are present, the plurality of R₁₅₃ are mutually the same or different;
when a plurality of R₉₅₁ are present, the plurality of R₉₅₁ are mutually the same or different;
when a plurality of R₉₅₂ are present, the plurality of R₉₅₂ are mutually the same or different;
when a plurality of R₉₅₃ are present, the plurality of R₉₅₃ are mutually the same or different;
when a plurality of R₉₅₄ are present, the plurality of R₉₅₄ are mutually the same or different;
when a plurality of R₉₅₅ are present, the plurality of R₉₅₅ are mutually the same or different;
when a plurality of R₉₅₆ are present, the plurality of R₉₅₆ are mutually the same or different;
when a plurality of R₉₅₇ are present, the plurality of R₉₅₇ are mutually the same or different;
when a plurality of R₉₅₈ are present, the plurality of R₉₅₈ are mutually the same or different; and
when a plurality of R₉₅₉ are present, the plurality of R₉₅₉ are mutually the same or different.

12. The organic electroluminescence device according to claim 11, wherein R₁₂₁ to R₁₃₂, R₁₅₀, and R_{Q} are each independently a hydrogen atom, a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon atoms.

13. The organic electroluminescence device according to claim 11, wherein R₁₂₁ to R₁₃₂, R₁₅₀, and R_{Q} are each independently a hydrogen atom or a substituted or unsubstituted aryl group having 6 to 30 ring carbon atoms.

14. The organic electroluminescence device according to claim 11, wherein R₁₂₁ to R₁₃₂, R₁₅₀, and R_{Q} are each a hydrogen atom.

15. The organic electroluminescence device according to claim 11, wherein the first compound is a compound represented by a formula (151) below, 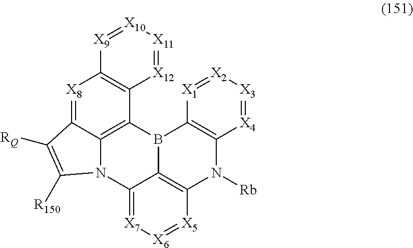
where:
X₁ to X₁₂ respectively represent the same as X₁ to X₁₂ in the formula (15); and
R₁₅₀, R_{Q}, and Rb respectively represent the same as R₁₅₀, R_{Q} and Rb in the formula (15).

16. The organic electroluminescence device according to claim 15, wherein the first compound is a compound represented by a formula (152) below, 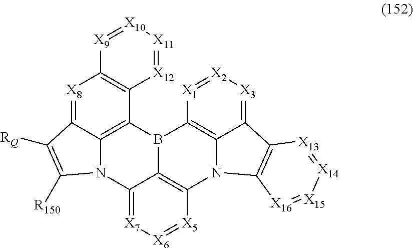
where:
X₁ to X₃ and X₅ to X₁₂ respectively represent the same as X₁ to X₃ and X₅ to X₁₂ in the formula (15);
X₁₃ is CR₁₃₃ or a nitrogen atom:
X₁₄ is CR₁₃₄ or a nitrogen atom:
X₁₅ is CR₁₃₅ or a nitrogen atom;
X₁₆ is CR₁₃₆ or a nitrogen atom;
R₁₃₃ to R₁₃₆ are each independently a hydrogen atom, a substituted or unsubstituted alkyl group having 1 to 30 carbon atoms, a substituted or unsubstituted cycloalkyl group having 3 to 50 ring carbon atoms, a group represented by —Si(R₉₅₁)(R₉₅₂)(R₉₅₃), a group represented by —O—(R₉₅₄), a group represented by —S—(R₉₅₅), a group represented by —N(R₉₅₆)(R₉₅₇), a substituted or unsubstituted aralkyl group having 7 to 50 carbon atoms, a group represented by —C(═O)R₉₅₈, a group represented by —COOR₉₅₉, a halogen atom, a cyano group, a nitro group, a substituted or unsubstituted aryl group having 6 to 50 ring carbon atoms, or a substituted or unsubstituted heterocyclic group having 5 to 50 ring atoms; and
R₁₅₀ to R_{Q} respectively represent the same as R₁₅₀ to R_{Q} in the formula (15).

17. The organic electroluminescence device according to claim 16,
wherein R₁₃₃ to R₁₃₆ are each independently a hydrogen atom, a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon atoms.

18. The organic electroluminescence device according to claim 16, wherein R₁₃₃ to R₁₃₆ are each independently a hydrogen atom or a substituted or unsubstituted aryl group having 6 to 30 ring carbon atoms.

19. The organic electroluminescence device according to claim 16, wherein R₁₃₃ to R₁₃₆ are each a hydrogen atom.

20. The organic electroluminescence device according to claim 1, wherein the first compound is a compound represented by a formula (16) below, 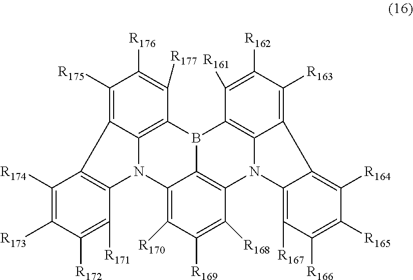
where:
at least one combination of adjacent two or more of R₁₆₁ to R₁₇₇ are mutually bonded to form a substituted or unsubstituted monocyclic ring, mutually bonded to form a substituted or unsubstituted fused ring, or not mutually bonded;
R₁₆₁ to R₁₇₇ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aralkyl group having 7 to 50 carbon atoms, a group represented by —Si(R₉₆₁)(R₉₆₂)(R₉₆₃), a group represented by —O—(R₉₆₄), a group represented by —S—(R₉₆₅), a group represented by —N(R₉₆₆)(R₉₆₇), a group represented by —C(═O)R₉₆₈, a group represented by —COOR₉₆₉, a halogen atom, a cyano group, a nitro group, a substituted or unsubstituted aryl group having 6 to 50 ring carbon atoms, or a substituted or unsubstituted heterocyclic group having 5 to 50 ring atoms;
R₉₆₁ to R₉₆₉ are each independently a hydrogen atom, a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms;
when a plurality of R₉₆₁ are present, the plurality of R₉₆₁ are mutually the same or different;
when a plurality of R₉₆₂ are present, the plurality of R₉₆₂ are mutually the same or different;
when a plurality of R₉₆₃ are present, the plurality of R₉₆₃ are mutually the same or different;
when a plurality of R₉₆₄ are present, the plurality of R₉₆₄ are mutually the same or different;
when a plurality of R₉₆₅ are present, the plurality of R₉₆₅ are mutually the same or different;
when a plurality of R₉₆₆ are present, the plurality of R₉₆₆ are mutually the same or different;
when a plurality of R₉₆₇ are present, the plurality of R₉₆₇ are mutually the same or different;
when a plurality of R₉₆₈ are present, the plurality of R₉₆₈ are mutually the same or different; and
when a plurality of R₉₆₉ are present, the plurality of R₉₆₉ are mutually the same or different.

21. The organic electroluminescence device according to claim 20, wherein R₁₆₁ to R₁₇₇ are each independently a hydrogen atom, a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon atoms.

22. The organic electroluminescence device according to claim 20, wherein at least one of R₁₆₈ to R₁₇₀ is a substituted or unsubstituted aryl group having 6 to 50 ring carbon atoms, or a substituted or unsubstituted heterocyclic group having 5 to 50 ring atoms.

23. The organic electroluminescence device according to claim 20, wherein R₁₆₁ to R₁₇₇ are each independently a hydrogen atom or a substituted or unsubstituted aryl group having 6 to 30 ring carbon atoms.

24. The organic electroluminescence device according to claim 20, wherein R₁₆₁ to R₁₇₇ are each a hydrogen atom.

25. The organic electroluminescence device according to claim 20, wherein at least one combination of adjacent two or more of R₁₆₁ to R₁₇₇ are mutually bonded to form a ring represented by a formula (16A) below, 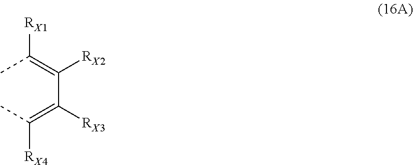
where: a dotted line represents a bonding position; and
R_{X1} to R_{X4} are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aralkyl group having 7 to 50 carbon atoms, a group represented by —Si(R₉₆₁)(R₉₆₂)(963), a group represented by —O—(R₉₆₄), a group represented by —S—(R₉₆₅), a group represented by —N(R₉₆₆)(967), a group represented by —C(═O)R₉₆₈, a group represented by —COOR₉₆₉, a halogen atom, a cyano group, a nitro group, a substituted or unsubstituted aryl group having 6 to 50 ring carbon atoms, or a substituted or unsubstituted heterocyclic group having 5 to 10 ring atoms.

26. The organic electroluminescence device according to claim 25, wherein at least one combination of a combination of R₁₆₁ and R₁₆₂, a combination of R₁₆₅ and R₁₆₆, a combination of R₁₇₂ and R₁₇₃, or a combination of R₁₇₆ and R₁₇₇ are mutually bonded to form a ring represented by the formula (16A).

27. The organic electroluminescence device according to claim 26, wherein the first compound is a compound represented by a formula (161) below, 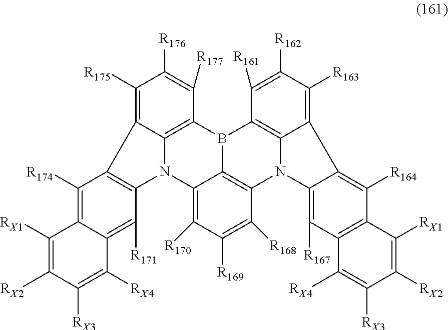
where:
R₁₆₁ to R₁₆₄, R₁₆₇ to R₁₇₁, R₁₇₄ to R₁₇₇, and R_{X1} and R_{X4} each independently represent the same as R₁₆₁ to R₁₆₄, R₁₆₇ to R₁₇₁, and R₁₇₄ to R₁₇₇ in the formula (16) and R_{X1} to R_{X4} in the formula (16A).

28. The organic electroluminescence device according to claim 1, wherein the first compound is a compound represented by a formula (1.71) or a formula (172) below 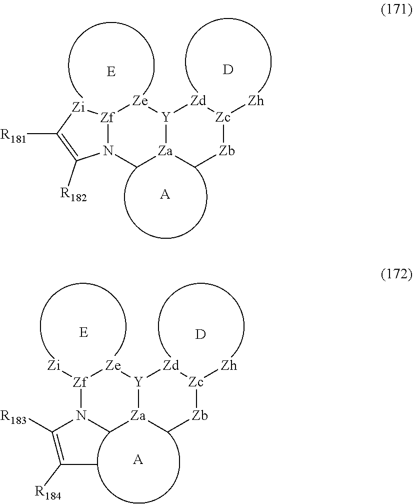
where, in the formulae (171) and (172):
a ring A, a ring D, and a ring E are each independently a cyclic structure selected from the group consisting of a substituted or unsubstituted aryl ring having 6 to 30 ring carbon atoms, and a substituted or unsubstituted heterocyclic ring having 5 to 30 ring atoms;
Za is a nitrogen atom or a carbon atom;
Zb is an oxygen atom, a sulfur atom, or NRb;
Ze is a nitrogen atom or a carbon atom;
Zd Is a carbon atom or a nitrogen atom;
Ze is a carbon atom or a nitrogen atom;
Zf is a nitrogen atom or a carbon atom;
Zh is a nitrogen atom or a carbon atom;
Zi is a nitrogen atom or a carbon atom;
Y is a boron atom, a phosphorus atom, SiRh, P═O, or P═S;
Rb and Rh are each independently a hydrogen atom or a substituent;
Rb and Rh as a substituent are each independently; selected from the group consisting of a substituted or unsubstituted aryl group having 6 to 30 ring carbon atoms, a substituted or unsubstituted heterocyclic group having 5 to 30 ring atoms; and a substituted or unsubstituted alkyl group having 1 to 30 carbon atoms;
a combination of R₁₈₁ and R₁₈₂ are mutually bonded to form a substituted or unsubstituted monocyclic ring, mutually bonded to form a substituted or unsubstituted fused ring, or not mutually bonded;
a combination of R₁₈₃ and R₁₈₄ are mutually bonded to form a substituted or unsubstituted monocyclic ring, mutually bonded to form a substituted or unsubstituted fused ring, or not mutually bonded;
R₁₈₁ to R₁₈₄ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aralkyl group having 7 to 50 carbon atoms, a group represented by —Si(R₉₇₁)(R₉₇₂)(R₉₇₃), a group represented by —O—(R₉₇₄), a group represented by —S—(R₉₇₅), a group represented by —N(R₉₇₆)(R₉₇₇), a group represented by —(═O)R₉₇₈, a group represented by —COOR₉₇₉, a halogen atom, a cyano group, a nitro group, a substituted or unsubstituted aryl group having 6 to 50 ring carbon atoms, or a substituted or unsubstituted heterocyclic group having 5 to 50 ring atoms;
R₉₇₁ to R₉₇₉ are each independently a hydrogen atom, a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms;
when a plurality of R₉₇₁ are present, the plurality of R₉₇₁ are mutually the same or different;
when a plurality of R₉₇₂ are present, the plurality of R₉₇₂ are mutually the same or different;
when a plurality of R₉₇₃ are present, the plurality of R₉₇₃ are mutually the same or different;
when a plurality of R₉₇₄ are present, the plurality of R₉₇₄ are mutually the same or different;
when a plurality of R₉₇₅ are present, the plurality of R₉₇₅ are mutually the same or different;
when a plurality of R₉₇₆ are present, the plurality of R₉₇₆ are mutually the same or different;
when a plurality of R₉₇₇ are present, the plurality of R₉₇₇ are mutually the same or different;
when a plurality of R₉₇₈ are present, the plurality of R₉₇₈ are mutually the same or different; and
when a plurality of R₉₇₉ are present, the plurality of R₉₇₉ are mutually the same or different.

29. The organic electroluminescence device according to claim 28, wherein R₁₈₁ to R₁₈₄ are each independently a hydrogen atom, a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon atoms.

30. The organic electroluminescence device according to claim 28, wherein R₁₈₁ to R₁₈₄ are each independently a hydrogen atom or a substituted or unsubstituted aryl group having 6 to 30 ring carbon atoms.

31. The organic electroluminescence device according to claim 28, wherein R₁₈₁ to R₁₈₄ are each a hydrogen atom.

32. The organic electroluminescence device according to claim 1, wherein the first compound is a compound represented by a formula (18) below, 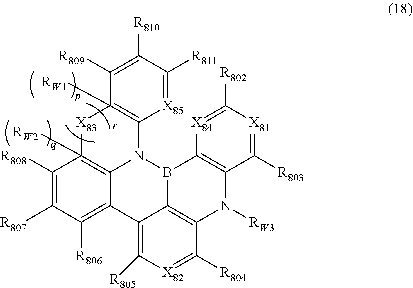
where:
r is 0 or 1;
when r is 0, p and q are 1 and R_{W1} and R_{W2} are present;
when r is 1, p and q are 0 and R_{W1} and R_{W2} are not present;
X₈₁ is a nitrogen atom or CR₁₉₁;
X₈₂ is a nitrogen atom or CR₁₉₂;
X₈₃ is a single bond, an oxygen atom, a sulfur atom, Si(R₁₉₃)(R₁₉₄), C(R₁₉₅)(R₁₉₆) or BR₁₉₇;
X₈₄ is R₈₀₁ or a carbon atom bonded to X₈₅ with a single bond;
X₈₅ is R₈₁₂ or a carbon atom bonded to X₈₄ with a single bond;
at least one combination of adjacent two or more of R₁₉₁, R₁₉₂, R₁₉₃, R₁₉₄, R₁₉₅, R₁₉₆, R₁₉₇, R₈₀₁, R₈₀₂, R₈₀₃, R₈₀₄, R₈₀₅, R₈₀₆, R₈₀₇, R₈₀₈, R₈₀₉, R₈₁₀, R₈₁₁, R₈₁₂, R_{W1}, R_{W2}, and R_{W3} are mutually bonded to form a substituted or unsubstituted monocyclic ring, mutually bonded to form a substituted or unsubstituted fused ring, or not mutually bonded;
R_{W3} forming neither the substituted or unsubstituted monocyclic ring nor the substituted or unsubstituted fused ring is a substituted or unsubstituted aryl group having 6 to 50 ring carbon atoms, a substituted or unsubstituted heterocyclic group having 5 to 50 ring atoms, or a substituted or unsubstituted alkyl group having 1 to 5 carbon atoms;
R₁₉₁, R₁₉₂, R₁₉₃, R₁₉₄, R₁₉₅, R₁₉₆, R₁₉₇, R₈₀₁, R₈₀₂, R₈₀₃, R₈₀₄, R₈₀₅, R₈₀₆, R₈₀₇, R₈₀₈, R₈₀₉, R₈₁₀, R₈₁₁, R₈₁₂, R_{W1}, and R_{W2}, forming neither the substituted or unsubstituted monocyclic ring nor the substituted or unsubstituted fused ring are each independently a hydrogen atom, a deuterium atom, a group represented by —Si(R₉₈₁)(R₉₈₂)(R₉₈₃), a group represented by —O—(R₉₈₄), a group represented by —S—(R₉₈₅), a group represented by —N(R₉₈₆)(R₉₈₇), a group represented by —B(R₉₈₈)(R₉₈₉), a group represented by —OSO₂(R₉₉₀), a cyano group, a halogen atom, a substituted or unsubstituted alkyl group having 1 to 50 carbon atoms, a substituted or unsubstituted cycloalkyl group having 3 to 50 ring carbon atoms, a substituted or unsubstituted fluoroalkyl group having 1 to 50 carbon atoms, a substituted or unsubstituted thioalkoxy group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted aryl group having 6 to 50 ring carbon atoms, or a substituted or unsubstituted heterocyclic group having 5 to 50 ring atoms;
R₉₈₁ to R₉₉₀ are each independently a hydrogen atom, a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms;
when a plurality of R₉₈₁ are present, the plurality of R₉₈₁ are mutually the same or different;
when a plurality of R₉₈₂ are present, the plurality of R₉₈₂ are mutually the same or different;
when a plurality of R₉₈₃ are present, the plurality of R₉₈₃ are mutually the same or different;
when a plurality of R₉₈₄ are present, the plurality of R₉₈₄ are mutually the same or different;
when a plurality of R₉₈₅ are present, the plurality of R₉₈₅ are mutually the same or different;
when a plurality of R₉₈₆ are present, the plurality of R₉₈₆ are mutually the same or different;
when a plurality of R₉₈₇ are present, the plurality of R₉₈₇ are mutually the same or different;
when a plurality of R₉₈₈ are present, the plurality of R₉₈₈ are mutually the same or different;
when a plurality of R₉₈₉ are present, the plurality of R₉₈₉ are mutually the same or different; and
when a plurality of R₉₉₀ are present, the plurality of R₉₉₀ are mutually the same or different.

33. The organic electroluminescence device according to claim 32, wherein R₁₉₁ to R₁₉₇, R₈₀₁ to R₈₁₂, R_{W1}, and R_{W2} are each independently a hydrogen atom, a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon atoms.

34. The organic electroluminescence device according to claim 32, wherein R₁₉₁ to R₁₉₇, R₈₀₁ to R₈₁₂, R_{W1}, and R_{W2} are each independently a hydrogen atom or a substituted or unsubstituted aryl group having 6 to 30 ring carbon atoms.

35. The organic electroluminescence device according to claim 32, wherein R₁₉₁ to R₁₉₇, R₈₀₁ to R₈₁₂, R_{W1}, and R_{W2} are each a hydrogen atom.

36. The organic electroluminescence device according to claim 32, wherein the first compound is a compound represented by a formula (181) below 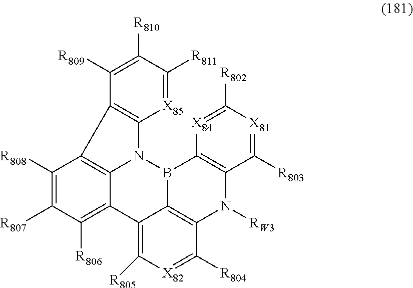
where:
X₈₁, X₈₂, X₈₄, and X₈₅ respectively represent the same as X₈₁, X₈₂, X₈₄, and X₈₅ in the formula (18), and
R₈₀₂ to R₈₁₁ and R_{W3} respectively represent the same as R₈₀₂ to R₈₁₁ and R_{W3} in the formula (18).

37. The organic electroluminescence device according to claim 32, wherein the first compound is a compound represented by a formula (182) below, 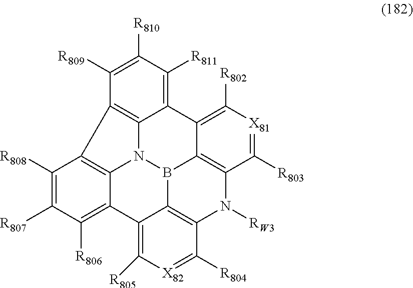
where:
X₈₁ and X₈₂ respectively represent the same as X₈₁ and X₈₂ in the formula (18); and
R₈₀₂ to R₈₁₁ and R_{W3} respectively represent the same as R₈₀₂ to R₈₁₁ and R_{W3} in the formula (18).

38. The organic electroluminescence device according to claim 1, wherein m in the formula (2) is 2.

39. The organic electroluminescence device according to claim 1, wherein a compound represented by the formula (2) is represented by a formula (210), (220) or (230) below, 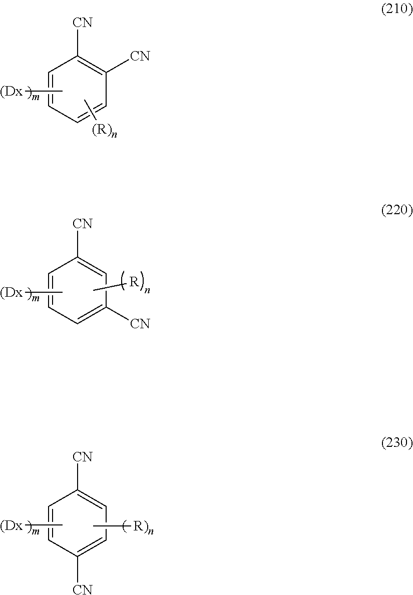
where, in the formulae (210), (220), and (230):
Dx, m, R, and n respectively represent the same as Dx, m, R, and n in the formula (2).

40. The organic electroluminescence device according to claim 1, wherein a compound represented by the formula (2) is a compound selected from the group consisting of compounds represented by formulae (21) to (218) below, 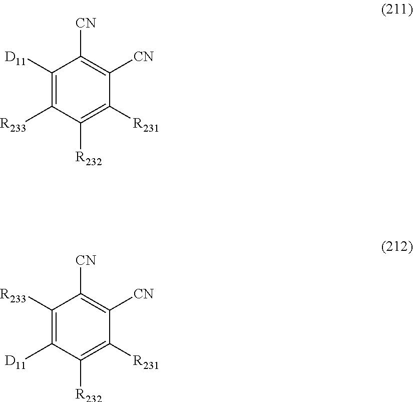
where, in the formulae (211) and (212):
D₁₁ is a group represented by the formula (22) or (23); and
R₂₃₁ to R₂₃₃ each independently represent the same as R in the formula (2), at least one of R₂₃₁ to R₂₃₃ being a substituent, R₂₃₁ to R₂₃₃ as a substituent representing the same as R as a substituent in the formula (2), 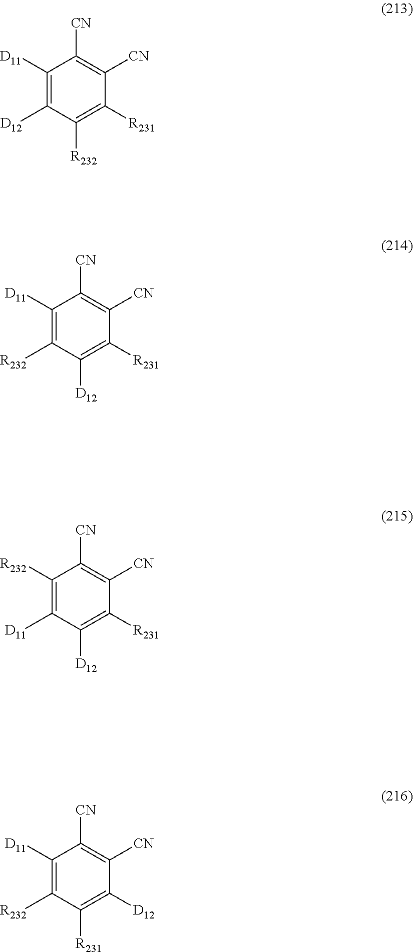
where, in the formulae (213) to (216):
D₁₁ and D₁₂ each independently represent the same as Dx in the formula (2), at least one of D₁₁ or D₁₂ being a group represented by the formula (22) or (23); and
R₂₃₁ and R₂₃₂ each independently represent the same as R in the formula (2), at least one of R₂₃₁ or R₂₃₂ being a substituent, R₂₃₁ and R₂₃₂ as a substituent representing the same as R as a substituent in the formula (2), 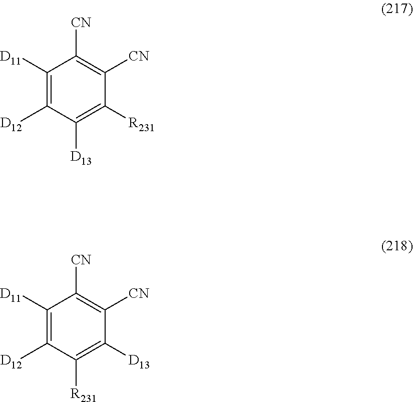
where, in the formulae (217) and (218):
D₁₁ to D₁₃ each independently represent the same as Dx in the formula (2), at least one of D₁₁ to D₁₃ being a group represented by the formula (22) or (23); and
R₂₃₁ is a substituent and R₂₃₁ as a substituent represents the same as R as a substituent in the formula (2).

41. The organic electroluminescence device according to claim 1, wherein a compound represented by the formula (2) is a compound selected from the group consisting of compounds represented by formulae (221) to (229) below, 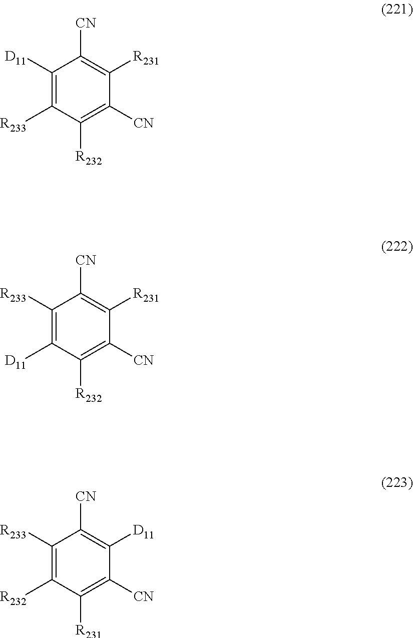
where, in the formulae (221) to (223):
D₁₁ is a group represented by the formula (22) or (23); and
R₂₃₁ to R₂₃₃ each independently represent the same as R in the formula (2), at least one of R₂₃₁ to R₂₃₃ being a substituent, R₂₃₁ to R₂₃₃ as a substituent representing the same as R as a substituent in the formula (2), 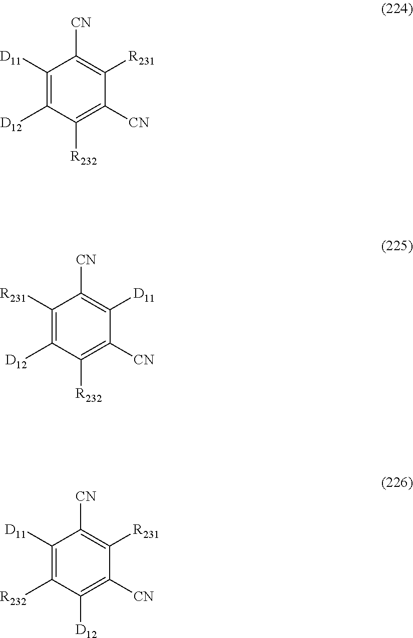
where, in the formulae (224) to (226):
D₁₁ and D₁₂ each independently represent the same as Dx in the formula (2), at least one of D₁₁ or D₁₂ being a group represented by the formula (22) or (23); and
R₂₃₁ and R₂₃₂ each independently represent the same as R in the formula (2), at least one of R₂₃₁ or R₂₃₂ being a substituent, R₂₃₁ and R₂₃₂ as a substituent representing the same as R as a substituent in the formula (2) 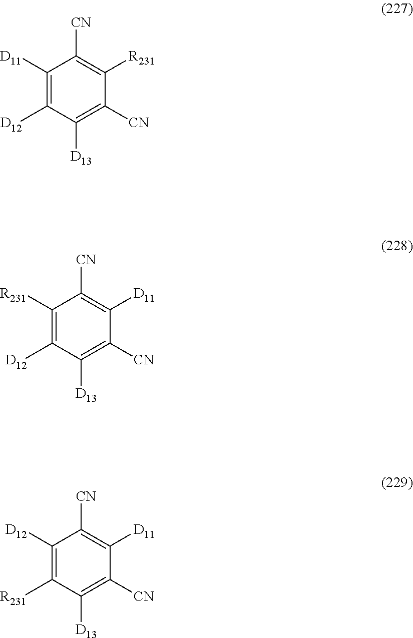
where, in the formulae (227) to (229):
D₁₁ to D₁₃ each independently represent the same as Dx in the formula (2), at least one of D₁₁ to D₁₃ being a group represented by the formula (22) or formula (22); and
R₂₃₁ is a substituent and R₂₃₁ as a substituent represents the same as R as a substituent in the formula (2).

42. The organic electroluminescence device according to claim 41, wherein a compound represented by the formula (2) is a compound represented by a formula (226) below.

43. The organic electroluminescence device according to claim 1, wherein a compound represented by the formula (2) is a compound selected from the group consisting of compounds represented by formulae (23 1) to (235) below, 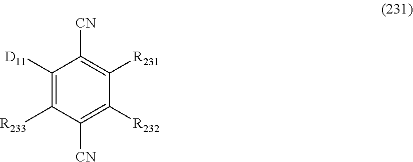
where:
D₁₁ is a group represented by the formula (22) or (23); and
R₂₃₁ to R₂₃₃ each independently represent the same as R in the formula (2), at least one of R₂₃₁ to R₂₃₃ being a substituent, R₂₃₁ to R₂₃₃ as a substituent representing the same as R as a substituent in the formula (2), 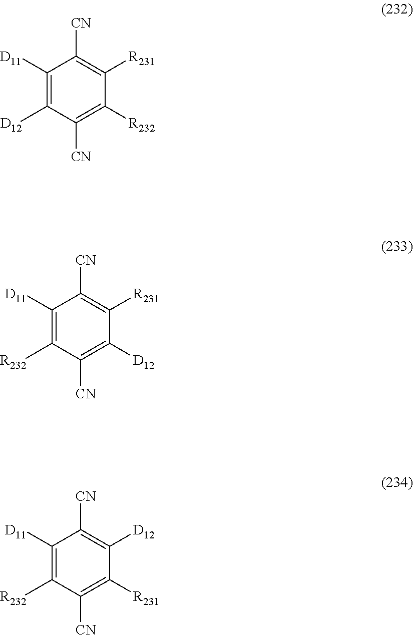
where, in the formulae (232) to (234):
D₁₁ and D₁₂ each independently represent the same as Dx in the formula (2), at least one of D₁₁ or D₁₂ being a group represented by the formula (22) or (23); and
R₂₃₁ and R₂₃₂ each independently represent the same as R in the formula (2), at least one of R₂₃₁ or R₂₃₂ being a substituent, R₂₃₁ and R₂₃₂ as a substituent representing the same as R as a substituent in the formula (2), 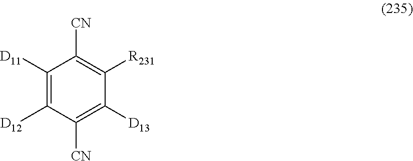
where:
D₁₁ to D₁₃ each independently represent the same as Dx in the formula (2), at least one of D₁₁ to D₁₃ being a group represented by the formula (22) or (23); and
R₂₃₁ is a substituent and R₂₃₁ as a substituent represents the same as R as a substituent in the formula (2).

44. The organic electroluminescence device according to claim 43, wherein a compound represented by the formula (2) is a compound represented by the formula (234).

45. The organic electroluminescence device according to claim 1, wherein a compound represented by the formula (2) is a compound represented by a formula (236) below, 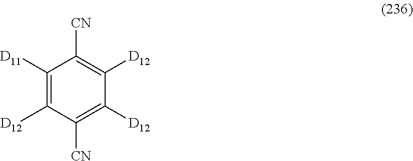
where:
D₁₁ is a group represented by a formula (22A), (22B), (22C), (22D), (22E) or (22F) below;
three D₁₂ are each independently a group represented by the formula (21); and
D₁₁ and D₁₂ are mutually different, 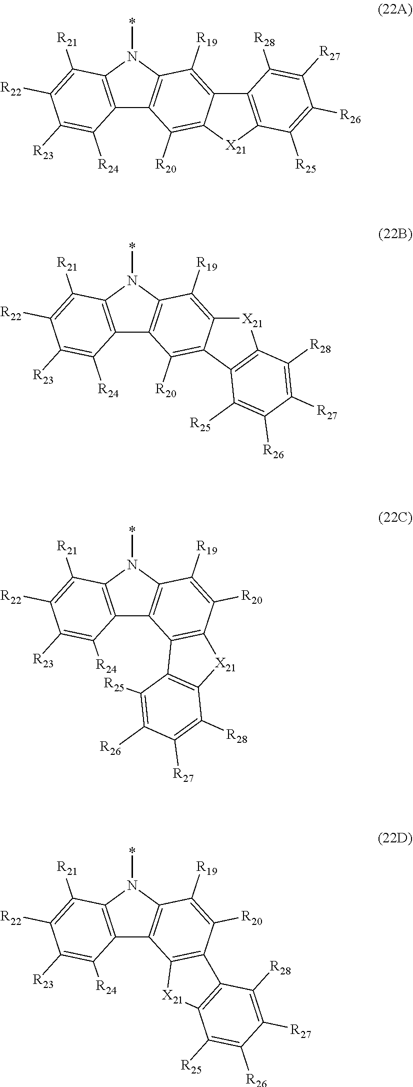 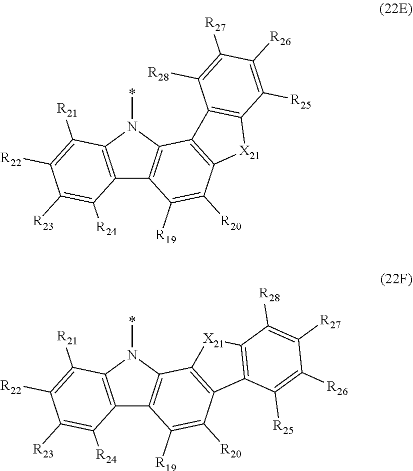
where, in the formulae (22A), (22B), (22C), (22D), (22E), and (22F):
R₂₁ to R₂₈ each independently represent the same as R₂₁ to R₂₈ in the formula (22);
R₁₉ and R₂₀ each independently represent the same as R₁₉ and R₂₀ in the formula (24):
X₂₁ represents the same as X₂₁ in the formula (25); and
each * in the formulae (22A), (22B), (220), (22D), (22E), and (22F) represents a bonding position to a benzene ring in the formula (2).

46. The organic electroluminescence device according to claim 1, wherein each R as a substituent of the second compound is independently a substituted or unsubstituted aryl group having 6 to 30 ring carbon atoms, a substituted or unsubstituted heterocyclic group having 5 to 30 ring atoms, or a substituted or unsubstituted alkyl group having 1 to 6 carbon atoms.

47. The organic electroluminescence device according to claim 1, wherein R₁ to R₈, R₂₁ to R₂₈, and R₂₁₁ to R₂₁₈ of the second compound are each independently a hydrogen atom, a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon atoms.

48. The organic electroluminescence device according to claim 1, wherein R₁ to R₈, R₂₁ to R₂₈, and R₂₁₁ to R₂₁₈ of the second compound are each independently a hydrogen atom or a substituted or unsubstituted aryl group having 6 to 30 ring carbon atoms.

49. The organic electroluminescence device according to claim 1, wherein R₁ to R₈, R₂₁ to R₂₈, and R₂₁₁ to R₂₁₈ are each a hydrogen atom.

50. The organic electroluminescence device according to claim 1, wherein
the emitting layer further comprises a third compound, and
a singlet energy S1(M3) of the third compound and the singlet energy S1(M2) of the second compound satisfy a relationship of a numerical formula (Numerical Formula 2) below,
S1(M3)>S1(M2) (Numerical Formula 2).

51. An electronic device comprising the organic electroluminescence device according to claim 1.
